(19) United States
(12) Patent Application Publication
(10) Pub. No.: US 2011/0212968 A1
(43) Pub. Date: Sep. 1, 2011

(54) PHENETHYLAMIDE DERIVATIVES AND THEIR HETEROCYCLIC ANALOGUES

(76) Inventors: Hamed Aissaoui, Allschwil (CH); Christoph Boss, Allschwil (CH); Christine Brotschi, Allschwil (CH); Ralf Koberstein, Allschwil (CH); Romain Siegrist, Allschwil (CH); Thierry Sifferlen, Allschwil (CH); Daniel Trachsel, Allschwil (CH); Jodi T. Williams, Allschwil (CH)

(21) Appl. No.: 13/124,233

(22) PCT Filed: Oct. 13, 2009
(86) PCT No.: PCT/IB2009/054493

§ 371 Date: Apr. 14, 2011

(30) Foreign Application Priority Data
Oct. 14, 2008 (IB) PCT/IB2008/054218

(51) Int. Cl. A61K 31/502; A61K 31/4965; A61K 31/437; A61K 31/44; A61K 31/4192; A61K 31/426; A61K 31/421; C07D 237/28; C07D 241/18; C07D 471/04; C07D 213/81; C07D 285/06; C07D 277/28; C07D 263/34; A61P 25/32; A61P 25/20; A61P 25/30; A61P 25/22; A61P 25/00; A61P 25/28

(52) U.S. Cl. 514/248; 514/255.06; 514/300; 514/354; 514/361; 514/365; 514/374; 544/235; 544/406; 546/121; 546/323; 548/127; 548/205; 548/236

(57) ABSTRACT

The invention relates to novel phenethylamide derivatives and their wherein A, B, R¹, R² and R³ are as described in the application, and to the use of such compounds, or of pharmaceutically acceptable salts of such compounds, as medicaments, especially as orexin receptor antagonists.

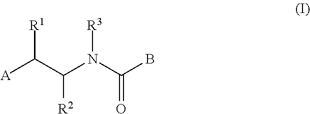

The present invention relates to novel phenethylamide derivatives and their heterocyclic analogues of formula (I) and their use as pharmaceuticals. The invention also concerns related aspects including processes for the preparation of the compounds, pharmaceutical compositions containing one or more compounds of formula (I), and especially their use as orexin receptor antagonists.

Orexins (orexin A or OX-A and orexin B or OX-B) are novel neuropeptides found in 1998 by two research groups, orexin A is a 33 amino acid peptide and orexin B is a 28 amino acid peptide (Sakurai T. et al., Cell, 1998, 92, 573-585). Orexins are produced in discrete neurons of the lateral hypothalamus and bind to G-protein-coupled receptors (OX₁ and OX₂ receptors). The orexin-1 receptor (OX₁) is selective for OX-A, and the orexin-2 receptor (OX₂) is capable to bind OX-A as well as OX-B. Orexins are found to stimulate food consumption in rats suggesting a physiological role for these peptides as mediators in the central feedback mechanism that regulates feeding behavior (Sakurai T. et al., Cell, 1998, 92, 573-585). On the other hand, it was also observed that orexins regulate states of sleep and wakefulness opening potentially novel therapeutic approaches to narcolepsy as well as insomnia and other sleep disorders (Chemelli R. M. et al., Cell, 1999, 98, 437-451).

Orexin receptors are found in the mammalian brain and may have numerous implications in pathologies as known from the literature.

The present invention provides phenethylamide derivatives and their heterocyclic analogues, which are non-peptide antagonists of human orexin receptors. These compounds are in particular of potential use in the treatment of e.g. eating disorders, drinking disorders, sleep disorders, or cognitive dysfunctions in psychiatric and neurologic disorders.

Up to now, several low molecular weight compounds are known having a potential to antagonise either specifically OX₁ or OX₂, or both receptors at the same time. Piperidine derivatives useful as orexin receptor antagonists are disclosed in WO01/096302. Benzamide derivatives are disclosed in WO03/037847. Pyrimidine derivatives are disclosed in WO05/075458.

The present invention describes for the first time phenethylamide derivatives and their heterocyclic analogues of formula (I) as orexin receptor antagonists.

i) A first aspect of the invention relates to compounds of formula (I)

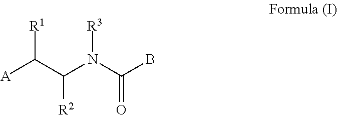

wherein
R¹ represents hydrogen, hydroxy or (C₃₋₆)cycloalkyl-amino;
R² represents hydrogen or (C₁₋₄)alkyl;
R³ represents (C₃₋₆)cycloalkyl or (C₃₋₆)cycloalkyl-(C₁₋₄alkyl; or a (C₁₋₄alkyl-group, which group is unsubstituted or monosubstituted with (C₁₋₄)alkoxy, hydroxy, NR⁴R⁵, C(O)NR⁴R⁵ or COOR⁶; or a (C₁₋₄-fluoroalkyl-group;
R⁴ represents hydrogen or (C₁₋₄)alkyl;
R⁵ represents hydrogen or (C₁₋₄)alkyl;
R⁶ represents (C₁₋₄)alkyl;
A represents aryl or heterocyclyl, wherein the aryl or heterocyclyl is independently unsubstituted or mono-, di-, or tri-substituted, wherein the substituents are independently selected from the group consisting of (C₁₋₄alkyl, (C₁₋₄alkoxy, (C₁₋₄alkylthio, hydroxy, amino, halogen, (C₁₋₄)fluoroalkyl, and (C₁₋₄)fluoroalkoxy; or A represents a benzo[1,3]dioxolyl- or a 2,3-dihydro-benzo[1,4]dioxinyl-group wherein said groups are unsubstituted, mono- or di-substituted with halogen; or A represents a 5H-[1,3]dioxolo[4,5-f]indole group;
B represents a group selected from

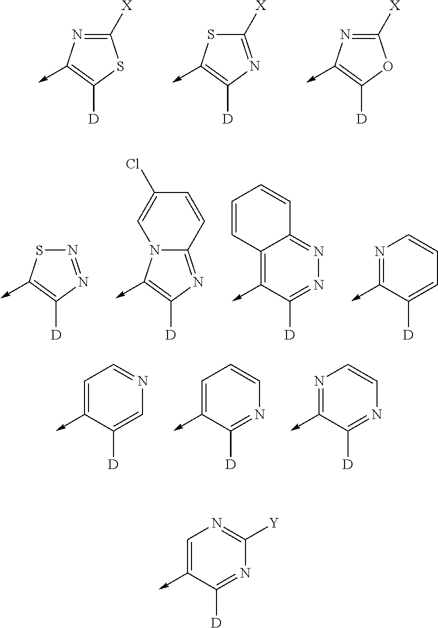

wherein
X represents hydrogen, (C₁₋₄alkyl, (C₃₋₆)cycloalkyl, (C₁₋₄alkoxy, R⁴R⁵N—CH₂—, NR⁴R⁵, or halogen;
Y represents hydrogen or (C₁₋₄)alkyl;
D represents aryl, wherein the aryl is unsubstituted or mono-, di-, or tri-substituted, wherein the substituents are independently selected from the group consisting of (C₁₋₄)alkyl, (C₁₋₄)alkoxy, hydroxy-(C₁₋₄)alkyl, (C₁₋₂)alkoxy-(C₁₋₄)alkoxy, halogen, (C₁₋₄)fluoroalkyl, NMe₂, (C₁₋₄)alkyl-C(O)NH— and cyano; or D represents heterocyclyl, wherein the heterocyclyl is unsubstituted or mono- or di-substituted, wherein the substituents are independently selected from the group consisting of (C₁₋₄)alkyl, (C₁₋₄)alkoxy, hydroxy-(C₁₋₄)alkyl, halogen, and (C₁₋₄)alkyl-thio;
with the proviso that A represents an optionally mono- or disubstituted indol-3-yl group, wherein the substituents are independently selected from the group consisting of (C₁₋₄)alkyl, (C₁₋₄)alkoxy and halogen, if B represents a group of formula

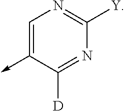

The compounds of formula (I) may contain one or more stereogenic or asymmetric centers, such as one or more asymmetric carbon atoms. The compounds of formula (I) may thus be present as mixtures of stereoisomers or preferably as pure stereoisomers. Mixtures of stereoisomers may be separated in a manner known to a person skilled in the art.

The following paragraphs provide definitions of the various chemical moieties for the compounds according to the invention and are intended to apply uniformly throughout the specification and claims, unless an otherwise expressly set out definition provides a broader or narrower definition.

In this patent application, an arrow shows the point of attachment of the radical drawn. For example, the radical drawn below

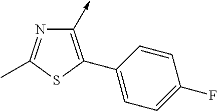

is the 5-(4-fluoro-phenyl)-2-methyl-thiazol-4-yl group.

The term “halogen” means fluorine, chlorine, bromine, and iodine, preferably fluorine and chlorine, and most preferably fluorine.

The term “(C₁₋₄)alkyl”, alone or in combination, means a straight-chain or branched-chain alkyl group with 1 to 4 carbon atoms. Examples of (C₁₋₄)alkyl groups are methyl, ethyl, n-propyl, isopropyl, n-butyl, isobutyl, sec.-butyl and tert.-butyl. Preferred are methyl, ethyl and n-propyl and especially methyl. The term “(C₃₋₆)cycloalkyl”, alone or in combination, means a cycloalkyl group with 3 to 6 carbon atoms. Examples of (C₃₋₆)cycloalkyl groups are cyclopropyl, cyclobutyl, cyclopentyl and cyclohexyl. Preferred are cyclopropyl and cyclohexyl. Most preferred is cyclopropyl.

The term “(C₃₋₆)cycloalkyl-amino” means an amino group (—NH₂) wherein one hydrogen atom has been replaced by a (C₃₋₆)cycloalkyl group as previously defined. Examples of (C₃₋₆)cycloalkyl-amino groups are cyclopropyl-amino, cyclobutyl-amino, cyclopentyl-amino and cyclohexyl-amino. Preferred is cyclopropyl-amino.

The term “(C₃₋₆)cycloalkyl-(C₁₋₄)alkyl” means a (C₁₋₄)alkyl group as previously defined wherein one hydrogen atom has been replaced by a (C₃₋₆)cycloalkyl group as previously defined. Selected examples are cyclopropyl-methyl, cyclopropyl-ethyl, cyclobutyl-methyl, cyclopentyl-methyl and cyclohexyl-methyl. Preferred is cyclopropyl-methyl.

The term “hydroxy-(C₁₋₄)alkyl” means a (C₁₋₄)alkyl group as previously defined wherein one hydrogen atom has been replaced by a hydroxy group. Preferred examples of hydroxy-(C₁₋₄)alkyl groups are hydroxy-methyl and hydroxy-ethyl, especially hydroxy-methyl.

The term “(C₁₋₄)alkoxy”, alone or in combination, means a group of the formula (C₁₋₄)alkyl-O— in which the term “(C₁₋₄)alkyl” has the previously given significance, such as methoxy, ethoxy, n-propoxy, isopropoxy, n-butoxy, isobutoxy, sec.-butoxy or tert.-butoxy. Preferred are methoxy and ethoxy, especially methoxy.

The term “(C₁₋₂)alkoxy-(C₁₋₄)alkoxy” means a (C₁₋₄)alkoxy group as previously defined wherein one hydrogen atom has been replaced by methoxy or ethoxy. Selected examples of (C₁₋₂)alkoxy-(C₁₋₄)alkoxy groups are 2-methoxy-ethoxy, 2-ethoxy-ethoxy and 3-methoxy-propoxy. Preferred is 2-methoxy-ethoxy.

The term “(C₁₋₄)alkylthio”, alone or in combination, means a group of the formula (C₁₋₄)alkyl-S— in which the term “(C₁₋₄)alkyl” has the previously given significance, such as methylthio, ethylthio, n-propylthio, isopropylthio, n-butylthio, isobutylthio, sec.-butylthio or tert.-butylthio. Preferred is methylthio.

The term “fluoroalkyl” means an alkyl group as defined before containing one to four (preferably one or two) carbon atoms in which one or more (and possibly all) hydrogen atoms have been replaced with fluorine. The term “(C_{x-y})fluoroalkyl” (x and y each being an integer) means a fluoroalkyl group as defined before containing x to y carbon atoms. For example a (C₁₋₄)fluoroalkyl group contains from one to four carbon atoms in which one to nine hydrogen atoms have been replaced with fluorine. Representative examples of fluoroalkyl groups include trifluoromethyl, 2,2-difluoroethyl and 2,2,2-trifluoroethyl. In case “R³” represents “(C₁₋₄)fluoroalkyl” the term preferably means 2,2-difluoroethyl and 2,2,2-trifluoroethyl (and most preferably 2,2,2-trifluoroethyl); in case “(C₁₋₄)fluoroalkyl” is substituent for “A” or “D” the term preferably means trifluoromethyl.

The term “fluoroalkoxy” means an alkoxy group as defined before containing one to four (preferably one or two) carbon atoms in which one or more (and possibly all) hydrogen atoms have been replaced with fluorine. The term “(C_{x-y})fluoroalkoxy” (x and y each being an integer) means a fluoroalkoxy group as defined before containing x to y carbon atoms. For example a (C₁₋₄)fluoroalkoxy group contains from one to four carbon atoms in which one to nine hydrogen atoms have been replaced with fluorine. Representative examples of fluoroalkoxy groups include trifluoromethoxy, difluoromethoxy and 2,2,2-trifluoroethoxy. Preferred are (C₁)fluoroalkoxy groups such as trifluoromethoxy and difluoromethoxy. Most preferred is difluoromethoxy.

The term “NR⁴R⁵” represents for example —NH₂, —NHMe or NMe₂.

The term “C(O)NR⁴R⁵” represents for example —C(O)NH₂ or —C(O)NMe₂ and preferably —C(O)NH₂.

The term “R⁴R⁵N—CH₂-” represents for example —CH₂NH₂ or —CH₂NMe₂.

The term “(C₁₋₄)alkyl-C(O)NH-” represents an amino group (—NH₂) wherein one hydrogen atom has been replaced by an alkanoyl group of formula (C₁₋₄)alkyl-C(O)— wherein the term “(C₁₋₄)alkyl” has the meaning as defined above. Examples of (C₁₋₄)alkyl-C(O)NH— groups are CH₃C(O)NH—, CH₃CH₂C(O)NH— and (CH₃)₂CHC(O)NH—. Preferred is CH₃CH₂C(O)NH—.

The term “COOR⁶” represents for example —COOMe.

The term “aryl”, alone or in combination, means a phenyl or a naphthyl group. Preferred is a phenyl group. In one embodiment, the aryl group may be unsubstituted or mono-, di-, or tri-substituted wherein the substituents are independently selected from the group consisting of (C₁₋₄)alkyl, (C₁₋₄)alkoxy, (C₁₋₄)alkylthio, hydroxy, amino, halogen, (C₁₋₄)fluoro alkyl, (C₁₋₄)fluoroalkoxy, hydroxy-(C₁₋₄)alkyl, (C₁₋₂)alkoxy-(C₁₋₄)alkoxy, NMe₂, (C₁₋₄)alkyl-C(O)NH—, and cyano. In another embodiment, the aryl group may be unsubstituted or mono-, di-, or tri-substituted wherein the substituents are independently selected from the group consisting of (C₁₋₄)alkyl, (C₁₋₄)alkoxy, (C₁₋₄)alkylthio, hydroxy, amino, halogen, (C₁₋₄)fluoro alkyl, (C₁₋₄)fluoroalkoxy, hydroxy-(C₁₋₄)alkyl, NMe₂, and cyano.

In case “A” represents “aryl” the term means the above-mentioned group which is unsubstituted or mono-, di-, or tri-substituted wherein the substituents are independently selected from the group consisting of (C₁₋₄)alkyl, (C₁₋₄)alkoxy, (C₁₋₄)alkylthio, hydroxy, amino, halogen, (C₁₋₄)fluoroalkyl, and (C₁₋₄)fluoroalkoxy. Preferred examples wherein “A” represents “aryl” are unsubstituted or mono-, di- or tri-substituted phenyl (preferred di- or tri-substituted phenyl), wherein the substituents are independently selected from the group consisting of (C₁₋₄)alkyl, (C₁₋₄)alkoxy, (C₁₋₄)alkylthio, hydroxy, halogen, and (C₁₋₄)fluoroalkoxy. Examples are phenyl, 2-naphthyl, 2-methylphenyl, 3-methylphenyl, 4-methylphenyl, 4-ethylphenyl, 2,4-dimethylphenyl, 3,4-dimethylphenyl, 2,5-dimethylphenyl, 3-methyl-4-methoxyphenyl, 2,5-dimethoxy-4-methylphenyl, 2-fluorophenyl, 4-fluorophenyl, 2-chlorophenyl, 4-chlorophenyl, 3-bromophenyl, 2,6-dichlorophenyl, 3-bromo-4-methoxyphenyl, 5-bromo-2-methoxyphenyl, 4-hydroxyphenyl, 4-hydroxy-3-methoxyphenyl, 2-methoxyphenyl, 3-methoxyphenyl, 4-methoxyphenyl, 2,5-dimethoxyphenyl, 3,4-dimethoxyphenyl, 3,5-dimethoxyphenyl, 3,4,5-trimethoxy-phenyl, 3-ethoxy-4-methoxyphenyl, 4-ethoxy-3-methoxyphenyl, 3,5-dimethoxy-4-isopropoxyphenyl, 3-difluoromethoxy-4-methoxyphenyl, 4-difluoromethoxy-3-methoxyphenyl, 4-methoxy-3-methylthiophenyl, 4-methylthiophenyl, 4-trifluoromethylphenyl, and 4-trifluoromethoxyphenyl. Preferred examples are 3-methyl-4-methoxyphenyl, 3-bromo-4-methoxyphenyl, 4-hydroxy-3-methoxyphenyl, 3,4-dimethoxyphenyl, 3,5-dimethoxyphenyl, 3,4,5-trimethoxyphenyl, 3-ethoxy-4-methoxyphenyl, 4-ethoxy-3-methoxyphenyl, 3,5-dimethoxy-4-isopropoxyphenyl, 3-difluoromethoxy-4-methoxyphenyl, 4-difluoromethoxy-3-methoxyphenyl, and 4-methoxy-3-methylthiophenyl.

In one embodiment, in case “D” represents “aryl” the term means the above-mentioned group which is unsubstituted or mono-, di-, or tri-substituted (preferred unsubstituted or mono- or di-substituted), wherein the substituents are independently selected from the group consisting of (C₁₋₄)alkyl, (C₁₋₄)alkoxy, hydroxy-(C₁₋₄)alkyl, (C₁₋₂)alkoxy-(C₁₋₄)alkoxy, halogen, (C₁₋₄)fluoro alkyl, NMe₂, (C₁₋₄)alkyl-C(O)NH— and cyano. In another embodiment, in case “D” represents “aryl” the term means the above-mentioned group which is unsubstituted or mono-, di-, or tri-substituted (preferred mono- or di-substituted), wherein the substituents are independently selected from the group consisting of (C₁₋₄)alkyl, (C₁₋₄)alkoxy, hydroxy-(C₁₋₄alkyl, halogen, (C₁₋₄)fluoroalkyl, NMe₂, and cyano. Preferably the substituents are selected from (C₁₋₄)alkyl, (C₁₋₄)alkoxy, and halogen. Preferred examples wherein “D” represents “aryl” are unsubstituted or mono-, di-, or tri-substituted phenyl (preferred mono- or di-substituted), wherein the substituents are independently selected from the group consisting of (C₁₋₄)alkyl, (C₁₋₄)alkoxy, and halogen. Examples are phenyl, 3-methylphenyl, 4-methylphenyl, 2,3-dimethylphenyl, 2,4-dimethylphenyl, 3,5-dimethylphenyl, 3,4-dimethylphenyl, 4-ethylphenyl, 3-fluoro-2-methylphenyl, 3-fluoro-4-methylphenyl, 4-fluoro-3-methylphenyl, 2,3-difluoro-4-methylphenyl, 3-chloro-4-methylphenyl, 3-methyl-4-methoxyphenyl, 2-fluorophenyl, 3-fluorophenyl, 4-fluorophenyl, 3-chlorophenyl, 4-chlorophenyl, 3,4-difluorophenyl, 3,5-difluorophenyl, 3,4-dichlorophenyl, 3-chloro-4-fluorophenyl, 4-chloro-3-fluorophenyl, 3-fluoro-4-methoxyphenyl, 4-fluoro-3-methoxyphenyl, 3-chloro-4-methoxyphenyl, 4-fluoro-3-hydroxymethylphenyl, 3-fluoro-4-cyanophenyl, 4-fluoro-3-cyanophenyl, 4-chloro-3-cyanophenyl, 3-fluoro-5-trifluoromethylphenyl, 3-methoxyphenyl, 4-methoxyphenyl, 3-dimethylaminophenyl, 3-cyanophenyl, 4-cyanophenyl, 3-trifluoromethylphenyl, and 4-trifluoromethylphenyl. Further examples are 3-fluoro-5-methylphenyl, 2,3-dichlorophenyl, 3,5-dichlorophenyl, 3-bromophenyl, 4-bromophenyl, 2-chloro-6-fluorophenyl, 3-bromo-4-fluorophenyl, 4-bromo-3-chlorophenyl, 4-ethoxyphenyl, 3-(2-methoxy-ethoxy)-phenyl, 2-fluoro-5-methoxyphenyl, and 4-propionylamino-phenyl. In one embodiment, preferred examples are phenyl, 3-methylphenyl, 4-methylphenyl, 2,3-dimethylphenyl, 3,4-dimethylphenyl, 4-ethylphenyl, 3-fluoro-2-methylphenyl, 3-fluoro-4-methylphenyl, 2-fluorophenyl, 3-fluorophenyl, 4-fluorophenyl, 3-chlorophenyl, 4-chlorophenyl, 3,4-difluorophenyl, 3,4-dichlorophenyl, 3-fluoro-4-methoxyphenyl, 4-fluoro-3-hydroxymethylphenyl, 3-methoxyphenyl, 4-methoxyphenyl, and 3-trifluoromethylphenyl. In another embodiment, preferred examples are phenyl, 3-methylphenyl, 4-methylphenyl, 2,3-dimethylphenyl, 3,4-dimethylphenyl, 4-ethylphenyl, 3-fluoro-2-methylphenyl, 3-fluoro-4-methylphenyl, 2-fluorophenyl, 3-fluorophenyl, 4-fluorophenyl, 3-chlorophenyl, 4-chlorophenyl, 3,4-difluorophenyl, 3,4-dichlorophenyl, 3-fluoro-4-methoxyphenyl, 4-fluoro-3-hydroxymethylphenyl, 3-methoxyphenyl, 4-methoxyphenyl, and 3-trifluoromethylphenyl, 3-fluoro-5-methylphenyl, 3-bromophenyl, 3-bromo-4-fluorophenyl, and 4-bromo-3-chlorophenyl. In still another embodiment, preferred examples are 3-fluoro-5-methylphenyl, 3-bromophenyl, 3-bromo-4-fluorophenyl, and 4-bromo-3-chlorophenyl.

The term “heterocyclyl”, alone or in combination, means a 5- to 10-membered monocyclic or bicyclic aromatic ring containing 1, 2 or 3 heteroatoms independently selected from oxygen, nitrogen and sulfur. Examples of such heterocyclyl groups are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzisoxazolyl, benzothiazolyl, benzotriazolyl, benzoxadiazolyl, benzothiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidyl, imidazo[1,2-a]pyridyl, pyrrolo[2,1-b]thiazolyl, imidazo[2,1-b]thiazolyl, benzo[2,1,3]thiadiazolyl, and benzo[2,1,3]oxadiazolyl. The above-mentioned heterocyclyl groups are unsubstituted or mono-, di-, or tri-substituted wherein the substituents are independently selected from the group consisting of (C₁₋₄)alkyl, (C₁₋₄)alkoxy, (C₁₋₄)alkylthio, hydroxy, amino, halogen, (C₁₋₄)fluoro alkyl, (C₁₋₄)fluoroalkoxy, and hydroxy-(C₁₋₄)alkyl (and preferably (C₁₋₄)alkyl, (C₁₋₄)alkoxy, and halogen).

In case “A” represents “heterocyclyl” the term preferably means the above-mentioned groups which are unsubstituted or mono- or di-substituted (preferred mono-substituted) wherein the substituents are independently selected from the group consisting of (C₁₋₄)alkyl, (C₁₋₄)alkoxy, (C₁₋₄)alkylthio, hydroxy, amino, halogen, (C₁₋₄)fluoroalkyl, and (C₁₋₄)fluoroalkoxy. In a further preferred embodiment, in case “A” represents “heterocyclyl” the term means the above-mentioned groups which are unsubstituted or mono- or di-substituted (preferred mono-substituted), wherein the substituents are independently selected from the group consisting of (C₁₋₄)alkyl, (C₁₋₄)alkoxy, amino, and halogen. In a further preferred embodiment, in case “A” represents “heterocyclyl” the term means an unsubstituted or mono-, or di-substituted group selected from imidazolyl (especially imidazol-1-yl), thiazolyl (especially thiazol-4-yl), pyridyl (especially pyridin-3-yl), indolyl (especially indol-3-yl) and benzimidazolyl (especially benzimidazol-2-yl), wherein the substituents are independently selected from the group consisting of (C₁₋₄)alkyl, (C₁₋₄)alkoxy, (C₁₋₄)alkylthio, hydroxy, amino, halogen, (C₁₋₄)fluoroalkyl, and (C₁₋₄)fluoroalkoxy. In a most preferred embodiment, in case “A” represents “heterocyclyl” the term means an unsubstituted or mono-, or di-substituted group selected from indol-3-yl and benzimidazol-2-yl, wherein the substituents are independently selected from the group consisting of (C₁₋₄)alkyl, (C₁₋₄)alkoxy, and halogen. Examples are di-substituted imidazol-1-yl such as 2-ethyl-4-iodo-imidazol-1-yl; mono-substituted thiazol-4-yl such as 2-amino-thiazol-4-yl; mono-substituted pyridin-3-yl such as 6-methoxy-pyridin-3-yl; unsubstituted benzimidazol-2-yl; mono-substituted benzimidazol-2-yl such as 6-methyl-benzimidazol-2-yl, 6-chloro-benzimidazol-2-yl and 6-methoxy-benzimidazol-2-yl; di-substituted benzimidazol-2-yl such as 5,6-dimethyl-benzimidazol-2-yl; unsubstituted indol-1-yl; unsubstituted indol-3-yl; mono-substituted indol-3-yl such as 1-methyl-indol-3-yl, 5-methyl-indol-3-yl, 6-methyl-indol-3-yl, 7-methyl-indol-3-yl, 5-methoxy-indol-3-yl, 6-methoxy-indol-3-yl, 7-methoxy-indol-3-yl, 4-fluoro-indol-3-yl, 5-fluoro-indol-3-yl, 6-fluoro-indol-3-yl, 7-fluoro-indol-3-yl, 6-chloro-indol-3-yl, and 5-bromo-indol-3-yl; and di-substituted indol-3-yl such as 4-methyl-5-methoxy-indol-3-yl, 5,6-difluoro-indol-3-yl, and 5-chloro-6-fluoro-indol-3-yl. Preferred examples are 6-methoxy-benzimidazol-2-yl, 5,6-dimethyl-benzimidazol-2-yl, indol-3-yl, 1-methyl-indol-3-yl, 5-methyl-indol-3-yl, 6-methyl-indol-3-yl, 7-methyl-indol-3-yl, 5-methoxy-indol-3-yl, 6-methoxy-indol-3-yl, 7-methoxy-indol-3-yl, 4-fluoro-indol-3-yl, 5-fluoro-indol-3-yl, 6-fluoro-indol-3-yl, 7-fluoro-indol-3-yl, 6-chloro-indol-3-yl, 5-bromo-indol-3-yl, 5,6-difluoro-indol-3-yl, and 5-chloro-6-fluoro-indol-3-yl. Most preferred examples are 1-methyl-indol-3-yl, 5-methyl-indol-3-yl, 7-methyl-indol-3-yl, 5-methoxy-indol-3-yl, 6-methoxy-indol-3-yl, 5-fluoro-indol-3-yl, 6-fluoro-indol-3-yl, and 7-fluoro-indol-3-yl.

In case “D” represents “heterocyclyl” the term means the above-mentioned groups which are unsubstituted or mono- or di-substituted (preferred unsubstituted or mono-substituted) wherein the substituents are independently selected from the group consisting of (C₁₋₄)alkyl, (C₁₋₄)alkoxy, hydroxy-(C₁₋₄)alkyl, halogen, and (C₁₋₄)alkyl-thio. In a further preferred embodiment, in case “D” represents “heterocyclyl” the term means an unsubstituted or mono-, or di-substituted group selected from pyridyl (especially pyridin-3-yl and pyridin-4-yl), pyrimidyl (especially pyrimidin-5-yl), indolyl (especially indol-2-yl, indol-5-yl and indol-6-yl) and quinolinyl (especially quinolin-3-yl), wherein the substituents are independently selected from the group consisting of (C₁₋₄alkyl, (C₁₋₄)alkoxy, hydroxy-(C₁₋₄)alkyl, halogen, and (C₁₋₄)alkyl-thio. In a most preferred embodiment, in case “D” represents “heterocyclyl” the term means an unsubstituted or mono-, or di-substituted group selected from pyridin-3-yl, pyridin-4-yl, pyrimidin-5-yl, indol-2-yl, indol-5-yl, indol-6-yl and quinolin-3-yl, wherein the substituents are independently selected from the group consisting of (C₁₋₄alkyl, (C₁₋₄alkoxy, (C₁₋₄alkylthio, halogen, and hydroxy-(C₁₋₄alkyl. Examples are 5-methyl-pyridin-3-yl, 6-methyl-pyridin-3-yl, 5-fluoro-pyridin-3-yl, 6-fluoro-pyridin-3-yl, 5-methoxy-pyridin-3-yl, 6-methoxy-pyridin-3-yl, 5-methylthio-pyridin-3-yl, 6-hydroxymethyl-pyridin-3-yl, 2-fluoro-5-chloro-pyridin-3-yl, 3-chloro-2-methoxy-pyridin-4-yl, pyrimidin-5-yl, 2-methoxy-pyrimidin-5-yl, 1-methyl-indol-2-yl, indol-5-yl, indol-6-yl and quinolin-3-yl. Preferred examples are 6-methoxy-pyridin-3-yl, and quinolin-3-yl.

In the following, further embodiments of the invention are described:

ii) A further embodiment of the invention relates to compounds according to embodiment i), wherein

R¹ represents hydrogen, hydroxy or (C₃₋₆)cycloalkyl-amino;
R² represents hydrogen or (C₁₋₄alkyl;
R³ represents (C₃₋₆)cycloalkyl- or (C₃₋₆)cycloalkyl-(C₁₋₄)alkyl; or a (C₁₋₄)alkyl-group, which group is unsubstituted or monosubstituted with (C₁₋₄alkoxy, hydroxy, NR⁴R⁵, C(O)NR⁴R⁵ or COOR⁶; or a (C₁₋₄-fluoroalkyl-group;
R⁴ represents hydrogen or (C₁₋₄alkyl;
R⁵ represents hydrogen or (C₁₋₄alkyl;
R⁶ represents (C₁₋₄alkyl;
A represents aryl or heterocyclyl, wherein the aryl or heterocyclyl is independently unsubstituted or mono-, di-, or tri-substituted, wherein the substituents are independently selected from the group consisting of (C₁₋₄)alkyl, (C₁₋₄alkoxy, (C₁₋₄)alkylthio, hydroxy, amino, halogen, (C₁₋₄-fluoroalkyl, and (C₁₋₄-fluoroalkoxy; or A represents a benzo[1,3]dioxolyl- or a 2,3-dihydro-benzo[1,4]dioxinyl-group wherein said groups are unsubstituted, mono- or di-substituted with halogen; or A represents a 5H-[1,3]dioxolo[4,5-f]indole group;
B represents a group selected from

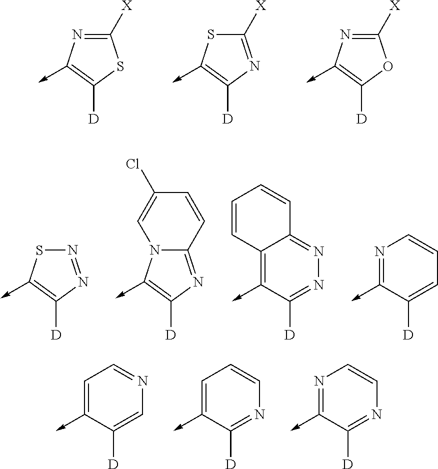

wherein
X represents hydrogen, (C₁₋₄alkyl, (C₃₋₆)cycloalkyl, (C₁₋₄alkoxy, R⁴R⁵N—CH₂—, NR⁴R⁵, or halogen;
D represents aryl, wherein the aryl is unsubstituted or mono-, di-, or tri-substituted, wherein the substituents are independently selected from the group consisting of (C₁₋₄alkyl, (C₁₋₄alkoxy, hydroxy-(C₁₋₄alkyl, halogen, (C₁₋₄-fluoroalkyl, NMe₂, and cyano; or D represents heterocyclyl, wherein the heterocyclyl is unsubstituted or mono- or di-substituted, wherein the substituents are independently selected from the group consisting of (C₁₋₄alkyl, (C₁₋₄alkoxy, hydroxy-(C₁₋₄alkyl, halogen, and (C₁₋₄alkyl-thio.

iii) A further embodiment of the invention relates to compounds according to embodiment i), wherein at least one, preferably all of the following characteristics are present:

R¹ represents hydrogen;
R² represents hydrogen or (C₁₋₄)alkyl;
R³ represents (C₃₋₆)cycloalkyl-(C₁₋₄alkyl; or a (C₁₋₄alkyl-group, which group is unsubstituted or monosubstituted with hydroxy, NR⁴R⁵, C(O)NR⁴R⁵ or COOR⁶; or a (C₁₋₄)fluoroalkyl group;
R⁴ represents hydrogen or (C₁₋₄)alkyl;
R⁵ represents hydrogen or (C₁₋₄)alkyl;
R⁶ represents (C₁₋₄alkyl;
A represents heterocyclyl, wherein the heterocyclyl is unsubstituted or mono-, or di-substituted, wherein the substituents are independently selected from the group consisting of (C₁₋₄alkyl, (C₁₋₄alkoxy, amino, and halogen; or A represents a 5H-[1,3]dioxolo[4,5-f]indole group;
B represents a group selected from

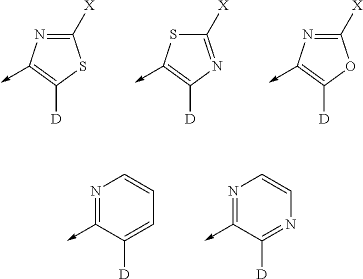

wherein
X represents hydrogen, (C₁₋₄alkyl, (C₃₋₆)cycloalkyl, (C₁₋₄alkoxy, R⁴R⁵N—CH₂—, or NR⁴R⁵;
D represents aryl, wherein the aryl is unsubstituted or mono-, di-, or tri-substituted,

wherein the substituents are independently selected from the group consisting of (C₁₋₄)alkyl, (C₁₋₄alkoxy, hydroxy-(C₁₋₄)alkyl, (C₁₋₂)alkoxy-(C₁₋₄)alkoxy, halogen, (C₁₋₄-fluoroalkyl, NMe₂, (C₁₋₄)alkyl-C(O)NH— and cyano; or D represents heterocyclyl, wherein the heterocyclyl is unsubstituted or mono- or di-substituted, wherein the substituents are independently selected from the group consisting of (C₁₋₄)alkyl, (C₁₋₄alkoxy, hydroxy-(C₁₋₄)alkyl, halogen, and (C₁₋₄alkyl-thio.

iv) A further embodiment of the invention relates to compounds according to any one of embodiments i) or ii), wherein at least one, preferably all of the following characteristics are present:

R¹ represents hydrogen;
R² represents hydrogen or (C₁₋₄)alkyl;
R³ represents (C₃₋₆)cycloalkyl-(C₁₋₄)alkyl; or a (C₁₋₄)alkyl-group, which group is unsubstituted or monosubstituted with hydroxy, NR⁴R⁵, C(O)NR⁴R⁵ or COOR⁶; or a (C₁₋₄-fluoroalkyl group;
R⁴ represents hydrogen or (C₁₋₄)alkyl;
R⁵ represents hydrogen or (C₁₋₄)alkyl;
R⁶ represents (C₁₋₄)alkyl;
A represents heterocyclyl, wherein the heterocyclyl is unsubstituted or mono-, or di-substituted, wherein the substituents are independently selected from the group consisting of (C₁₋₄)alkyl, (C₁₋₄)alkoxy, amino, and halogen; or A represents a 5H-[1,3]dioxolo[4,5-f]indole group;
B represents a group selected from

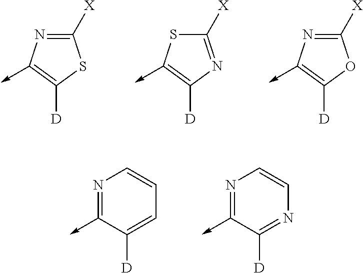

wherein
X represents hydrogen, (C₁₋₄)alkyl, (C₃₋₆)cycloalkyl, (C₁₋₄)alkoxy, R⁴R⁵N—CH₂—, or NR⁴R⁵;
D represents aryl, wherein the aryl is unsubstituted or mono-, di-, or tri-substituted,

wherein the substituents are independently selected from the group consisting of (C₁₋₄)alkyl, (C₁₋₄)alkoxy, hydroxy-(C₁₋₄)alkyl, halogen, NMe₂, and cyano; or D represents heterocyclyl, wherein the heterocyclyl is unsubstituted or mono- or di-substituted, wherein the substituents are independently selected from the group consisting of (C₁₋₄)alkyl, (C₁₋₄)alkoxy, hydroxy-(C₁₋₄)alkyl, halogen, and (C₁₋₄)alkyl-thio.

v) A further embodiment of the invention relates to compounds according to any one of embodiments i) or ii), wherein at least one, preferably all of the following characteristics are present:

R¹ represents hydrogen, hydroxy or (C₃₋₆)cycloalkyl-amino;
R² represents hydrogen or (C₁₋₄)alkyl;
R³ represents (C₃₋₆)cycloalkyl or (C₃₋₆)cycloalkyl-(C₁₋₄)alkyl; or a (C₁₋₄)alkyl-group, which group is unsubstituted or mono-substituted with (C₁₋₄)alkoxy, hydroxy, NR⁴R⁵ or C(O)NR⁴R⁵; or a (C₁₋₄)fluoroalkyl group;
R⁴ represents hydrogen or (C₁₋₄)alkyl;
R⁵ represents hydrogen or (C₁₋₄)alkyl;
A represents aryl (especially phenyl), wherein the aryl is unsubstituted or mono-, di-, or tri-substituted, wherein the substituents are independently selected from the group consisting of (C₁₋₄)alkyl, (C₁₋₄)alkoxy, (C₁₋₄)alkylthio, hydroxy, halogen, (C₁₋₄)fluoroalkyl, and (C₁₋₄)fluoroalkoxy;
B represents a group selected from

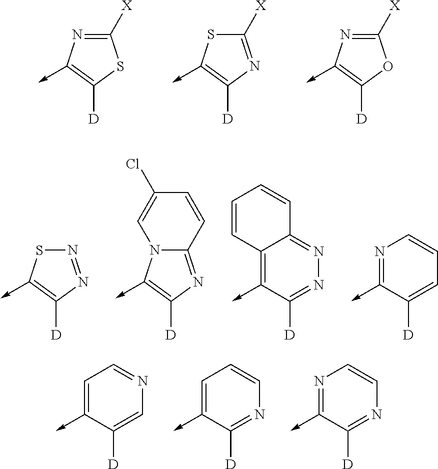

wherein
X represents hydrogen, (C₁₋₄)alkyl, (C₃₋₆)cycloalkyl, (C₁₋₄alkoxy, NR⁴R⁵, or halogen; D represents aryl, wherein the aryl is unsubstituted or mono-, di-, or tri-substituted, wherein the substituents are independently selected from the group consisting of (C₁₋₄)alkyl, (C₁₋₄)alkoxy, halogen, (C₁₋₄-fluoroalkyl, and cyano.

vi) A further embodiment of the invention relates to compounds according to any one of embodiments i) or ii), wherein

R¹ represents hydrogen, hydroxy or cyclopropyl-amino;
R² represents hydrogen or (C₁₋₄)alkyl (especially hydrogen, methyl or ethyl);
R³ represents (C₃₋₆)cycloalkyl (especially cyclopropyl) or (C₃₋₆)cycloalkyl-(C₁₋₄)alkyl (especially cyclopropyl-methyl); or an unsubstituted (C₁₋₄)alkyl-group (especially methyl, ethyl, n-propyl, isopropyl or isobutyl); or a (C₁₋₄)alkyl-group (especially methyl or ethyl), which group is monosubstituted with (C₁₋₄alkoxy (especially methoxy), hydroxy, NR⁴R⁵ (especially dimethylamino), C(O)NR⁴R⁵ or COOR⁶; or a (C₁₋₄)fluoroalkyl-group (especially 2,2-difluoroethyl or 2,2,2-trifluoroethyl);
R⁴ represents hydrogen or (C₁₋₄)alkyl (especially hydrogen or methyl);
R⁵ represents hydrogen or (C₁₋₄)alkyl (especially hydrogen or methyl);
R⁶ represents (C₁₋₄)alkyl (especially methyl);
A represents aryl (especially phenyl), wherein the aryl is unsubstituted or mono-, di-, or tri-substituted (especially di-substituted), wherein the substituents are independently selected from the group consisting of (C₁₋₄)alkyl (especially methyl and ethyl), (C₁₋₄alkoxy (especially methoxy, ethoxy and isopropoxy), (C₁₋₄)alkylthio (especially methylthio), hydroxy, halogen (especially fluoro, chloro and bromo), (C₁₋₄-fluoroalkyl (especially trifluoromethyl), and (C₁₋₄-fluoroalkoxy (especially difluoromethoxy and trifluoromethoxy); or A represents heterocyclyl (especially indol-3-yl or benzimidazol-2-yl), wherein the heterocyclyl is unsubstituted or mono-, di-, or tri-substituted (especially unsubstituted or mono-, or di-substituted), wherein the substituents are independently selected from the group consisting of (C₁₋₄)alkyl (especially methyl and ethyl), (C₁₋₄)alkoxy (especially methoxy), amino, and halogen (especially fluoro and chloro); or A represents a benzo[1,3]dioxolyl- or a 2,3-dihydro-benzo[1,4]dioxinyl-group wherein said groups are unsubstituted or di-substituted with halogen (especially unsubstituted or di-substituted at a saturated carbon atom with fluorine); or A represents a 5H-[1,3]dioxolo[4,5-f]indole group;
B represents

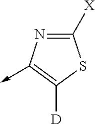

wherein
X represents hydrogen, (C₁₋₄)alkyl (especially methyl), (C₃₋₆)cycloalkyl (especially cyclopropyl), (C₁₋₄)alkoxy (especially methoxy), R⁴R⁵N—CH₂—, NR⁴R⁵, or halogen (especially bromine);
D represents phenyl, wherein the phenyl is unsubstituted or mono-, di-, or tri-substituted (especially unsubstituted or mono-, or di-substituted), wherein the substituents are independently selected from the group consisting of (C₁₋₄)alkyl (especially methyl and ethyl), (C₁₋₄)alkoxy (especially methoxy), hydroxy-(C₁₋₄)alkyl (especially hydroxy-methyl), (C₁₋₂)alkoxy-(C₁₋₄)alkoxy (especially 2-methoxy-ethoxy), halogen (especially fluoro, chloro and bromo), (C₁₋₄)fluoroalkyl (especially trifluoromethyl), (C₁₋₄)alkyl-C(O)NH— (especially C₂H₅—C(O)NH—) and cyano; or D represents heterocyclyl (especially pyridyl, indolyl, or quinolinyl), wherein the heterocyclyl is unsubstituted or mono- or di-substituted, wherein the substituents are independently selected from the group consisting of (C₁₋₄)alkyl (especially methyl), (C₁₋₄alkoxy (especially methoxy), hydroxy-(C₁₋₄)alkyl (especially hydroxy-methyl), halogen (especially fluoro and chloro), and (C₁₋₄)alkyl-thio (especially methylthio).
vii) A further embodiment of the invention relates to compounds according to any one of embodiments i) or ii), wherein
R¹ represents hydrogen;
R² represents hydrogen;
R³ represents (C₃₋₆)cycloalkyl-(C₁₋₄)alkyl (especially cyclopropyl-methyl); or an unsubstituted (C₁₋₄)alkyl-group (especially methyl, ethyl, n-propyl, or isopropyl); or a (C₁₋₄)alkyl-group (especially methyl or ethyl), which group is monosubstituted with hydroxy, C(O)NR⁴R⁵ or COOR⁶; or a (C₁₋₄)fluoroalkyl-group (especially 2,2-difluoroethyl or 2,2,2-trifluoroethyl);
R⁴ represents hydrogen or (C₁₋₄)alkyl (especially hydrogen or methyl);
R⁵ represents hydrogen or (C₁₋₄)alkyl (especially hydrogen or methyl);
R⁶ represents (C₁₋₄)alkyl (especially methyl);
A represents aryl (especially phenyl), wherein the aryl is unsubstituted or mono-, di-, or tri-substituted (especially di-substituted) with (C₁₋₄)alkoxy (especially methoxy); or
A represents heterocyclyl (especially indol-3-yl or benzimidazol-2-yl), wherein the heterocyclyl is unsubstituted or mono-, di-, or tri-substituted (especially mono-, or di-substituted), wherein the substituents are independently selected from the group consisting of (C₁₋₄)alkyl (especially methyl), (C₁₋₄)alkoxy (especially methoxy) and halogen (especially fluoro and chloro);
B represents

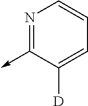

wherein
D represents phenyl, wherein the phenyl is unsubstituted or mono- or di-substituted (especially mono- or di-substituted), wherein the substituents are independently selected from the group consisting of (C₁₋₄)alkyl (especially methyl) and (C₁₋₄)alkoxy (especially methoxy); or D represents heterocyclyl (especially pyridyl, or quinolinyl), wherein the heterocyclyl is unsubstituted or mono- or di-substituted, wherein the substituents are independently selected from the group consisting of (C₁₋₄)alkyl (especially methyl), (C₁₋₄)alkoxy (especially methoxy), and halogen (especially fluoro and chloro).

viii) A further embodiment of the invention relates to compounds according to any one of embodiments i) or ii), wherein

R¹ represents hydrogen or hydroxy;
R² represents hydrogen;
R³ represents (C₃₋₆)cycloalkyl-(C₁₋₄)alkyl (especially cyclopropyl-methyl); or an unsubstituted (C₁₋₄)alkyl-group (especially methyl, ethyl, n-propyl or isopropyl); or a (C₁₋₄)alkyl-group (especially methyl or ethyl), which group is monosubstituted with hydroxy, amino, C(O)NH₂ or COOR⁶; or a (C₁₋₄)fluoroalkyl-group (especially 2,2-difluoroethyl or 2,2,2-trifluoroethyl);
R⁶ represents (C₁₋₄)alkyl (especially methyl);
A represents aryl (especially phenyl), wherein the aryl is unsubstituted or mono-, di-, or tri-substituted (especially di-substituted) with (C₁₋₄)alkoxy (especially methoxy); or
A represents heterocyclyl (especially indol-3-yl or benzimidazol-2-yl), wherein the heterocyclyl is unsubstituted or mono-, di-, or tri-substituted (especially unsubstituted or mono-, or di-substituted), wherein the substituents are independently selected from the group consisting of (C₁₋₄)alkyl (especially methyl), (C₁₋₄)alkoxy (especially methoxy) and halogen (especially fluoro and chloro);
B represents

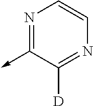

wherein
D represents phenyl, wherein the phenyl is unsubstituted or mono-, di-, or tri-substituted (especially unsubstituted or mono-, or di-substituted), wherein the substituents are independently selected from the group consisting of (C₁₋₄)alkyl (especially methyl), (C₁₋₄)alkoxy (especially methoxy and ethoxy), halogen (especially fluoro) and (C₁₋₄)fluoroalkyl (especially trifluoromethyl); or D represents heterocyclyl (especially pyridyl or pyrimidyl), wherein the heterocyclyl is unsubstituted or mono- or di-substituted (especially unsubstituted or mono-substituted) with (C₁₋₄)alkoxy (especially methoxy).

ix) A further embodiment of the invention relates to compounds according to embodiment i), wherein

R¹ represents hydrogen;
R² represents hydrogen;
R³ represents (C₃₋₆)cycloalkyl-(C₁₋₄)alkyl (especially cyclopropyl-methyl); or an unsubstituted (C₁₋₄)alkyl-group (especially ethyl); or a (C₁₋₄)alkyl-group (especially methyl), which group is monosubstituted with COOR⁶; or a (C₁₋₄)fluoroalkyl-group (especially 2,2,2-trifluoroethyl);
R⁶ represents (C₁₋₄)alkyl (especially methyl);
A represents an indol-3-yl group which is unsubstituted or mono- or disubstituted, wherein the substituents are independently selected from the group consisting of (C₁₋₄)alkyl (especially methyl), (C₁₋₄)alkoxy (especially methoxy) and halogen (especially fluoro and chloro);
B represents

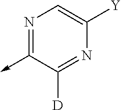

wherein
Y represents hydrogen or (C₁₋₄)alkyl (especially hydrogen or methyl);
D represents phenyl, wherein the phenyl is unsubstituted or mono-, di-, or tri-substituted (especially unsubstituted or mono-, or di-substituted), wherein the substituents are independently selected from the group consisting of (C₁₋₄)alkyl (especially methyl), (C₁₋₄)alkoxy (especially methoxy) and halogen (especially fluoro, chloro and bromo).

x) A further embodiment of the invention relates to compounds according to any one of embodiments i), ii), v), vi or viii), wherein

R¹ represents hydrogen or hydroxy.

xi) A further embodiment of the invention relates to compounds according to any one of embodiments i) to x), wherein

R¹ represents hydrogen.

xii) A further embodiment of the invention relates to compounds according to any one of embodiments i), ii), v), vi), viii) or x), wherein

R¹ represents hydroxy.

xiii) A further embodiment of the invention relates to compounds according to any one of embodiments i) to xii), wherein

R² represents hydrogen.

xiv) A further embodiment of the invention relates to compounds according to any one of embodiments i) to vi) or x) to xii), wherein

R² represents (C₁₋₄)alkyl.

xv) A further embodiment of the invention relates to compounds according to any one of embodiments i), ii), vi) or x) to xiv), wherein

R³ represents (C₃₋₆)cycloalkyl or (C₃₋₆)cycloalkyl-(C₁₋₄)alkyl; or a (C₁₋₄)alkyl-group, which group is monosubstituted with (C₁₋₄)alkoxy, hydroxy, NR⁴R⁵, C(O)NR⁴R⁵ or COOR⁶; or a (C₁₋₄)fluoroalkyl group.

xvi) A further embodiment of the invention relates to compounds according to any one of embodiments i) to vi), viii) or x) to xv), wherein

R³ represents (C₃₋₆)cycloalkyl-(C₁₋₄)alkyl; or a (C₁₋₄)alkyl-group, which group is monosubstituted with hydroxy, NR⁴R⁵ or C(O)NR⁴R⁵; or a (C₁₋₄)fluoroalkyl group.

xvii) A further embodiment of the invention relates to compounds according to any one of embodiments i), ii), v), vi) or x) to xv), wherein

R³ represents (C₃₋₆)cycloalkyl or (C₃₋₆)cycloalkyl-(C₁₋₄)alkyl.

xviii) A further embodiment of the invention relates to compounds according to any one of embodiments i), ii), v), vi), x) to xv) or xvii), wherein

R³ represents (C₃₋₆)cycloalkyl (especially cyclopropyl).

xix) A further embodiment of the invention relates to compounds according to any one of embodiments i) to xvii), wherein

R³ represents (C₃₋₆)cycloalkyl-(C₁₋₄)alkyl (especially cyclopropylmethyl).

xx) A further embodiment of the invention relates to compounds according to any one of embodiments i), ii), vi) or x) to xiv), wherein

R³ represents a (C₁₋₄)alkyl-group, which group is unsubstituted or monosubstituted with (C₁₋₄)alkoxy, hydroxy, NR⁴R⁵, C(O)NR⁴R⁵ or COOR⁶.

xxi) A further embodiment of the invention relates to compounds according to any one of embodiments i) to xiv) or xx), wherein

R³ represents a (C₁₋₄)alkyl-group.

xxii) A further embodiment of the invention relates to compounds according to any one of embodiments i) to vi), viii), x) to xvi) or xx), wherein

R³ represents a (C₁₋₄)alkyl-group, which group is monosubstituted with hydroxy, NR⁴R⁵ or C(O)NR⁴R⁵.

xxiii) A further embodiment of the invention relates to compounds according to any one of embodiments i) to xvi), wherein

R³ represents a (C₁₋₄)fluoroalkyl group (especially a 2,2-difluoroethyl- or a 2,2,2-trifluoroethyl-group).

xxiv) A further embodiment of the invention relates to compounds according to any one of embodiments i) to xvi) or xxiii), wherein

R³ represents 2,2,2-trifluoroethyl.

xxv) A further embodiment of the invention relates to compounds according to any one of embodiments i), ii) or x) to xxiv), wherein

A represents aryl or heterocyclyl, wherein the aryl or heterocyclyl is independently unsubstituted or mono-, di-, or tri-substituted, wherein the substituents are independently selected from the group consisting of (C₁₋₄)alkyl (especially methyl), (C₁₋₄)alkoxy (especially methoxy), (C₁₋₄)alkylthio (especially methylthio), halogen, and (C₁₋₄)fluoroalkoxy (especially difluoromethoxy).

xxvi) A further embodiment of the invention relates to compounds according to any one of embodiments i), ii) or x) to xxiv), wherein

A represents aryl, wherein the aryl is unsubstituted or mono-, di-, or tri-substituted,

wherein the substituents are independently selected from the group consisting of (C₁₋₄)alkyl (especially methyl), (C₁₋₄)alkoxy (especially methoxy), (C₁₋₄)alkylthio (especially methylthio), hydroxy, halogen, (C₁₋₄)fluoroalkyl (especially trifluoro-methyl), and (C₁₋₄)fluoroalkoxy (especially difluoromethoxy); or A represents a benzo[1,3]dioxolyl- or a 2,3-dihydro-benzo[1,4]dioxinyl-group wherein said groups are unsubstituted, mono- or di-substituted with halogen (especially di-substituted at a saturated carbon atom with fluorine).

xxvii) A further embodiment of the invention relates to compounds according to any one of embodiments i), ii), v), vi) or x) to xxvi), wherein

A represents phenyl, wherein the phenyl is di- or tri-substituted, wherein the substituents are independently selected from the group consisting of (C₁₋₄)alkyl (especially methyl), (C₁₋₄)alkoxy (especially methoxy), (C₁₋₄)alkylthio (especially methylthio), halogen, and (C₁₋₄)fluoroalkoxy (especially difluoromethoxy).

xxviii) A further embodiment of the invention relates to compounds according to any one of embodiments i), ii), v) to viii) or x) to xxvii), wherein

A represents 3,4-dimethoxyphenyl.

xxix) A further embodiment of the invention relates to compounds according to any one of embodiments i), ii), v), vi) or x) to xxvii), wherein

A represents 3-difluoromethoxy-4-methoxyphenyl or 4-difluoromethoxy-3-methoxyphenyl (especially 4-difluoromethoxy-3-methoxyphenyl).

xxx) A further embodiment of the invention relates to compounds according to any one of embodiments i) to iv), vi) or x) to xxiv), wherein

A represents heterocyclyl, wherein the heterocyclyl is unsubstituted or mono-, or di-substituted, wherein the substituents are independently selected from the group consisting of (C₁₋₄)alkyl (especially methyl), (C₁₋₄)alkoxy (especially methoxy), amino, and halogen; or A represents a 5H-[1,3]dioxolo[4,5-f]indole group.

xxxi) A further embodiment of the invention relates to compounds according to any one of embodiments i) to iv), vi) to viii), x) to xxv) or xxx), wherein

A represents an indolyl radical (especially indol-3-yl) or a benzimidazolyl radical (especially benzimidazol-2-yl) which radicals are unsubstituted or mono-, or di-substituted, wherein the substituents are independently selected from the group consisting of (C₁₋₄)alkyl (especially methyl), (C₁₋₄)alkoxy (especially methoxy), and halogen (especially fluorine).

xxxii) A further embodiment of the invention relates to compounds according to any one of embodiments i) to iv), vi) to xxv), xxx) or xxxi), wherein

A represents an indol-3-yl radical which radical is unsubstituted or mono-, or di-substituted, wherein the substituents are independently selected from the group consisting of (C₁₋₄)alkyl (especially methyl), (C₁₋₄)alkoxy (especially methoxy), and halogen (especially fluorine).

xxxiii) A further embodiment of the invention relates to compounds according to any one of embodiments i) or x) to xxxii), wherein

B represents a group selected from

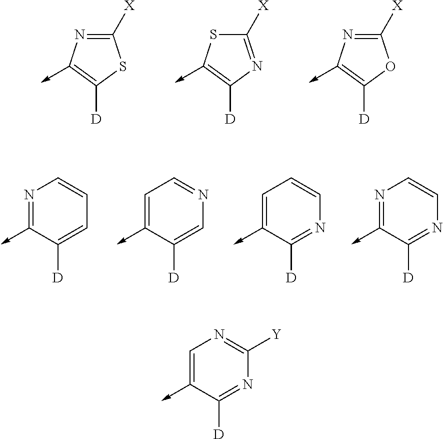

xxxiv) A further embodiment of the invention relates to compounds according to any one of embodiments i), ii), v) or x) to xxxiii), wherein

B represents a group selected from

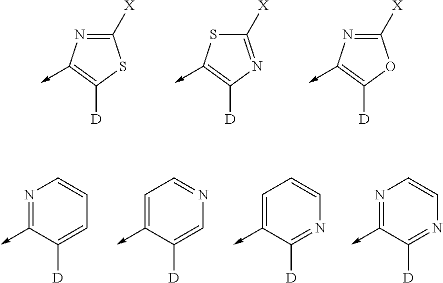

xxxv) A further embodiment of the invention relates to compounds according to any one of embodiments i) to v) or x) to xxxiv), wherein

B represents a group selected from

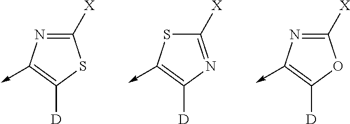

xxxvi) A further embodiment of the invention relates to compounds according to any one of embodiments i), ii), v) or x) to xxxiv), wherein

B represents a group selected from

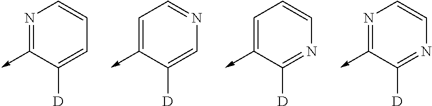

xxxvii) A further embodiment of the invention relates to compounds according to any one of embodiments i), ii), v) or x) to xxxiv), wherein

B represents a group selected from

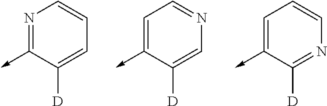

xxxviii) A further embodiment of the invention relates to compounds according to any one of embodiments i) to v) or x) to xxxiv), wherein

B represents a group selected from

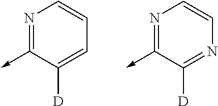

xxxix) A further embodiment of the invention relates to compounds according to any one of embodiments i) to v) or x) to xxxiv), wherein

B represents a group selected from

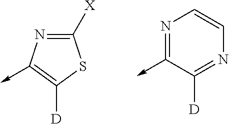

xl) A further embodiment of the invention relates to compounds according to any one of embodiments i) to vi), x) to xxxv) or xxxix), wherein

B represents

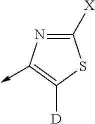

xli) A further embodiment of the invention relates to compounds according to any one of embodiments i) to v), viii), x) to xxxiv) or xxxix), wherein

B represents

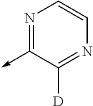

xlii) A further embodiment of the invention relates to compounds according to any one of embodiments i) to v) or x) to xxxv), wherein

B represents

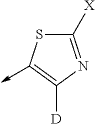

xliii) A further embodiment of the invention relates to compounds according to any one of embodiments i) to v), vii) or x) to xxxiv), wherein

B represents

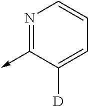

xliv) A further embodiment of the invention relates to compounds according to any one of embodiments i), ii), v) or x) to xxxiv), wherein

B represents

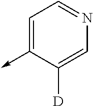

xlv) A further embodiment of the invention relates to compounds according to any one of embodiments i), ii), v) or x) to xxxiv), wherein

B represents

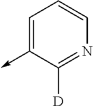

xlvi) A further embodiment of the invention relates to compounds according to any one of embodiments i) to vi), x) to xxxv), xxxix), xl) or xlii), wherein

X represents hydrogen, (C₁₋₄alkyl (especially methyl), (C₃₋₆)cycloalkyl (especially cyclopropyl), or NR⁴R⁵ (especially NH₂).

xlvii) A further embodiment of the invention relates to compounds according to any one of embodiments i) to vi), x) to xxxv), xxxix), xl) or xlii), wherein

X represents hydrogen, (C₁₋₄)alkyl (especially methyl), or NR⁴R⁵ (especially NH₂).

xlviii) A further embodiment of the invention relates to compounds according to any one of embodiments i) to vi), x) to xxxv), xxxix), xl) or xlii), wherein

X represents hydrogen.

xlix) A further embodiment of the invention relates to compounds according to any one of embodiments i) to vi), x) to xxxv), xxxix), xl) or xlii), wherein

X represents (C₁₋₄)alkyl (especially methyl).

l) A further embodiment of the invention relates to compounds according to any one of embodiments i) to vi), x) to xxxv), xxxix), xl) or xlii), wherein

X represents NR⁴R⁵ (especially NH₂).

Iii) A further embodiment of the invention relates to compounds according to any one of embodiments i) or ix) to xxxiii), wherein

Y represents hydrogen.

lii) A further embodiment of the invention relates to compounds according to any one of embodiments i) or ix) to xxxiii), wherein

Y represents (C₁₋₄)alkyl (especially methyl).

liii) A further embodiment of the invention relates to compounds according to any one of embodiments i) to iii) or x) to lii), wherein

D represents aryl, wherein the aryl is unsubstituted or mono-, di-, or tri-substituted, wherein the substituents are independently selected from the group consisting of (C₁₋₄)alkyl (especially methyl), (C₁₋₄)alkoxy (especially methoxy), hydroxy-(C₁₋₄)alkyl (especially hydroxy-methyl), (C₁₋₂)alkoxy-(C₁₋₄)alkoxy (especially 2-methoxy-ethoxy), halogen (especially fluorine, chlorine and bromine), (C₁₋₄)fluoroalkyl (especially trifluoromethyl), NMe₂, (C₁₋₄)alkyl-C(O)NH— (especially C₂H₅—C(O)NH—) and cyano.

liv) A further embodiment of the invention relates to compounds according to any one of embodiments i) to iii) or x) to lii), wherein

D represents aryl, wherein the aryl is unsubstituted or mono-, di-, or tri-substituted, wherein the substituents are independently selected from the group consisting of (C₁₋₄)alkyl (especially methyl), (C₁₋₄)alkoxy (especially methoxy), hydroxy-(C₁₋₄)alkyl (especially hydroxy-methyl), halogen (especially fluorine and chlorine), (C₁₋₄)fluoroalkyl (especially trifluoromethyl), NMe₂, and cyano.

lv) A further embodiment of the invention relates to compounds according to any one of embodiments i) to vi) or viii) to liv), wherein

D represents phenyl, wherein the phenyl is unsubstituted or mono-, di-, or tri-substituted, wherein the substituents are independently selected from the group consisting of (C₁₋₄)alkyl (especially methyl), (C₁₋₄)alkoxy (especially methoxy), and halogen (especially fluorine and chlorine).

lvi) A further embodiment of the invention relates to compounds according to any one of embodiments i) to lv), wherein

D represents phenyl, wherein the phenyl is unsubstituted or mono- or di-substituted, wherein the substituents are independently selected from the group consisting of (C₁₋₄)alkyl (especially methyl) and (C₁₋₄)alkoxy (especially methoxy).

lvii) A further embodiment of the invention relates to compounds according to any one of embodiments i) to iv), vi) or x) to lii), wherein

D represents heterocyclyl, wherein the heterocyclyl is unsubstituted or mono- or di-substituted, wherein the substituents are independently selected from the group consisting of (C₁₋₄)alkyl (especially methyl), (C₁₋₄)alkoxy (especially methoxy), hydroxy-(C₁₋₄)alkyl (especially hydroxy-methyl), halogen (especially fluorine and chlorine), and (C₁₋₄)alkyl-thio (especially methyl-thio).

lviii) A further embodiment of the invention relates to compounds according to any one of embodiments i) to iv), vi), x) to lii) or lvii), wherein

D represents heterocyclyl, wherein the heterocyclyl is unsubstituted or mono- or di-substituted, wherein the substituents are independently selected from the group consisting of (C₁₋₄)alkyl (especially methyl), (C₁₋₄)alkoxy (especially methoxy), and (C₁₋₄)alkyl-thio (especially methyl-thio).

lix) A further embodiment of the invention relates to compounds according to any one of embodiments i) to iv), vi), x) to lii) or lvii), wherein

D represents pyridyl, pyrimidyl or quinolinyl (especially pyridyl or quinolinyl) which are independently unsubstituted or mono- or di-substituted (especially unsubstituted or mono-substituted), wherein the substituents are independently selected from the group consisting of (C₁₋₄)alkyl (especially methyl), (C₁₋₄)alkoxy (especially methoxy), halogen (especially fluoro and chloro) and (C₁₋₄)alkyl-thio (especially methyl-thio).

lx) A further embodiment of the invention relates to compounds according to any one of embodiments i) to iv), vi) to viii), x) to lii) or lvii), wherein

D represents pyridyl or quinolinyl (especially pyridin-3-yl or quinolin-3-yl) which are independently unsubstituted or mono-substituted with (C₁₋₄)alkoxy (especially methoxy).

lxi) A further embodiment of the invention relates to compounds according to any one of embodiments i) to iv), vi) to viii), x) to lii) or lvii), wherein

D represents quinolinyl (especially quinolin-3-yl).

lxii) A further embodiment of the invention relates to compounds according to any one of embodiments i) to iv), vi), x) to lii) or lvii), wherein

D represents pyridyl (especially pyridin-3-yl), wherein the pyridyl is mono- or di-substituted (preferably mono-substituted), wherein the substituents are independently selected from the group consisting of (C₁₋₄)alkyl (especially methyl), (C₁₋₄)alkoxy (especially methoxy), and (C₁₋₄)alkyl-thio (especially methyl-thio).

lxiii) A further embodiment of the invention relates to compounds according to any one of embodiments i), ix) to xxiv), xxxii), xxxiii) or li) to lxii), wherein

B represents

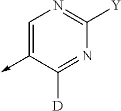

lxiv) Preferred compounds of formula (I) according to embodiment i) are selected from the group consisting of:
2-Amino-5-(3-fluoro-phenyl)-thiazole-4-carboxylic acid [2-(3-bromo-phenyl)-ethyl]-cyclopropylmethyl-amide;
5-(3-Fluoro-phenyl)-2-methyl-thiazole-4-carboxylic acid cyclopropylmethyl-[2-(3,4-dimethoxy-phenyl)-ethyl]-amide;
2-Methyl-5-m-tolyl-thiazole-4-carboxylic acid cyclopropylmethyl-[2-(3,4-dimethoxy-phenyl)-ethyl]-amide;
2-Bromo-5-m-tolyl-thiazole-4-carboxylic acid cyclopropylmethyl-[2-(3,4-dimethoxy-phenyl)-ethyl]-amide;
2-Amino-5-(3-fluoro-phenyl)-thiazole-4-carboxylic acid cyclopropylmethyl-[2-(3,4-dimethoxy-phenyl)-ethyl]-amide;
2-Amino-5-m-tolyl-thiazole-4-carboxylic acid cyclopropylmethyl-[2-(3,4-dimethoxy-phenyl)-ethyl]-amide;
2-Amino-5-(3-chloro-phenyl)-thiazole-4-carboxylic acid cyclopropylmethyl-[2-(3,4-dimethoxy-phenyl)-ethyl]-amide;
5-(4-Cyano-phenyl)-2-methyl-thiazole-4-carboxylic acid cyclopropylmethyl-[2-(3,4-dimethoxy-phenyl)-ethyl]-amide;
5-(3,5-Dimethyl-phenyl)-2-methyl-thiazole-4-carboxylic acid cyclopropylmethyl-[2-(3,4-dimethoxy-phenyl)-ethyl]-amide;
5-(3,5-Difluoro-phenyl)-2-methyl-thiazole-4-carboxylic acid cyclopropylmethyl-[2-(3,4-dimethoxy-phenyl)-ethyl]-amide;
5-(3-Fluoro-5-trifluoromethyl-phenyl)-2-methyl-thiazole-4-carboxylic acid cyclopropylmethyl-[2-(3,4-dimethoxy-phenyl)-ethyl]-amide;
5-(2,4-Dimethyl-phenyl)-2-methyl-thiazole-4-carboxylic acid cyclopropylmethyl-[2-(3,4-dimethoxy-phenyl)-ethyl]-amide;
5-(3-Fluoro-2-methyl-phenyl)-2-methyl-thiazole-4-carboxylic acid cyclopropylmethyl-[2-(3,4-dimethoxy-phenyl)-ethyl]-amide;
5-(2,3-Dimethyl-phenyl)-2-methyl-thiazole-4-carboxylic acid cyclopropylmethyl-[2-(3,4-dimethoxy-phenyl)-ethyl]-amide;
5-(3,4-Dichloro-phenyl)-2-methyl-thiazole-4-carboxylic acid cyclopropylmethyl-[2-(3,4-dimethoxy-phenyl)-ethyl]-amide;
5-(3-Fluoro-4-methyl-phenyl)-2-methyl-thiazole-4-carboxylic acid cyclopropylmethyl-[2-(3,4-dimethoxy-phenyl)-ethyl]-amide;
5-(3,4-Dimethyl-phenyl)-2-methyl-thiazole-4-carboxylic acid cyclopropylmethyl-[2-(3,4-dimethoxy-phenyl)-ethyl]-amide;
2-Methyl-5-phenyl-thiazole-4-carboxylic acid cyclopropylmethyl-[2-(3,4-dimethoxy-phenyl)-ethyl]-amide;
5-(3-Cyano-phenyl)-2-methyl-thiazole-4-carboxylic acid cyclopropylmethyl-[2-(3,4-dimethoxy-phenyl)-ethyl]-amide;
5-(4-Ethyl-phenyl)-2-methyl-thiazole-4-carboxylic acid cyclopropylmethyl-[2-(3,4-dimethoxy-phenyl)-ethyl]-amide;
5-(3,4-Difluoro-phenyl)-2-methyl-thiazole-4-carboxylic acid cyclopropylmethyl-[2-(3,4-dimethoxy-phenyl)-ethyl]-amide;
2-Cyclopropyl-5-phenyl-thiazole-4-carboxylic acid cyclopropylmethyl-[2-(3,4-dimethoxy-phenyl)-ethyl]-amide;
2-Cyclopropyl-5-p-tolyl-thiazole-4-carboxylic acid cyclopropylmethyl-[2-(3,4-dimethoxy-phenyl)-ethyl]-amide;
2-Cyclopropyl-5-(4-fluoro-phenyl)-thiazole-4-carboxylic acid cyclopropylmethyl-[2-(3,4-dimethoxy-phenyl)-ethyl]-amide;
2-Cyclopropyl-5-(3-fluoro-phenyl)-thiazole-4-carboxylic acid cyclopropylmethyl-[2-(3,4-dimethoxy-phenyl)-ethyl]-amide;
2-Cyclopropyl-5-(3-trifluoromethyl-phenyl)-thiazole-4-carboxylic acid cyclopropylmethyl-[2-(3,4-dimethoxy-phenyl)-ethyl]-amide;
2-Cyclopropyl-5-(3-fluoro-4-methyl-phenyl)-thiazole-4-carboxylic acid cyclopropylmethyl-[2-(3,4-dimethoxy-phenyl)-ethyl]-amide;
2-Cyclopropyl-5-(3-fluoro-5-trifluoromethyl-phenyl)-thiazole-4-carboxylic acid cyclopropyl-methyl-[2-(3,4-dimethoxy-phenyl)-ethyl]-amide;
2-Methoxy-5-m-tolyl-thiazole-4-carboxylic acid cyclopropylmethyl-[2-(3,4-dimethoxy-phenyl)-ethyl]-amide;
2-Dimethylamino-5-m-tolyl-thiazole-4-carboxylic acid cyclopropylmethyl-[2-(3,4-dimethoxy-phenyl)-ethyl]-amide;
2-Amino-5-(2-fluoro-phenyl)-thiazole-4-carboxylic acid cyclopropylmethyl-[2-(3,4-dimethoxy-phenyl)-ethyl]-amide;
2-Amino-5-phenyl-thiazole-4-carboxylic acid cyclopropylmethyl-[2-(3,4-dimethoxy-phenyl)-ethyl]-amide;
2-Amino-5-p-tolyl-thiazole-4-carboxylic acid cyclopropylmethyl-[2-(3,4-dimethoxy-phenyl)-ethyl]-amide;
5-m-Tolyl-thiazole-4-carboxylic acid cyclopropylmethyl-[2-(3,4-dimethoxy-phenyl)-ethyl]-amide;
5-(3-Chloro-phenyl)-thiazole-4-carboxylic acid cyclopropylmethyl-[2-(3,4-dimethoxy-phenyl)-ethyl]-amide;
5-(3-Trifluoromethyl-phenyl)-thiazole-4-carboxylic acid cyclopropylmethyl-[2-(3,4-dimethoxy-phenyl)-ethyl]-amide;
5-(2-Fluoro-phenyl)-thiazole-4-carboxylic acid cyclopropylmethyl-[2-(3,4-dimethoxy-phenyl)-ethyl]-amide;
5-(4-Fluoro-phenyl)-thiazole-4-carboxylic acid cyclopropylmethyl-[2-(3,4-dimethoxy-phenyl)-ethyl]-amide;
5-(3-Methoxy-phenyl)-thiazole-4-carboxylic acid cyclopropylmethyl-[2-(3,4-dimethoxy-phenyl)-ethyl]-amide;
5-Phenyl-thiazole-4-carboxylic acid cyclopropylmethyl-[2-(3,4-dimethoxy-phenyl)-ethyl]-amide;
5-(3-Fluoro-phenyl)-thiazole-4-carboxylic acid cyclopropylmethyl-[2-(3,4-dimethoxy-phenyl)-ethyl]-amide;
5-(3-Methoxy-phenyl)-2-methyl-oxazole-4-carboxylic acid cyclopropylmethyl-[2-(3,4-dimethoxy-phenyl)-ethyl]-amide;
2-Methyl-5-(3-trifluoromethyl-phenyl)-oxazole-4-carboxylic acid cyclopropylmethyl-[2-(3,4-dimethoxy-phenyl)-ethyl]-amide;
4-(3-Chloro-phenyl)-2-methyl-thiazole-5-carboxylic acid cyclopropylmethyl-[2-(3,4-dimethoxy-phenyl)-ethyl]-amide;
2-Methyl-4-(3-trifluoromethyl-phenyl)-thiazole-5-carboxylic acid cyclopropylmethyl-[2-(3,4-dimethoxy-phenyl)-ethyl]-amide;
4-(3-Methoxy-phenyl)-2-methyl-thiazole-5-carboxylic acid cyclopropylmethyl-[2-(3,4-dimethoxy-phenyl)-ethyl]-amide;
2-Methyl-4-(4-trifluoromethyl-phenyl)-thiazole-5-carboxylic acid cyclopropylmethyl-[2-(3,4-dimethoxy-phenyl)-ethyl]-amide;
4-(4-Chloro-phenyl)-2-methyl-thiazole-5-carboxylic acid cyclopropylmethyl-[2-(3,4-dimethoxy-phenyl)-ethyl]-amide;
2-Methyl-4-p-tolyl-thiazole-5-carboxylic acid cyclopropylmethyl-[2-(3,4-dimethoxy-phenyl)-ethyl]-amide;
4-(4-Fluoro-phenyl)-2-methyl-thiazole-5-carboxylic acid cyclopropylmethyl-[2-(3,4-dimethoxy-phenyl)-ethyl]-amide;
3-Phenyl-cinnoline-4-carboxylic acid cyclopropylmethyl-[2-(3,4-dimethoxy-phenyl)-ethyl]-amide;
6-Chloro-2-phenyl-imidazo[1,2-a]pyridine-3-carboxylic acid cyclopropylmethyl-[2-(3,4-dimethoxy-phenyl)-ethyl]-amide;
4-Phenyl-[1,2,3]thiadiazole-5-carboxylic acid cyclopropylmethyl-[2-(3,4-dimethoxy-phenyl)-ethyl]-amide;
3-Phenyl-pyrazine-2-carboxylic acid cyclopropylmethyl-[2-(3,4-dimethoxy-phenyl)-ethyl]-amide;
2-Methyl-5-m-tolyl-thiazole-4-carboxylic acid cyclopropylmethyl-[2-(3,4-dimethoxy-phenyl)-2-hydroxy-ethyl]-amide;
2-Bromo-5-m-tolyl-thiazole-4-carboxylic acid cyclopropylmethyl-[2-(3,4-dimethoxy-phenyl)-2-hydroxy-ethyl]-amide;
2-Amino-5-(3-fluoro-phenyl)-thiazole-4-carboxylic acid cyclopropylmethyl-[2-(3,4-dimethoxy-phenyl)-2-hydroxy-ethyl]-amide;
2-Amino-5-m-tolyl-thiazole-4-carboxylic acid cyclopropylmethyl-[2-(3,4-dimethoxy-phenyl)-2-hydroxy-ethyl]-amide;
2-Amino-5-(3-chloro-phenyl)-thiazole-4-carboxylic acid cyclopropylmethyl-[2-(3,4-dimethoxy-phenyl)-2-hydroxy-ethyl]-amide;
5-(3,5-Dimethyl-phenyl)-2-methyl-thiazole-4-carboxylic acid cyclopropylmethyl-[2-(3,4-dimethoxy-phenyl)-2-hydroxy-ethyl]-amide;
5-(3,5-Difluoro-phenyl)-2-methyl-thiazole-4-carboxylic acid cyclopropylmethyl-[2-(3,4-dimethoxy-phenyl)-2-hydroxy-ethyl]-amide;
5-(2,4-Dimethyl-phenyl)-2-methyl-thiazole-4-carboxylic acid cyclopropylmethyl-[2-(3,4-dimethoxy-phenyl)-2-hydroxy-ethyl]-amide;
5-(3-Fluoro-2-methyl-phenyl)-2-methyl-thiazole-4-carboxylic acid cyclopropylmethyl-[2-(3,4-dimethoxy-phenyl)-2-hydroxy-ethyl]-amide;
5-(2,3-Dimethyl-phenyl)-2-methyl-thiazole-4-carboxylic acid cyclopropylmethyl-[2-(3,4-dimethoxy-phenyl)-2-hydroxy-ethyl]-amide;
5-(3,4-Dichloro-phenyl)-2-methyl-thiazole-4-carboxylic acid cyclopropylmethyl-[2-(3,4-dimethoxy-phenyl)-2-hydroxy-ethyl]-amide;
5-(3-Fluoro-4-methyl-phenyl)-2-methyl-thiazole-4-carboxylic acid cyclopropylmethyl-[2-(3,4-dimethoxy-phenyl)-2-hydroxy-ethyl]-amide;
5-(3,4-Dimethyl-phenyl)-2-methyl-thiazole-4-carboxylic acid cyclopropylmethyl-[2-(3,4-dimethoxy-phenyl)-2-hydroxy-ethyl]-amide;
2-Methyl-5-phenyl-thiazole-4-carboxylic acid cyclopropylmethyl-[2-(3,4-dimethoxy-phenyl)-2-hydroxy-ethyl]-amide;
5-(4-Ethyl-phenyl)-2-methyl-thiazole-4-carboxylic acid cyclopropylmethyl-[2-(3,4-dimethoxy-phenyl)-2-hydroxy-ethyl]-amide;
5-(3,4-Difluoro-phenyl)-2-methyl-thiazole-4-carboxylic acid cyclopropylmethyl-[2-(3,4-dimethoxy-phenyl)-2-hydroxy-ethyl]-amide;
2-Cyclopropyl-5-phenyl-thiazole-4-carboxylic acid cyclopropylmethyl-[2-(3,4-dimethoxy-phenyl)-2-hydroxy-ethyl]-amide;
2-Cyclopropyl-5-p-tolyl-thiazole-4-carboxylic acid cyclopropylmethyl-[2-(3,4-dimethoxy-phenyl)-2-hydroxy-ethyl]-amide;
2-Cyclopropyl-5-(4-fluoro-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-phenyl)-thiazole-4-carboxylic acid cyclopropylmethyl-[2-(3,4-dimethoxy-phenyl)-2-hydroxy-ethyl]-amide;
2-Cyclopropyl-5-(3-trifluoromethyl-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-4-methyl-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-5-trifluoromethyl-phenyl)-thiazole-4-carboxylic acid cyclopropylmethyl-[2-(3,4-dimethoxy-phenyl)-2-hydroxy-ethyl]-amide;
2-Methoxy-5-m-tolyl-thiazole-4-carboxylic acid cyclopropylmethyl-[2-(3,4-dimethoxy-phenyl)-2-hydroxy-ethyl]-amide;
2-Dimethylamino-5-m-tolyl-thiazole-4-carboxylic acid cyclopropylmethyl-[2-(3,4-dimethoxy-phenyl)-2-hydroxy-ethyl]-amide;
2-Amino-5-(2-fluoro-phenyl)-thiazole-4-carboxylic acid cyclopropylmethyl-[2-(3,4-dimethoxy-phenyl)-2-hydroxy-ethyl]-amide;
2-Amino-5-phenyl-thiazole-4-carboxylic acid cyclopropylmethyl-[2-(3,4-dimethoxy-phenyl)-2-hydroxy-ethyl]-amide;
2-Amino-5-p-tolyl-thiazole-4-carboxylic acid cyclopropylmethyl-[2-(3,4-dimethoxy-phenyl)-2-hydroxy-ethyl]-amide;
5-m-Tolyl-thiazole-4-carboxylic acid cyclopropylmethyl-[2-(3,4-dimethoxy-phenyl)-2-hydroxy-ethyl]-amide;
5-(3-Chloro-phenyl)-thiazole-4-carboxylic acid cyclopropylmethyl-[2-(3,4-dimethoxy-phenyl)-2-hydroxy-ethyl]-amide;
5-(3-Trifluoromethyl-phenyl)-thiazole-4-carboxylic acid cyclopropylmethyl-[2-(3,4-dimethoxy-phenyl)-2-hydroxy-ethyl]-amide;
5-(2-Fluoro-phenyl)-thiazole-4-carboxylic acid cyclopropylmethyl-[2-(3,4-dimethoxy-phenyl)-2-hydroxy-ethyl]-amide;
5-(4-Fluoro-phenyl)-thiazole-4-carboxylic acid cyclopropylmethyl-[2-(3,4-dimethoxy-phenyl)-2-hydroxy-ethyl]-amide;
5-(3-M ethoxy-phenyl)-thiazole-4-carboxylic acid cyclopropylmethyl-[2-(3,4-dimethoxy-phenyl)-2-hydroxy-ethyl]-amide;
5-Phenyl-thiazole-4-carboxylic acid cyclopropylmethyl-[2-(3,4-dimethoxy-phenyl)-2-hydroxy-ethyl]-amide;
5-(3-Fluoro-phenyl)-thiazole-4-carboxylic acid cyclopropylmethyl-[2-(3,4-dimethoxy-phenyl)-2-hydroxy-ethyl]-amide;
4-(3-Chloro-phenyl)-2-methyl-thiazole-5-carboxylic acid cyclopropylmethyl-[2-(3,4-dimethoxy-phenyl)-2-hydroxy-ethyl]-amide;
2-Methyl-4-(3-trifluoromethyl-phenyl)-thiazole-5-carboxylic acid cyclopropylmethyl-[2-(3,4-dimethoxy-phenyl)-2-hydroxy-ethyl]-amide;
4-(3-Methoxy-phenyl)-2-methyl-thiazole-5-carboxylic acid cyclopropylmethyl-[2-(3,4-dimethoxy-phenyl)-2-hydroxy-ethyl]-amide;
4-(4-Chloro-phenyl)-2-methyl-thiazole-5-carboxylic acid cyclopropylmethyl-[2-(3,4-dimethoxy-phenyl)-2-hydroxy-ethyl]-amide;
2-Methyl-4-p-tolyl-thiazole-5-carboxylic acid cyclopropylmethyl-[2-(3,4-dimethoxy-phenyl)-2-hydroxy-ethyl]-amide;
4-(4-Fluoro-phenyl)-2-methyl-thiazole-5-carboxylic acid cyclopropylmethyl-[2-(3,4-dimethoxy-phenyl)-2-hydroxy-ethyl]-amide;
6-Chloro-2-phenyl-imidazo[1,2-a]pyridine-3-carboxylic acid cyclopropylmethyl-[2-(3,4-dimethoxy-phenyl)-2-hydroxy-ethyl]-amide;
4-Phenyl-[1,2,3]thiadiazole-5-carboxylic acid cyclopropylmethyl-[2-(3,4-dimethoxy-phenyl)-2-hydroxy-ethyl]-amide;
3-Phenyl-pyrazine-2-carboxylic acid cyclopropylmethyl-[2-(3,4-dimethoxy-phenyl)-2-hydroxy-ethyl]-amide;
5-(3-Fluoro-phenyl)-2-methyl-thiazole-4-carboxylic acid cyclopropylmethyl-[2-(3,4-dimethoxy-phenyl)-1-methyl-ethyl]-amide;
2-Amino-5-m-tolyl-thiazole-4-carboxylic acid cyclopropylmethyl-[2-(3,4-dimethoxy-phenyl)-1-methyl-ethyl]-amide;
5-(3,4-Dimethyl-phenyl)-2-methyl-thiazole-4-carboxylic acid cyclopropylmethyl-[2-(3,4-dimethoxy-phenyl)-1-methyl-ethyl]-amide;
5-(3-Fluoro-phenyl)-2-methyl-thiazole-4-carboxylic acid [2-(3,4-dimethoxy-phenyl)-ethyl]-methyl-amide;
2-Amino-5-m-tolyl-thiazole-4-carboxylic acid [2-(3,4-dimethoxy-phenyl)-ethyl]-methyl-amide;
5-(3,4-Dimethyl-phenyl)-2-methyl-thiazole-4-carboxylic acid [2-(3,4-dimethoxy-phenyl)-ethyl]-methyl-amide;
5-(3-Fluoro-phenyl)-2-methyl-thiazole-4-carboxylic acid [2-(3,4-dimethoxy-phenyl)-ethyl]-ethyl-amide;
2-Amino-5-m-tolyl-thiazole-4-carboxylic acid [2-(3,4-dimethoxy-phenyl)-ethyl]-ethyl-amide;
5-(3,4-Dimethyl-phenyl)-2-methyl-thiazole-4-carboxylic acid [2-(3,4-dimethoxy-phenyl)-ethyl]-ethyl-amide;
5-(3-Fluoro-phenyl)-2-methyl-thiazole-4-carboxylic acid [2-(3,4-dimethoxy-phenyl)-ethyl]-propyl-amide;
2-Amino-5-m-tolyl-thiazole-4-carboxylic acid [2-(3,4-dimethoxy-phenyl)-ethyl]-propyl-amide;
5-(3,4-Dimethyl-phenyl)-2-methyl-thiazole-4-carboxylic acid [2-(3,4-dimethoxy-phenyl)-ethyl]-propyl-amide;
5-(3-Fluoro-phenyl)-2-methyl-thiazole-4-carboxylic acid [2-(3,4-dimethoxy-phenyl)-ethyl]-isobutyl-amide;
2-Amino-5-m-tolyl-thiazole-4-carboxylic acid [2-(3,4-dimethoxy-phenyl)-ethyl]-isobutyl-amide;
5-(3,4-Dimethyl-phenyl)-2-methyl-thiazole-4-carboxylic acid [2-(3,4-dimethoxy-phenyl)-ethyl]-isobutyl-amide;
5-(3-Fluoro-phenyl)-2-methyl-thiazole-4-carboxylic acid [2-(3,4-dimethoxy-phenyl)-ethyl]-isopropyl-amide;
2-Amino-5-m-tolyl-thiazole-4-carboxylic acid [2-(3,4-dimethoxy-phenyl)-ethyl]-isopropyl-amide;
5-(3,4-Dimethyl-phenyl)-2-methyl-thiazole-4-carboxylic acid [2-(3,4-dimethoxy-phenyl)-ethyl]-isopropyl-amide;
5-(3-Fluoro-phenyl)-2-methyl-thiazole-4-carboxylic acid [2-(3,4-dimethoxy-phenyl)-ethyl]-(2,2,2-trifluoro-ethyl)-amide;
2-Amino-5-m-tolyl-thiazole-4-carboxylic acid [2-(3,4-dimethoxy-phenyl)-ethyl]-(2,2,2-trifluoro-ethyl)-amide;
5-(3,4-Dimethyl-phenyl)-2-methyl-thiazole-4-carboxylic acid [2-(3,4-dimethoxy-phenyl)-ethyl]-(2,2,2-trifluoro-ethyl)-amide;
5-(3-Fluoro-phenyl)-2-methyl-thiazole-4-carboxylic acid cyclopropyl-[2-(3,4-dimethoxy-phenyl)-ethyl]-amide;
2-Amino-5-m-tolyl-thiazole-4-carboxylic acid cyclopropyl-[2-(3,4-dimethoxy-phenyl)-ethyl]-amide;
5-(3,4-Dimethyl-phenyl)-2-methyl-thiazole-4-carboxylic acid cyclopropyl-[2-(3,4-dimethoxy-phenyl)-ethyl]-amide;
5-(3-Fluoro-phenyl)-2-methyl-thiazole-4-carboxylic acid [2-(3,4-dimethoxy-phenyl)-ethyl]-(2-hydroxy-ethyl)-amide;
2-Amino-5-m-tolyl-thiazole-4-carboxylic acid [2-(3,4-dimethoxy-phenyl)-ethyl]-(2-hydroxy-ethyl)-amide;
5-(3,4-Dimethyl-phenyl)-2-methyl-thiazole-4-carboxylic acid [2-(3,4-dimethoxy-phenyl)-ethyl]-(2-hydroxy-ethyl)-amide;
5-(3-Fluoro-phenyl)-2-methyl-thiazole-4-carboxylic acid [2-(3,4-dimethoxy-phenyl)-ethyl]-(2-methoxy-ethyl)-amide;
2-Amino-5-m-tolyl-thiazole-4-carboxylic acid [2-(3,4-dimethoxy-phenyl)-ethyl]-(2-methoxy-ethyl)-amide;
5-(3,4-Dimethyl-phenyl)-2-methyl-thiazole-4-carboxylic acid [2-(3,4-dimethoxy-phenyl)-ethyl]-(2-methoxy-ethyl)-amide;
2-Amino-5-m-tolyl-thiazole-4-carboxylic acid [2-(3,4-dimethoxy-phenyl)-ethyl]-(2-dimethylamino-ethyl)-amide;
5-(3-Fluoro-phenyl)-2-methyl-thiazole-4-carboxylic acid carbamoylmethyl-[2-(3,4-dimethoxy-phenyl)-ethyl]-amide;
2-Amino-5-m-tolyl-thiazole-4-carboxylic acid carbamoylmethyl-[2-(3,4-dimethoxy-phenyl)-ethyl]-amide;
5-(3,4-Dimethyl-phenyl)-2-methyl-thiazole-4-carboxylic acid carbamoylmethyl-[2-(3,4-dimethoxy-phenyl)-ethyl]-amide;
5-(3-Fluoro-phenyl)-2-methyl-thiazole-4-carboxylic acid [2-(3,4-dimethoxy-phenyl)-ethyl]-dimethylcarbamoylmethyl-amide;
2-Amino-5-m-tolyl-thiazole-4-carboxylic acid [2-(3,4-dimethoxy-phenyl)-ethyl]-dimethylcarbamoylmethyl-amide;
5-(3,4-Dimethyl-phenyl)-2-methyl-thiazole-4-carboxylic acid [2-(3,4-dimethoxy-phenyl)-ethyl]-dimethylcarbamoylmethyl-amide;
2-Amino-5-m-tolyl-thiazole-4-carboxylic acid cyclopropylmethyl-phenethyl-amide;
2-Amino-5-m-tolyl-thiazole-4-carboxylic acid [2-(2-chloro-phenyl)-ethyl]-cyclopropylmethyl-amide;
2-Amino-5-m-tolyl-thiazole-4-carboxylic acid cyclopropylmethyl-[2-(2-methoxy-phenyl)-ethyl]-amide;
2-Amino-5-m-tolyl-thiazole-4-carboxylic acid cyclopropylmethyl-[2-(2-fluoro-phenyl)-ethyl]-amide;
2-Amino-5-m-tolyl-thiazole-4-carboxylic acid cyclopropylmethyl-(2-o-tolyl-ethyl)-amide;
2-Amino-5-m-tolyl-thiazole-4-carboxylic acid cyclopropylmethyl-(2-m-tolyl-ethyl)-amide;
2-Amino-5-m-tolyl-thiazole-4-carboxylic acid cyclopropylmethyl-[2-(3-methoxy-phenyl)-ethyl]-amide;
2-Amino-5-m-tolyl-thiazole-4-carboxylic acid [2-(4-chloro-phenyl)-ethyl]-cyclopropylmethyl-amide;
2-Amino-5-m-tolyl-thiazole-4-carboxylic acid cyclopropylmethyl-(2-p-tolyl-ethyl)-amide;
2-Amino-5-m-tolyl-thiazole-4-carboxylic acid cyclopropylmethyl-[2-(4-ethyl-phenyl)-ethyl]-amide;
2-Amino-5-m-tolyl-thiazole-4-carboxylic acid cyclopropylmethyl-[2-(4-methoxy-phenyl)-ethyl]-amide;
2-Amino-5-m-tolyl-thiazole-4-carboxylic acid cyclopropylmethyl-[2-(4-hydroxy-phenyl)-ethyl]-amide;
2-Amino-5-m-tolyl-thiazole-4-carboxylic acid cyclopropylmethyl-[2-(4-methylsulfanyl-phenyl)-ethyl]-amide;
2-Amino-5-m-tolyl-thiazole-4-carboxylic acid cyclopropylmethyl-[2-(4-trifluoromethyl-phenyl)-ethyl]-amide;
2-Amino-5-m-tolyl-thiazole-4-carboxylic acid cyclopropylmethyl-[2-(4-trifluoromethoxy-phenyl)-ethyl]-amide;
2-Amino-5-m-tolyl-thiazole-4-carboxylic acid cyclopropylmethyl-[2-(2,4-dimethyl-phenyl)-ethyl]-amide;
2-Amino-5-m-tolyl-thiazole-4-carboxylic acid cyclopropylmethyl-[2-(2,5-dimethoxy-phenyl)-ethyl]-amide;
2-Amino-5-m-tolyl-thiazole-4-carboxylic acid cyclopropylmethyl-[2-(2,5-dimethyl-phenyl)-ethyl]-amide;
2-Amino-5-m-tolyl-thiazole-4-carboxylic acid [2-(5-bromo-2-methoxy-phenyl)-ethyl]-cyclopropylmethyl-amide;
2-Amino-5-m-tolyl-thiazole-4-carboxylic acid (2-benzo[1,3]dioxol-5-yl-ethyl)-cyclopropylmethyl-amide;
2-Amino-5-m-tolyl-thiazole-4-carboxylic acid cyclopropylmethyl-[2-(2,2-difluoro-benzo[1,3]dioxol-5-yl)-ethyl]-amide;
2-Amino-5-m-tolyl-thiazole-4-carboxylic acid cyclopropylmethyl-[2-(2,3-dihydro-benzo[1,4]dioxin-6-yl)-ethyl]-amide;
2-Amino-5-m-tolyl-thiazole-4-carboxylic acid cyclopropylmethyl-[2-(4-ethoxy-3-methoxy-phenyl)-ethyl]-amide;
2-Amino-5-m-tolyl-thiazole-4-carboxylic acid cyclopropylmethyl-[2-(3-ethoxy-4-methoxy-phenyl)-ethyl]-amide;
2-Amino-5-m-tolyl-thiazole-4-carboxylic acid cyclopropylmethyl-[2-(4-methoxy-3-methylsulfanyl-phenyl)-ethyl]-amide;
2-Amino-5-m-tolyl-thiazole-4-carboxylic acid cyclopropylmethyl-[2-(4-methoxy-3-methyl-phenyl)-ethyl]-amide;
2-Amino-5-m-tolyl-thiazole-4-carboxylic acid [2-(3-bromo-4-methoxy-phenyl)-ethyl]-cyclopropylmethyl-amide;
2-Amino-5-m-tolyl-thiazole-4-carboxylic acid cyclopropylmethyl-[2-(3,4-dimethyl-phenyl)-ethyl]-amide;
2-Amino-5-m-tolyl-thiazole-4-carboxylic acid cyclopropylmethyl-[2-(3-difluoromethoxy-4-methoxy-phenyl)-ethyl]-amide;
2-Amino-5-m-tolyl-thiazole-4-carboxylic acid cyclopropylmethyl-[2-(4-difluoromethoxy-3-methoxy-phenyl)-ethyl]-amide;
2-Amino-5-m-tolyl-thiazole-4-carboxylic acid cyclopropylmethyl-(2-naphthalen-2-yl-ethyl)-amide;
2-Amino-5-m-tolyl-thiazole-4-carboxylic acid cyclopropylmethyl-[2-(4-hydroxy-3-methoxy-phenyl)-ethyl]-amide;
2-Amino-5-m-tolyl-thiazole-4-carboxylic acid cyclopropylmethyl-[1-(3,4-dimethoxy-benzyl)-propyl]-amide;
2-Amino-5-m-tolyl-thiazole-4-carboxylic acid cyclopropylmethyl-[2-(3,5-dimethoxy-phenyl)-ethyl]-amide;
2-Amino-5-m-tolyl-thiazole-4-carboxylic acid cyclopropylmethyl-[2-(2,6-dichloro-phenyl)-ethyl]-amide;
2-Amino-5-m-tolyl-thiazole-4-carboxylic acid cyclopropylmethyl-[2-(3,4,5-trimethoxy-phenyl)-ethyl]-amide;
2-Amino-5-m-tolyl-thiazole-4-carboxylic acid cyclopropylmethyl-[2-(4-isopropoxy-3,5-dimethoxy-phenyl)-ethyl]-amide;
2-Amino-5-m-tolyl-thiazole-4-carboxylic acid cyclopropylmethyl-[2-(4-iodo-2,5-dimethoxy-phenyl)-ethyl]-amide;
5-(3,4-Dimethyl-phenyl)-2-methyl-thiazole-4-carboxylic acid cyclopropylmethyl-phenethyl-amide;
5-(3,4-Dimethyl-phenyl)-2-methyl-thiazole-4-carboxylic acid [2-(2-chloro-phenyl)-ethyl]-cyclopropylmethyl-amide;
5-(3,4-Dimethyl-phenyl)-2-methyl-thiazole-4-carboxylic acid cyclopropylmethyl-[2-(2-methoxy-phenyl)-ethyl]-amide;
5-(3,4-Dimethyl-phenyl)-2-methyl-thiazole-4-carboxylic acid cyclopropylmethyl-[2-(2-fluoro-phenyl)-ethyl]-amide;
5-(3,4-Dimethyl-phenyl)-2-methyl-thiazole-4-carboxylic acid cyclopropylmethyl-(2-m-tolyl-ethyl)-amide;
5-(3,4-Dimethyl-phenyl)-2-methyl-thiazole-4-carboxylic acid cyclopropylmethyl-[2-(3-methoxy-phenyl)-ethyl]-amide;
5-(3,4-Dimethyl-phenyl)-2-methyl-thiazole-4-carboxylic acid cyclopropylmethyl-[2-(4-fluoro-phenyl)-ethyl]-amide;
5-(3,4-Dimethyl-phenyl)-2-methyl-thiazole-4-carboxylic acid [2-(4-chloro-phenyl)-ethyl]-cyclopropylmethyl-amide;
5-(3,4-Dimethyl-phenyl)-2-methyl-thiazole-4-carboxylic acid cyclopropylmethyl-(2-p-tolyl-ethyl)-amide;
5-(3,4-Dimethyl-phenyl)-2-methyl-thiazole-4-carboxylic acid cyclopropylmethyl-[2-(4-ethyl-phenyl)-ethyl]-amide;
5-(3,4-Dimethyl-phenyl)-2-methyl-thiazole-4-carboxylic acid cyclopropylmethyl-[2-(4-methoxy-phenyl)-ethyl]-amide;
5-(3,4-Dimethyl-phenyl)-2-methyl-thiazole-4-carboxylic acid cyclopropylmethyl-[2-(4-hydroxy-phenyl)-ethyl]-amide;
5-(3,4-Dimethyl-phenyl)-2-methyl-thiazole-4-carboxylic acid cyclopropylmethyl-[2-(4-methylsulfanyl-phenyl)-ethyl]-amide;
5-(3,4-Dimethyl-phenyl)-2-methyl-thiazole-4-carboxylic acid cyclopropylmethyl-[2-(4-trifluoromethyl-phenyl)-ethyl]-amide;
5-(3,4-Dimethyl-phenyl)-2-methyl-thiazole-4-carboxylic acid cyclopropylmethyl-[2-(2,4-dimethyl-phenyl)-ethyl]-amide;
5-(3,4-Dimethyl-phenyl)-2-methyl-thiazole-4-carboxylic acid cyclopropylmethyl-[2-(2,5-dimethoxy-phenyl)-ethyl]-amide;
5-(3,4-Dimethyl-phenyl)-2-methyl-thiazole-4-carboxylic acid cyclopropylmethyl-[2-(2,5-dimethyl-phenyl)-ethyl]-amide;
5-(3,4-Dimethyl-phenyl)-2-methyl-thiazole-4-carboxylic acid [2-(5-bromo-2-methoxy-phenyl)-ethyl]-cyclopropylmethyl-amide;
5-(3,4-Dimethyl-phenyl)-2-methyl-thiazole-4-carboxylic acid (2-benzo[1,3]dioxol-5-yl-ethyl)-cyclopropylmethyl-amide;
5-(3,4-Dimethyl-phenyl)-2-methyl-thiazole-4-carboxylic acid cyclopropylmethyl-[2-(2,3-dihydro-benzo[1,4]dioxin-6-yl)-ethyl]-amide;
5-(3,4-Dimethyl-phenyl)-2-methyl-thiazole-4-carboxylic acid cyclopropylmethyl-[2-(4-ethoxy-3-methoxy-phenyl)-ethyl]-amide;
5-(3,4-Dimethyl-phenyl)-2-methyl-thiazole-4-carboxylic acid cyclopropylmethyl-[2-(3-ethoxy-4-methoxy-phenyl)-ethyl]-amide;
5-(3,4-Dimethyl-phenyl)-2-methyl-thiazole-4-carboxylic acid cyclopropylmethyl-[2-(4-methoxy-3-methylsulfanyl-phenyl)-ethyl]-amide;
5-(3,4-Dimethyl-phenyl)-2-methyl-thiazole-4-carboxylic acid cyclopropylmethyl-[2-(4-methoxy-3-methyl-phenyl)-ethyl]-amide;
5-(3,4-Dimethyl-phenyl)-2-methyl-thiazole-4-carboxylic acid [2-(3-bromo-4-methoxy-phenyl)-ethyl]-cyclopropylmethyl-amide;
5-(3,4-Dimethyl-phenyl)-2-methyl-thiazole-4-carboxylic acid cyclopropylmethyl-[2-(3,4-dimethyl-phenyl)-ethyl]-amide;
5-(3,4-Dimethyl-phenyl)-2-methyl-thiazole-4-carboxylic acid cyclopropylmethyl-[2-(3-difluoromethoxy-4-methoxy-phenyl)-ethyl]-amide;
5-(3,4-Dimethyl-phenyl)-2-methyl-thiazole-4-carboxylic acid cyclopropylmethyl-[2-(4-difluoromethoxy-3-methoxy-phenyl)-ethyl]-amide;
5-(3,4-Dimethyl-phenyl)-2-methyl-thiazole-4-carboxylic acid cyclopropylmethyl-[2-(4-hydroxy-3-methoxy-phenyl)-ethyl]-amide;
5-(3,4-Dimethyl-phenyl)-2-methyl-thiazole-4-carboxylic acid cyclopropylmethyl-[1-(3,4-dimethoxy-benzyl)-propyl]-amide;
5-(3,4-Dimethyl-phenyl)-2-methyl-thiazole-4-carboxylic acid cyclopropylmethyl-[2-(3,5-dimethoxy-phenyl)-ethyl]-amide;
5-(3,4-Dimethyl-phenyl)-2-methyl-thiazole-4-carboxylic acid cyclopropylmethyl-[2-(2,6-dichloro-phenyl)-ethyl]-amide;
5-(3,4-Dimethyl-phenyl)-2-methyl-thiazole-4-carboxylic acid cyclopropylmethyl-[2-(3,4,5-trimethoxy-phenyl)-ethyl]-amide;
5-(3,4-Dimethyl-phenyl)-2-methyl-thiazole-4-carboxylic acid cyclopropylmethyl-[2-(4-isopropoxy-3,5-dimethoxy-phenyl)-ethyl]-amide;
5-(3,4-Dimethyl-phenyl)-2-methyl-thiazole-4-carboxylic acid cyclopropylmethyl-[2-(4-iodo-2,5-dimethoxy-phenyl)-ethyl]-amide;
N-Cyclopropylmethyl-N-[2-(3,4-dimethoxy-phenyl)-ethyl]-3-m-tolyl-isonicotinamide;
N-Cyclopropylmethyl-N-[2-(3,4-dimethoxy-phenyl)-ethyl]-3-p-tolyl-isonicotinamide;
N-Cyclopropylmethyl-N-[2-(3,4-dimethoxy-phenyl)-ethyl]-3-(3,4-dimethyl-phenyl)-isonicotinamide;
N-Cyclopropylmethyl-N-[2-(3,4-dimethoxy-phenyl)-ethyl]-3-(3-methoxy-phenyl)-isonicotinamide;
3-m-Tolyl-pyridine-2-carboxylic acid cyclopropylmethyl-[2-(3,4-dimethoxy-phenyl)-ethyl]-amide;
3-p-Tolyl-pyridine-2-carboxylic acid cyclopropylmethyl-[2-(3,4-dimethoxy-phenyl)-ethyl]-amide;
3-(3,4-Dimethyl-phenyl)-pyridine-2-carboxylic acid cyclopropylmethyl-[2-(3,4-dimethoxy-phenyl)-ethyl]-amide;
3-(3-Methoxy-phenyl)-pyridine-2-carboxylic acid cyclopropylmethyl-[2-(3,4-dimethoxy-phenyl)-ethyl]-amide;
N-Cyclopropylmethyl-N-[2-(3,4-dimethoxy-phenyl)-ethyl]-2-m-tolyl-nicotinamide;
N-Cyclopropylmethyl-N-[2-(3,4-dimethoxy-phenyl)-ethyl]-2-p-tolyl-nicotinamide;
N-Cyclopropylmethyl-N-[2-(3,4-dimethoxy-phenyl)-ethyl]-2-(3,4-dimethyl-phenyl)-nicotinamide;
N-Cyclopropylmethyl-N-[2-(3,4-dimethoxy-phenyl)-ethyl]-2-(3-methoxy-phenyl)-nicotinamide; and
2-Methyl-5-m-tolyl-thiazole-4-carboxylic acid [2-cyclopropyl-amino-2-(3,4-dimethoxy-phenyl)-ethyl]-cyclopropylmethyl-amide;
   wherein it is well understood that any stereogenic center of the above listed compounds may be in absolute (R)- or (S)-configuration.

lxv) In addition to the above-listed compounds, further preferred compounds of formula (I) according to embodiment i) are selected from the group consisting of:
2-Amino-5-m-tolyl-thiazole-4-carboxylic acid cyclopropylmethyl-[2-(1H-indol-3-yl)-ethyl]-amide;
2-Amino-5-m-tolyl-thiazole-4-carboxylic acid [2-(1H-benzoimidazol-2-yl)-ethyl]-cyclopropylmethyl-amide;
2-Amino-5-m-tolyl-thiazole-4-carboxylic acid [2-(2-amino-thiazol-4-yl)-ethyl]-cyclopropylmethyl-amide;
2-Amino-5-m-tolyl-thiazole-4-carboxylic acid cyclopropylmethyl-[2-(2-ethyl-4-iodo-imidazol-1-yl)-ethyl]-amide;
5-(3,4-Dimethyl-phenyl)-2-methyl-thiazole-4-carboxylic acid cyclopropylmethyl-[2-(1H-indol-3-yl)-ethyl]-amide;
5-(3,4-Dimethyl-phenyl)-2-methyl-thiazole-4-carboxylic acid [2-(1H-benzoimidazol-2-yl)-ethyl]-cyclopropylmethyl-amide;
5-(3,4-Dimethyl-phenyl)-2-methyl-thiazole-4-carboxylic acid cyclopropylmethyl-[2-(2-ethyl-4-iodo-imidazol-1-yl)-ethyl]-amide;
2-Amino-5-m-tolyl-thiazole-4-carboxylic acid cyclopropylmethyl-[2-(6-methoxy-1H-benzoimidazol-2-yl)-ethyl]-amide;
2-Amino-5-m-tolyl-thiazole-4-carboxylic acid cyclopropylmethyl-[2-(5,6-dimethyl-1H-benzoimidazol-2-yl)-ethyl]-amide;
2-Amino-5-m-tolyl-thiazole-4-carboxylic acid cyclopropylmethyl-[2-(6-methyl-1H-benzoimidazol-2-yl)-ethyl]-amide;
2-Amino-5-m-tolyl-thiazole-4-carboxylic acid [2-(6-chloro-1H-benzoimidazol-2-yl)-ethyl]-cyclopropylmethyl-amide;
2-Amino-5-m-tolyl-thiazole-4-carboxylic acid cyclopropylmethyl-(2-indol-1-yl-ethyl)-amide;
2-Amino-5-m-tolyl-thiazole-4-carboxylic acid cyclopropylmethyl-[2-(1-methyl-1H-indol-3-yl)-ethyl]-amide;
2-Amino-5-m-tolyl-thiazole-4-carboxylic acid [2-(5-bromo-1H-indol-3-yl)-ethyl]-cyclopropylmethyl-amide;
2-Amino-5-m-tolyl-thiazole-4-carboxylic acid [2-(6-chloro-1H-indol-3-yl)-ethyl]-cyclopropylmethyl-amide;
2-Amino-5-m-tolyl-thiazole-4-carboxylic acid cyclopropylmethyl-[2-(7-methoxy-1H-indol-3-yl)-ethyl]-amide;
2-Amino-5-m-tolyl-thiazole-4-carboxylic acid cyclopropylmethyl-[2-(5-methoxy-1H-indol-3-yl)-ethyl]-amide;
2-Amino-5-m-tolyl-thiazole-4-carboxylic acid cyclopropylmethyl-[2-(6-methoxy-1H-indol-3-yl)-ethyl]-amide;
2-Amino-5-m-tolyl-thiazole-4-carboxylic acid cyclopropylmethyl-[2-(5-methyl-1H-indol-3-yl)-ethyl]-amide;
2-Amino-5-m-tolyl-thiazole-4-carboxylic acid cyclopropylmethyl-[2-(6-methyl-1H-indol-3-yl)-ethyl]-amide;
2-Amino-5-m-tolyl-thiazole-4-carboxylic acid cyclopropylmethyl-[2-(7-methyl-1H-indol-3-yl)-ethyl]-amide;
2-Amino-5-m-tolyl-thiazole-4-carboxylic acid cyclopropylmethyl-[2-(4-fluoro-1H-indol-3-yl)-ethyl]-amide;
2-Amino-5-m-tolyl-thiazole-4-carboxylic acid cyclopropylmethyl-[2-(5-fluoro-1H-indol-3-yl)-ethyl]-amide;
2-Amino-5-m-tolyl-thiazole-4-carboxylic acid cyclopropylmethyl-[2-(6-fluoro-1H-indol-3-yl)-ethyl]-amide;
2-Amino-5-m-tolyl-thiazole-4-carboxylic acid cyclopropylmethyl-[2-(7-fluoro-1H-indol-3-yl)-ethyl]-amide;
2-Amino-5-m-tolyl-thiazole-4-carboxylic acid cyclopropylmethyl-[2-(6-methoxy-pyridin-3-yl)-ethyl]-amide;
5-(3,4-Dimethyl-phenyl)-2-methyl-thiazole-4-carboxylic acid cyclopropylmethyl-[2-(6-methoxy-1H-benzoimidazol-2-yl)-ethyl]-amide;
5-(3,4-Dimethyl-phenyl)-2-methyl-thiazole-4-carboxylic acid cyclopropylmethyl-[2-(5,6-dimethyl-1H-benzoimidazol-2-yl)-ethyl]-amide;
5-(3,4-Dimethyl-phenyl)-2-methyl-thiazole-4-carboxylic acid cyclopropylmethyl-[2-(6-methyl-1H-benzoimidazol-2-yl)-ethyl]-amide;
5-(3,4-Dimethyl-phenyl)-2-methyl-thiazole-4-carboxylic acid [2-(6-chloro-1H-benzoimidazol-2-yl)-ethyl]-cyclopropylmethyl-amide;
5-(3,4-Dimethyl-phenyl)-2-methyl-thiazole-4-carboxylic acid cyclopropylmethyl-(2-indol-1-yl-ethyl)-amide;
5-(3,4-Dimethyl-phenyl)-2-methyl-thiazole-4-carboxylic acid cyclopropylmethyl-[2-(1-methyl-1H-indol-3-yl)-ethyl]-amide;
5-(3,4-Dimethyl-phenyl)-2-methyl-thiazole-4-carboxylic acid [2-(5-bromo-1H-indol-3-yl)-ethyl]-cyclopropylmethyl-amide;
5-(3,4-Dimethyl-phenyl)-2-methyl-thiazole-4-carboxylic acid [2-(6-chloro-1H-indol-3-yl)-ethyl]-cyclopropylmethyl-amide;
5-(3,4-Dimethyl-phenyl)-2-methyl-thiazole-4-carboxylic acid cyclopropylmethyl-[2-(7-methoxy-1H-indol-3-yl)-ethyl]-amide;
5-(3,4-Dimethyl-phenyl)-2-methyl-thiazole-4-carboxylic acid cyclopropylmethyl-[2-(5-methoxy-1H-indol-3-yl)-ethyl]-amide;
5-(3,4-Dimethyl-phenyl)-2-methyl-thiazole-4-carboxylic acid cyclopropylmethyl-[2-(6-methoxy-1H-indol-3-yl)-ethyl]-amide;
5-(3,4-Dimethyl-phenyl)-2-methyl-thiazole-4-carboxylic acid cyclopropylmethyl-[2-(5-methyl-1H-indol-3-yl)-ethyl]-amide;
5-(3,4-Dimethyl-phenyl)-2-methyl-thiazole-4-carboxylic acid cyclopropylmethyl-[2-(6-methyl-1H-indol-3-yl)-ethyl]-amide;
5-(3,4-Dimethyl-phenyl)-2-methyl-thiazole-4-carboxylic acid cyclopropylmethyl-[2-(7-methyl-1H-indol-3-yl)-ethyl]-amide;
5-(3,4-Dimethyl-phenyl)-2-methyl-thiazole-4-carboxylic acid cyclopropylmethyl-[2-(4-fluoro-1H-indol-3-yl)-ethyl]-amide;
5-(3,4-Dimethyl-phenyl)-2-methyl-thiazole-4-carboxylic acid cyclopropylmethyl-[2-(5-fluoro-1H-indol-3-yl)-ethyl]-amide;
5-(3,4-Dimethyl-phenyl)-2-methyl-thiazole-4-carboxylic acid cyclopropylmethyl-[2-(6-fluoro-1H-indol-3-yl)-ethyl]-amide;
5-(3,4-Dimethyl-phenyl)-2-methyl-thiazole-4-carboxylic acid cyclopropylmethyl-[2-(7-fluoro-1H-indol-3-yl)-ethyl]-amide;
5-(3,4-Dimethyl-phenyl)-2-methyl-thiazole-4-carboxylic acid cyclopropylmethyl-[2-(6-methoxy-pyridin-3-yl)-ethyl]-amide;
3-p-Tolyl-pyrazine-2-carboxylic acid cyclopropylmethyl-[2-(5-fluoro-1H-indol-3-yl)-ethyl]-amide;
3-(3,4-Dimethyl-phenyl)-pyrazine-2-carboxylic acid cyclopropylmethyl-[2-(5-fluoro-1H-indol-3-yl)-ethyl]-amide;
3-m-Tolyl-pyrazine-2-carboxylic acid cyclopropylmethyl-[2-(5-fluoro-1H-indol-3-yl)-ethyl]-amide;
3-(3-M ethoxy-phenyl)-pyrazine-2-carboxylic acid cyclopropylmethyl-[2-(5-fluoro-1H-indol-3-yl)-ethyl]-amide;
5-(3,4-Dimethyl-phenyl)-2-methyl-oxazole-4-carboxylic acid cyclopropylmethyl-[2-(5-fluoro-1H-indol-3-yl)-ethyl]-amide;
5-(3,4-Dimethyl-phenyl)-oxazole-4-carboxylic acid cyclopropylmethyl-[2-(5-fluoro-1H-indol-3-yl)-ethyl]-amide;
5-(3-Dimethylamino-phenyl)-oxazole-4-carboxylic acid cyclopropylmethyl-[2-(5-fluoro-1H-indol-3-yl)-ethyl]-amide;
4-(4-Chloro-phenyl)-2-methyl-thiazole-5-carboxylic acid cyclopropylmethyl-[2-(5-fluoro-1H-indol-3-yl)-ethyl]-amide;
5-(4-Fluoro-phenyl)-2-methyl-thiazole-4-carboxylic acid cyclopropylmethyl-[2-(5-fluoro-1H-indol-3-yl)-ethyl]-amide;
5-(4-Ethyl-phenyl)-2-methyl-thiazole-4-carboxylic acid cyclopropylmethyl-[2-(5-fluoro-1H-indol-3-yl)-ethyl]-amide;
5-(3-Chloro-phenyl)-2-methyl-thiazole-4-carboxylic acid cyclopropylmethyl-[2-(5-fluoro-1H-indol-3-yl)-ethyl]-amide;
2-Methyl-5-p-tolyl-thiazole-4-carboxylic acid cyclopropylmethyl-[2-(5-fluoro-1H-indol-3-yl)-ethyl]-amide;
5-(3,5-Dimethyl-phenyl)-2-methyl-thiazole-4-carboxylic acid cyclopropylmethyl-[2-(5-fluoro-1H-indol-3-yl)-ethyl]-amide;
5-(3-Cyano-phenyl)-2-methyl-thiazole-4-carboxylic acid cyclopropylmethyl-[2-(5-fluoro-1H-indol-3-yl)-ethyl]-amide;
5-(4-Chloro-phenyl)-2-methyl-thiazole-4-carboxylic acid cyclopropylmethyl-[2-(5-fluoro-1H-indol-3-yl)-ethyl]-amide;
5-(3,4-Difluoro-phenyl)-2-methyl-thiazole-4-carboxylic acid cyclopropylmethyl-[2-(5-fluoro-1H-indol-3-yl)-ethyl]-amide;
5-(3,4-Dichloro-phenyl)-2-methyl-thiazole-4-carboxylic acid cyclopropylmethyl-[2-(5-fluoro-1H-indol-3-yl)-ethyl]-amide;
5-(3-Fluoro-4-methyl-phenyl)-2-methyl-thiazole-4-carboxylic acid cyclopropylmethyl-[2-(5-fluoro-1H-indol-3-yl)-ethyl]-amide;
5-(2,3-Difluoro-4-methyl-phenyl)-2-methyl-thiazole-4-carboxylic acid cyclopropylmethyl-[2-(5-fluoro-1H-indol-3-yl)-ethyl]-amide;
5-(3,4-Dimethyl-phenyl)-thiazole-4-carboxylic acid cyclopropylmethyl-[2-(5-fluoro-1H-indol-3-yl)-ethyl]-amide;
2-Methoxy-5-m-tolyl-thiazole-4-carboxylic acid cyclopropylmethyl-[2-(5-fluoro-1H-indol-3-yl)-ethyl]-amide;
2-Cyclopropyl-5-(3-fluoro-4-methyl-phenyl)-thiazole-4-carboxylic acid cyclopropylmethyl-[2-(5-fluoro-1H-indol-3-yl)-ethyl]-amide;
2-Dimethylamino-5-m-tolyl-thiazole-4-carboxylic acid cyclopropylmethyl-[2-(5-fluoro-1H-indol-3-yl)-ethyl]-amide;
2-Dimethylamino-5-(3,4-dimethyl-phenyl)-thiazole-4-carboxylic acid cyclopropylmethyl-[2-(5-fluoro-1H-indol-3-yl)-ethyl]-amide;
2-Dimethylaminomethyl-5-m-tolyl-thiazole-4-carboxylic acid cyclopropylmethyl-[2-(5-fluoro-1H-indol-3-yl)-ethyl]-amide;
3-Phenyl-pyrazine-2-carboxylic acid cyclopropylmethyl-[2-(5-fluoro-1H-indol-3-yl)-ethyl]-amide;
3-Phenyl-pyrazine-2-carboxylic acid [2-(5-fluoro-1H-indol-3-yl)-ethyl]-methyl-amide;
3-Phenyl-pyrazine-2-carboxylic acid ethyl-[2-(5-fluoro-1H-indol-3-yl)-ethyl]-amide;
3-Phenyl-pyrazine-2-carboxylic acid [2-(5-fluoro-1H-indol-3-yl)-ethyl]-propyl-amide;
3-Phenyl-pyrazine-2-carboxylic acid [2-(5-fluoro-1H-indol-3-yl)-ethyl]-(2,2,2-trifluoro-ethyl)-amide;
3-Phenyl-pyrazine-2-carboxylic acid carbamoylmethyl-[2-(5-fluoro-1H-indol-3-yl)-ethyl]-amide;
[[2-(5-Fluoro-1H-indol-3-yl)-ethyl]-(3-phenyl-pyrazine-2-carbonyl)-amino]-acetic acid methyl ester;
3-Phenyl-pyrazine-2-carboxylic acid [2-(5-fluoro-1H-indol-3-yl)-ethyl]-isopropyl-amide;
3-Phenyl-pyrazine-2-carboxylic acid (2,2-difluoro-ethyl)-[2-(5-fluoro-1H-indol-3-yl)-ethyl]-amide;
3-Phenyl-pyrazine-2-carboxylic acid [2-(5-fluoro-1H-indol-3-yl)-ethyl]-(2-hydroxy-ethyl)-amide;
3-(3,4-Dimethyl-phenyl)-pyrazine-2-carboxylic acid [2-(5-fluoro-1H-indol-3-yl)-ethyl]-methyl-amide;
3-(3,4-Dimethyl-phenyl)-pyrazine-2-carboxylic acid ethyl-[2-(5-fluoro-1H-indol-3-yl)-ethyl]-amide;
3-(3,4-Dimethyl-phenyl)-pyrazine-2-carboxylic acid [2-(5-fluoro-1H-indol-3-yl)-ethyl]-propyl-amide;
3-(3,4-Dimethyl-phenyl)-pyrazine-2-carboxylic acid [2-(5-fluoro-1H-indol-3-yl)-ethyl]-(2,2,2-trifluoro-ethyl)-amide;
3-(3,4-Dimethyl-phenyl)-pyrazine-2-carboxylic acid carbamoylmethyl-[2-(5-fluoro-1H-indol-3-yl)-ethyl]-amide;
{[3-(3,4-Dimethyl-phenyl)-pyrazine-2-carbonyl]-[2-(5-fluoro-1H-indol-3-yl)-ethyl]-amino}-acetic acid methyl ester;
3-(3,4-Dimethyl-phenyl)-pyrazine-2-carboxylic acid [2-(5-fluoro-1H-indol-3-yl)-ethyl]-isopropyl-amide;
3-(3,4-Dimethyl-phenyl)-pyrazine-2-carboxylic acid (2,2-difluoro-ethyl)-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-methyl-amide;
5-(6-Methoxy-pyridin-3-yl)-2-methyl-thiazole-4-carboxylic acid ethyl-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-propyl-amide;
5-(6-Methoxy-pyridin-3-yl)-2-methyl-thiazole-4-carboxylic acid [2-(5-fluoro-1H-indol-3-yl)-ethyl]-(2,2,2-trifluoro-ethyl)-amide;
5-(6-Methoxy-pyridin-3-yl)-2-methyl-thiazole-4-carboxylic acid carbamoylmethyl-[2-(5-fluoro-1H-indol-3-yl)-ethyl]-amide;
5-(6-Methoxy-pyridin-3-yl)-2-methyl-thiazole-4-carboxylic acid dimethylcarbamoylmethyl-[2-(5-fluoro-1H-indol-3-yl)-ethyl]-amide;
5-(6-Methoxy-pyridin-3-yl)-2-methyl-thiazole-4-carboxylic acid (2-dimethylamino-ethyl)-[2-(5-fluoro-1H-indol-3-yl)-ethyl]-amide;
{[2-(5-Fluoro-1H-indol-3-yl)-ethyl]-[5-(6-methoxy-pyridin-3-yl)-2-methyl-thiazole-4-carbonyl]-amino}-acetic acid methyl ester;
5-(6-Methoxy-pyridin-3-yl)-2-methyl-thiazole-4-carboxylic acid [2-(5-fluoro-1H-indol-3-yl)-ethyl]-isopropyl-amide;
5-(6-Methoxy-pyridin-3-yl)-2-methyl-thiazole-4-carboxylic acid (2,2-difluoro-ethyl)-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-(2-hydroxy-ethyl)-amide;
6′-Methoxy-[3,3]bipyridinyl-2-carboxylic acid [2-(5-fluoro-1H-indol-3-yl)-ethyl]-methyl-amide;
6′-Methoxy-[3,3]bipyridinyl-2-carboxylic acid ethyl-[2-(5-fluoro-1H-indol-3-yl)-ethyl]-amide;
6′-Methoxy-[3,3]bipyridinyl-2-carboxylic acid [2-(5-fluoro-1H-indol-3-yl)-ethyl]-propyl-amide;
6′-Methoxy-[3,3]bipyridinyl-2-carboxylic acid [2-(5-fluoro-1H-indol-3-yl)-ethyl]-(2,2,2-trifluoro-ethyl)-amide;
6′-Methoxy-[3,3]bipyridinyl-2-carboxylic acid carbamoylmethyl-[2-(5-fluoro-1H-indol-3-yl)-ethyl]-amide;
6′-Methoxy-[3,3]bipyridinyl-2-carboxylic acid dimethylcarbamoylmethyl-[2-(5-fluoro-1H-indol-3-yl)-ethyl]-amide;
[[2-(5-Fluoro-1H-indol-3-yl)-ethyl]-(6′-methoxy-[3,3]bipyridinyl-2-carbonyl)-amino]-acetic acid methyl ester;
6′-Methoxy-[3,3]bipyridinyl-2-carboxylic acid [2-(5-fluoro-1H-indol-3-yl)-ethyl]-isopropyl-amide;
6′-Methoxy-[3,3]bipyridinyl-2-carboxylic acid (2,2-difluoro-ethyl)-[2-(5-fluoro-1H-indol-3-yl)-ethyl]-amide;
6′-Methoxy-[3,3]bipyridinyl-2-carboxylic acid [2-(5-fluoro-1H-indol-3-yl)-ethyl]-(2-hydroxy-ethyl)-amide;
3-Phenyl-pyrazine-2-carboxylic acid cyclopropylmethyl-[2-(1-methyl-1H-indol-3-yl)-ethyl]-amide;
3-Phenyl-pyrazine-2-carboxylic acid [2-(6-chloro-1H-indol-3-yl)-ethyl]-cyclopropylmethyl-amide;
3-Phenyl-pyrazine-2-carboxylic acid cyclopropylmethyl-[2-(7-methoxy-1H-indol-3-yl)-ethyl]-amide;
3-Phenyl-pyrazine-2-carboxylic acid cyclopropylmethyl-[2-(5-methoxy-1H-indol-3-yl)-ethyl]-amide;
3-Phenyl-pyrazine-2-carboxylic acid cyclopropylmethyl-[2-(6-methoxy-1H-indol-3-yl)-ethyl]-amide;
3-Phenyl-pyrazine-2-carboxylic acid cyclopropylmethyl-[2-(5-methyl-1H-indol-3-yl)-ethyl]-amide;
3-Phenyl-pyrazine-2-carboxylic acid cyclopropylmethyl-[2-(6-methyl-1H-indol-3-yl)-ethyl]-amide;
3-Phenyl-pyrazine-2-carboxylic acid cyclopropylmethyl-[2-(7-methyl-1H-indol-3-yl)-ethyl]-amide;
3-Phenyl-pyrazine-2-carboxylic acid cyclopropylmethyl-[2-(4-fluoro-1H-indol-3-yl)-ethyl]-amide;
3-Phenyl-pyrazine-2-carboxylic acid cyclopropylmethyl-[2-(6-fluoro-1H-indol-3-yl)-ethyl]-amide;
3-Phenyl-pyrazine-2-carboxylic acid cyclopropylmethyl-[2-(7-fluoro-1H-indol-3-yl)-ethyl]-amide;
3-(3,4-Dimethyl-phenyl)-pyrazine-2-carboxylic acid cyclopropylmethyl-[2-(1-methyl-1H-indol-3-yl)-ethyl]-amide;
3-(3,4-Dimethyl-phenyl)-pyrazine-2-carboxylic acid [2-(6-chloro-1H-indol-3-yl)-ethyl]-cyclopropylmethyl-amide;
3-(3,4-Dimethyl-phenyl)-pyrazine-2-carboxylic acid cyclopropylmethyl-[2-(7-methoxy-1H-indol-3-yl)-ethyl]-amide;
3-(3,4-Dimethyl-phenyl)-pyrazine-2-carboxylic acid cyclopropylmethyl-[2-(5-methoxy-1H-indol-3-yl)-ethyl]-amide;
3-(3,4-Dimethyl-phenyl)-pyrazine-2-carboxylic acid cyclopropylmethyl-[2-(6-methoxy-1H-indol-3-yl)-ethyl]-amide;
3-(3,4-Dimethyl-phenyl)-pyrazine-2-carboxylic acid cyclopropylmethyl-[2-(5-methyl-1H-indol-3-yl)-ethyl]-amide;
3-(3,4-Dimethyl-phenyl)-pyrazine-2-carboxylic acid cyclopropylmethyl-[2-(6-methyl-1H-indol-3-yl)-ethyl]-amide;
3-(3,4-Dimethyl-phenyl)-pyrazine-2-carboxylic acid cyclopropylmethyl-[2-(7-methyl-1H-indol-3-yl)-ethyl]-amide;
3-(3,4-Dimethyl-phenyl)-pyrazine-2-carboxylic acid cyclopropylmethyl-[2-(4-fluoro-1H-indol-3-yl)-ethyl]-amide;
3-(3,4-Dimethyl-phenyl)-pyrazine-2-carboxylic acid cyclopropylmethyl-[2-(6-fluoro-1H-indol-3-yl)-ethyl]-amide;
3-(3,4-Dimethyl-phenyl)-pyrazine-2-carboxylic acid cyclopropylmethyl-[2-(7-fluoro-1H-indol-3-yl)-ethyl]-amide;
5-(6-Methoxy-pyridin-3-yl)-2-methyl-thiazole-4-carboxylic acid cyclopropylmethyl-[2-(1-methyl-1H-indol-3-yl)-ethyl]-amide;
5-(6-Methoxy-pyridin-3-yl)-2-methyl-thiazole-4-carboxylic acid [2-(6-chloro-1H-indol-3-yl)-ethyl]-cyclopropylmethyl-amide;
5-(6-Methoxy-pyridin-3-yl)-2-methyl-thiazole-4-carboxylic acid cyclopropylmethyl-[2-(7-methoxy-1H-indol-3-yl)-ethyl]-amide;
5-(6-Methoxy-pyridin-3-yl)-2-methyl-thiazole-4-carboxylic acid cyclopropylmethyl-[2-(5-methoxy-1H-indol-3-yl)-ethyl]-amide;
5-(6-Methoxy-pyridin-3-yl)-2-methyl-thiazole-4-carboxylic acid cyclopropylmethyl-[2-(6-methoxy-1H-indol-3-yl)-ethyl]-amide;
5-(6-Methoxy-pyridin-3-yl)-2-methyl-thiazole-4-carboxylic acid cyclopropylmethyl-[2-(5-methyl-1H-indol-3-yl)-ethyl]-amide;
5-(6-Methoxy-pyridin-3-yl)-2-methyl-thiazole-4-carboxylic acid cyclopropylmethyl-[2-(6-methyl-1H-indol-3-yl)-ethyl]-amide;
5-(6-Methoxy-pyridin-3-yl)-2-methyl-thiazole-4-carboxylic acid cyclopropylmethyl-[2-(7-methyl-1H-indol-3-yl)-ethyl]-amide;
5-(6-Methoxy-pyridin-3-yl)-2-methyl-thiazole-4-carboxylic acid cyclopropylmethyl-[2-(4-fluoro-1H-indol-3-yl)-ethyl]-amide;
5-(6-Methoxy-pyridin-3-yl)-2-methyl-thiazole-4-carboxylic acid cyclopropylmethyl-[2-(6-fluoro-1H-indol-3-yl)-ethyl]-amide;
5-(6-Methoxy-pyridin-3-yl)-2-methyl-thiazole-4-carboxylic acid cyclopropylmethyl-[2-(7-fluoro-1H-indol-3-yl)-ethyl]-amide;
6′-Methoxy-[3,3]bipyridinyl-2-carboxylic acid cyclopropylmethyl-[2-(1-methyl-1H-indol-3-yl)-ethyl]-amide;
6′-Methoxy-[3,3]bipyridinyl-2-carboxylic acid [2-(6-chloro-1H-indol-3-yl)-ethyl]-cyclopropylmethyl-amide;
6′-Methoxy-[3,3]bipyridinyl-2-carboxylic acid cyclopropylmethyl-[2-(7-methoxy-1H-indol-3-yl)-ethyl]-amide;
6′-Methoxy-[3,3]bipyridinyl-2-carboxylic acid cyclopropylmethyl-[2-(5-methoxy-1H-indol-3-yl)-ethyl]-amide;
6′-Methoxy-[3,3]bipyridinyl-2-carboxylic acid cyclopropylmethyl-[2-(6-methoxy-1H-indol-3-yl)-ethyl]-amide;
6′-Methoxy-[3,3]bipyridinyl-2-carboxylic acid cyclopropylmethyl-[2-(5-methyl-1H-indol-3-yl)-ethyl]-amide;
6′-Methoxy-[3,3]bipyridinyl-2-carboxylic acid cyclopropylmethyl-[2-(6-methyl-1H-indol-3-yl)-ethyl]-amide;
6′-Methoxy-[3,3]bipyridinyl-2-carboxylic acid cyclopropylmethyl-[2-(7-methyl-1H-indol-3-yl)-ethyl]-amide;
6′-Methoxy-[3,3]bipyridinyl-2-carboxylic acid cyclopropylmethyl-[2-(4-fluoro-1H-indol-3-yl)-ethyl]-amide;
6′-Methoxy-[3,3]bipyridinyl-2-carboxylic acid cyclopropylmethyl-[2-(6-fluoro-1H-indol-3-yl)-ethyl]-amide;
6′-Methoxy-[3,3]bipyridinyl-2-carboxylic acid cyclopropylmethyl-[2-(7-fluoro-1H-indol-3-yl)-ethyl]-amide;
3-Phenyl-pyrazine-2-carboxylic acid cyclopropylmethyl-[2-(6-methoxy-1H-benzoimidazol-2-yl)-ethyl]-amide;
3-m-Tolyl-pyrazine-2-carboxylic acid cyclopropylmethyl-[2-(6-methoxy-1H-benzoimidazol-2-yl)-ethyl]-amide;
3-(3,4-Dimethyl-phenyl)-pyrazine-2-carboxylic acid cyclopropylmethyl-[2-(6-methoxy-1H-benzoimidazol-2-yl)-ethyl]-amide;
2-Methyl-5-m-tolyl-thiazole-4-carboxylic acid cyclopropylmethyl-[2-(6-methoxy-1H-benzoimidazol-2-yl)-ethyl]-amide;
2-Dimethylamino-5-(3-methoxy-phenyl)-thiazole-4-carboxylic acid cyclopropylmethyl-[2-(6-methoxy-1H-benzoimidazol-2-yl)-ethyl]-amide;
2-Dimethylamino-5-(3,4-dimethyl-phenyl)-thiazole-4-carboxylic acid cyclopropylmethyl-[2-(6-methoxy-1H-benzoimidazol-2-yl)-ethyl]-amide;
2-Dimethylamino-5-m-tolyl-thiazole-4-carboxylic acid cyclopropylmethyl-[2-(6-methoxy-1H-benzoimidazol-2-yl)-ethyl]-amide;
5-(6-Methoxy-pyridin-3-yl)-2-methyl-thiazole-4-carboxylic acid cyclopropylmethyl-[2-(6-methoxy-1H-benzoimidazol-2-yl)-ethyl]-amide;
6′-Methoxy-[3,3]bipyridinyl-2-carboxylic acid cyclopropylmethyl-[2-(6-methoxy-1H-benzoimidazol-2-yl)-ethyl]-amide;
3-Phenyl-pyrazine-2-carboxylic acid cyclopropylmethyl-[2-(5,6-dimethyl-1H-benzoimidazol-2-yl)-ethyl]-amide;
3-m-Tolyl-pyrazine-2-carboxylic acid cyclopropylmethyl-[2-(5,6-dimethyl-1H-benzoimidazol-2-yl)-ethyl]-amide;
3-(3,4-Dimethyl-phenyl)-pyrazine-2-carboxylic acid cyclopropylmethyl-[2-(5,6-dimethyl-1H-benzoimidazol-2-yl)-ethyl]-amide;
2-Methyl-5-m-tolyl-thiazole-4-carboxylic acid cyclopropylmethyl-[2-(5,6-dimethyl-1H-benzoimidazol-2-yl)-ethyl]-amide;
2-Dimethylamino-5-(3-methoxy-phenyl)-thiazole-4-carboxylic acid cyclopropylmethyl-[2-(5,6-dimethyl-1H-benzoimidazol-2-yl)-ethyl]-amide;
2-Dimethylamino-5-(3,4-dimethyl-phenyl)-thiazole-4-carboxylic acid cyclopropylmethyl-[2-(5,6-dimethyl-1H-benzoimidazol-2-yl)-ethyl]-amide;
2-Dimethylamino-5-m-tolyl-thiazole-4-carboxylic acid cyclopropylmethyl-[2-(5,6-dimethyl-1H-benzoimidazol-2-yl)-ethyl]-amide;
5-(6-Methoxy-pyridin-3-yl)-2-methyl-thiazole-4-carboxylic acid cyclopropylmethyl-[2-(5,6-dimethyl-1H-benzoimidazol-2-yl)-ethyl]-amide;
6′-Methoxy-[3,3]bipyridinyl-2-carboxylic acid cyclopropylmethyl-[2-(5,6-dimethyl-1H-benzoimidazol-2-yl)-ethyl]-amide;
5-(6-Methoxy-pyridin-3-yl)-2-methyl-thiazole-4-carboxylic acid cyclopropylmethyl-[2-(5-methoxy-4-methyl-1H-indol-3-yl)-ethyl]-amide;
5-(6-Methoxy-pyridin-3-yl)-2-methyl-thiazole-4-carboxylic acid cyclopropylmethyl-[2-(5H-[1,3]dioxolo[4,5-f]indol-7-yl)-ethyl]-amide;
5-(6-Methoxy-pyridin-3-yl)-2-methyl-thiazole-4-carboxylic acid cyclopropylmethyl-[2-(5,6-difluoro-1H-indol-3-yl)-ethyl]-amide;
5-(6-Methoxy-pyridin-3-yl)-2-methyl-thiazole-4-carboxylic acid [2-(5-chloro-6-fluoro-1H-indol-3-yl)-ethyl]-cyclopropylmethyl-amide;
5-(6-Methoxy-pyridin-3-yl)-2-methyl-thiazole-4-carboxylic acid cyclopropylmethyl-[2-(5-methoxy-1H-indol-3-yl)-1-methyl-ethyl]-amide;
3-m-Tolyl-pyridine-2-carboxylic acid cyclopropylmethyl-[2-(5-fluoro-1H-indol-3-yl)-ethyl]-amide;
3-(3,4-Dimethyl-phenyl)-pyridine-2-carboxylic acid cyclopropylmethyl-[2-(5-fluoro-1H-indol-3-yl)-ethyl]-amide;
6′-Methoxy-[3,3]bipyridinyl-2-carboxylic acid cyclopropylmethyl-[2-(5-fluoro-1H-indol-3-yl)-ethyl]-amide;
6′-Fluoro-[3,3]bipyridinyl-2-carboxylic acid cyclopropylmethyl-[2-(5-fluoro-1H-indol-3-yl)-ethyl]-amide;
5′-Methyl-[3,3]bipyridinyl-2-carboxylic acid cyclopropylmethyl-[2-(5-fluoro-1H-indol-3-yl)-ethyl]-amide;
5′-Chloro-2′-fluoro-[3,3]bipyridinyl-2-carboxylic acid cyclopropylmethyl-[2-(5-fluoro-1H-indol-3-yl)-ethyl]-amide;
3-Quinolin-3-yl-pyridine-2-carboxylic acid cyclopropylmethyl-[2-(5-fluoro-1H-indol-3-yl)-ethyl]-amide;
6′-Methyl-[3,3]bipyridinyl-2-carboxylic acid cyclopropylmethyl-[2-(5-fluoro-1H-indol-3-yl)-ethyl]-amide;
5′-Methoxy-[3,3]bipyridinyl-2-carboxylic acid cyclopropylmethyl-[2-(5-fluoro-1H-indol-3-yl)-ethyl]-amide;
5-(3-Chloro-4-methyl-phenyl)-2-methyl-thiazole-4-carboxylic acid cyclopropylmethyl-[2-(5-fluoro-1H-indol-3-yl)-ethyl]-amide;
5-(3-Chloro-4-methoxy-phenyl)-2-methyl-thiazole-4-carboxylic acid cyclopropylmethyl-[2-(5-fluoro-1H-indol-3-yl)-ethyl]-amide;
2-Methyl-5-(6-methyl-pyridin-3-yl)-thiazole-4-carboxylic acid cyclopropylmethyl-[2-(5-fluoro-1H-indol-3-yl)-ethyl]-amide;
5-(4-Methoxy-3-methyl-phenyl)-2-methyl-thiazole-4-carboxylic acid cyclopropylmethyl-[2-(5-fluoro-1H-indol-3-yl)-ethyl]-amide;
5-(3-Chloro-4-fluoro-phenyl)-2-methyl-thiazole-4-carboxylic acid cyclopropylmethyl-[2-(5-fluoro-1H-indol-3-yl)-ethyl]-amide;
5-(4-Fluoro-3-methyl-phenyl)-2-methyl-thiazole-4-carboxylic acid cyclopropylmethyl-[2-(5-fluoro-1H-indol-3-yl)-ethyl]-amide;
5-(3-Fluoro-4-methoxy-phenyl)-2-methyl-thiazole-4-carboxylic acid cyclopropylmethyl-[2-(5-fluoro-1H-indol-3-yl)-ethyl]-amide;
5-(4-Chloro-3-fluoro-phenyl)-2-methyl-thiazole-4-carboxylic acid cyclopropylmethyl-[2-(5-fluoro-1H-indol-3-yl)-ethyl]-amide;
5-(3-Cyano-4-fluoro-phenyl)-2-methyl-thiazole-4-carboxylic acid cyclopropylmethyl-[2-(5-fluoro-1H-indol-3-yl)-ethyl]-amide;
5-(4-Fluoro-3-methoxy-phenyl)-2-methyl-thiazole-4-carboxylic acid cyclopropylmethyl-[2-(5-fluoro-1H-indol-3-yl)-ethyl]-amide;
5-(4-Chloro-3-cyano-phenyl)-2-methyl-thiazole-4-carboxylic acid cyclopropylmethyl-[2-(5-fluoro-1H-indol-3-yl)-ethyl]-amide;
5-(4-Fluoro-3-hydroxymethyl-phenyl)-2-methyl-thiazole-4-carboxylic acid cyclopropylmethyl-[2-(5-fluoro-1H-indol-3-yl)-ethyl]-amide;
5-(4-Cyano-3-fluoro-phenyl)-2-methyl-thiazole-4-carboxylic acid cyclopropylmethyl-[2-(5-fluoro-1H-indol-3-yl)-ethyl]-amide;
5-(3-Chloro-2-methoxy-pyridin-4-yl)-2-methyl-thiazole-4-carboxylic acid cyclopropylmethyl-[2-(5-fluoro-1H-indol-3-yl)-ethyl]-amide;
5-(6-Methoxy-pyridin-3-yl)-2-methyl-thiazole-4-carboxylic acid cyclopropylmethyl-[2-(5-fluoro-1H-indol-3-yl)-ethyl]-amide;
5-(6-Fluoro-pyridin-3-yl)-2-methyl-thiazole-4-carboxylic acid cyclopropylmethyl-[2-(5-fluoro-1H-indol-3-yl)-ethyl]-amide;
5-(6-Hydroxymethyl-pyridin-3-yl)-2-methyl-thiazole-4-carboxylic acid cyclopropylmethyl-[2-(5-fluoro-1H-indol-3-yl)-ethyl]-amide;
2-Methyl-5-(5-methylsulfanyl-pyridin-3-yl)-thiazole-4-carboxylic acid cyclopropylmethyl-[2-(5-fluoro-1H-indol-3-yl)-ethyl]-amide;
5-(5-Fluoro-pyridin-3-yl)-2-methyl-thiazole-4-carboxylic acid cyclopropylmethyl-[2-(5-fluoro-1H-indol-3-yl)-ethyl]-amide;
2-Methyl-5-(5-methyl-pyridin-3-yl)-thiazole-4-carboxylic acid cyclopropylmethyl-[2-(5-fluoro-1H-indol-3-yl)-ethyl]-amide;
5-(5-Chloro-2-fluoro-pyridin-3-yl)-2-methyl-thiazole-4-carboxylic acid cyclopropylmethyl-[2-(5-fluoro-1H-indol-3-yl)-ethyl]-amide;
2-Methyl-5-quinolin-3-yl-thiazole-4-carboxylic acid cyclopropylmethyl-[2-(5-fluoro-1H-indol-3-yl)-ethyl]-amide;
5-(1H-Indol-5-yl)-2-methyl-thiazole-4-carboxylic acid cyclopropylmethyl-[2-(5-fluoro-1H-indol-3-yl)-ethyl]-amide;
5-(1H-Indol-6-yl)-2-methyl-thiazole-4-carboxylic acid cyclopropylmethyl-[2-(5-fluoro-1H-indol-3-yl)-ethyl]-amide;
2-Methyl-5-(1-methyl-1H-indol-2-yl)-thiazole-4-carboxylic acid cyclopropylmethyl-[2-(5-fluoro-1H-indol-3-yl)-ethyl]-amide;
2-Aminomethyl-5-m-tolyl-thiazole-4-carboxylic acid cyclopropylmethyl-[2-(5-fluoro-1H-indol-3-yl)-ethyl]-amide;
3-(3,4-Dimethyl-phenyl)-pyrazine-2-carboxylic acid (2-amino-ethyl)-[2-(5-fluoro-1H-indol-3-yl)-ethyl]-amide;
2-Methylamino-5-m-tolyl-thiazole-4-carboxylic acid cyclopropylmethyl-[2-(5-fluoro-1H-indol-3-yl)-ethyl]-amide;
3-(4-Methoxy-phenyl)-pyrazine-2-carboxylic acid cyclopropylmethyl-[2-(7-methyl-1H-indol-3-yl)-ethyl]-amide;
3-(6-Methoxy-pyridin-3-yl)-pyrazine-2-carboxylic acid cyclopropylmethyl-[2-(7-methyl-1H-indol-3-yl)-ethyl]-amide;
3-Pyrimidin-5-yl-pyrazine-2-carboxylic acid cyclopropylmethyl-[2-(7-methyl-1H-indol-3-yl)-ethyl]-amide; and
3-(2-Methoxy-pyrimidin-5-yl)-pyrazine-2-carboxylic acid cyclopropylmethyl-[2-(7-methyl-1H-indol-3-yl)-ethyl]-amide;
   wherein it is well understood that any stereogenic center of the above listed compounds may be in absolute (R)- or (S)-configuration.

lxvi) Further preferred compounds of formula (I) according to embodiment i) are selected from the group consisting of:
3-(4-Fluoro-phenyl)-pyrazine-2-carboxylic acid cyclopropylmethyl-[2-(7-methoxy-1H-indol-3-yl)-ethyl]-amide;
3-(4-Fluoro-3-methyl-phenyl)-pyrazine-2-carboxylic acid cyclopropylmethyl-[2-(7-methoxy-1H-indol-3-yl)-ethyl]-amide;
3-(2-Fluoro-5-methoxy-phenyl)-pyrazine-2-carboxylic acid cyclopropylmethyl-[2-(7-methoxy-1H-indol-3-yl)-ethyl]-amide;
3-(3-Fluoro-5-methyl-phenyl)-pyrazine-2-carboxylic acid cyclopropylmethyl-[2-(7-methoxy-1H-indol-3-yl)-ethyl]-amide;
3-(3-Trifluoromethyl-phenyl)-pyrazine-2-carboxylic acid cyclopropylmethyl-[2-(7-methoxy-1H-indol-3-yl)-ethyl]-amide;
3-(2,3-Dimethyl-phenyl)-pyrazine-2-carboxylic acid cyclopropylmethyl-[2-(7-methoxy-1H-indol-3-yl)-ethyl]-amide;
3-(3-Methoxy-phenyl)-pyrazine-2-carboxylic acid cyclopropylmethyl-[2-(7-methoxy-1H-indol-3-yl)-ethyl]-amide;
3-m-Tolyl-pyrazine-2-carboxylic acid cyclopropylmethyl-[2-(7-methoxy-1H-indol-3-yl)-ethyl]-amide;
3-(4-Fluoro-phenyl)-pyrazine-2-carboxylic acid cyclopropylmethyl-[2-(5-fluoro-1H-indol-3-yl)-ethyl]-amide;
3-(4-Fluoro-3-methyl-phenyl)-pyrazine-2-carboxylic acid cyclopropylmethyl-[2-(5-fluoro-1H-indol-3-yl)-ethyl]-amide;
3-(2-Fluoro-5-methoxy-phenyl)-pyrazine-2-carboxylic acid cyclopropylmethyl-[2-(5-fluoro-1H-indol-3-yl)-ethyl]-amide;
3-(3-Fluoro-5-methyl-phenyl)-pyrazine-2-carboxylic acid cyclopropylmethyl-[2-(5-fluoro-1H-indol-3-yl)-ethyl]-amide;
3-(3-Trifluoromethyl-phenyl)-pyrazine-2-carboxylic acid cyclopropylmethyl-[2-(5-fluoro-1H-indol-3-yl)-ethyl]-amide;
3-(2,3-Dimethyl-phenyl)-pyrazine-2-carboxylic acid cyclopropylmethyl-[2-(5-fluoro-1H-indol-3-yl)-ethyl]-amide;
2-Methyl-4-phenyl-pyrimidine-5-carboxylic acid cyclopropylmethyl-[2-(5-fluoro-1H-indol-3-yl)-ethyl]-amide;
4-Phenyl-pyrimidine-5-carboxylic acid cyclopropylmethyl-[2-(5-fluoro-1H-indol-3-yl)-ethyl]-amide;
3-(4-Fluoro-phenyl)-pyrazine-2-carboxylic acid cyclopropylmethyl-[2-(5,6-difluoro-1H-indol-3-yl)-ethyl]-amide;
3-(4-Fluoro-3-methyl-phenyl)-pyrazine-2-carboxylic acid cyclopropylmethyl-[2-(5,6-difluoro-1H-indol-3-yl)-ethyl]-amide;
3-(2-Fluoro-5-methoxy-phenyl)-pyrazine-2-carboxylic acid cyclopropylmethyl-[2-(5,6-difluoro-1H-indol-3-yl)-ethyl]-amide;
3-(3-Fluoro-5-methyl-phenyl)-pyrazine-2-carboxylic acid cyclopropylmethyl-[2-(5,6-difluoro-1H-indol-3-yl)-ethyl]-amide;
3-(3-Trifluoromethyl-phenyl)-pyrazine-2-carboxylic acid cyclopropylmethyl-[2-(5,6-difluoro-1H-indol-3-yl)-ethyl]-amide;
3-(2,3-Dimethyl-phenyl)-pyrazine-2-carboxylic acid cyclopropylmethyl-[2-(5,6-difluoro-1H-indol-3-yl)-ethyl]-amide;
2-Methyl-4-phenyl-pyrimidine-5-carboxylic acid cyclopropylmethyl-[2-(5,6-difluoro-1H-indol-3-yl)-ethyl]-amide;
4-Phenyl-pyrimidine-5-carboxylic acid cyclopropylmethyl-[2-(5,6-difluoro-1H-indol-3-yl)-ethyl]-amide;
3-(4-Fluoro-phenyl)-pyrazine-2-carboxylic acid [2-(5-chloro-6-fluoro-1H-indol-3-yl)-ethyl]-cyclopropylmethyl-amide;
3-(4-Fluoro-3-methyl-phenyl)-pyrazine-2-carboxylic acid [2-(5-chloro-6-fluoro-1H-indol-3-yl)-ethyl]-cyclopropylmethyl-amide;
3-(2-Fluoro-5-methoxy-phenyl)-pyrazine-2-carboxylic acid [2-(5-chloro-6-fluoro-1H-indol-3-yl)-ethyl]-cyclopropylmethyl-amide;
3-(3-Fluoro-5-methyl-phenyl)-pyrazine-2-carboxylic acid [2-(5-chloro-6-fluoro-1H-indol-3-yl)-ethyl]-cyclopropylmethyl-amide;
3-(3-Trifluoromethyl-phenyl)-pyrazine-2-carboxylic acid [2-(5-chloro-6-fluoro-1H-indol-3-yl)-ethyl]-cyclopropylmethyl-amide;
3-(2,3-Dimethyl-phenyl)-pyrazine-2-carboxylic acid [2-(5-chloro-6-fluoro-1H-indol-3-yl)-ethyl]-cyclopropylmethyl-amide;
2-Methyl-4-phenyl-pyrimidine-5-carboxylic acid [2-(5-chloro-6-fluoro-1H-indol-3-yl)-ethyl]-cyclopropylmethyl-amide;
4-Phenyl-pyrimidine-5-carboxylic acid [2-(5-chloro-6-fluoro-1H-indol-3-yl)-ethyl]-cyclopropylmethyl-amide;
2-Dimethylamino-5-phenyl-thiazole-4-carboxylic acid [2-(5-chloro-6-fluoro-1H-indol-3-yl)-ethyl]-cyclopropylmethyl-amide;
2-Dimethylamino-5-m-tolyl-thiazole-4-carboxylic acid [2-(5-chloro-6-fluoro-1H-indol-3-yl)-ethyl]-cyclopropylmethyl-amide;
2-Dimethylamino-5-(3-fluoro-4-methyl-phenyl)-thiazole-4-carboxylic acid [2-(5-chloro-6-fluoro-1H-indol-3-yl)-ethyl]-cyclopropylmethyl-amide;
2-Dimethylamino-5-(4-fluoro-phenyl)-thiazole-4-carboxylic acid [2-(5-chloro-6-fluoro-1H-indol-3-yl)-ethyl]-cyclopropylmethyl-amide;
3-(4-Fluoro-phenyl)-pyrazine-2-carboxylic acid cyclopropylmethyl-[2-(5-methoxy-4-methyl-1H-indol-3-yl)-ethyl]-amide;
3-(4-Fluoro-3-methyl-phenyl)-pyrazine-2-carboxylic acid cyclopropylmethyl-[2-(5-methoxy-4-methyl-1H-indol-3-yl)-ethyl]-amide;
3-(3-Fluoro-5-methyl-phenyl)-pyrazine-2-carboxylic acid cyclopropylmethyl-[2-(5-methoxy-4-methyl-1H-indol-3-yl)-ethyl]-amide;
2-Methyl-4-phenyl-pyrimidine-5-carboxylic acid cyclopropylmethyl-[2-(5-methoxy-4-methyl-1H-indol-3-yl)-ethyl]-amide;
4-Phenyl-pyrimidine-5-carboxylic acid cyclopropylmethyl-[2-(5-methoxy-4-methyl-1H-indol-3-yl)-ethyl]-amide;
2-Dimethylamino-5-phenyl-thiazole-4-carboxylic acid cyclopropylmethyl-[2-(5-methoxy-4-methyl-1H-indol-3-yl)-ethyl]-amide;
2-Dimethylamino-5-m-tolyl-thiazole-4-carboxylic acid cyclopropylmethyl-[2-(5-methoxy-4-methyl-1H-indol-3-yl)-ethyl]-amide;
2-Dimethylamino-5-(3-fluoro-4-methyl-phenyl)-thiazole-4-carboxylic acid cyclopropylmethyl-[2-(5-methoxy-4-methyl-1H-indol-3-yl)-ethyl]-amide;
2-Dimethylamino-5-(4-fluoro-phenyl)-thiazole-4-carboxylic acid cyclopropylmethyl-[2-(5-methoxy-4-methyl-1H-indol-3-yl)-ethyl]-amide;
2-Dimethylamino-5-(3-fluoro-phenyl)-thiazole-4-carboxylic acid cyclopropylmethyl-[2-(7-fluoro-1H-indol-3-yl)-ethyl]-amide;
2-Dimethylamino-5-(3-fluoro-phenyl)-thiazole-4-carboxylic acid cyclopropylmethyl-[2-(4-fluoro-1H-indol-3-yl)-ethyl]-amide;
2-Dimethylamino-5-(3-fluoro-phenyl)-thiazole-4-carboxylic acid cyclopropylmethyl-[2-(6-fluoro-1H-indol-3-yl)-ethyl]-amide;
3-(4-Fluoro-phenyl)-pyrazine-2-carboxylic acid cyclopropylmethyl-[2-(1H-indol-3-yl)-ethyl]-amide;
3-(4-Fluoro-3-methyl-phenyl)-pyrazine-2-carboxylic acid cyclopropylmethyl-[2-(1H-indol-3-yl)-ethyl]-amide;
3-(2-Fluoro-5-methoxy-phenyl)-pyrazine-2-carboxylic acid cyclopropylmethyl-[2-(1H-indol-3-yl)-ethyl]-amide;
3-(3-Fluoro-5-methyl-phenyl)-pyrazine-2-carboxylic acid cyclopropylmethyl-[2-(1H-indol-3-yl)-ethyl]-amide;
3-(3-Trifluoromethyl-phenyl)-pyrazine-2-carboxylic acid cyclopropylmethyl-[2-(1H-indol-3-yl)-ethyl]-amide;
3-(2,3-Dimethyl-phenyl)-pyrazine-2-carboxylic acid cyclopropylmethyl-[2-(1H-indol-3-yl)-ethyl]-amide;
3-(3-Methoxy-phenyl)-pyrazine-2-carboxylic acid cyclopropylmethyl-[2-(1H-indol-3-yl)-ethyl]-amide;
3-m-Tolyl-pyrazine-2-carboxylic acid cyclopropylmethyl-[2-(1H-indol-3-yl)-ethyl]-amide;
2-Methyl-4-phenyl-pyrimidine-5-carboxylic acid cyclopropylmethyl-[2-(1H-indol-3-yl)-ethyl]-amide;
4-Phenyl-pyrimidine-5-carboxylic acid cyclopropylmethyl-[2-(1H-indol-3-yl)-ethyl]-amide;
3-(3,4-Dimethyl-phenyl)-pyrazine-2-carboxylic acid cyclopropylmethyl-[2-(1H-indol-3-yl)-ethyl]-amide;
3-Phenyl-pyrazine-2-carboxylic acid cyclopropylmethyl-[2-(1H-indol-3-yl)-ethyl]-amide;
2-(Ethyl-methyl-amino)-5-(2-fluoro-phenyl)-thiazole-4-carboxylic acid cyclopropylmethyl-[2-(1H-indol-3-yl)-ethyl]-amide;
2-Methyl-5-(4-propionylamino-phenyl)-thiazole-4-carboxylic acid cyclopropylmethyl-[2-(1H-indol-3-yl)-ethyl]-amide;
4-(3-Chloro-phenyl)-pyrimidine-5-carboxylic acid cyclopropylmethyl-[2-(1H-indol-3-yl)-ethyl]-amide;
4-(3-Chloro-phenyl)-2-methyl-pyrimidine-5-carboxylic acid cyclopropylmethyl-[2-(1H-indol-3-yl)-ethyl]-amide;
4-(3,4-Dimethyl-phenyl)-pyrimidine-5-carboxylic acid cyclopropylmethyl-[2-(1H-indol-3-yl)-ethyl]-amide;
4-(3,4-Dimethyl-phenyl)-2-methyl-pyrimidine-5-carboxylic acid cyclopropylmethyl-[2-(1H-indol-3-yl)-ethyl]-amide;
4-(3-Methoxy-phenyl)-pyrimidine-5-carboxylic acid cyclopropylmethyl-[2-(1H-indol-3-yl)-ethyl]-amide;
4-(3-Methoxy-phenyl)-2-methyl-pyrimidine-5-carboxylic acid cyclopropylmethyl-[2-(1H-indol-3-yl)-ethyl]-amide;
4-(3,4-Dichloro-phenyl)-pyrimidine-5-carboxylic acid cyclopropylmethyl-[2-(1H-indol-3-yl)-ethyl]-amide;
4-(3,4-Dichloro-phenyl)-2-methyl-pyrimidine-5-carboxylic acid cyclopropylmethyl-[2-(1H-indol-3-yl)-ethyl]-amide;
4-(3-Fluoro-phenyl)-pyrimidine-5-carboxylic acid cyclopropylmethyl-[2-(1H-indol-3-yl)-ethyl]-amide;
4-(3-Fluoro-phenyl)-2-methyl-pyrimidine-5-carboxylic acid cyclopropylmethyl-[2-(1H-indol-3-yl)-ethyl]-amide;
4-(4-Bromo-3-chloro-phenyl)-2-methyl-pyrimidine-5-carboxylic acid cyclopropylmethyl-[2-(1H-indol-3-yl)-ethyl]-amide;
4-(4-Bromo-3-chloro-phenyl)-pyrimidine-5-carboxylic acid cyclopropylmethyl-[2-(1H-indol-3-yl)-ethyl]-amide;
4-m-Tolyl-pyrimidine-5-carboxylic acid cyclopropylmethyl-[2-(1H-indol-3-yl)-ethyl]-amide;
2-Methyl-4-m-tolyl-pyrimidine-5-carboxylic acid cyclopropylmethyl-[2-(1H-indol-3-yl)-ethyl]-amide;
2-Methyl-4-p-tolyl-pyrimidine-5-carboxylic acid cyclopropylmethyl-[2-(1H-indol-3-yl)-ethyl]-amide;
4-p-Tolyl-pyrimidine-5-carboxylic acid cyclopropylmethyl-[2-(1H-indol-3-yl)-ethyl]-amide;
4-(4-Fluoro-phenyl)-pyrimidine-5-carboxylic acid cyclopropylmethyl-[2-(1H-indol-3-yl)-ethyl]-amide;
4-(4-Fluoro-phenyl)-2-methyl-pyrimidine-5-carboxylic acid cyclopropylmethyl-[2-(1H-indol-3-yl)-ethyl]-amide;
3-Phenyl-pyrazine-2-carboxylic acid ethyl-[2-(1H-indol-3-yl)-ethyl]-amide;
4-Phenyl-pyrimidine-5-carboxylic acid ethyl-[2-(1H-indol-3-yl)-ethyl]-amide;
2-Methyl-4-phenyl-pyrimidine-5-carboxylic acid ethyl-[2-(1H-indol-3-yl)-ethyl]-amide;
3-m-Tolyl-pyrazine-2-carboxylic acid ethyl-[2-(1H-indol-3-yl)-ethyl]-amide;
4-m-Tolyl-pyrimidine-5-carboxylic acid ethyl-[2-(1H-indol-3-yl)-ethyl]-amide;
2-Methyl-4-m-tolyl-pyrimidine-5-carboxylic acid ethyl-[2-(1H-indol-3-yl)-ethyl]-amide;
3-(4-Fluoro-phenyl)-pyrazine-2-carboxylic acid ethyl-[2-(1H-indol-3-yl)-ethyl]-amide;
4-(4-Fluoro-phenyl)-pyrimidine-5-carboxylic acid ethyl-[2-(1H-indol-3-yl)-ethyl]-amide;
4-(4-Fluoro-phenyl)-2-methyl-pyrimidine-5-carboxylic acid ethyl-[2-(1H-indol-3-yl)-ethyl]-amide;
3-(4-Fluoro-3-methyl-phenyl)-pyrazine-2-carboxylic acid ethyl-[2-(1H-indol-3-yl)-ethyl]-amide;
4-(3-Fluoro-phenyl)-pyrimidine-5-carboxylic acid ethyl-[2-(1H-indol-3-yl)-ethyl]-amide;
4-(3-Fluoro-phenyl)-2-methyl-pyrimidine-5-carboxylic acid ethyl-[2-(1H-indol-3-yl)-ethyl]-amide;
3-(3-Fluoro-5-methyl-phenyl)-pyrazine-2-carboxylic acid ethyl-[2-(1H-indol-3-yl)-ethyl]-amide;
3-(3-Methoxy-phenyl)-pyrazine-2-carboxylic acid ethyl-[2-(1H-indol-3-yl)-ethyl]-amide;
3-(3,4-Dimethyl-phenyl)-pyrazine-2-carboxylic acid ethyl-[2-(1H-indol-3-yl)-ethyl]-amide;
4-(4-Bromo-3-chloro-phenyl)-2-methyl-pyrimidine-5-carboxylic acid ethyl-[2-(1H-indol-3-yl)-ethyl]-amide;
4-(4-Bromo-3-chloro-phenyl)-pyrimidine-5-carboxylic acid ethyl-[2-(1H-indol-3-yl)-ethyl]-amide;
2-Methyl-4-p-tolyl-pyrimidine-5-carboxylic acid ethyl-[2-(1H-indol-3-yl)-ethyl]-amide;
4-p-Tolyl-pyrimidine-5-carboxylic acid ethyl-[2-(1H-indol-3-yl)-ethyl]-amide;
4-(3,5-Dichloro-phenyl)-2-methyl-pyrimidine-5-carboxylic acid ethyl-[2-(1H-indol-3-yl)-ethyl]-amide;
4-(3,5-Dichloro-phenyl)-pyrimidine-5-carboxylic acid ethyl-[2-(1H-indol-3-yl)-ethyl]-amide;
4-(3-Methoxy-phenyl)-pyrimidine-5-carboxylic acid ethyl-[2-(1H-indol-3-yl)-ethyl]-amide;
4-(3,4-Dimethyl-phenyl)-2-methyl-pyrimidine-5-carboxylic acid ethyl-[2-(1H-indol-3-yl)-ethyl]-amide;
4-(3,4-Dimethyl-phenyl)-pyrimidine-5-carboxylic acid ethyl-[2-(1H-indol-3-yl)-ethyl]-amide;
4-(3,4-Dichloro-phenyl)-pyrimidine-5-carboxylic acid ethyl-[2-(1H-indol-3-yl)-ethyl]-amide;
3-Phenyl-pyrazine-2-carboxylic acid [2-(1H-indol-3-yl)-ethyl]-(2,2,2-trifluoro-ethyl)-amide;
4-Phenyl-pyrimidine-5-carboxylic acid [2-(1H-indol-3-yl)-ethyl]-(2,2,2-trifluoro-ethyl)-amide;
2-Methyl-4-phenyl-pyrimidine-5-carboxylic acid [2-(1H-indol-3-yl)-ethyl]-(2,2,2-trifluoro-ethyl)-amide;
3-m-Tolyl-pyrazine-2-carboxylic acid [2-(1H-indol-3-yl)-ethyl]-(2,2,2-trifluoro-ethyl)-amide;
4-m-Tolyl-pyrimidine-5-carboxylic acid [2-(1H-indol-3-yl)-ethyl]-(2,2,2-trifluoro-ethyl)-amide;
2-Methyl-4-m-tolyl-pyrimidine-5-carboxylic acid [2-(1H-indol-3-yl)-ethyl]-(2,2,2-trifluoro-ethyl)-amide;
3-(4-Fluoro-phenyl)-pyrazine-2-carboxylic acid [2-(1H-indol-3-yl)-ethyl]-(2,2,2-trifluoro-ethyl)-amide;
4-(4-Fluoro-phenyl)-pyrimidine-5-carboxylic acid [2-(1H-indol-3-yl)-ethyl]-(2,2,2-trifluoro-ethyl)-amide;
4-(4-Fluoro-phenyl)-2-methyl-pyrimidine-5-carboxylic acid [2-(1H-indol-3-yl)-ethyl]-(2,2,2-trifluoro-ethyl)-amide;
3-(4-Fluoro-3-methyl-phenyl)-pyrazine-2-carboxylic acid [2-(1H-indol-3-yl)-ethyl]-(2,2,2-trifluoro-ethyl)-amide;
4-(3-Fluoro-phenyl)-2-methyl-pyrimidine-5-carboxylic acid [2-(1H-indol-3-yl)-ethyl]-(2,2,2-trifluoro-ethyl)-amide;
3-(3-Fluoro-5-methyl-phenyl)-pyrazine-2-carboxylic acid [2-(1H-indol-3-yl)-ethyl]-(2,2,2-trifluoro-ethyl)-amide;
3-(3-Methoxy-phenyl)-pyrazine-2-carboxylic acid [2-(1H-indol-3-yl)-ethyl]-(2,2,2-trifluoro-ethyl)-amide;
3-(3,4-Dimethyl-phenyl)-pyrazine-2-carboxylic acid [2-(1H-indol-3-yl)-ethyl]-(2,2,2-trifluoro-ethyl)-amide;
4-(4-Bromo-3-chloro-phenyl)-2-methyl-pyrimidine-5-carboxylic acid [2-(1H-indol-3-yl)-ethyl]-(2,2,2-trifluoro-ethyl)-amide;
4-p-Tolyl-pyrimidine-5-carboxylic acid [2-(1H-indol-3-yl)-ethyl]-(2,2,2-trifluoro-ethyl)-amide;
4-(3,4-Dimethyl-phenyl)-2-methyl-pyrimidine-5-carboxylic acid [2-(1H-indol-3-yl)-ethyl]-(2,2,2-trifluoro-ethyl)-amide;
4-(3,4-Dimethyl-phenyl)-pyrimidine-5-carboxylic acid [2-(1H-indol-3-yl)-ethyl]-(2,2,2-trifluoro-ethyl)-amide;
{[2-Dimethylamino-5-(3-fluoro-4-methyl-phenyl)-thiazole-4-carbonyl]-[2-(1H-indol-3-yl)-ethyl]-amino}-acetic acid methyl ester;
{[5-(3-Bromo-4-fluoro-phenyl)-2-dimethylamino-thiazole-4-carbonyl]-[2-(1H-indol-3-yl)-ethyl]-amino}-acetic acid methyl ester;
{(2-Dimethylamino-5-p-tolyl-thiazole-4-carbonyl)-[2-(1H-indol-3-yl)-ethyl]-amino}-acetic acid methyl ester;
{[2-Dimethylamino-5-(2-fluoro-phenyl)-thiazole-4-carbonyl]-[2-(1H-indol-3-yl)-ethyl]-amino}-acetic acid methyl ester;
{[2-Dimethylamino-5-(4-fluoro-phenyl)-thiazole-4-carbonyl]-[2-(1H-indol-3-yl)-ethyl]-amino}-acetic acid methyl ester;

{[2-(Ethyl-methyl-amino)-5-(4-fluoro-phenyl)-thiazole-4-carbonyl]-[2-(1H-indol-3-yl)-ethyl]-amino}-acetic acid methyl ester;
{[2-(Ethyl-methyl-amino)-5-(3-methoxy-phenyl)-thiazole-4-carbonyl]-[2-(1H-indol-3-yl)-ethyl]-amino}-acetic acid methyl ester;
{(2-Dimethylamino-5-m-tolyl-thiazole-4-carbonyl)-[2-(1H-indol-3-yl)-ethyl]-amino}-acetic acid methyl ester;
{[5-(3-Fluoro-5-trifluoromethyl-phenyl)-2-methyl-thiazole-4-carbonyl]-[2-(1H-indol-3-yl)-ethyl]-amino}-acetic acid methyl ester;
{[2-Cyclopropyl-5-(3-fluoro-5-trifluoromethyl-phenyl)-thiazole-4-carbonyl]-[2-(1H-indol-3-yl)-ethyl]-amino}-acetic acid methyl ester;

{[2-Cyclopropyl-5-(3-fluoro-phenyl)-thiazole-4-carbonyl]-[2-(1H-indol-3-yl)-ethyl]-amino}-acetic acid methyl ester;
[[2-(1H-Indol-3-yl)-ethyl]-(2-methyl-5-p-tolyl-thiazole-4-carbonyl)-amino]-acetic acid methyl ester;
{[2-Cyclopropyl-5-(3-trifluoromethyl-phenyl)-thiazole-4-carbonyl]-[2-(1H-indol-3-yl)-ethyl]-amino}-acetic acid methyl ester;
{[5-(4-Bromo-phenyl)-2-methyl-thiazole-4-carbonyl]-[2-(1H-indol-3-yl)-ethyl]-amino}-acetic acid methyl ester;
{[2-(1H-Indol-3-yl)-ethyl]-[2-methyl-5-(3-trifluoromethyl-phenyl)-thiazole-4-carbonyl]-amino}-acetic acid methyl ester;

{[5-(3,5-Dimethyl-phenyl)-2-methyl-thiazole-4-carbonyl]-[2-(1H-indol-3-yl)-ethyl]-amino}-acetic acid methyl ester;
{[5-(2,4-Dimethyl-phenyl)-2-methyl-thiazole-4-carbonyl]-[2-(1H-indol-3-yl)-ethyl]-amino}-acetic acid methyl ester;
{[5-(3-Cyano-phenyl)-2-methyl-thiazole-4-carbonyl]-[2-(1H-indol-3-yl)-ethyl]-amino}-acetic acid methyl ester;
{[5-(3,4-Difluoro-phenyl)-2-methyl-thiazole-4-carbonyl]-[2-(1H-indol-3-yl)-ethyl]-amino}-acetic acid methyl ester;
{[5-(2,3-Dichloro-phenyl)-2-methyl-thiazole-4-carbonyl]-[2-(1H-indol-3-yl)-ethyl]-amino}-acetic acid methyl ester;
{[5-(2-Chloro-6-fluoro-phenyl)-2-methyl-thiazole-4-carbonyl]-[2-(1H-indol-3-yl)-ethyl]-amino}-acetic acid methyl ester;
{[2-Cyclopropyl-5-(4-fluoro-phenyl)-thiazole-4-carbonyl]-[2-(1H-indol-3-yl)-ethyl]-amino}-acetic acid methyl ester;

{[5-(3,4-Dichloro-phenyl)-2-methyl-thiazole-4-carbonyl]-[2-(1H-indol-3-yl)-ethyl]-amino}-acetic acid methyl ester;
{[5-(3,5-Difluoro-phenyl)-2-methyl-thiazole-4-carbonyl]-[2-(1H-indol-3-yl)-ethyl]-amino}-acetic acid methyl ester;
([2-(1H-Indol-3-yl)-ethyl]-{5-[3-(2-methoxy-ethoxy)-phenyl]-2-methyl-thiazole-4-carbonyl}-amino)-acetic acid methyl ester;
{[5-(3-Fluoro-4-methyl-phenyl)-2-methyl-thiazole-4-carbonyl]-[2-(1H-indol-3-yl)-ethyl]-amino}-acetic acid methyl ester;
{[5-(3-Bromo-phenyl)-2-cyclopropyl-thiazole-4-carbonyl]-[2-(1H-indol-3-yl)-ethyl]-amino}-acetic acid methyl ester;

{[5-(3-Bromo-phenyl)-2-methyl-thiazole-4-carbonyl]-[2-(1H-indol-3-yl)-ethyl]-amino}-acetic acid methyl ester;
{[2-Dimethylamino-5-(3,4-dimethyl-phenyl)-thiazole-4-carbonyl]-[2-(1H-indol-3-yl)-ethyl]-amino}-acetic acid methyl ester;
{[2-Dimethylamino-5-(3-fluoro-phenyl)-thiazole-4-carbonyl]-[2-(1H-indol-3-yl)-ethyl]-amino}-acetic acid methyl ester;
{[2-Dimethylamino-5-(3-trifluoromethyl-phenyl)-thiazole-4-carbonyl]-[2-(1H-indol-3-yl)-ethyl]-amino}-acetic acid methyl ester;
{[5-(3-Chloro-phenyl)-2-dimethylamino-thiazole-4-carbonyl]-[2-(1H-indol-3-yl)-ethyl]-amino}-acetic acid methyl ester;

{[5-(3,4-Dimethyl-phenyl)-2-methyl-thiazole-4-carbonyl]-[2-(5-fluoro-1H-indol-3-yl)-ethyl]-amino}-acetic acid methyl ester;
{[2-(5-Fluoro-1H-indol-3-yl)-ethyl]-[3-(4-fluoro-3-methyl-phenyl)-pyrazine-2-carbonyl]-amino}-acetic acid methyl ester;
{[4-(3,4-Dichloro-phenyl)-pyrimidine-5-carbonyl]-[2-(5-fluoro-1H-indol-3-yl)-ethyl]-amino}-acetic acid methyl ester;
{[2-Dimethylamino-5-(4-fluoro-phenyl)-thiazole-4-carbonyl]-[2-(5-fluoro-1H-indol-3-yl)-ethyl]-amino}-acetic acid methyl ester;
{[3-(4-Ethoxy-phenyl)-pyrazine-2-carbonyl]-[2-(5-fluoro-1H-indol-3-yl)-ethyl]-amino}-acetic acid methyl ester;
{[2-Dimethylamino-5-(3,4-dimethyl-phenyl)-thiazole-4-carbonyl]-[2-(5-fluoro-1H-indol-3-yl)-ethyl]-amino}-acetic acid methyl ester;
[[2-(5-Fluoro-1H-indol-3-yl)-ethyl]-(3-p-tolyl-pyrazine-2-carbonyl)-amino]-acetic acid methyl ester;

{[2-(5-Fluoro-1H-indol-3-yl)-ethyl]-[3-(6-methoxy-pyridin-3-yl)-pyrazine-2-carbonyl]-amino}-acetic acid methyl ester;
[[2-(5-Fluoro-1H-indol-3-yl)-ethyl]-(2-methyl-5-phenyl-thiazole-4-carbonyl)-amino]-acetic acid methyl ester;
{[4-(3,4-Dichloro-phenyl)-2-methyl-pyrimidine-5-carbonyl]-[2-(5-fluoro-1H-indol-3-yl)-ethyl]-amino}-acetic acid methyl ester;
{[2-(5-Fluoro-1H-indol-3-yl)-ethyl]-[3-(4-fluoro-phenyl)-pyrazine-2-carbonyl]-amino}-acetic acid methyl ester;
[[2-(5-Fluoro-1H-indol-3-yl)-ethyl]-(4-p-tolyl-pyrimidine-5-carbonyl)-amino]-acetic acid methyl ester; and
2-Cyclopropyl-5-m-tolyl-oxazole-4-carboxylic acid cyclopropylmethyl-[2-(1H-indol-3-yl)-ethyl]-amide.

Any reference to a compound of formula (I) is to be understood as referring also to the salts (and especially the pharmaceutically acceptable salts) of such a compound, as appropriate and expedient.

The term “pharmaceutically acceptable salts” refers to non-toxic, inorganic or organic acid and/or base addition salts. Reference can be made to “Salt selection for basic drugs”, Int. J. Pharm. 1986, 33, 201-217.

The present invention also includes isotopically labelled, especially ²H (deuterium) labelled compounds of formula (I), which compounds are identical to the compounds of formula (I) except that one or more atoms have each been replaced by an atom having the same atomic number but an atomic mass different from the atomic mass usually found in nature. Isotopically labelled, especially ²H (deuterium) labelled compounds of formula (I) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula (I) are not isotopically labelled, or they are labelled only with one or more deuterium atoms. In a sub-embodiment, the compounds of formula (I) are not isotopically labelled at all. Isotopically labelled compounds of formula (I) may be prepared in analogy to the methods described hereinafter, but using the appropriate isotopic variation of suitable reagents or starting materials.

A further aspect of the invention is a pharmaceutical composition containing at least one compound according to formula (I), or a pharmaceutically acceptable salt thereof, and a pharmaceutically acceptable carrier material.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The compounds of formula (I) and their pharmaceutically acceptable salts can be used as medicaments, e.g. in the form of pharmaceutical compositions for enteral or parenteral administration.

The compounds according to formula (I) may be used for the preparation of a medicament, and are suitable, for the prevention or treatment of diseases selected from the group consisting of dysthymic disorders including major depression and cyclothymia, affective neurosis, all types of manic depressive disorders, delirium, psychotic disorders, schizophrenia, catatonic schizophrenia, delusional paranoia, adjustment disorders and all clusters of personality disorders; schizoaffective disorders; anxiety disorders including generalized anxiety, obsessive compulsive disorder, posttraumatic stress disorder, panic attacks, all types of phobic anxiety and avoidance; separation anxiety; all psychoactive substance use, abuse, seeking and reinstatement; all types of psychological or physical addictions, dissociative disorders including multiple personality syndromes and psychogenic amnesias; sexual and reproductive dysfunction; psychosexual dysfunction and addiction; tolerance to narcotics or withdrawal from narcotics; increased anaesthetic risk, anaesthetic responsiveness; hypothalamic-adrenal dysfunctions; disturbed biological and circadian rhythms; sleep disturbances associated with diseases such as neurological disorders including neuropathic pain and restless leg syndrome; sleep apnea; narcolepsy; chronic fatigue syndrome; insomnias related to psychiatric disorders; all types of idiopathic insomnias and parasomnias; sleep-wake schedule disorders including jet-lag; all dementias and cognitive dysfunctions in the healthy population and in psychiatric and neurological disorders; mental dysfunctions of aging; all types of amnesia; severe mental retardation; dyskinesias and muscular diseases; muscle spasticity, tremors, movement disorders; spontaneous and medication-induced dyskinesias; neurodegenerative disorders including Huntington's, Creutzfeld-Jacob's, Alzheimer's diseases and Tourette syndrome; Amyotrophic lateral sclerosis; Parkinson's disease; Cushing's syndrome; traumatic lesions; spinal cord trauma; head trauma; perinatal hypoxia; hearing loss; tinnitus; demyelinating diseases; spinal and cranial nerve diseases; ocular damage; retinopathy; epilepsy; seizure disorders; absence seizures, complex partial and generalized seizures; Lennox-Gastaut syndrome; migraine and headache; pain disorders; anaesthesia and analgesia; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; dental pain; pain related to infection e.g. by HIV; post-chemotherapy pain; post-stroke pain; post-operative pain; neuralgia; osteoarthritis; conditions associated with visceral pain such as irritable bowel syndrome; eating disorders; diabetes; toxic and dysmetabolic disorders including cerebral anoxia, diabetic neuropathies and alcoholism; appetite, taste, eating, or drinking disorders; somatoform disorders including hypochondriasis; vomiting/nausea; emesis; gastric dyskinesia; gastric ulcers; Kallman's syndrome (anosmia); impaired glucose tolerance; intestinal motility dyskinesias; hypothalamic diseases; hypophysis diseases; hyperthermia syndromes, pyrexia, febrile seizures, idiopathic growth deficiency; dwarfism; gigantism; acromegaly; basophil adenoma; prolactinoma; hyperprolactinemia; brain tumors, adenomas; benign prostatic hypertrophy, prostate cancer; endometrial, breast, colon cancer; all types of testicular dysfunctions, fertility control; reproductive hormone abnormalities; hot flashes; hypothalamic hypogonadism, functional or psychogenic amenorrhea; urinary bladder incontinence; asthma; allergies; all types of dermatitis, acne and cysts, sebaceous gland dysfunctions; cardiovascular disorders; heart and lung diseases, acute and congestive heart failure; hypotension; hypertension; dyslipidemias, hyperlipidemias, insulin resistance; urinary retention; osteoporosis; angina pectoris; myocardial infarction; arrhythmias, coronary diseases, left ventricular hypertrophy; ischemic or haemorrhagic stroke; all types of cerebrovascular disorders including subarachnoid haemorrhage, ischemic and hemorrhagic stroke and vascular dementia; chronic renal failure and other renal diseases; gout; kidney cancer; urinary incontinence; and other diseases related to general orexin system dysfunctions.

In a preferred embodiment, the compounds according to formula (I) may be used for the preparation of a medicament, and are suitable, for the prevention or treatment of diseases selected from the group consisting of all types of sleep disorders, of stress-related syndromes, of psychoactive substance use, abuse, seeking and reinstatement, of cognitive dysfunctions in the healthy population and in psychiatric and neurologic disorders, of eating or drinking disorders.

Eating disorders may be defined as comprising metabolic dysfunction; dysregulated appetite control; compulsive obesities; emeto-bulimia or anorexia nervosa. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance. Drinking disorders include polydipsias in psychiatric disorders and all other types of excessive fluid intake. Sleep disorders include all types of parasomnias, insomnias, narcolepsy and other disorders of excessive sleepiness, sleep-related dystonias; restless leg syndrome; sleep apneas; jet-lag syndrome; shift-work syndrome, delayed or advanced sleep phase syndrome or insomnias related to psychiatric disorders. Insomnias are defined as comprising sleep disorders associated with aging; intermittent treatment of chronic insomnia; situational transient insomnia (new environment, noise) or short-term insomnia due to stress; grief; pain or illness. Insomnia also include stress-related syndromes including post-traumatic stress disorders as well as other types and subtypes of anxiety disorders such as generalized anxiety, obsessive compulsive disorder, panic attacks and all types of phobic anxiety and avoidance. Psychoactive substance use, abuse, seeking and reinstatement are defined as all types of psychological or physical addictions and their related tolerance and dependence components. Cognitive dysfunctions include deficits in all types of attention, learning and memory functions occurring transiently or chronically in the normal, healthy, young, adult or aging population, and also occurring transiently or chronically in psychiatric, neurologic, cardiovascular and immune disorders.

In a further preferred embodiment of the invention, the compounds according to formula (I) may be used for the preparation of a medicament, and are suitable, for the prevention or treatment of diseases selected from the group consisting of sleep disorders that comprises all types of insomnias, narcolepsy and other disorders of excessive sleepiness, sleep-related dystonias, restless leg syndrome, sleep apneas, jet-lag syndrome, shift-work syndrome, delayed or advanced sleep phase syndrome or insomnias related to psychiatric disorders.

In another preferred embodiment of the invention, the compounds according to formula (I) may be used for the preparation of a medicament, and are suitable, for the prevention or treatment of diseases selected from the group consisting of cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders.

In another preferred embodiment of the invention, the compounds according to formula (I) may be used for the preparation of a medicament, and are suitable, for the prevention or treatment of diseases selected from the group consisting of eating disorders that comprise metabolic dysfunction; dysregulated appetite control; compulsive obesities; emeto-bulimia or anorexia nervosa.

In another preferred embodiment of the invention, the compounds according to formula (I) may be used for the preparation of a medicament, and are suitable, for the prevention or treatment of diseases selected from the group consisting of psychoactive substance use, abuse, seeking and reinstatement that comprise all types of psychological or physical addictions and their related tolerance and dependence components.

The present invention also relates to a method for the prevention or treatment of a disease or disorder mentioned herein comprising administering to a subject a pharmaceutically active amount of a compound of formula (I).

Where the plural form is used for compounds, salts, pharmaceutical compositions, diseases or the like, this is intended to mean also a single compound, salt, disease or the like.

A further aspect of the invention is a process for the preparation of compounds of formula (I). Compounds of formula (I) of the present invention can be prepared according to the general sequence of reactions outlined in the schemes below wherein A, B, D, X, Y, R¹, R² and R³ are as defined for formula (I). The compounds obtained may also be converted into pharmaceutically acceptable salts thereof in a manner known per se.

In general, all chemical transformations can be performed according to well-known standard methodologies as described in the literature or as described in the procedures below or in the experimental part.

### Preparation of Compounds of Formula (I):

Compounds of formula (I) can be prepared by reaction of an amine (1) with an acid B—COOH in the presence of an amide-coupling reagent such as TBTU and a base like DIPEA in a solvent like DMF (scheme 1). Alternatively amines (1) can be coupled with acids B*—COOH bearing a chlorine or bromine atom in ortho-position to the acid function under standard amide-coupling conditions like TBTU/DIPEA in DMF and subsequent Suzuki-coupling with boronic acids D-B(OH)₂ using Pd(OAc)₂ in the presence of triphenylphosphine and aqueous K₂CO₃ solution in a solvent like DME or using Pd(PPh₃)₄ in the presence of aqueous Na₂CO₃ solution in a solvent mixture like toluene/ethanol to give the respective compounds of formula (I).

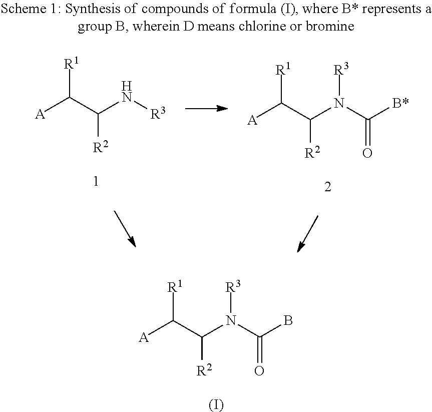

Compounds of formula (I), wherein R¹ represents (C₃₋₆)cycloalkyl-amino, which are also compounds of formula (Ia) can be prepared from alcohols (3) by activation with a sulfonyl chloride like MsCl in the presence of a base like TEA and subsequent substitution with an amine R—NH₂ [R=(C₃₋₆)cycloalkyl] in a solvent like EtOH (scheme 2).

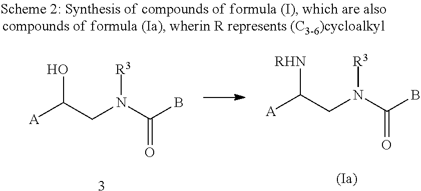

Compounds of formula (I) bearing a primary amino-function, which are also compounds of formula (Ib) or (Ic) (X=CH₂NH₂) can be prepared by removal of a nitrogen-protecting group under conditions known in the art, e.g. by removal of the Boc-group of compounds (4) or (5) (X=CH₂NHBoc) under acidic conditions like hydrochloric acid in a solvent like dioxane (scheme 3). Compounds of formula (Ic) (X=NR⁴R⁵) can be prepared from the respective bromides (5) (X=Br) by substitution with the respective amine HNR⁴R⁵ in a solvent like THF at elevated temperatures of around 70° C. in a closed vial.

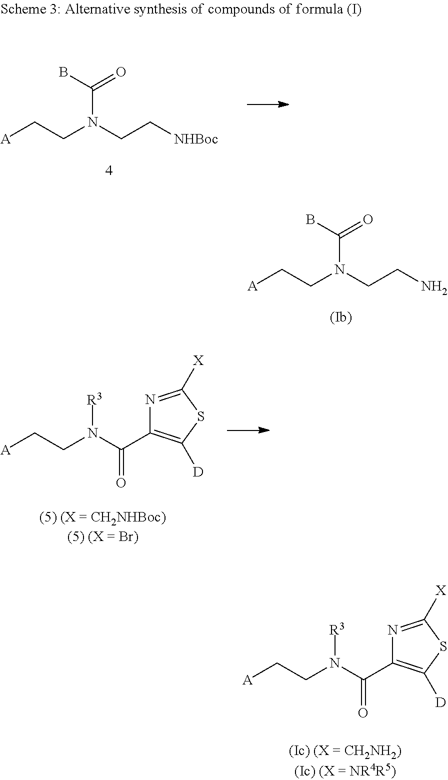

### Preparation of Intermediates:

Pyridine- and pyrazine-carboxylic acid derivatives of formula B—COOH can be prepared for instance according to one of the pathways shown for the examples in scheme 4.

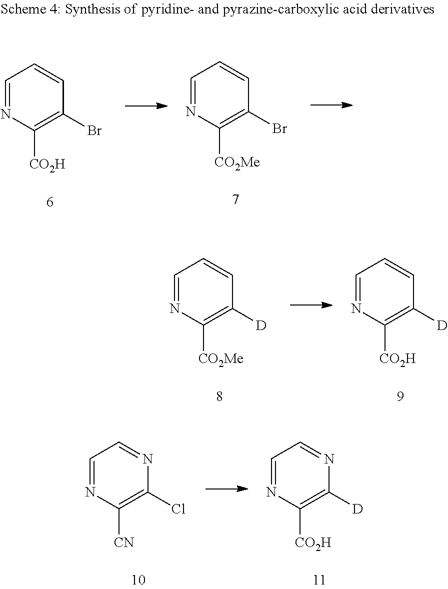

After esterification of the respective pyridine-carboxylic acid (6) with an alcohol like MeOH in the presence of conc sulfuric acid at higher temperatures (e.g. reflux) the coupled ester derivatives (8) can be obtained for instance under Suzuki conditions using a boronic acid derivative D-B(OH)₂ in the presence of a catalyst like Pd(PPh₃)₄ and a base like aq Na₂CO₃ solution in a solvent mixture like EtOH/toluene. After saponification of the ester (8) with a base like aq NaOH solution in a solvent mixture like THF/MeOH the desired pyridine-carboxylic acid derivatives (9) are obtained. Alternatively pyrazine-carboxylic acid derivatives (11) can be obtained by coupling the respective chlorides (10) with a boronic acid derivative D-B(OH)₂ in the presence of a catalyst like Pd(OAc)₂ and triphenylphosphine in a solvent like DME at elevated temperatures of around 90° C. and subsequent saponification with a base like NaOH in a solvent or solvent mixture like water and methanol at elevated temperatures. Thiazole-4-carboxylic acid derivatives of formula B—COOH are for instance synthesised according to scheme 5.

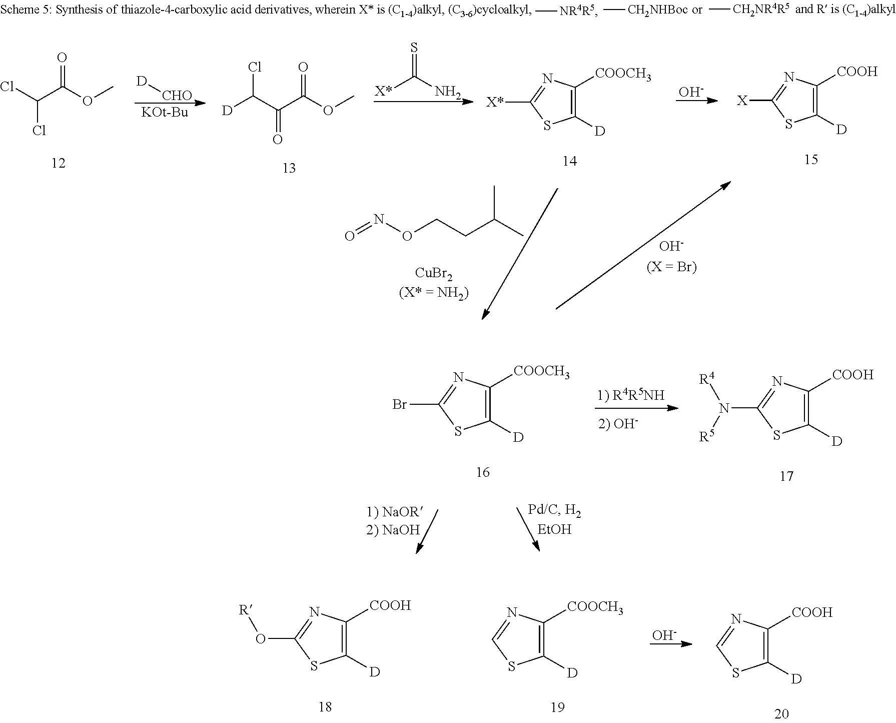

By reaction of methyl dichloroacetate (12; commercially available) with an aldehyde D-CHO in the presence of a base like KOtBu in a solvent like THF the 3-chloro-2-oxo-propionic ester derivatives (13) are obtained which are transformed in a reaction with thioamides [X*=(C₁₋₄)alkyl, (C₃₋₆)cycloalkyl, —CH₂NHBoc or —CH₂NR⁴R⁵] to the respective 2-substituted thiazole derivatives (14) or in a reaction with thioureas (X*=—NR⁴R⁵) to 2-amino-substituted thiazole derivatives (14). Saponification of the ester function with an aq. solution of e.g. NaOH in a solvent like MeOH, isopropanol or MeOH/THF mixtures results in the formation of the desired carboxylic acids (15, X=(C₁₋₄)alkyl, (C₃₋₆)cycloalkyl, —NR⁴R⁵, or —CH₂NR⁴R⁵). 2-Bromo-thiazole derivatives (16) are for instance obtained by reaction of the respective 2-amino-thiazole derivative (14, X*=NH₂) with isoamylnitrite in the presence of CuBr₂ in a solvent such as MeCN. The ester derivatives (16) are either transferred to 2-amino-substituted thiazole derivatives (17) by reaction of (16) with amines HNR⁴R⁵ in a solvent like MeCN and subsequent saponification or to 2-alkoxy substituted analogues (18) by reaction with a sodium alkoxide and subsequent saponification with NaOH solution. Saponification of ester (16) as described above results in the formation of carboxylic acids (15, X=Br). In addition compounds (20) which are unsubstituted in 2-position are synthesized by hydrogenation of (16) in the presence of a catalyst like palladium on charcoal and subsequent saponification of the intermediate ester (19).

Aldehydes D-CHO are commercially available or may be synthesized by procedures known from the literature like for instance reduction of the respective carboxylic acid or their different derivatives with a reducing agent, by reduction of the respective nitrile or by oxidation of benzylic alcohols and their heterocyclic analogues with oxidating agents (e.g.: J. March, Advanced Organic Chemistry, 4^{th} edition, John Wiley & Sons, p. 447-449, 919-920 and 1167-1171).

(C₃₋₆)Cycloalkyl-thioamides may be synthesized by treatment of (C₃₋₆)cycloalkyl-carboxamides with Lawesson's reagent.

Alternatively, thiazole-4-carboxylic acid derivatives of formula B—COOH can be synthesised according to scheme 6.

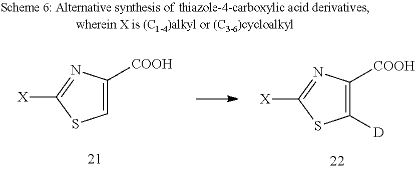

5-Bromo-thiazole-4-carboxylic acid derivatives can be obtained by deprotonation of the respective thiazole-4-carboxylic acid derivative (21) in 5-position with a base like n-BuLi in a solvent like THF at a temperature of around −78° C. and subsequent bromination with a solution of bromine in a solvent like cyclohexane. The obtained bromide can be coupled with a boronic acid derivative D-B(OH)₂ under Suzuki conditions using a catalyst like Pd(PPh₃)₄ and a base like aq Na₂CO₃ solution in a solvent mixture like EtOH/toluene to give the desired carboxylic acid derivatives (22).

Thiazole-5-carboxylic acid derivatives of formula B—COOH are for instance synthesised according to scheme 7.

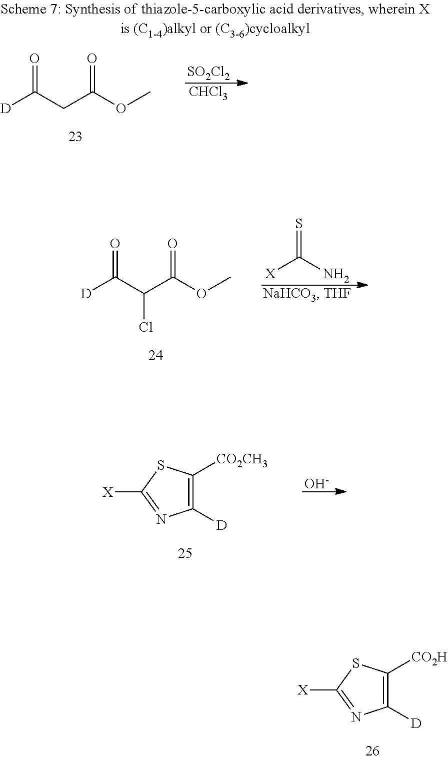

By chlorination of β-keto ester derivatives (23) with sulfuryl chloride in chloroform α-chloro ester derivatives (24) are obtained which by reaction with thioamides in a solvent like THF give the respective thiazole-5-carboxylic acid esters (25). These are transferred to the desired acids (26) by saponification with for instance KOH in a solvent mixture like water and EtOH.

Oxazole-4-carboxylic acid derivatives of formula B—COOH are for instance synthesised according to scheme 8.

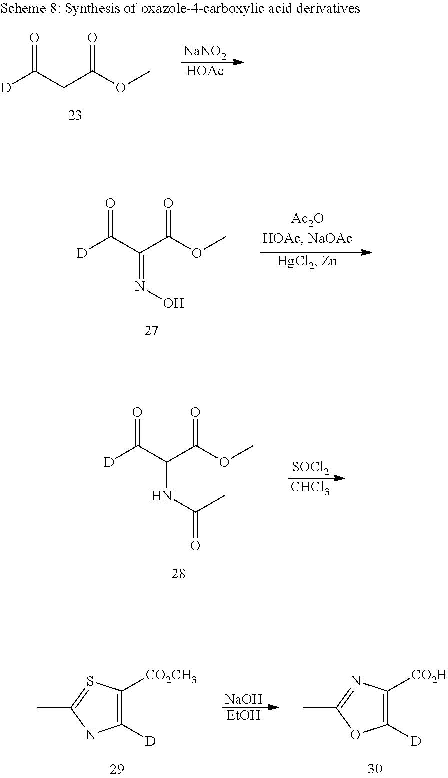

By reaction of β-keto ester derivatives (23) with NaNO₂ in the presence of acetic acid α-hydroxyimino ester derivatives (27) are obtained which are transformed to α-acetylamino ester derivatives (28) in a reaction with Ac₂O in the presence of HgCl₂ and zinc. By cyclisation of these intermediates with SOCl₂ in a solvent like CHCl₃ the respective oxazole-4-carboxylic ester derivatives (29) are synthesized which are saponified as described above to give the desired acids (30).

Alternatively oxazole-4-carboxylic acid derivatives of formula B—COOH can be obtained from β-keto ester derivatives (23) by reaction with 4-acetylamino-benzene-sulfonyl azide in the presence of a base like TEA in a solvent like MeCN and subsequent reaction with formamide in the presence of dirhodium tetraacetate in a solvent like DCM to give the formamide derivative (32), which can be cyclised to ester derivatives (34) with iodine in the presence of triphenylphosphine and a base like TEA in a solvent like DCM (scheme 9). After saponification of (34) with a base like NaOH in a solvent mixture like water/EtOH the desired carboxylic acid derivatives (35) are obtained. The intermediate ester derivatives (34) can also be prepared by reaction of methyl isocyanoacetate (33) with the respective acid derivative D-COOH in the presence of K₂CO₃ in a solvent like DMF and subsequent treatment with DPPA.

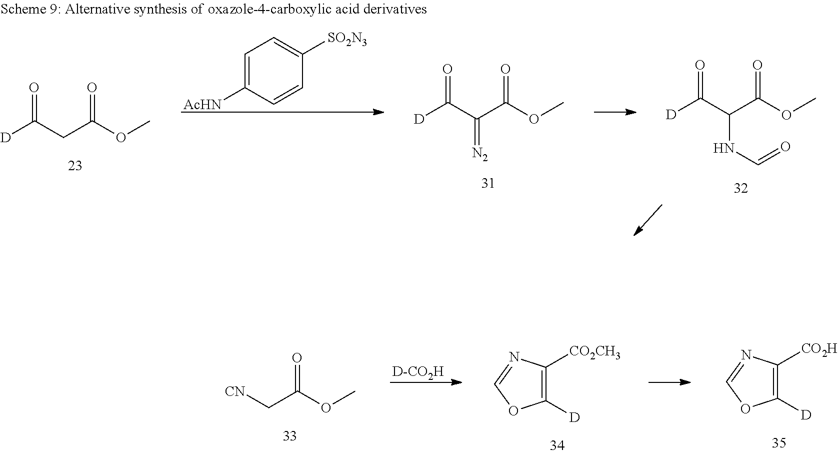

β-Keto ester derivatives (23) are commercially available or may be synthesized by procedures known in the literature like for instance Claisen condensation, reaction of aromatic and heteroaromatic ester derivatives with acetic ester derivatives in the presence of strong bases, reaction of acetophenones and their heterocyclic analogues with methyl cyanoformate or diethyl dicarbonate in the presence of bases or a Reformatsky-type reaction (e.g.: J. March, Advanced Organic Chemistry, 4^{th} edition, John Wiley & Sons, p. 491-493 and 931).

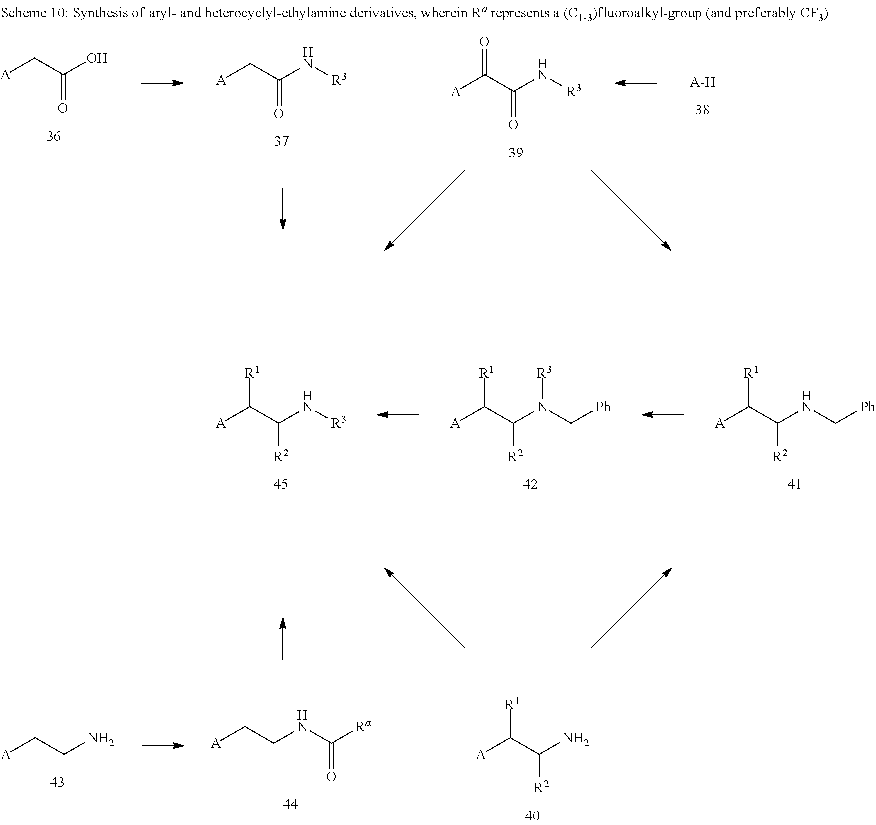

Aryl- and heterocyclyl-ethylamine derivatives (45) can be prepared from starting materials which are commercially available, prepared as described below or known in the art following different pathways (scheme 10). Starting from acids (36) the respective amides (37) can be obtained by standard amide-coupling reactions with an amine R³NH₂ using for example a coupling reagent like TBTU in the presence of a base like DIPEA in a solvent like DMF. The obtained amides (37) can be reduced to the desired amine (45) (R¹=R²=H) by reduction of the amide-function with a reducing agent like LAH in a solvent like THF at elevated temperatures. Alternatively 2-oxo-acetamide derivatives (39) are prepared from compounds (38), wherein A-H represents an indole derivative, by reaction with oxalyl chloride in a solvent like ether and subsequent addition of an amine R³NH₂. The amides (39) can be reduced to the respective amines (45) (R¹=R²=H) or, in case R³ represents benzyl, (41) (R¹=R²=H) by reduction with a reducing agent such as LAH in a solvent like THF at elevated temperatures. An alternative pathway to amines (41) is the reductive amination of the primary amines (40), wherein A preferably represents an unsubstituted or substituted phenyl, with benzaldehyde in presence or absence of molecular sieves in a solvent like MeOH and subsequent reduction with a reducing agent like sodium borohydride. Amines (41) can be transferred to tertiary amines (42) in either an alkylation reaction with alkyl halides R³Hal (Hal=Cl, Br, or I) or alkyl sulfonates like R³OS(O)₂CF₃; or in a reductive amination reaction with an aldehyde in the presence of a reducing agent like NaBH(OAc)₃ in a solvent like DCM with or without addition of water. By removal of the benzyl group of amines (42) in a hydrogenation reaction using a catalyst like Pd/C or the like in a solvent like EtOH under a hydrogen atmosphere the desired amines (45) are obtained. In still another approach amines (45) can be obtained by either reductive amination of primary amines (40) with an aldehyde in a solvent like MeOH using a reducing agent like NaBH₄ or by alkylation of amines (40) with an alkyl halide (especially an alkyl iodide) in the presence of a base like TEA or DIPEA in a solvent like THF or DMF with or without addition of MeOH at elevated temperatures of around 50° C. to 60° C. In addition, amines (45) are prepared by reduction of amides (44) with a reducing agent like borane (preferably as a THF-complex) in a solvent like THF at elevated temperatures (preferably reflux). The amides (44) can be obtained from amines (43) and the respective acids R^{a}—COOH using known amide coupling conditions or by reaction of (43) with an ester derivative R^{a}—COOR(R represents methyl or ethyl) in the presence of a base like TEA in a solvent like MeOH.

Amines (40), wherein R¹ represents hydrogen and R² represents hydrogen [identical to amines (43)] or (C₁₋₄)alkyl, can be prepared by reaction of an aldehyde A-CHO (46) with the respective nitroalkane in the presence of a base like n-butylamine and of an acid like acetic acid at a temperature of around 95° C. followed by reduction of the obtained nitro-vinyl derivative (47) (scheme 11). The reduction may be performed with a reducing agent like LAH in the presence of conc sulfuric acid in a solvent like THF under heating or by a hydrogenation reaction using a catalyst like Pd/C in the presence of aqueous hydrochloric acid in a solvent like EtOH.

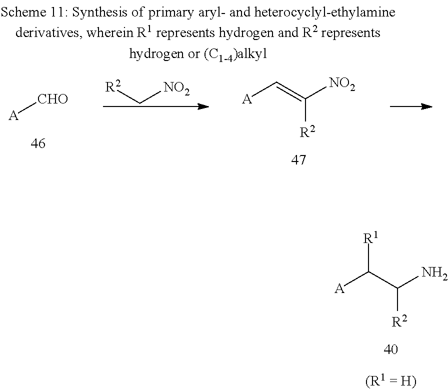

Amines (40), wherein R¹ represents hydroxy, are commercially available or may be prepared from aldehydes (46) by reaction with trimethylsilyl cyanide in the presence of a Lewis acid like zinc iodide in a solvent like DCM and subsequent reduction with a reducing agent like LAH in a solvent like ether (e.g. R. Viswanathan et al. J. Am. Chem. Soc. 2003, 125, 163-168 or K. Kirk et al. J. Med. Chem. 1986, 29, 1982-86) or with potassium cyanide in the presence of a 18-crown-6 and subsequent reduction with LAH (J. Swenton et al. J. Org. Chem. 1990, 55, 2019-26). Alternatively amines (40) (R¹=OH) may be obtained by ring opening of aryl-epoxides with an azide source like sodium azide and subsequent hydrogenation with a catalyst like PtO₂ in a solvent like MeOH (A. Cordova et al. Chemistry 2004, 10, 3673-84).

Pyrimidine-5-carboxylic acid derivatives of formula B—COOH are for instance synthesised according to scheme 12.

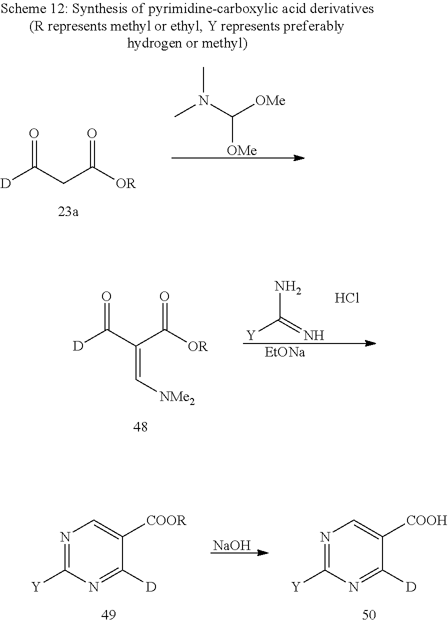

By reaction of β-keto ester derivatives (23a) with N,N-dimethylformamid-dimethylacetale in a solvent like cyclohexane at reflux the respective dimethylamino-acrylic ester derivatives (48) are obtained which are transformed to pyrimidine derivatives (49) by treatment with the respective amidine hydrochloride (like formamidine hydrochloride or acetamidine hydrochloride) in the presence of a base like sodium ethylate in a solvent like ethanol at reflux. By saponification of the ester (49) with a base like NaOH in a solvent or solvent mixture like water and methanol the respective pyrimidine-5-carboxylic acid derivatives are obtained.

Besides, the term “room temperature” as used herein refers to a temperature of around 25° C.

Unless used regarding temperatures, the term “around” placed before a numerical value “X” refers in the current application to an interval extending from X minus 10% of X to X plus 10% of X, and preferably to an interval extending from X minus 5% of X to X plus 5% of X. In the particular case of temperatures, the term “around” placed before a temperature “Y” refers in the current application to an interval extending from the temperature Y minus 10° C. to Y plus 10° C., and preferably to an interval extending from Y minus 5° C. to Y plus 5° C.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range or if a variable is defined as being an integer between 1 and 4, this means that the variable is the integer 1, 2, 3, or 4.

### Experimental Section

### Abbrevations (as Used Herein and in the Description Above):

Ac Acetyl (e.g. in HOAc=acetic acid or Ac₂O=acetic acid anhydride)

### aq Aqueous

Boc tert-Butoxycarbonyl
BSA Bovine serum albumine
CHO Chinese hamster ovary
conc Concentrated

### d Day(s)

DBU 1,8-Diazabicyclo[5.4.0]undec-7-ene DCM Dichloromethane

### DIPEA Diisopropylethylamine

### DMAP 4-Dimethylaminopyridine

### DME 1,2-Dimethoxyethane

### DMF N,N-Dimethylformamide

### DMS Dimethylsulfide

### DMSO Dimethylsulfoxide

DPPA Diphenyl phosphoryl azide

### eq Equivalent(s)

ES Electron spray
Et Ethyl (e.g. in NaOEt=sodium ethoxide)

### Ether Diethylether

EtOAc Ethyl acetate

### EtOH Ethanol

FC flash column chromatography on silica gel
FCS Foatal calf serum
FLIPR Fluorescent imaging plate reader

### h Hour(s)

HBSS Hank's balanced salt solution
HEPES 4-(2-hydroxyethyl)-piperazine-1-ethanesulfonic acid
HPLC High performance liquid chromatography
KOtBu Potassium tert. butoxide
LAH Lithium aluminum hydride
LC Liquid chromatography

### M Molar(ity)

### Me Methyl

### MeCN Acetonitrile

### MeOH Methanol

### min Minute(s)

MS Mass spectroscopy

### NBS N-Bromosuccinimide

### Ph Phenyl

### PPTS Pyridinium-para-toluenesulfonate

prep Preparative

### PTFE Polytetrafluorethylen

PTSA para-Toluenesulfonic acid monohydrate
RT Room temperature

### sat Saturated

t_{R} Retention time
TBME tert-Butyl methyl ether
TBTU O-Benzotriazol-1-yl-N,N,N′,N′-tetramethyluronium tetrafluoroborate

### TEA Triethylamine

Tf trifluoromethanesulfonyl (e.g. in TfO=trifluoromethanesulfonyloxy)
TFA Trifluoroacetic acid

### THF Tetrahydrofuran

### TMS Trimethylsilyl

### I-Chemistry

The following examples illustrate the preparation of pharmacologically active compounds of the invention but do not at all limit the scope thereof.

All temperatures are stated in ° C.

Compounds are characterized by:

¹H-NMR: 300 MHz Varian Oxford or 400 MHz Bruker Avance; chemical shifts are given in ppm relative to the solvent used; multiplicities: s=singlet, d=doublet, t=triplet, m=multiplet, b=broad, coupling constants are given in Hz;

### LC-MS:

Method A (A):
Agilent 1100 series with DAD and MS detection (MS: Finnigan single quadrupole); columns (4.6×50 mm, 5 μm): Zorbax SB-AQ, Zorbax Extend C18 or Waters XBridge C18; eluent A: MeCN, eluent B: TFA in water (0.4 mL/L), 5% to 95% CH₃CN, flow rate 4.5 mL/min;

method B (B):
Agilent 1100 series with DAD and MS detection (MS: Finnigan single quadrupole); columns (4.6×50 mm, 5 μm): Zorbax SB-AQ, Zorbax Extend C18 or Waters XBridge C18; eluent A: MeCN, eluent B: conc. NH₃ in water (1.0 mL/L), 5% to 95% CH₃CN, flow rate 4.5 mL/min;

Method C(C):
Dionex UltiMate 3000 with DAD, ELSD (Sedex 85) and MS detection (MS: Finnigan single quadrupole); column: Supelco Ascentis Express C18 (4.6×30 mm, 2.7 μm); eluent A: MeCN, eluent B: TFA in water (0.4 mL/L), 2% to 95% CH₃CN, flow rate 4.5 mL/min;

t_{R} is given in min;
In case of a partial separation of rotamers, as seen for several examples of compounds of formula (I), two retention times are given.

Compounds are purified by FC or by prep HPLC using RP-C₁₈ based columns with MeCN/water gradients and formic acid or ammonia additives. Preparative thin layer chromatography (TLC) is performed with 0.2 or 0.5 mm plates: Merck, Silica gel 60 F₂₅₄.

### A. Preparation of Precursors and Intermediates

### A.1 Synthesis of Thiazole-4-Carboxylic Acid Derivatives

A.1.1 Synthesis of 3-chloro-2-oxo-propionic ester derivatives (General Procedure)

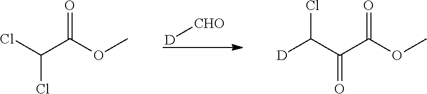

A solution of the respective aldehyde (338 mmol, 1.0 eq) and methyl dichloroacetate (338 mmol, 1.0 eq) in THF (100 mL) is added dropwise to a cold (−60° C.) suspension of KOtBu (335 mmol, 1.0 eq) in THF (420 mL). After 4 h the mixture is allowed to reach RT, stirred over night and concentrated in vacuo. DCM and ice-cold water are added, the layers are separated and the aq. layer is extracted twice with DCM. The combined organic layers are washed with ice-cold water and brine, dried over MgSO₄ and concentrated in vacuo to give the desired 3-chloro-2-oxo-propionic ester derivative which is used without further purification.

3-Chloro-2-oxo-3-m-tolyl-propionic acid methyl ester

prepared by reaction of 3-methyl-benzaldehyde with methyl dichloroacetate

3-Chloro-2-oxo-3-p-tolyl-propionic acid methyl ester

prepared by reaction of 4-methyl-benzaldehyde with methyl dichloroacetate.

3-Chloro-3-(4-ethyl-phenyl)-2-oxo-propionic acid methyl ester

prepared by reaction of 4-ethyl-benzaldehyde with methyl dichloroacetate.

3-Chloro-3-(3-methoxy-phenyl)-2-oxo-propionic acid methyl ester

prepared by reaction of 3-methoxy-benzaldehyde with methyl dichloro-acetate.

3-Chloro-3-(2-fluoro-phenyl)-2-oxo-propionic acid methyl ester

prepared by reaction of 2-fluoro-benzaldehyde with methyl dichloro-acetate.

3-Chloro-3-(3-fluoro-phenyl)-2-oxo-propionic acid methyl ester

prepared by reaction of 3-fluoro-benzaldehyde with methyl dichloroacetate.
3-Chloro-3-(4-fluoro-phenyl)-2-oxo-propionic acid methyl ester

prepared by reaction of 4-fluoro-benzaldehyde with methyl dichloroacetate.

3-Chloro-3-(3-chloro-phenyl)-2-oxo-propionic acid methyl ester

prepared by reaction of 3-chloro-benzaldehyde with methyl dichloro-acetate.
3-Chloro-3-(4-chloro-phenyl)-2-oxo-propionic acid methyl ester

prepared by reaction of 4-chloro-benzaldehyde with methyl dichloro-acetate.
3-Chloro-2-oxo-3-(3-trifluoromethyl-phenyl)-propionic acid methyl ester

prepared by reaction of 3-trifluoromethyl-benzaldehyde with methyl dichloro-acetate.
3-Chloro-3-(3,4-dimethyl-phenyl)-2-oxo-propionic acid methyl ester

prepared by reaction of 3,4-dimethyl-benzaldehyde with methyl dichloro-acetate.

3-Chloro-3-(2,3-dimethyl-phenyl)-2-oxo-propionic acid methyl ester

prepared by reaction of 2,3-dimethyl-benzaldehyde with methyl dichloro-acetate.
3-Chloro-3-(2,4-dimethyl-phenyl)-2-oxo-propionic acid methyl ester

prepared by reaction of 2,4-dimethyl-benzaldehyde with methyl dichloro-acetate.

3-Chloro-3-(3,5-dimethyl-phenyl)-2-oxo-propionic acid methyl ester

prepared by reaction of 3,5-dimethyl-benzaldehyde with methyl dichloro-acetate.

3-Chloro-3-(3,4-dichloro-phenyl)-2-oxo-propionic acid methyl ester

prepared by reaction of 3,4-dichloro-benzaldehyde with methyl dichloro-acetate.

3-Chloro-3-(3,4-difluoro-phenyl)-2-oxo-propionic acid methyl ester

prepared by reaction of 3,4-difluoro-benzaldehyde with methyl dichloro-acetate.
3-Chloro-3-(3-fluoro-4-methyl-phenyl)-2-oxo-propionic acid methyl ester

prepared by reaction of 3-fluoro-4-methyl-benzaldehyde with methyl dichloro-acetate.
3-Chloro-3-(3-fluoro-5-trifluoromethyl-phenyl)-2-oxo-propionic acid methyl ester

prepared by reaction of 3-fluoro-5-trifluoromethyl-benzaldehyde with methyl dichloro-acetate.

3-Chloro-3-(3-fluoro-2-methyl-phenyl)-2-oxo-propionic acid methyl ester

prepared by reaction of 3-fluoro-2-methyl-benzaldehyde with methyl dichloro-acetate.
3-Chloro-2-oxo-3-phenyl-propionic acid methyl ester

prepared by reaction of benzaldehyde with methyl dichloro-acetate.

3-Chloro-3-(4-cyano-phenyl)-2-oxo-propionic acid methyl ester

prepared by reaction of 4-cyano-benzaldehyde with methyl dichloro-acetate.
3-Chloro-3-(3,5-difluoro-phenyl)-2-oxo-propionic acid methyl ester

prepared by reaction of 3,5-difluoro-benzaldehyde with methyl dichloro-acetate.
3-Chloro-3-(3-cyano-phenyl)-2-oxo-propionic acid methyl ester

prepared by reaction of 3-cyano-benzaldehyde with methyl dichloro-acetate.
3-Chloro-3-(2,3-difluoro-4-methyl-phenyl)-2-oxo-propionic acid methyl ester

prepared by reaction of 2,3-difluoro-4-methyl-benzaldehyde with methyl dichloro-acetate.

A.1.2 Synthesis of thiazole-4-carboxylic acid methyl ester derivatives (General Procedure)

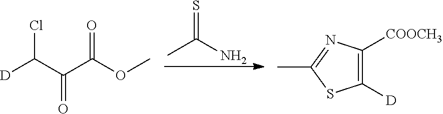

A solution of thioacetamide (132 mmol, 1.0 eq) in MeCN (250 mL) is added to a mixture of the respective 3-chloro-2-oxo-propionic ester derivative (132 mmol, 1.0 eq) and molecular sieves (4 Å, 12 g) in MeCN (60 mL). After stirring for 5 h the mixture is cooled in an ice-bath and the obtained precipitate is filtered off. The residue is washed with cold MeCN, dried, dissolved in MeOH (280 mL) and stirred at 50° C. for 6 h. The solvents are removed in vacuo to give the desired thiazole derivative as a white solid. The presence of molecular sieve is often not necessary for successful reactions.

2-Methyl-5-m-tolyl-thiazole-4-carboxylic acid methyl ester

prepared by reaction of 3-chloro-2-oxo-3-m-tolyl-propionic acid methyl ester with thioacetamide. LC-MS (A): t_{R}=0.94 min; [M+H]⁺=248.0.

2-Methyl-5-p-tolyl-thiazole-4-carboxylic acid methyl ester

prepared by reaction of 3-chloro-2-oxo-3-p-tolyl-propionic acid methyl ester with thioacetamide. LC-MS (A): t_{R}=0.92 min; [M+H]⁺=248.2.

5-(4-Ethyl-phenyl)-2-methyl-thiazole-4-carboxylic acid methyl ester

prepared by reaction of 3-chloro-3-(4-ethyl-phenyl)-2-oxo-propionic acid methyl ester with thioacetamide. LC-MS (A): t_{R}=0.98 min; [M+H]⁺=262.1.

5-(3-Fluoro-phenyl)-2-methyl-thiazole-4-carboxylic acid methyl ester

prepared by reaction of 3-chloro-3-(3-fluoro-phenyl)-2-oxo-propionic acid methyl ester with thioacetamide. LC-MS (A): t_{R}=0.91 min; [M+H]⁺=252.1.

5-(4-Fluoro-phenyl)-2-methyl-thiazole-4-carboxylic acid methyl ester

prepared by reaction of 3-chloro-3-(4-fluoro-phenyl)-2-oxo-propionic acid methyl ester with thioacetamide. ¹H-NMR (CDCl₃): δ=2.75 (s, 3H), 3.84 (s, 3H), 7.10 (m, 2H), 7.47 (m, 2H).

5-(3-chloro-phenyl)-2-methyl-thiazole-4-carboxylic acid methyl ester

prepared by reaction of 3-chloro-3-(3-chloro-phenyl)-2-oxo-propionic acid methyl ester with thioacetamide. LC-MS (A): t_{R}=0.95 min; [M+H]⁺=268.0.

5-(4-chloro-phenyl)-2-methyl-thiazole-4-carboxylic acid methyl ester

prepared by reaction of 3-chloro-3-(4-chloro-phenyl)-2-oxo-propionic acid methyl ester with thioacetamide. LC-MS (A): t_{R}=0.94 min; [M+H]⁺=268.0.

5-(3,4-Dimethyl-phenyl)-2-methyl-thiazole-4-carboxylic acid methyl ester

prepared by reaction of 3-chloro-3-(3,4-dimethyl-phenyl)-2-oxo-propionic acid methyl ester with thioacetamide. LC-MS (A): t_{R}=0.96 min; [M+H]⁺=262.3.

2-Methyl-5-phenyl-thiazole-4-carboxylic acid methyl ester

prepared by reaction of 3-chloro-2-oxo-3-phenyl-propionic acid methyl ester with thioacetamide. LC-MS (A): t_{R}=0.87 min; [M+H]⁺=234.3.

5-(4-Cyano-phenyl)-2-methyl-thiazole-4-carboxylic acid methyl ester

prepared by reaction of 3-chloro-3-(4-cyano-phenyl)-2-oxo-propionic acid methyl ester with thioacetamide. LC-MS (A): t_{R}=0.92 min; [M+H]⁺=259.0.

5-(2,3-Dimethyl-phenyl)-2-methyl-thiazole-4-carboxylic acid methyl ester

prepared by reaction of 3-chloro-3-(2,3-dimethyl-phenyl)-2-oxo-propionic acid methyl ester with thioacetamide. LC-MS (A): t_{R}=0.95 min; [M+H]⁺=262.3.

5-(3-Fluoro-2-methyl-phenyl)-2-methyl-thiazole-4-carboxylic acid methyl ester

prepared by reaction of 3-chloro-3-(3-fluoro-2-methyl-phenyl)-2-oxo-propionic acid methyl ester with thioacetamide. LC-MS (A): t_{R}=0.93 min; [M+H]⁺=266.3.

5-(3,4-Dichloro-phenyl)-2-methyl-thiazole-4-carboxylic acid methyl ester

prepared by reaction of 3-chloro-3-(3,4-dichloro-phenyl)-2-oxo-propionic acid methyl ester with thioacetamide. LC-MS (A): t_{R}=0.99 min; [M+H]⁺=302.2.

5-(3,4-Difluoro-phenyl)-2-methyl-thiazole-4-carboxylic acid methyl ester

prepared by reaction of 3-chloro-3-(3,4-difluoro-phenyl)-2-oxo-propionic acid methyl ester with thioacetamide. LC-MS (A): t_{R}=0.92 min; [M+H]⁺=270.3.

5-(3-Fluoro-4-methyl-phenyl)-2-methyl-thiazole-4-carboxylic acid methyl ester

prepared by reaction of 3-chloro-3-(3-fluoro-4-methyl-phenyl)-2-oxo-propionic acid methyl ester with thioacetamide. LC-MS (A): t_{R}=1.00 min; [M+H]⁺=266.0.

5-(3,5-Dimethyl-phenyl)-2-methyl-thiazole-4-carboxylic acid methyl ester

prepared by reaction of 3-chloro-3-(3,5-dimethyl-phenyl)-2-oxo-propionic acid methyl ester with thioacetamide. LC-MS (A): t_{R}=0.97 min; [M+H]⁺=262.3.

5-(3-Fluoro-5-trifluoromethyl-phenyl)-2-methyl-thiazole-4-carboxylic acid methyl ester

prepared by reaction of 3-chloro-3-(3-fluoro-5-trifluoromethyl-phenyl)-2-oxo-propionic acid methyl ester with thioacetamide. LC-MS (A): t_{R}=1.03 min; [M+H]⁺=319.8.

5-(2,4-Dimethyl-phenyl)-2-methyl-thiazole-4-carboxylic acid methyl ester

prepared by reaction of 3-chloro-3-(2,4-dimethyl-phenyl)-2-oxo-propionic acid methyl ester with thioacetamide. LC-MS (A): t_{R}=0.96 min; [M+H]⁺=262.3.

5-(3,5-Difluoro-phenyl)-2-methyl-thiazole-4-carboxylic acid methyl ester

prepared by reaction of 3-chloro-3-(3,5-difluoro-phenyl)-2-oxo-propionic acid methyl ester with thioacetamide. LC-MS (A): t_{R}=0.92 min; [M+H]⁺=270.3.

5-(3-Cyano-phenyl)-2-methyl-thiazole-4-carboxylic acid methyl ester

prepared by reaction of 3-chloro-3-(3-cyano-phenyl)-2-oxo-propionic acid methyl ester with thioacetamide. LC-MS (A): t_{R}=0.86 min; [M+H]⁺=259.3.

5-(2,3-Difluoro-4-methyl-phenyl)-2-methyl-thiazole-4-carboxylic acid methyl ester

prepared by reaction of 3-chloro-3-(2,3-difluoro-4-methyl-phenyl)-2-oxo-propionic acid methyl ester with thioacetamide. LC-MS (A): t_{R}=0.95 min; [M+H]⁺=284.3.

A.1.3 Synthesis of 2-Cyclopropyl-Thiazole-4-Carboxylic Acid Methyl Ester derivatives

Synthesis of Cyclopropanecarbothioic Acid Amide

2,4-Bis-(4-methoxyphenyl)-1,3-dithia-2,4-diphosphetane 2,4-disulfide (Lawesson reagent, 173 mmol) is added to a mixture of cyclopropanecarboxamide (173 mmol) and Na₂CO₃ (173 mmol) in THF (750 mL). The reaction mixture is stirred at reflux for 3 h, concentrated in vacuo and diluted with ether (500 mL) and water (500 mL). The layers are separated and the aq. layer is extracted with ether (250 mL). The combined organic layers are washed with brine (100 mL), dried over MgSO₄ and concentrated in vacuo to give a crude product which is used without further purification. ¹H-NMR (DMSO-d₆): δ=0.81-0.88 (m, 2H); 0.96-1.00 (m, 2H); 2.00 (tt, J=8.0 Hz, J=4.3 Hz, 1H); 9.23 (bs, 1H); 9.33 (bs, 1H).

Synthesis of 2-cyclopropyl-thiazole-4-carboxylic acid methyl ester derivatives (General Procedure)

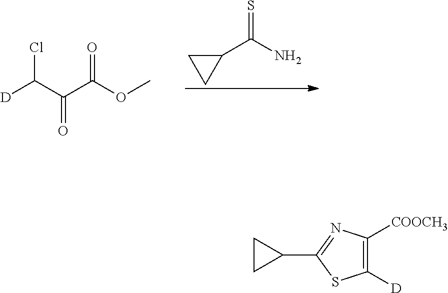

A solution of cyclopropanecarbothioic acid amide (33.9 mmol, 1.0 eq) in MeCN (45 mL) is added to a mixture of the respective 3-chloro-2-oxo-propionic ester derivative (33.9 mmol, 1.0 eq) and NaHCO₃ (102 mmol, 3.0 eq) in MeCN (45 mL). After stirring for 2d at RT the mixture is concentrated in vacuo and the residue is diluted with EtOAc (150 mL) and water (150 mL). The layers are separated and the aq. layer is extracted with EtOAc (100 mL). The combined organic layers are washed with brine (100 mL), dried over MgSO₄ and concentrated in vacuo. The residue is dissolved in MeOH (70 mL) and treated with concentrated H₂SO₄ (0.18 mL). The mixture is stirred at 60° C. for 16 h and concentrated in vacuo to give the respective crude product which is used without further purification.

2-Cyclopropyl-5-phenyl-thiazole-4-carboxylic acid methyl ester

prepared by reaction of 3-chloro-2-oxo-3-phenyl-propionic acid methyl ester with cyclopropanecarbothioic acid amide. LC-MS (A): t_{R}=0.99 min; [M+H]⁺=260.5.

2-Cyclopropyl-5-(3-fluoro-phenyl)-thiazole-4-carboxylic acid methyl ester

prepared by reaction of 3-chloro-3-(3-fluoro-phenyl)-2-oxo-propionic acid methyl ester with cyclopropanecarbothioic acid amide. LC-MS (A): t_{R}=1.02 min; [M+H]⁺=278.0.

2-Cyclopropyl-5-(3-fluoro-4-methyl-phenyl)-thiazole-4-carboxylic acid methyl ester

prepared by reaction of 3-chloro-3-(3-fluoro-4-methyl-phenyl)-2-oxo-propionic acid methyl ester with cyclopropanecarbothioic acid amide. LC-MS (A): t_{R}=1.06 min; [M+H]⁺=292.1.

2-Cyclopropyl-5-p-tolyl-thiazole-4-carboxylic acid methyl ester

prepared by reaction of 3-chloro-2-oxo-3-p-tolyl-propionic acid methyl ester with cyclopropanecarbothioic acid amide. LC-MS (A): t_{R}=1.04 min; [M+H]⁺=274.4.

2-Cyclopropyl-5-(4-fluoro-phenyl)-thiazole-4-carboxylic acid methyl ester

prepared by reaction of 3-chloro-3-(4-fluoro-phenyl)-2-oxo-propionic acid methyl ester with cyclopropanecarbothioic acid amide.

LC-MS (A): t_{R}=1.01 min; [M+H]⁺=278.3.

2-Cyclopropyl-5-(3-trifluoromethyl-phenyl)-thiazole-4-carboxylic acid methyl ester

prepared by reaction of 3-chloro-2-oxo-3-(3-trifluoromethyl-phenyl)-propionic acid methyl ester with cyclopropanecarbothioic acid amide.

LC-MS (A): t_{R}=1.07 min; [M+H]⁺=328.2.

2-Cyclopropyl-5-(3-fluoro-5-trifluoromethyl-phenyl)-thiazole-4-carboxylic acid methyl ester

prepared by reaction of 3-chloro-3-(3-fluoro-5-trifluoromethyl-phenyl)-2-oxo-propionic acid methyl ester with cyclopropanecarbothioic acid amide.

LC-MS (A): t_{R}=1.09 min; [M+H]⁺=346.0.

A.1.4 Synthesis of 2-amino-thiazole-4-carboxylic acid methyl ester derivatives (General Procedure)

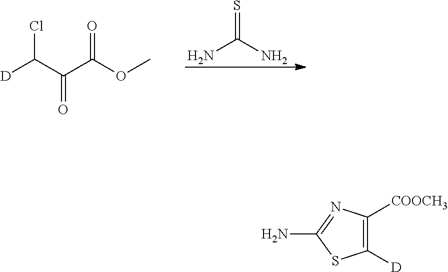

A solution of the respective 3-chloro-2-oxo-propionic ester derivative (22.1 mmol, 1.0 eq) in acetone (25 mL) is added to a suspension of thiourea (22.1 mmol, 1.0 eq) in acetone (45 mL). The mixture is heated to 57° C. (bath temperature), stirred for 24 h and concentrated to half of the volume. The obtained suspension is filtered and the residue is washed with acetone. After drying the desired amino-thiazole derivative is obtained as a solid.

2-Amino-5-m-tolyl-thiazole-4-carboxylic acid methyl ester

prepared by reaction of 3-chloro-2-oxo-3-m-tolyl-propionic acid methyl ester with thiourea. LC-MS (A): t_{R}=0.78 min; [M+H]⁺=249.0.

2-Amino-5-(3-fluoro-phenyl)-thiazole-4-carboxylic acid methyl ester

prepared by reaction of 3-chloro-3-(3-fluoro-phenyl)-2-oxo-propionic acid methyl ester with thiourea. LC-MS (A): t_{R}=0.78 min; [M+H]⁺=252.9.

2-Amino-5-(2-fluoro-phenyl)-thiazole-4-carboxylic acid methyl ester

prepared by reaction of 3-chloro-3-(2-fluoro-phenyl)-2-oxo-propionic acid methyl ester with thiourea. LC-MS (A): t_{R}=0.76 min; [M+H]⁺=253.2.

2-Amino-5-(3-methoxy-phenyl)-thiazole-4-carboxylic acid methyl ester

prepared by reaction of 3-chloro-3-(3-methoxy-phenyl)-2-oxo-propionic acid methyl ester with thiourea. LC-MS (A): t_{R}=0.75 min; [M+H]⁺=265.3.

2-Amino-5-(3-chloro-phenyl)-thiazole-4-carboxylic acid methyl ester

prepared by reaction of 3-chloro-3-(3-chloro-phenyl)-2-oxo-propionic acid methyl ester with thiourea. LC-MS (A): t_{R}=0.82 min; [M+H]⁺=269.2.

2-Amino-5-(3-trifluoromethyl-phenyl)-thiazole-4-carboxylic acid methyl ester

prepared by reaction of 3-chloro-3-(3-trifluoromethyl-phenyl)-2-oxo-propionic acid methyl ester with thiourea. LC-MS (A): t_{R}=0.86 min; [M+H]⁺=303.3.

2-Amino-5-(4-fluoro-phenyl)-thiazole-4-carboxylic acid methyl ester

prepared by reaction of 3-chloro-3-(4-fluoro-phenyl)-2-oxo-propionic acid methyl ester with thiourea. LC-MS (A): t_{R}=0.75 min; [M+H]⁺=253.2.

2-Amino-5-phenyl-thiazole-4-carboxylic acid methyl ester

prepared by reaction of 3-chloro-2-oxo-3-phenyl-propionic acid methyl ester with thiourea. LC-MS (A): t_{R}=0.77 min; [M+H]⁺=235.1.

2-Amino-5-p-tolyl-thiazole-4-carboxylic acid methyl ester

prepared by reaction of 3-chloro-2-oxo-3-p-tolyl-propionic acid methyl ester with thiourea. LC-MS (A): t_{R}=0.76 min; [M+H]⁺=249.3.

2-Amino-5-(3,4-dimethyl-phenyl)-thiazole-4-carboxylic acid methyl ester

prepared by reaction of 3-chloro-3-(3,4-dimethyl-phenyl)-2-oxo-propionic acid methyl ester with thiourea. ¹H NMR (DMSO-d₆): δ=2.06 (s, 2H), 2.21 (s, 3H), 2.22 (s, 3H), 3.63 (s, 3H), 7.13 (m, 2H), 7.18 (s, 1H).

A.1.5 Synthesis of 2-bromo-thiazole-4-carboxylic acid methyl ester derivatives (General Procedure)

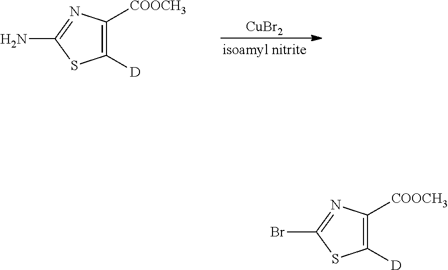

At 15° C. under an atmosphere of nitrogen the respective 2-amino-thiazole-4-carboxylic acid methyl ester (7.10 mmol) is added portionwise to a mixture of CuBr₂ (7.10 mmol) and isoamyl nitrite (10.6 mmol) in MeCN (30 mL). The mixture is stirred for 20 min at 15° C., for 30 min at 40° C. and for 90 min at 65° C. The solvents are removed in vacuo and the crude product is either purified by FC (DCM/MeOH or EtOAc/heptane) or used without further purification.

2-Bromo-5-m-tolyl-thiazole-4-carboxylic acid methyl ester

prepared by reaction of 2-amino-5-m-tolyl-thiazole-4-carboxylic acid methyl ester with CuBr₂ and isoamyl nitrite. LC-MS (A): t_{R}=1.01 min; [M+H]⁺=311.8.

2-Bromo-5-(2-fluoro-phenyl)-thiazole-4-carboxylic acid methyl ester

prepared by reaction of 2-amino-5-(2-fluoro-phenyl)-thiazole-4-carboxylic acid methyl ester with CuBr₂ and isoamyl nitrite. LC-MS (A): t_{R}=0.96 min; [M+H]⁺=316.1.

2-Bromo-5-(3-fluoro-phenyl)-thiazole-4-carboxylic acid methyl ester

prepared by reaction of 2-amino-5-(3-fluoro-phenyl)-thiazole-4-carboxylic acid methyl ester with CuBr₂ and isoamyl nitrite. LC-MS (A): t_{R}=1.08 min; [M+H]⁺=316.0.

2-Bromo-5-(3-methoxy-phenyl)-thiazole-4-carboxylic acid methyl ester

prepared by reaction of 2-amino-5-(3-methoxy-phenyl)-thiazole-4-carboxylic acid methyl ester with CuBr₂ and isoamyl nitrite. LC-MS (A): t_{R}=0.97 min; [M+H]⁺=328.2.

2-Bromo-5-(3-chloro-phenyl)-thiazole-4-carboxylic acid methyl ester

prepared by reaction of 2-amino-5-(3-chloro-phenyl)-thiazole-4-carboxylic acid methyl ester with CuBr₂ and isoamyl nitrite. LC-MS (A): t_{R}=1.00 min; [M+H]⁺=332.2.

2-Bromo-5-(3-trifluoromethyl-phenyl)-thiazole-4-carboxylic acid methyl ester

prepared by reaction of 2-amino-5-(3-trifluoromethyl-phenyl)-thiazole-4-carboxylic acid methyl ester with CuBr₂ and isoamyl nitrite. LC-MS (A): t_{R}=1.03 min; [M+H]⁺=366.2.

2-Bromo-5-(4-fluoro-phenyl)-thiazole-4-carboxylic acid methyl ester

prepared by reaction of 2-amino-5-(4-fluoro-phenyl)-thiazole-4-carboxylic acid methyl ester with CuBr₂ and isoamyl nitrite. LC-MS (A): t_{R}=0.97 min; [M+H]⁺=316.1.

2-Bromo-5-phenyl-thiazole-4-carboxylic acid methyl ester

prepared by reaction of 2-amino-5-phenyl-thiazole-4-carboxylic acid methyl ester with CuBr₂ and isoamyl nitrite. LC-MS (A): t_{R}=1.07 min; [M+H]⁺=297.9.

2-Bromo-5-(3,4-dimethyl-phenyl)-thiazole-4-carboxylic acid methyl ester

prepared by reaction of 2-amino-5-(3,4-dimethyl-phenyl)-thiazole-4-carboxylic acid methyl ester with CuBr₂ and isoamyl nitrite. ¹H NMR (CDCl₃): δ=2.30 (s, 6H), 3.84 (s, 3H), 7.20 (s, 1H), 7.21 (m, 1H), 7.23 (m, 1H).

A.1.6 Synthesis of thiazole-4-carboxylic acid methyl ester derivatives lacking a substituent in 2-position (General Procedure)

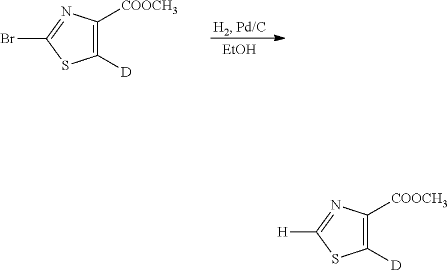

A solution/suspension of the respective 2-bromo-thiazole-4-carboxylic acid methyl ester (3.17 mmol) in EtOH (20 mL) is added to a suspension of Pd/C (600 mg, 10%) in EtOH (20 mL) and stirred under a hydrogen atmosphere (1 bar) for 18 h. After filtration through celite and removal of the solvents the desired product is obtained which is used without further purification.

5-m-Tolyl-thiazole-4-carboxylic acid methyl ester

prepared by hydrogenation of 2-bromo-5-m-tolyl-thiazole-4-carboxylic acid methyl ester. LC-MS (A): t_{R}=0.90 min; [M+H]⁺=233.9.

5-(2-Fluoro-phenyl)-thiazole-4-carboxylic acid methyl ester

prepared by hydrogenation of 2-bromo-5-(2-fluoro-phenyl)-thiazole-4-carboxylic acid methyl ester. LC-MS (A): t_{R}=0.91 min; [M+H]⁺=238.0.

5-(3-Fluoro-phenyl)-thiazole-4-carboxylic acid methyl ester

prepared by hydrogenation of 2-bromo-5-(3-fluoro-phenyl)-thiazole-4-carboxylic acid methyl ester. LC-MS (A): t_{R}=0.92 min; [M+H]⁺=238.1.

5-Phenyl-thiazole-4-carboxylic acid methyl ester

prepared by hydrogenation of 2-bromo-5-phenyl-thiazole-4-carboxylic acid methyl ester. LC-MS (A): t_{R}=0.89 min; [M+H]⁺=220.1.

5-(3-Methoxy-phenyl)-thiazole-4-carboxylic acid methyl ester

prepared by hydrogenation of 2-bromo-5-(3-methoxy-phenyl)-thiazole-4-carboxylic acid methyl ester. LC-MS (A): t_{R}=0.92 min; [M+H]⁺=250.1.

5-(3-Chloro-phenyl)-thiazole-4-carboxylic acid methyl ester

prepared by hydrogenation of 2-bromo-5-(3-chloro-phenyl)-thiazole-4-carboxylic acid methyl ester. LC-MS (A): t_{R}=0.91 min; [M+H]⁺=253.9.

5-(3-Trifluoromethyl-phenyl)-thiazole-4-carboxylic acid methyl ester

prepared by hydrogenation of 2-bromo-5-(3-trifluoromethyl-phenyl)-thiazole-4-carboxylic acid methyl ester. LC-MS (A): t_{R}=0.99 min; [M+H]⁺=288.0.

5-(4-Fluoro-phenyl)-thiazole-4-carboxylic acid methyl ester

prepared by hydrogenation of 2-bromo-5-(4-fluoro-phenyl)-thiazole-4-carboxylic acid methyl ester. LC-MS (A): t_{R}=0.92 min; [M+H]⁺=238.1.

5-(3,4-Dimethyl-phenyl)-thiazole-4-carboxylic acid methyl ester

prepared by hydrogenation of 2-bromo-5-(3,4-dimethyl-phenyl)-thiazole-4-carboxylic acid methyl ester. ¹H NMR (CDCl₃): δ=2.33 (s, 6H), 3.97 (s, 3H), 7.26 (m, 1H), 7.34 (m, 2H).

A.1.7 Synthesis of 2-Dimethylaminomethyl-5-m-tolyl-thiazole-4-carboxylic acid methyl ester

DIPEA (11.4 mmol) is added to a mixture of 3-chloro-2-oxo-3-m-tolyl-propionic acid methyl ester (11.4 mmol) and N,N-dimethylamino-thioacetamide hydrochloride (11.4 mmol) in acetonitrile (100 mL). After 5 h the suspension is filtered and the filtrate is concentrated in vacuo. The residue is dissolved in MeOH (100 mL) and treated with a solution of HCl in ether (2.0 M, 2.5 mL). The mixture is heated to 50° C., stirred for 8 h, cooled to RT and stirred additional 16 h. The solvents are removed in vacuo, the residue is diluted with EtOAc and hydrochloric acid (1.0 M) and the layers are separated. The aqueous layer is washed three times with EtOAc (50 mL each), made basic (pH ˜10) by addition of aqueous NaOH solution (1.0 M) and extracted three times with EtOAc (50 mL each). The combined organic layers are dried over MgSO₄ and concentrated in vacuo to give the desired product which is used without further purification in the next step. LC-MS (C): t_{R}=0.47 min; [M+H]⁺=291.1.

A.1.8 Synthesis of 2-(tert-Butoxycarbonylamino-methyl)-5-m-tolyl-thiazole-4-carboxylic acid methyl ester

A solution of 3-chloro-2-oxo-3-m-tolyl-propionic acid methyl ester (1.52 mmol) in acetonitrile (2.5 mL) is added to a mixture of tert-butyl 2-amino-2-thioxoethylcarbamate (1.52 mmol) in acetonitrile. The mixture is stirred for 3 h at RT, the suspension is filtered and the residue is washed twice with acetonitrile (2×1 mL). The combined filtrates are concentrated in vacuo and the residue is purified by prep. thin layer chromatography (DCM/MeOH 97/3) to give the desired product. LC-MS (C): t_{R}=0.81 min; [M+H]⁺=363.2.

A.1.9 Synthesis of thiazole-4-carboxylic acid derivatives (General Procedure)

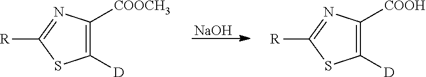

A solution of the respective ester (96.2 mmol) in a mixture of THF (150 mL) and MeOH (or isopropanol, 50 mL) is treated with an aqueous NaOH solution (1.0 M, 192 mL; or 2.0 M, 96 mL). After stirring for 3 h a white suspension is formed and the organic volatiles are removed in vacuo. The remaining mixture is diluted with water (100 mL), cooled in an ice-bath and made acidic (pH=3-4) by addition of aqueous HCl solution (1.0 M). In case of precipitation, the suspension is filtered and the residue is washed with cold water and dried in vacuo to give the desired acid. In other cases, the mixture is extracted twice with EtOAc and the organic layers are combined, dried over MgSO₄ and concentrated in vacuo to give the respective acid.

2-Methyl-5-m-tolyl-thiazole-4-carboxylic acid

prepared by saponification of 2-methyl-5-m-tolyl-thiazole-4-carboxylic acid methyl ester. LC-MS (A): t_{R}=0.83 min; [M+H]⁺=234.0.

2-Methyl-5-p-tolyl-thiazole-4-carboxylic acid

prepared by saponification of 2-methyl-5-p-tolyl-thiazole-4-carboxylic acid methyl ester. LC-MS (A): t_{R}=0.83 min; [M+H]⁺=234.0.

5-(4-Ethyl-phenyl)-2-methyl-thiazole-4-carboxylic acid

prepared by saponification of 5-(4-ethyl-phenyl)-2-methyl-thiazole-4-carboxylic acid methyl ester. LC-MS (A): t_{R}=0.88 min; [M+H]⁺=248.0.

5-(3-Fluoro-phenyl)-2-methyl-thiazole-4-carboxylic acid

prepared by saponification of 5-(3-fluoro-phenyl)-2-methyl-thiazole-4-carboxylic acid methyl ester. LC-MS (A): t_{R}=0.82 min; [M+H]⁺=238.1.

5-(4-Fluoro-phenyl)-2-methyl-thiazole-4-carboxylic acid

prepared by saponification of 5-(4-fluoro-phenyl)-2-methyl-thiazole-4-carboxylic acid methyl ester. ¹H-NMR (DMSO-d₆): δ=2.67 (s, 3H), 7.27 (m, 2H), 7.53 (m, 2H), 12.89 (bs, 1H).

5-(3-chloro-phenyl)-2-methyl-thiazole-4-carboxylic acid

prepared by saponification of 5-(3-chloro-phenyl)-2-methyl-thiazole-4-carboxylic acid methyl ester. LC-MS (A): t_{R}=0.84 min; [M+H]⁺=254.0.

5-(4-chloro-phenyl)-2-methyl-thiazole-4-carboxylic acid

prepared by saponification of 5-(4-chloro-phenyl)-2-methyl-thiazole-4-carboxylic acid methyl ester. LC-MS (A): t_{R}=0.85 min; [M+H]⁺=254.0.

5-(3,4-Dimethyl-phenyl)-2-methyl-thiazole-4-carboxylic acid

prepared by saponification of 5-(3,4-dimethyl-phenyl)-2-methyl-thiazole-4-carboxylic acid methyl ester. LC-MS (A): t_{R}=0.86 min; [M+H]⁺=248.3.

2-Amino-5-m-tolyl-thiazole-4-carboxylic acid

prepared by saponification of 2-amino-5-m-tolyl-thiazole-4-carboxylic acid methyl ester. LC-MS (A): t_{R}=0.65 min; [M+H]⁺=235.0.

2-Amino-5-(3-fluoro-phenyl)-thiazole-4-carboxylic acid

prepared by saponification of 2-amino-5-(3-fluoro-phenyl)-thiazole-4-carboxylic acid methyl ester. LC-MS (A): t_{R}=0.62 min; [M+H]⁺=239.1.

2-Bromo-5-m-tolyl-thiazole-4-carboxylic acid

prepared by saponification of 2-Bromo-5-m-tolyl-thiazole-4-carboxylic acid methyl ester. LC-MS (B): t_{R}=0.57 min; [M+H]⁺=297.8.

2-Methyl-5-phenyl-thiazole-4-carboxylic acid

prepared by saponification of 2-methyl-5-phenyl-thiazole-4-carboxylic acid methyl ester. LC-MS (A): t_{R}=0.77 min; [M+H]⁺=220.3.

5-(4-Cyano-phenyl)-2-methyl-thiazole-4-carboxylic acid

prepared by saponification of 5-(4-cyano-phenyl)-2-methyl-thiazole-4-carboxylic acid methyl ester. LC-MS (A): t_{R}=0.82 min; [M+H]⁺=245.1.

5-(2,3-Dimethyl-phenyl)-2-methyl-thiazole-4-carboxylic acid

prepared by saponification of 5-(2,3-dimethyl-phenyl)-2-methyl-thiazole-4-carboxylic acid methyl ester. LC-MS (A): t_{R}=0.84 min; [M+H]⁺=248.3.

5-(3-Fluoro-2-methyl-phenyl)-2-methyl-thiazole-4-carboxylic acid

prepared by saponification of 5-(3-fluoro-2-methyl-phenyl)-2-methyl-thiazole-4-carboxylic acid methyl ester. LC-MS (A): t_{R}=0.83 min; [M+H]⁺=252.2.

5-(3,4-Dichloro-phenyl)-2-methyl-thiazole-4-carboxylic acid

prepared by saponification of 5-(3,4-dichloro-phenyl)-2-methyl-thiazole-4-carboxylic acid methyl ester. LC-MS (A): t_{R}=0.88 min; [M+H]⁺=288.2.

5-(3,4-Difluoro-phenyl)-2-methyl-thiazole-4-carboxylic acid

prepared by saponification of 5-(3,4-difluoro-phenyl)-2-methyl-thiazole-4-carboxylic acid methyl ester. LC-MS (A): t_{R}=0.82 min; [M+H]⁺=256.3.

5-(3-Fluoro-4-methyl-phenyl)-2-methyl-thiazole-4-carboxylic acid

prepared by saponification of 5-(3-fluoro-4-methyl-phenyl)-2-methyl-thiazole-4-carboxylic acid methyl ester. LC-MS (A): t_{R}=0.89 min; [M+H]⁺=252.0.

5-(3,5-Dimethyl-phenyl)-2-methyl-thiazole-4-carboxylic acid

prepared by saponification of 5-(3,5-dimethyl-phenyl)-2-methyl-thiazole-4-carboxylic acid methyl ester. LC-MS (A): t_{R}=0.86 min; [M+H]⁺=248.3.

5-(3-Fluoro-5-trifluoromethyl-phenyl)-2-methyl-thiazole-4-carboxylic acid

prepared by saponification of 5-(3-fluoro-5-trifluoromethyl-phenyl)-2-methyl-thiazole-4-carboxylic acid methyl ester. LC-MS (A): t_{R}=0.94 min; [M+H]⁺=306.0.

5-(2,4-Dimethyl-phenyl)-2-methyl-thiazole-4-carboxylic acid

prepared by saponification of 5-(2,4-dimethyl-phenyl)-2-methyl-thiazole-4-carboxylic acid methyl ester. LC-MS (A): t_{R}=0.85 min; [M+H]⁺=248.3.

5-m-tolyl-thiazole-4-carboxylic acid

prepared by saponification of 5-m-tolyl-thiazole-4-carboxylic acid methyl ester. LC-MS (B): t_{R}=0.54 min; [M+H]⁺=218.3.

5-(2-Fluoro-phenyl)-thiazole-4-carboxylic acid

prepared by saponification of 5-(2-fluoro-phenyl)-thiazole-4-carboxylic acid methyl ester. LC-MS (A): t_{R}=0.80 min; [M+H]⁺=224.1.

5-(3-Fluoro-phenyl)-thiazole-4-carboxylic acid

prepared by saponification of 5-(3-fluoro-phenyl)-thiazole-4-carboxylic acid methyl ester. LC-MS (A): t_{R}=0.80 min; [M+H]⁺=224.0.

5-Phenyl-thiazole-4-carboxylic acid

prepared by saponification of 5-phenyl-thiazole-4-carboxylic acid methyl ester.

LC-MS (A): t_{R}=0.78 min; [M+H]⁺=206.2.

5-(3-Methoxy-phenyl)-thiazole-4-carboxylic acid

prepared by saponification of 5-(3-methoxy-phenyl)-thiazole-4-carboxylic acid methyl ester. LC-MS (A): t_{R}=0.81 min; [M+H]⁺=236.1.

5-(3-Chloro-phenyl)-thiazole-4-carboxylic acid

prepared by saponification of 5-(3-chloro-phenyl)-thiazole-4-carboxylic acid methyl ester. LC-MS (A): t_{R}=0.85 min; [M+H]⁺=240.0.

5-(3-Trifluoromethyl-phenyl)-thiazole-4-carboxylic acid

prepared by saponification of 5-(3-trifluoromethyl-phenyl)-thiazole-4-carboxylic acid methyl ester. LC-MS (A): t_{R}=0.89 min; [M+H]⁺=274.0.

5-(4-Fluoro-phenyl)-thiazole-4-carboxylic acid

prepared by saponification of 5-(4-fluoro-phenyl)-thiazole-4-carboxylic acid methyl ester. LC-MS (A): t_{R}=0.80 min; [M+H]⁺=224.1.

2-Cyclopropyl-5-phenyl-thiazole-4-carboxylic acid

prepared by saponification of 2-cyclopropyl-5-phenyl-thiazole-4-carboxylic acid methyl ester. LC-MS (A): t_{R}=0.91 min; [M+H]⁺=246.4.

2-Cyclopropyl-5-(3-fluoro-phenyl)-thiazole-4-carboxylic acid

prepared by saponification of 2-cyclopropyl-5-(3-fluoro-phenyl)-thiazole-4-carboxylic acid methyl ester. LC-MS (A): t_{R}=0.92 min; [M+H]⁺=264.0.

2-Cyclopropyl-5-(3-fluoro-4-methyl-phenyl)-thiazole-4-carboxylic acid

prepared by saponification of 2-cyclopropyl-5-(3-fluoro-4-methyl-phenyl)-thiazole-4-carboxylic acid methyl ester. LC-MS (A): t_{R}=0.97 min; [M+H]⁺=278.1.

2-Amino-5-(2-fluoro-phenyl)-thiazole-4-carboxylic acid

prepared by saponification of 2-amino-5-(2-fluoro-phenyl)-thiazole-4-carboxylic acid methyl ester. LC-MS (A): t_{R}=0.60 min; [M+H]⁺=239.2.

2-Amino-5-phenyl-thiazole-4-carboxylic acid

prepared by saponification of 2-amino-5-phenyl-thiazole-4-carboxylic acid methyl ester. LC-MS (A): t_{R}=0.63 min; [M+H]⁺=221.4.

2-Amino-5-(3-chloro-phenyl)-thiazole-4-carboxylic acid

prepared by saponification of 2-amino-5-(3-chloro-phenyl)-thiazole-4-carboxylic acid methyl ester. LC-MS (A): t_{R}=0.66 min; [M+H]⁺=255.2.

2-Amino-5-p-tolyl-thiazole-4-carboxylic acid

prepared by saponification of 2-amino-5-p-tolyl-thiazole-4-carboxylic acid methyl ester. LC-MS (A): t_{R}=0.64 min; [M+H]⁺=235.2.

2-Cyclopropyl-5-p-tolyl-thiazole-4-carboxylic acid

prepared by saponification of 2-cyclopropyl-5-p-tolyl-thiazole-4-carboxylic acid methyl ester. LC-MS (A): t_{R}=0.91 min; [M+H]⁺=260.0.

2-Cyclopropyl-5-(4-fluoro-phenyl)-thiazole-4-carboxylic acid

prepared by saponification of 2-cyclopropyl-5-(4-fluoro-phenyl)-thiazole-4-carboxylic acid methyl ester. LC-MS (A): t_{R}=0.88 min; [M+H]⁺=264.0.

2-Cyclopropyl-5-(3-trifluoromethyl-phenyl)-thiazole-4-carboxylic acid

prepared by saponification of 2-cyclopropyl-5-(3-trifluoromethyl-phenyl)-thiazole-4-carboxylic acid methyl ester. LC-MS (A): t_{R}=1.00 min; [M+H]⁺=314.3.

2-Cyclopropyl-5-(3-fluoro-5-trifluoromethyl-phenyl)-thiazole-4-carboxylic acid

prepared by saponification of 2-cyclopropyl-5-(3-fluoro-5-trifluoromethyl-phenyl)-thiazole-4-carboxylic acid methyl ester. LC-MS (A): t_{R}=1.01 min; [M+H]⁺=332.0.

5-(3,5-Difluoro-phenyl)-2-methyl-thiazole-4-carboxylic acid

prepared by saponification of 5-(3,5-difluoro-phenyl)-2-methyl-thiazole-4-carboxylic acid methyl ester. LC-MS (A): t_{R}=0.82 min; [M+H]⁺=256.3.

5-(3-Cyano-phenyl)-2-methyl-thiazole-4-carboxylic acid

prepared by saponification of 5-(3-cyano-phenyl)-2-methyl-thiazole-4-carboxylic acid methyl ester. LC-MS (A): t_{R}=0.76 min; [M+H]⁺=245.3.

5-(2,3-Difluoro-4-methyl-phenyl)-2-methyl-thiazole-4-carboxylic acid

prepared by saponification of 5-(2,3-difluoro-4-methyl-phenyl)-2-methyl-thiazole-4-carboxylic acid methyl ester. LC-MS (A): t_{R}=0.85 min; [M+H]⁺=270.2.

5-(3,4-Dimethyl-phenyl)-thiazole-4-carboxylic acid

prepared by saponification of 5-(3,4-dimethyl-phenyl)-thiazole-4-carboxylic acid methyl ester. ¹H NMR (CDCl₃): δ=2.31 (s, 6H), 7.20 (d, J=7.9 Hz, 1H), 7.37 (m, 2H), 8.70 (s, 1H).

2-Dimethylaminomethyl-5-m-tolyl-thiazole-4-carboxylic acid

prepared by saponification of 2-dimethylaminomethyl-5-m-tolyl-thiazole-4-carboxylic acid methyl ester. LC-MS (C): t_{R}=0.49 min; [M+H]⁺=277.1.

2-(tert-Butoxycarbonylamino-methyl)-5-m-tolyl-thiazole-4-carboxylic acid

prepared by saponification of 2-(tert-butoxycarbonylamino-methyl)-5-m-tolyl-thiazole-4-carboxylic acid methyl ester. LC-MS (C): t_{R}=0.71 min; [M+H]⁺=349.2.

A.1.10 Synthesis of 2-dimethylamino-thiazole-4-carboxylic acid derivatives (General Procedure)

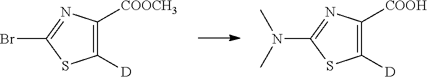

An aq. solution of dimethylamine (40%, 13 mL) is added to a solution of the respective 2-bromo-thiazole-4-carboxylic acid methyl ester derivative (6.71 mmol) in MeCN (38 mL). After 2 h an additional portion of an aq. dimethylamine solution (40%, 13 mL) is added. After stirring at RT for 2d THF (13.6 mL), MeOH (6.8 mL) and aq. NaOH solution (1.0 M, 13.4 mL) are added successively and the mixture is stirred for 16 h. The solvents are removed in vacuo and the residue is diluted with water (30 mL). The suspension is made acidic (pH 3) by addition of aq. citric acid (10%) and extracted three times with EtOAc. The combined organic layers are washed twice with brine, dried over MgSO₄ and concentrated in vacuo to give the desired acid which is used without further purification.

2-Dimethylamino-5-m-tolyl-thiazole-4-carboxylic acid

prepared by reaction of 2-bromo-5-m-tolyl-thiazole-4-carboxylic acid methyl ester with dimethylamine. LC-MS (A): t_{R}=0.85 min; [M+H]⁺=263.1.

2-Dimethylamino-5-(3,4-dimethyl-phenyl)-thiazole-4-carboxylic acid

prepared by reaction of 2-bromo-5-(3,4-dimethyl-phenyl)-thiazole-4-carboxylic acid methyl ester with dimethylamine. ¹H NMR (CDCl₃): δ=2.27 (s, 6H), 3.11 (s, 6H), 7.14 (d, J=8.2 Hz, 1H), 7.36 (m, 2H).

A.1.11 Synthesis of 2-dimethylamino-5-(3-methoxy-phenyl)-thiazole-4-carboxylic acid

An aq. solution of dimethylamine (40%, 37 mL) is added to a solution of 2-bromo-5-(3-methoxy-phenyl)-thiazole-4-carboxylic acid methyl ester (9.15 mmol) in MeCN (20 mL). After stirring at RT for 16 h the suspension is made acidic (pH=3-4) by addition of water (30 mL) and solid citric acid monohydrate. EtOAc is added, the layers are separated and the aqueous layer is extracted twice with EtOAc. The combined organic layers are washed with water, dried over MgSO₄ and concentrated in vacuo to give crude 2-dimethylamino-5-(3-methoxy-phenyl)-thiazole-4-carboxylic acid methyl ester (LC-MS (A): t_{R}=0.95 min; [M+H]⁺=293.4). The ester is dissolved in MeOH (13 mL) and THF (18 mL), treated with aq. NaOH solution (1.0 M, 19 mL) and stirred for 18 h. The solvents are removed in vacuo and the residue is diluted with water. The mixture is made acidic (pH=1-2) by addition of hydrochloric acid (2.0 M). DCM is added, the layers are separated and the aqueous layer is extracted twice with DCM. The combined organic layers are dried over MgSO₄ and concentrated in vacuo to give the desired acid which is used without further purification. LC-MS (A): t_{R}=0.82 min; [M+H]⁺=279.3.

A.1.12 Synthesis of 2-alkoxy-thiazole-4-carboxylic acid derivatives (General Procedure)

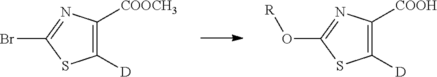

At 0° C. under an atmosphere of nitrogen the respective alcohol (0.96 mmol) is added to a suspension of sodium hydride (0.96 mmol) in THF (2.0 mL). After 5 min a solution of the respective 2-bromo-thiazole-4-carboxylic acid methyl ester (0.48 mmol) in DMF (0.2 mL) and THF (1.0 mL) is added dropwise. The mixture is stirred for 16 h at RT, cooled to 0° C. and treated with water (0.5 mL) and aq. NaOH solution (1.0 M, 0.5 mL). After 2 h the solvents are removed in vacuo and the residue is dissolved in warm water (1.0 mL). Ether is added, the layers are separated and the aq. layer is concentrated partially in vacuo to remove traces of ether. The mixture is cooled to 0° C. and made acidic (pH 4) by addition of aq. HCl (2.0 M). The precipitate is filtered off, washed with water and dried in vacuo to give the desired product.

2-Methoxy-5-m-tolyl-thiazole-4-carboxylic acid

prepared by reaction of 2-bromo-5-m-tolyl-thiazole-4-carboxylic acid methyl ester with MeOH. LC-MS (A): t_{R}=0.88 min; [M+H]⁺=250.3.

A.1.13 Synthesis of 5-(6-methoxy-pyridin-3-yl)-2-methyl-thiazole-4-carboxylic acid

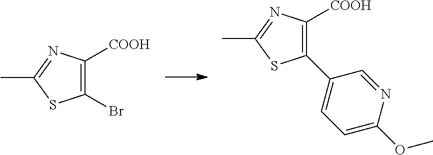

5-Bromo-2-methyl-thiazole-4-carboxylic acid

At −78° C. under an atmosphere of nitrogen a solution of n-BuLi in hexane (1.6 M, 20 mL) is added drop wise to a solution of 2-methyl-thiazole-4-carboxylic acid (15.2 mmol) in THF (125 mL). A solution of bromine (16.8 mmol) in cyclohexane (3.5 mL) is added drop wise at −78° C. and the mixture is stirred for 60 min at RT. Water (3.4 mL) is added and the organic volatiles are removed in vacuo. The mixture is made acidic (pH 2) by addition of hydrochloric acid (2.0 M) and extracted three times with EtOAc (3×50 mL). The combined organic layers are dried over MgSO₄ and concentrated in vacuo to give the desired product which is used without further purification. LC-MS (C): t_{R}=0.39 min; [M+H]⁺=222.1.

5-(6-Methoxy-pyridin-3-yl)-2-methyl-thiazole-4-carboxylic acid

A freshly prepared aqueous Na₂CO₃ solution (2.0 M, 18 mL) is added to a suspension of 5-bromo-2-methyl-thiazole-4-carboxylic acid (2.93 mmol) and 2-methoxypyridine-5-boronic acid (2.93 mmol) in a mixture of toluene (12 mL) and EtOH (12 mL). Argon is passed through the mixture to remove oxygen, tetrakis(triphenyl-phosphine)palladium(0) (94.4 mg) is added under argon and the mixture is vigorously stirred at 75° C. for 22 h. The layers are separated and the aqueous layer is washed twice with toluene (2×20 mL). Acetic acid (2.1 mL) is added (pH ˜6-7) and the aqueous layer is extracted four times with EtOAc (4×20 mL). The combined organic layers are dried over MgSO₄ and concentrated in vacuo. TBME is added, the suspension is filtered and the residue is dried in vacuo to give the desired product as a beige solid. LC-MS (C): t_{R}=0.48 min; [M+H]⁺=251.2.

A.2 Synthesis of thiazole-5-carboxylic acid derivatives
A.2.1 Synthesis of 2-chloro-3-oxo-propionic ester derivatives (General Procedure)

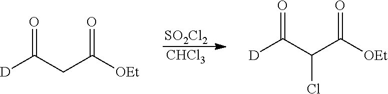

A mixture of the respective β-keto ester (5.52 mmol) and sulfuryl chloride (5.52 mmol) in chloroform (3.3 mL) is heated at reflux for 14 h, cooled to RT and washed with water. The solution is dried over MgSO₄ and concentrated in vacuo to give the desired product which is used immediately in the next step without further purification.

2-Chloro-3-(4-fluoro-phenyl)-3-oxo-propionic acid ethyl ester

prepared by chlorination of 3-(4-fluoro-phenyl)-3-oxo-propionic acid ethyl ester.

2-Chloro-3-oxo-3-p-tolyl-propionic acid ethyl ester

prepared by chlorination of 3-p-tolyl-3-oxo-propionic acid ethyl ester.

2-Chloro-3-oxo-3-(4-trifluoromethyl-phenyl)-propionic acid ethyl ester

prepared by chlorination of 3-oxo-3-(4-trifluoromethyl-phenyl)-propionic acid ethyl ester.

2-Chloro-3-(4-chloro-phenyl)-3-oxo-propionic acid ethyl ester

prepared by chlorination of 3-(4-chloro-phenyl)-3-oxo-propionic acid ethyl ester.

2-Chloro-3-(3-chloro-phenyl)-3-oxo-propionic acid ethyl ester

prepared by chlorination of 3-(3-chloro-phenyl)-3-oxo-propionic acid ethyl ester.

2-Chloro-3-oxo-3-(3-trifluoromethyl-phenyl)-propionic acid ethyl ester

prepared by chlorination of 3-oxo-3-(3-trifluoromethyl-phenyl)-propionic acid ethyl ester.

2-Chloro-3-(3-methoxy-phenyl)-3-oxo-propionic acid ethyl ester

prepared by chlorination of 3-(3-methoxy-phenyl)-3-oxo-propionic acid ethyl ester.

A.2.2 Synthesis of thiazole-5-carboxylic acid ethyl ester derivatives (General Procedure)

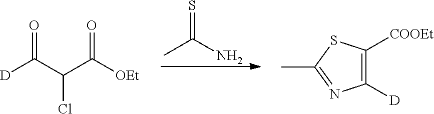

A mixture of the respective 2-chloro-3-oxo-propionic ester derivatives (5.52 mmol), thioacetamide (6.75 mmol) and NaHCO₃ (6.07 mmol) in THF (12 mL) is heated at reflux for 6 h, filtered and concentrated in vacuo to give a crude product which is purified by FC (heptane to heptane/EtOAc 6/4).

4-(4-Fluoro-phenyl)-2-methyl-thiazole-5-carboxylic acid ethyl ester

prepared by reaction of 2-chloro-3-(4-fluoro-phenyl)-3-oxo-propionic acid ethyl ester with thioacetamide. LC-MS (A): t_{R}=0.95 min; [M+H]⁺=266.1.

2-Methyl-4-p-tolyl-thiazole-5-carboxylic acid ethyl ester

prepared by reaction of 2-chloro-3-oxo-3-p-tolyl-propionic acid ethyl ester with thioacetamide. LC-MS (A): t_{R}=1.00 min; [M+H]⁺=262.0.

2-Methyl-4-(4-trifluoromethyl-phenyl)-thiazole-5-carboxylic acid ethyl ester

prepared by reaction of 2-chloro-3-oxo-3-(4-trifluoromethyl-phenyl)-propionic acid ethyl ester with thioacetamide. LC-MS (B): t_{R}=1.01 min; [M+CH₃CN+H]⁺=357.1.

4-(4-Chloro-phenyl)-2-methyl-thiazole-5-carboxylic acid ethyl ester

prepared by reaction of 2-chloro-3-(4-chloro-phenyl)-3-oxo-propionic acid ethyl ester with thioacetamide. LC-MS (B): t_{R}=1.00 min; [M+H]⁺=281.9.

4-(3-Chloro-phenyl)-2-methyl-thiazole-5-carboxylic acid ethyl ester

prepared by reaction of 2-chloro-3-(3-chloro-phenyl)-3-oxo-propionic acid ethyl ester with thioacetamide. LC-MS (B): t_{R}=1.00 min; [M+H]⁺=282.1.

2-Methyl-4-(3-trifluoromethyl-phenyl)-thiazole-5-carboxylic acid ethyl ester

prepared by reaction of 2-chloro-3-oxo-3-(3-trifluoromethyl-phenyl)-propionic acid ethyl ester with thioacetamide. LC-MS (B): t_{R}=1.02 min; [M+CH₃CN+H]⁺=357.2.

4-(3-Methoxy-phenyl)-2-methyl-thiazole-5-carboxylic acid ethyl ester

prepared by reaction of 2-chloro-3-(3-methoxy-phenyl)-3-oxo-propionic acid ethyl ester with thioacetamide. LC-MS (B): t_{R}=0.92 min; [M+H]⁺=278.1.

A.2.3 Synthesis of thiazole-5-carboxylic acid derivatives (General Procedure)

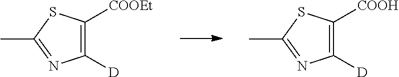

A mixture of the respective thiazole-5-carboxylic acid ethyl ester derivatives (3.38 mmol) and KOH (6.76 mmol) in EtOH (8.5 mL) and water (2.1 mL) is heated to reflux for 3 h, cooled to RT and concentrated in vacuo. Ice-cold water and hexane is added, the layers are separated and the aq. layer is made acidic by addition of aq. HCl (1.0 M). The obtained precipitate is filtered off, washed with water and dried in vacuo to give the desired acid.

4-(4-Fluoro-phenyl)-2-methyl-thiazole-5-carboxylic acid

prepared by saponification of 4-(4-fluoro-phenyl)-2-methyl-thiazole-5-carboxylic acid ethyl ester. LC-MS (A): t_{R}=0.81 min; [M+H]⁺=238.0.

2-Methyl-4-p-tolyl-thiazole-5-carboxylic acid

prepared by saponification of 2-methyl-4-p-tolyl-thiazole-5-carboxylic acid ethyl ester. LC-MS (A): t_{R}=0.83 min; [M+H]⁺=234.0.

2-Methyl-4-(4-trifluoromethyl-phenyl)-thiazole-5-carboxylic acid

prepared by saponification of 2-methyl-4-(4-trifluoromethyl-phenyl)-thiazole-5-carboxylic acid ethyl ester. LC-MS (A): t_{R}=0.91 min; [M+H]⁺=288.5.

4-(4-Chloro-phenyl)-2-methyl-thiazole-5-carboxylic acid

prepared by saponification of 4-(4-chloro-phenyl)-2-methyl-thiazole-5-carboxylic acid ethyl ester. LC-MS (A): t_{R}=0.86 min; [M+H]⁺=253.9.

4-(3-Chloro-phenyl)-2-methyl-thiazole-5-carboxylic acid

prepared by saponification of 4-(3-chloro-phenyl)-2-methyl-thiazole-5-carboxylic acid ethyl ester. LC-MS (A): t_{R}=0.85 min; [M+H]⁺=254.2.

2-Methyl-4-(3-trifluoromethyl-phenyl)-thiazole-5-carboxylic acid

prepared by saponification of 2-methyl-4-(3-trifluoromethyl-phenyl)-thiazole-5-carboxylic acid ethyl ester. LC-MS (A): t_{R}=0.90 min; [M+H]⁺=288.3.

4-(3-Methoxy-phenyl)-2-methyl-thiazole-5-carboxylic acid

prepared by saponification of 4-(3-methoxy-phenyl)-2-methyl-thiazole-5-carboxylic acid ethyl ester. LC-MS (A): t_{R}=0.78 min; [M+H]⁺=250.3.

A.3 Synthesis of oxazole-4-carboxylic acid derivatives
A.3.1 Synthesis of 2-acetylamino-3-oxo-3-phenyl-propionic acid ethyl ester derivatives (General Procedure)

A solution of the respective 3-oxo-3-phenyl-propionic acid ethyl ester derivative (4.85 mmol) in acetic acid (1.90 mL) is cooled to 10° C. and a solution of sodium nitrite (5.63 mmol) in water (0.68 mL) is added dropwise. The mixture is allowed to reach RT, stirred for 2 h, poured into water (10 mL) and cooled to 0° C. The precipitate is filtered off and dried by azeotropic removal of water with toluene to give the respective 2-hydroxyimino-3-oxo-3-phenyl-propionic acid ethyl ester. In case no precipitation occurred, the reaction mixture is extracted with ether, the organic layer is washed with sat. aqueous NaHCO₃ solution and water and the solvents are removed in vacuo to give a crude 2-hydroxyimino-3-oxo-3-phenyl-propionic acid ethyl ester derivative. The obtained intermediate is dissolved in a mixture of acetic anhydride (1.38 mL) and acetic acid (1.80 mL). Sodium acetate (0.30 mmol), HgCl₂ (0.01 mmol) and zinc powder (14.6 mmol) are added successively. The mixture is stirred under reflux for 1 h, cooled to RT and filtered and the residue is washed with ether. The filtrate is washed three times with water and once with aq. K₂CO₃ solution (1.0 M). The organic layer is dried over MgSO₄ and concentrated in vacuo to give the desired crude product which is purified by FC (heptane/EtOAc 1/1 or gradient: heptane to heptane/EtOAc 3/7).

2-Acetylamino-3-oxo-3-(3-trifluoromethyl-phenyl)-propionic acid ethyl ester

prepared by reaction of 3-oxo-3-(3-trifluoromethyl-phenyl)-propionic acid ethyl ester. LC-MS (A): t_{R}=0.90 min; [M+H]⁺=318.0.

2-Acetylamino-3-(3-methoxy-phenyl)-3-oxo-propionic acid ethyl ester

prepared by reaction of 3-(3-methoxy-phenyl)-3-oxo-propionic acid ethyl ester. LC-MS (A): t_{R}=0.82 min; [M+H]⁺=280.1.

2-Acetylamino-3-(3,4-dimethyl-phenyl)-3-oxo-propionic acid methyl ester

prepared by reaction of 3-(3,4-dimethyl-phenyl)-3-oxo-propionic acid methyl ester. LC-MS (A): t_{R}=0.89 min; [M+H]⁺=264.1.

A.3.2 Synthesis of 2-Methyl-5-phenyl-oxazole-4-carboxylic acid ethyl ester derivatives (General Procedure)

At 0° C. SOCl₂ (1.76 mmol) is added to a stirred solution of the respective 2-acetyl-amino-3-oxo-3-phenyl-propionic acid ethyl ester derivative (1.26 mmol) in CHCl₃ (0.76 mL). After 30 min the mixture is heated to reflux for 60 min. An additional portion of SOCl₂ (0.32 mmol) is added and the mixture is heated to reflux for further 60 min. An aq. K₂CO₃ solution (1.0 M) is added, the layers are separated and the aq. layer is extracted twice with ether. The combined organic layers are washed with water, dried over MgSO₄, filtered and concentrated in vacuo to give the desired ester which is used without further purification.

2-Methyl-5-(3-trifluoromethyl-phenyl)-oxazole-4-carboxylic acid ethyl ester

prepared by cyclisation of 2-acetylamino-3-oxo-3-(3-trifluoromethyl-phenyl)-propionic acid ethyl ester. LC-MS (A): t_{R}=0.99 min; [M+H]⁺=300.3.

5-(3-Methoxy-phenyl)-2-methyl-oxazole-4-carboxylic acid ethyl ester

prepared by cyclisation of 2-acetylamino-3-(3-methoxy-phenyl)-3-oxo-propionic acid ethyl ester. LC-MS (A): t_{R}=0.92 min; [M+H]⁺=262.3.

5-(3,4-Dimethyl-phenyl)-2-methyl-oxazole-4-carboxylic acid methyl ester

prepared by cyclisation of 2-acetylamino-3-(3,4-dimethyl-phenyl)-3-oxo-propionic acid methyl ester. LC-MS (A): t_{R}=1.00 min; [M+H]⁺=246.1.

A.3.3 Synthesis of 5-phenyl-oxazole-4-carboxylic acid methyl ester derivatives via cyclisation of isocyanides (General Procedure)

To a suspension of the respective benzoic acid derivative (5.81 mmol) and potassium carbonate (13.9 mmol) in DMF (12 mL) is added a solution of methyl isocyanoacetate (11.6 mmol, 2 eq) in DMF (7.5 mL). The resulting mixture is stirred at RT for 5 min and then cooled to 0° C. A solution of DPPA (5.81 mmol) in DMF (7.5 mL) is added dropwise. The resulting mixture is stirred for 2 h at 0° C. and for 16 h at RT and diluted with toluene-EtOAc 1:1 (200 mL). The layers are separated and the organic layer is washed with water (100 mL), aqueous citric acid solution (10%, 50 mL), water (50 mL) and aq. sat. NaHCO₃ solution (50 mL), dried over MgSO₄ and concentrated in vacuo. The residue is purified by FC on silica gel (EA/Hept 1:1) to give the desired product.

5-(3-Dimethylamino-phenyl)-oxazole-4-carboxylic acid methyl ester

prepared by cyclisation of 3-(dimethylamino)-benzoic acid with methyl isocyanoacetate. LC-MS (A): t_{R}=0.73 min; [M+H]⁺=247.4.

A.3.4 Synthesis of 5-(3,4-Dimethyl-phenyl)-oxazole-4-carboxylic acid methyl ester

### Step 1: 2-Diazo-3-(3,4-dimethyl-phenyl)-3-oxo-propionic acid methyl ester

At 0° C. TEA (13.2 mmol) is added dropwise to a solution of 3-(3,4-dimethylphenyl)-3-oxo-propionic acid methyl ester (4.39 mmol) and 4-acetamidobenzenesulfonyl azide (4.39 mmol) in acetonitrile (26 mL). The mixture is stirred at RT for 2 h and concentrated in vacuo. Three times a mixture of ether and petroleum ether is added to the residue and the suspension is filtered. The combined liquid phases are concentrated in vacuo and the residue is purified by FC (heptane/EtOAc 4/1) to give the desired product. LC-MS (A): t_{R}=0.98 min; [M+H]⁺=232.1.

### Step 2: 3-(3,4-Dimethyl-phenyl)-2-formylamino-3-oxo-propionic acid methyl ester

A solution of 2-diazo-3-(3,4-dimethyl-phenyl)-3-oxo-propionic acid methyl ester (3.67 mmol) in dichloroethane (7.3 mL) is added within 60 min to a refluxing solution of formamide (4.40 mmol) and dirhodium tetraacetate (0.183 mmol) in dichloroethane (8.8 mL). The mixture is stirred for further 60 min under reflux, cooled to RT and concentrated in vacuo. The residue is purified by FC (heptane/EtOAc 6/4) to give the desired product as a white solid. ¹H NMR (CDCl₃) δ=2.37 (s, 6H), 3.74 (s, 3H), 6.28 (d, J=7.8 Hz, 1H), 7.03 (bs, 1H), 7.30 (d, J=8.0 Hz, 1H), 7.90 (m, 2H), 8.33 (s, 1H).

### Step 3: 5-(3,4-Dimethyl-phenyl)-oxazole-4-carboxylic acid methyl ester

TEA (4.81 mmol) and a solution of 3-(3,4-dimethyl-phenyl)-2-formylamino-3-oxo-propionic acid methyl ester in DCM (6.0 mL) are added successively to a solution of triphenylphosphine (2.41 mmol) and iodine (2.28 mmol) in DCM (6.0 mL). The mixture is stirred for 45 min at RT, the solvents are removed in vacuo and the residue is purified by FC (heptane/EtOAc 6/4) to give the desired product.

¹H NMR (CDCl₃): δ=2.35 (s, 3H), 2.36 (s, 3H), 3.97 (s, 3H), 7.27 (d, J=7.8 Hz, 1H), 7.87 (m, 3H).

A.3.5 Synthesis of 5-phenyl-oxazole-4-carboxylic acid derivatives (General Procedure)

A mixture of the respective 5-phenyl-oxazole-4-carboxylic acid ester derivative (1.12 mmol), EtOH (1.25 mL) and aq. NaOH solution (2.0 M, 1.25 mL) is stirred for 2 h at RT and washed once with ether. The aq. layer is made acidic by addition of conc HCl and extracted twice with ether. The combined organic layers are dried over MgSO₄ and concentrated in vacuo to give the desired acid as a pure yellow solid. In an alternative procedure a solution of the respective ester (3.24 mmol) in THF (32 mL) is treated with aq. NaOH solution (1.0 M, 16 mL) and stirred for 16 h.

2-Methyl-5-(3-trifluoromethyl-phenyl)-oxazole-4-carboxylic acid

prepared by saponification of 2-methyl-5-(3-trifluoromethyl-phenyl)-oxazole-4-carboxylic acid ethyl ester. LC-MS (B): t_{R}=0.55 min; [M−H]⁻=270.2.

5-(3-Methoxy-phenyl)-2-methyl-oxazole-4-carboxylic acid

prepared by saponification of 5-(3-methoxy-phenyl)-2-methyl-oxazole-4-carboxylic acid ethyl ester. LC-MS (B): t_{R}=0.49 min; [M−H]⁻=232.3.

5-(3-Dimethylamino-phenyl)-oxazole-4-carboxylic acid

prepared by saponification of 5-(3-dimethylamino-phenyl)-oxazole-4-carboxylic acid methyl ester. LC-MS (A): t_{R}=0.60 min; [M+H]⁺=233.5.

5-(3,4-Dimethyl-phenyl)-2-methyl-oxazole-4-carboxylic acid

prepared by saponification of 5-(3,4-dimethyl-phenyl)-2-methyl-oxazole-4-carboxylic acid methyl ester. LC-MS (A): t_{R}=0.85 min; [M+H]⁺=232.0.

5-(3,4-Dimethyl-phenyl)-oxazole-4-carboxylic acid

prepared by saponification of 5-(3,4-dimethyl-phenyl)-oxazole-4-carboxylic acid methyl ester. LC-MS (A): t_{R}=0.87 min; [M+H]⁺=218.2.

A.4 Synthesis of 3-Phenyl-pyrazine-2-carboxylic acid derivatives (General Procedure)

An aqueous K₂CO₃ solution (2.0 M, 30 mL) is added to a solution of 3-chloro-pyrazine-2-carbonitrile (21.5 mmol) and the respective phenylboronic acid (21.5 mmol) in DME (65 mL). Triphenylphosphine (3.21 mmol) and palladium(II) acetate (1.06 mmol) are added and the mixture is stirred at 90° C. for 16 h and allowed to reach RT. EtOAc is added and the mixture is filtered through Celite, dried over MgSO₄ and concentrated in vacuo to give the respective carbonitrile derivative which is diluted with MeOH (100 mL) and aqueous NaOH solution (4.0 M, 160 mL). The mixture is stirred at 85° C. for 16 h, cooled to RT and concentrated partially in vacuo to remove methanol. Water and conc. hydrochloric acid are added (pH ˜2) and the obtained precipitate is filtered off. The residue is dissolved in a mixture of EtOAc and DCM, dried over MgSO₄ and concentrated in vacuo to give the desired acid derivative.

3-(3-Methoxy-phenyl)-pyrazine-2-carboxylic acid

prepared by reaction of 3-chloro-pyrazine-2-carbonitrile with 3-methoxybenzene-boronic acid. LC-MS (A): t_{R}=0.71 min; [M+H]⁺=231.5.

3-m-Tolyl-pyrazine-2-carboxylic acid

prepared by reaction of 3-chloro-pyrazine-2-carbonitrile with m-tolyl-boronic acid. LC-MS (B): t_{R}=0.28 min; [M−H]⁻=213.2.

3-p-Tolyl-pyrazine-2-carboxylic acid

prepared by reaction of 3-chloro-pyrazine-2-carbonitrile with p-tolyl-boronic acid. LC-MS (B): t_{R}=0.40 min; [M−H]⁻=213.1.

3-(3,4-Dimethyl-phenyl)-pyrazine-2-carboxylic acid

prepared by reaction of 3-chloro-pyrazine-2-carbonitrile with 3,4-dimethyl-phenyl-boronic acid. LC-MS (B): t_{R}=0.50 min; [M−H]⁻=227.2.

A.5 Synthesis of 6′-Methoxy-[3,3′]bipyridinyl-2-carboxylic acid

3-Bromo-pyridine-2-carboxylic acid methyl ester

Under inert gas a solution of 3-bromo-pyridine-2-carboxylic acid (4.95 mmol) in MeOH (8.0 mL) is treated dropwise with conc. sulfuric acid (0.50 mL) and heated subsequently to reflux for 150 min. The mixture is cooled to 0° C. and neutralized by addition of DIPEA. After removal of the volatiles EtOAc (30 mL) and water (10 mL) are added and the layers are separated. The organic layer is washed twice with sat. NaHCO₃ solution (2×10 mL) and once with water (10 mL), dried over MgSO₄ and concentrated in vacuo to give the crude product which is used without further purification. LC-MS (B): t_{R}=0.69 min; [M+H]⁺=216.0. ¹H-NMR (CDCl₃): δ=4.04 (s, 3H), 7.32 (m, 1H), 8.03 (d, J=8.0 Hz, 1H), 8.63 (m, 1H).

6′-Methoxy-[3,3′]bipyridinyl-2-carboxylic acid methyl ester

A freshly prepared aqueous Na₂CO₃ solution (2.0 M, 25 mL) is added to a suspension of 3-bromo-pyridine-2-carboxylic acid methyl ester (4.17 mmol) and 2-methoxy-pyridine-5-boronic acid (4.17 mmol) in a mixture of toluene (17 mL) and EtOH (17 mL). Argon is passed through the mixture to remove oxygen, tetrakis(triphenyl-phosphine)palladium(0) (134 mg) is added under argon and the mixture is vigorously stirred at 75° C. for 2 h. The layers are separated and the aqueous layer is extracted once with EtOAc. The combined organic layers are washed with water (10 mL), dried over MgSO₄ and concentrated in vacuo. The residue is purified by prep. TLC to give the desired product in a mixture with the respective ethyl ester. LC-MS (C): t_{R}=0.50 min; [M+H]⁺=245.3.

6′-Methoxy-[3,3]bipyridinyl-2-carboxylic acid

A solution of 6′-methoxy-[3,3′]bipyridinyl-2-carboxylic acid ester (mixture of methyl and ethyl ester; 1.33 mmol) in a mixture of THF (2.7 mL) and MeOH (4.2 mL) is treated with aq. NaOH solution (5.0 M, 0.53 mL) and stirred for 20 min at RT. The organic volatiles are removed in vacuo and the aq. layer is made acidic (pH ˜5) by addition of hydrochloric acid (25%). The mixture is concentrated in vacuo to dryness and the residue is treated with MeOH (5.0 mL). The suspension is filtered through Celite and the filtrate is concentrated in vacuo to give the desired product. LC-MS (C): t_{R}=0.27 min; [M+H]⁺=231.2.

### A.6 Synthesis of Aryl-Ethylamine Derivatives

### A.6.1 Synthesis of Difluoro-Methoxy Substituted Benzaldehyde Derivatives (General Procedure)

A mixture of the respective phenol (47.2 mmol), sodium chlorodifluoroacetate (94.4 mmol) and potassium carbonate (56.5 mmol) in DMF (85 mL) and water (10 mL) is heated under a nitrogen atmosphere to 100° C. for 4 h, cooled to RT and stirred for additional 16 h. Hydrochloric acid (12M, 13.5 mL) and water (19.5 mL) are added and the mixture is stirred for 3 h. An aqueous NaOH solution (2.0 M, 90 mL) is added, the mixture is diluted with ether (100 mL) and water (100 mL), the layers are separated and the aqueous layer is extracted three times with ether (3×75 mL). The combined organic layers are washed twice with aqueous NaOH solution (2.0 M), once with water and once with brine, dried over Na₂SO₄ and concentrated in vacuo to give the desired product which is used without further purification.

3-Difluoromethoxy-4-methoxy-benzaldehyde

prepared by reaction of 3-hydroxy-4-methoxy-benzaldehyde. ¹H NMR (CDCl₃): δ=3.97 (s, 3H), 6.57 (t, J=74.3 Hz, 1H), 7.08 (d, J=8.5 Hz, 1H), 7.68 (s, 1H), 7.74 (d, J=8.3 Hz, 1H), 9.86 (s, 1H).

4-Difluoromethoxy-3-methoxy-benzaldehyde

prepared by reaction of 4-hydroxy-3-methoxy-benzaldehyde. ¹H NMR (CDCl₃): δ=3.95 (s, 3H), 6.65 (t, J=74.3 Hz, 1H), 7.30 (d, J=8.0 Hz, 1H), 7.46 (dd, J=8.0, 1.5 Hz, 1H), 7.50 (d, J=1.3 Hz, 1H), 9.93 (s, 1H).

A.6.2 Synthesis of 4-Methoxy-3-methylsulfanyl-benzaldehyde 2-(3-Bromo-4-methoxy-phenyl)-5,5-dimethyl-[1,3]dioxane

A mixture of 3-bromo-4-methoxy-benzaldehyde (10.0 mmol), 2,2-dimethyl-propane-1,3-diol (12.0 mmol) and PTSA (0.20 mmol) in toluene (25 mL) is heated to reflux in the presence of a Dean-Stark water trap for 80 min. TEA (0.5 mmol) is added and the mixture is cooled to RT. The mixture is washed three times with water, diluted with EtOAc (25 mL), washed additional two times with water, dried over Na₂SO₄ and concentrated in vacuo to give the desired product as a white solid. LC-MS (A): t_{R}=1.02 min; [M+H]⁺=301.1.

2-(4-Methoxy-3-methylsulfanyl-phenyl)-5,5-dimethyl-[1,3]dioxane

At −78° C. a solution of n-butyllithium in hexane (1.6 M, 5.56 mmol) is added dropwise under a nitrogen atmosphere to a mixture of 2-(3-bromo-4-methoxy-phenyl)-5,5-dimethyl-[1,3]dioxane (5.00 mmol) and molecular sieve (4 Å, 1.5 g) in THF (10 mL). After 25 min the mixture is treated dropwise with dimethyl disulfide (5.00 mmol), stirred for additional 30 min, warmed up to −10° C. and poured into water (50 mL). EtOAc (40 mL) is added, the layers are separated and the aqueous layer is extracted twice with EtOAc (2×20 mL). The combined organic layers are washed with water (3×20 mL), dried over Na₂SO₄ and concentrated in vacuo to give a crude product which is recrystallized from isopropanol. LC-MS (A): t_{R}=0.99 min; [M+H]⁺=269.2.

4-Methoxy-3-methylsulfanyl-benzaldehyde

Hydrochloric acid (6.0 M, 250 mL) is added to a solution of 2-(4-methoxy-3-methylsulfanyl-phenyl)-5,5-dimethyl-[1,3]dioxane (16.7 mmol) in acetone (250 mL). The mixture is stirred for 30 min, concentrated in vacuo to remove acetone and extracted three times with DCM (3×50 mL). The combined organic layers are washed with sat. NaHCO₃ solution (50 mL), water (50 mL) and brine (50 mL), dried over MgSO₄ and concentrated in vacuo to give a crude product which is used without further purification. ¹H NMR (CDCl₃): δ=2.48 (s, 3H), 3.98 (s, 3H), 6.93 (d, J=8.3 Hz, 1H), 7.64 (m, 1H), 7.66 (m, 1H), 9.87 (s, 1H).

A.6.3 Synthesis of 2-nitro-vinyl-aryl derivatives (General Procedure)

To a solution of the respective benzaldehyde derivative (4.00 mmol) in nitromethane (2.5 mL) is added molecular sieve (3 Å), n-butylamine (0.27 mmol) and acetic acid (0.46 mmol). The mixture is heated to 95° C. until TLC indicated complete conversion (˜50 min) and filtered through Celite. The Celite pad is washed with DCM and the filtrate is concentrated in vacuo. The residue is recrystallized from isopropanol, isopropanol-methanol mixtures (5/2) or methanol-water mixtures (9/1) to give the desired product as a solid.

2-Difluoromethoxy-1-methoxy-4-((E)-2-nitro-vinyl)-benzene

prepared by reaction of 3-difluoromethoxy-4-methoxy-benzaldehyde. ¹H NMR (CDCl₃): δ=3.94 (s, 3H), 6.57 (t, J=74.5 Hz, 1H), 7.02 (d, J=8.5 Hz, 1H), 7.37 (s, 1H), 7.41 (d, J=8.5 Hz, 1H), 7.49 (d, J=13.6 Hz, 1H), 7.92 (d, J=13.6 Hz, 1 H).

1-Difluoromethoxy-2-methoxy-4-((E)-2-nitro-vinyl)-benzene

prepared by reaction of 4-difluoromethoxy-3-methoxy-benzaldehyde. ¹H NMR (CDCl₃): δ=3.93 (s, 3H), 6.61 (t, J=74.3 Hz, 1H), 7.09 (s, 1H), 7.15 (m, 1H), 7.22 (m, 1H), 7.54 (d, J=13.6 Hz, 1H), 7.95 (d, J=13.6 Hz, 1H).

2-((E)-2-Nitro-vinyl)-naphthalene

prepared by reaction of 2-naphthaldehyde. ¹H NMR (CDCl₃): δ=7.58 (m, 3H), 7.69 (d, J=13.6 Hz, 1H), 7.88 (m, 3H), 8.01 (s, 1H), 8.16 (d, J=13.8 Hz, 1H).

1-((E)-2-Nitro-vinyl)-4-trifluoromethyl-benzene

prepared by reaction of 4-trifluoromethyl-benzaldehyde. ¹H NMR (CDCl₃): δ=7.61 (d, J=13.8 Hz, 1H), 7.66 (d, J=8.3 Hz, 2H), 7.71 (d, J=8.3 Hz, 2H), 8.01 (d, J=13.8 Hz, 1H).

1-Methylsulfanyl-4-((E)-2-nitro-vinyl)-benzene

prepared by reaction of 4-(methylmercapto)-benzaldehyde. ¹H NMR (CDCl₃): δ=2.51 (s, 3H), 7.25 (d, J=8.3 Hz, 2H), 7.44 (d, J=8.3 Hz, 2H), 7.56 (d, J=13.8 Hz, 1H), 7.95 (d, J=13.6 Hz, 1H).

1-((E)-2-Nitro-vinyl)-4-trifluoromethoxy-benzene

prepared by reaction of 4-(trifluoromethoxy)-benzaldehyde. ¹H NMR (CDCl₃): δ=7.29 (d, J=8.3 Hz, 2H), 7.55 (d, J=13.8 Hz, 1H), 7.59 (d, J=8.8 Hz, 2H), 7.98 (d, J=13.8 Hz, 1H).

2,2-Difluoro-5-((E)-2-nitro-vinyl)-benzo[1,3]dioxole

prepared by reaction of 2,2-difluoro-benzo[1,3]dioxole-5-carbaldehyde. ¹H NMR (CDCl₃): δ=7.15 (d, J=8.3 Hz, 1H), 7.26 (d, J=1.5 Hz, 1H), 7.31 (dd, J=8.5, 1.3 Hz, 1H), 7.50 (d, J=13.6 Hz, 1H), 7.95 (d, J=13.8 Hz, 1H).

1-Methoxy-2-methylsulfanyl-4-((E)-2-nitro-vinyl)-benzene

prepared by reaction of 4-methoxy-3-methylsulfanyl-benzaldehyde. ¹H NMR (CDCl₃): δ=2.46 (s, 3H), 3.95 (s, 3H), 6.87 (d, J=8.5 Hz, 1H), 7.27 (d, J=1.8 Hz, 1H), 7.35 (dd, J=8.3, 2.0 Hz, 1H), 7.53 (d, J=13.8 Hz, 1H), 7.96 (d, J=13.6 Hz, 1H).

1,2-Dimethoxy-4-((E)-2-nitro-but-1-enyl)-benzene

prepared by reaction of 3,4-dimethoxy-benzaldehyde with 1-nitropropane (instead of nitromethane). ¹H NMR (CDCl₃): δ=1.29 (t, J=7.3 Hz, 3H), 2.90 (q, J=7.5 Hz, 2H), 3.90 (s, 3H), 3.93 (s, 3H), 6.93 (m, 2H), 7.07 (m, 1H), 8.00 (s, 1H).

1,2-Dimethoxy-4-((E)-2-nitro-prop-1-enyl)-benzene

prepared by reaction of 3,4-dimethoxy-benzaldehyde with nitroethane (instead of nitromethane). ¹H NMR (CDCl₃): δ=2.47 (s, 3H), 3.90 (s, 3H), 3.92 (s, 3H), 6.93 (m, 2H), 7.07 (d, J=8.3 Hz, 1H), 8.05 (s, 1H).

1-Bromo-3-((E)-2-nitro-vinyl)-benzene

prepared by reaction of 3-bromo-benzaldehyde. ¹H NMR (CDCl₃): δ=7.32 (t, J=7.6 Hz, 1H), 7.44 (d, J=7.6 Hz, 1H), 7.52 (d, J=13.5 Hz, 1H), 7.59 (d, J=7.6 Hz, 1H), 7.65 (bs, 1H), 7.88 (d, J=14.0 Hz, 1H).

2-Methoxy-5-((E)-2-nitro-vinyl)-pyridine

prepared by reaction of 6-methoxy-pyridine-3-carbaldehyde (the product precipitated already during cooling from 95° C. to RT and was not recrystallized). ¹H NMR (CDCl₃): δ=3.99 (s, 3H), 6.81 (d, J=8.8 Hz, 1H), 7.51 (d, J=13.8 Hz, 1H), 7.74 (dd, J=8.5, 2.3 Hz, 1H), 7.96 (d, J=13.6 Hz, 1H), 8.33 (d, J=2.0 Hz, 1H).

A.6.4 Synthesis of 2-aryl-ethylamine derivatives (General Procedure)

At 0° C. a suspension of LAH (14.0 mmol) in THF (18 mL) is treated dropwise with conc. sulfuric acid (95%, 0.37 mL). After 10 min a solution of the respective nitro-vinyl derivative (3.14 mmol) in THF (12 mL) is added dropwise at 0° C. The mixture is stirred for additional 10 min and heated slowly to reflux for 5 min. After cooling to 0° C. isopropanol (2.3 mL), aqueous NaOH solution (2.0 M, 1.6 mL) and THF are added dropwise and the mixture is filtered. The filtrate is concentrated in vacuo and the residue is diluted with ether (50 mL). Isopropanol (0.5 mL) and a solution of HCl in ether (2.0 M) are added and the obtained suspension is filtered to give the desired product as a hydrochloride salt.

2-(3-Difluoromethoxy-4-methoxy-phenyl)-ethylamine

prepared by reaction of 2-difluoromethoxy-1-methoxy-4-((E)-2-nitro-vinyl)-benzene. ¹H NMR (D₂O): δ=2.89 (t, J=7.5 Hz, 2H), 3.19 (t, J=7.3 Hz, 2H), 3.83 (s, 3H), 6.73 (t, J=74.3 Hz, 1H), 7.11 (m, 3H).

2-(4-Difluoromethoxy-3-methoxy-phenyl)-ethylamine

prepared by reaction of 1-difluoromethoxy-2-methoxy-4-((E)-2-nitro-vinyl)-benzene. ¹H NMR (D₂O): δ=2.94 (t, J=7.3 Hz, 2H), 3.23 (t, J=7.3 Hz, 2H), 3.84 (s, 3H), 6.72 (t, J=74.3 Hz, 1H), 6.88 (dd, J=8.3, 2.0 Hz, 1H), 7.05 (d, J=1.8 Hz, 1H), 7.17 (d, J=8.3 Hz, 1H).

2-Naphthalen-2-yl-ethylamine

prepared by reaction of 2-((E)-2-nitro-vinyl)-naphthalene. ¹H NMR (DMSO-d₆): δ=3.07 (m, 2H), 3.16 (m, 2H), 7.45 (dd, J=8.5, 1.8 Hz, 1H), 7.51 (m, 2H), 7.79 (s, 1 H), 7.90 (m, 3H).

2-(4-Trifluoromethyl-phenyl)-ethylamine

prepared by reaction of 1-((E)-2-nitro-vinyl)-4-trifluoromethyl-benzene. ¹H NMR (D₂O): δ=3.03 (t, J=7.5 Hz, 2H), 3.26 (t, J=7.3 Hz, 2H), 7.44 (d, J=8.0 Hz, 2 H), 7.66 (d, J=8.0 Hz, 2H).

2-(4-Methylsulfanyl-phenyl)-ethylamine

prepared by reaction of 1-methylsulfanyl-4-((E)-2-nitro-vinyl)-benzene. ¹H NMR (D₂O): δ=2.44 (s, 3H), 2.92 (t, J=7.5 Hz, 2H), 3.21 (t, J=7.3 Hz, 2H), 7.23 (m, 2 H), 7.29 (m, 2H).

2-(4-Trifluoromethoxy-phenyl)-ethylamine

prepared by reaction of 1-((E)-2-nitro-vinyl)-4-trifluoromethoxy-benzene. ¹H NMR (D₂O): δ=2.98 (t, J=7.3 Hz, 2H), 3.23 (t, J=7.3 Hz, 2H), 7.28 (m, 2H), 7.35 (m, 2H).

2-(2,2-Difluoro-benzo[1,3]dioxol-5-yl)-ethylamine

prepared by reaction of 2,2-difluoro-5-((E)-2-nitro-vinyl)-benzo[1,3]dioxole. ¹H NMR (D₂O): δ=2.95 (t, J=7.3 Hz, 2H), 3.21 (t, J=7.3 Hz, 2H), 7.02 (dd, J=8.0, 1.5 Hz, 1H), 7.10 (d, Jz 2 Hz, 1H), 7.11 (d, J≈8 Hz, 1H).

2-(4-Methoxy-3-methylsulfanyl-phenyl)-ethylamine

prepared by reaction of 1-methoxy-2-methylsulfanyl-4-((E)-2-nitro-vinyl)-benzene. ¹H NMR (D₂O): δ=2.40 (s, 3H), 2.90 (t, J=7.3 Hz, 2H), 3.20 (t, J=7.3 Hz, 2H), 3.83 (s, 3H), 6.96 (d, J=8.3 Hz, 1H), 7.10 (dd, J=8.4, 2.1 Hz, 1H), 7.13 (d, J=2.0 Hz, 1H).

1-(3,4-Dimethoxy-benzyl)-propylamine

prepared by reaction of 1,2-dimethoxy-4-((E)-2-nitro-but-1-enyl)-benzene. ¹H NMR (D₂O): δ=0.96 (t, J=7.3 Hz, 3H), 1.64 (m, 2H), 2.74 (dd, J=14.3, 8.3 Hz, 1H), 2.96 (dd, J=14.3, 6.0 Hz, 1H), 3.40 (m, 1H), 3.79 (s, 3H), 3.80 (s, 3H), 6.84 (dd, J=8.3, 2.0 Hz, 1H), 6.90 (d, J=2.0 Hz, 1H), 6.97 (d, J=8.3 Hz, 1H).

1-(3,4-Dimethoxy-phenyl)-prop-2-ylamine

prepared by reaction of 1,2-Dimethoxy-4-((E)-2-nitro-prop-1-enyl)-benzene. ¹H NMR (D₂O): δ=1.24 (d, J=6.8 Hz, 3H), 2.80 (dd, J=14.1, 7.4 Hz, 1H), 2.85 (dd, J=14.2, 7.2 Hz, 1H), 3.55 (hex, J=6.8 Hz, 1H), 3.79 (s, 3H), 3.80 (s, 3H), 6.83 (dd, J=8.0, 1.8 Hz, 1H), 6.89 (d, J=1.8 Hz, 1H), 6.97 (d, J=8.3 Hz, 1H).

2-(3-Bromo-phenyl)-ethylamine

prepared by reaction of 1-bromo-3-((E)-2-nitro-vinyl)-benzene. LC-MS (A): t_{R}=0.61 min; [M+CH₃CN+H]⁺=241.1.

A.6.5 Synthesis of 2-aryl-ethylamine derivatives by hydrogenation (General Procedure)

Hydrochloric acid (35%, 1.84 mL) is added to a mixture of the respective nitro-vinyl derivative (9.55 mmol) in EtOH (37 mL). The mixture is cooled to 0° C., treated with Pd/C (10%, 2.0 g) and stirred under a hydrogen atmosphere (1 bar) for 16 h under slow warming to RT. After filtration through Celite and removal of the solvents in vacuo the crude product is diluted with EtOH (30 mL) and stirred until precipitation occurred. The precipitate is filtered off, treated with warm EtOH (13 mL), cooled in an ice bath and filtered again to give the desired product as a white solid.

2-(6-Methoxy-pyridin-3-yl)-ethylamine

prepared by reduction of 2-methoxy-5-((E)-2-nitro-vinyl)-pyridine. ¹H NMR (D₂O): δ=3.03 (t, J=8.0 Hz, 2H), 3.24 (t, J=7.5 Hz, 2H), 4.09 (s, 3H), 7.37 (d, J=9.0 Hz, 1H), 8.14 (d, J=2.0 Hz, 1H), 8.26 (dd, J=9.0, 2.3 Hz, 1H).

A.6.6 Synthesis of 2-(2-ethyl-4-iodo-imidazol-1-yl)-ethylamine 4,5-diiodo-2-ethyl-1H-imidazole

To a slightly yellow homogeneous solution of 2-ethylimidazole (15.0 g, 156 mmol) in dioxane (250 ml) and distilled water (250 ml) is added successively, at RT (in one portion), sodium carbonate (49.6 g, 468 mmol), and iodine (87.1 g, 343 mmol). The resulting brown heterogeneous reaction mixture is further stirred at RT, under nitrogen, for 24 h. EtOAc (500 ml) is then added followed by an aq. solution of sodium thiosulfate (45 g Na₂S₂O₃ in 300 ml of water). The yellow homogeneous organic layer is separated and additionally washed with an aq. solution of sodium thiosulfate (30 g Na₂S₂O₃ in 300 ml of water), and finally with brine (200 ml). The yellow organic layer is then dried over MgSO₄, filtered, and concentrated to dryness under reduced pressure to give the pure product 4,5-diiodo-2-ethyl-1H-imidazole as a pale yellow solid. LC-MS (A): t_{R}=0.55 min; [M+H]⁺=349.2.

[2-(2-ethyl-4,5-diiodo-imidazol-1-yl)-ethyl]-carbamic acid tert-butyl ester

To a solution of 4,5-diiodo-2-ethyl-1H-imidazole (10.0 g, 28.7 mmol) in anhydrous DMF (140 ml) is added portionwise, at RT, sodium hydride moistened with oil (55-65%, 1.38 g, 34.5 mmol). The resulting mixture is further stirred at RT, under nitrogen, for 20 min. The mixture is then heated to 100° C., and a colorless homogeneous solution of 2-(Boc-amino)-ethylbromide (7.09 g, 31.6 mmol) in anhydrous DMF (100 ml) is added dropwise within 1 h. The resulting dark-orange homogeneous mixture is further heated at 100° C. for 90 min. The reaction mixture is cooled to RT and water (300 ml) is added slowly. This mixture is extracted with ether (7×100 ml). The combined organic layers are washed with brine (3×100 ml), dried over MgSO₄, filtered, and concentrated to dryness under reduced pressure to give a yellow oil. The crude product is purified by FC (DCM/MeOH=25/1) to give the desired product as a pale yellow solid. LC-MS (A): t_{R}=0.78 min; [M+H]⁺=492.3.

[2-(2-ethyl-4-iodo-imidazol-1-yl)-ethyl]-carbamic acid tert-butyl ester

A solution of [2-(2-ethyl-4,5-diiodo-imidazol-1-yl)-ethyl]-carbamic acid tert-butyl ester (23.0 g, 46.8 mmol) in anhydrous THF (280 ml), under nitrogen, is cooled to −40° C., and a solution of EtMgBr in ether (3.0 M, 15.6 ml, 46.8 mmol) is added dropwise over 15 min. After addition, the resulting solution is stirred between −40° C. and −30° C. for 10 min, and additional EtMgBr in ether (3.0 M, 10.0 ml, 30.0 mmol) is added. The reaction mixture is treated with water (10 ml) at −40° C. and allowed to warm-up to RT. Ether (300 ml) is added, and the resulting solution is washed with water (200 ml) and brine (200 ml). The organic layer is dried over MgSO₄, filtered, and concentrated to dryness under reduced pressure to give a crude product which is purified by FC (DCM/MeOH=20/1) to give the desired product as a yellow solid. LC-MS (A): t_{R}=0.65 min; [M+H]⁺=366.4.

2-(2-ethyl-4-iodo-imidazol-1-yl)-ethylamine

To an ice-cooled solution of [2-(2-ethyl-4-iodo-imidazol-1-yl)-ethyl]-carbamic acid tert-butyl ester (5.72 g, 15.7 mmol) in DCM (125 ml) is added slowly HCl in dioxane (4 M, 78 ml, 312 mmol). The resulting suspension is stirred at 0° C. for 15 min, then at RT for 1 h. After removal of the volatiles under reduced pressure the desired product is obtained as a hydrochloride salt. LC-MS (A): t_{R}=0.14 min; [M+H]⁺=266.2. ¹H NMR (CD₃OD): δ=1.43 (t, J=7.8 Hz, 3H), 3.08 (q, J=7.8 Hz, 2H), 3.47 (t, J=6.5 Hz, 2H), 4.49 (t, J=6.5 Hz, 2H), 7.73 (s, 1H).

### A.6.7 Synthesis of Sec.-Amines by Reductive Amination (General Procedure)

TEA (1.0 eq. for amines used as HCl salts) and the respective aldehyde (0.8 mmol) are successively added to a mixture of the respective amine (free base or HCl salt, 0.8 mmol) in MeOH (1.5 mL). After 20 min sodium borohydride (0.80 mmol) is added portionwise and the mixture is stirred for 30 min. Water (0.2 mL) and DMF (0.3 mL) are added, the mixture is filtered and the filtrate is purified by prep. HPLC using a basic (ammonia containing) gradient. The ammonia is removed in vacuo, hydrochloric acid (10%, 1.0 mL) is added and the solvents are removed in vacuo to give the desired product as a hydrochloride salt.

Cyclopropylmethyl-[2-(3-difluoromethoxy-4-methoxy-phenyl)-ethyl]-amine

prepared by reaction of 2-(3-difluoromethoxy-4-methoxy-phenyl)-ethylamine with cyclopropanecarbaldehyde. LC-MS (C): t_{R}=0.70 min; [M+H]⁺=272.3.

Cyclopropylmethyl-[2-(4-difluoromethoxy-3-methoxy-phenyl)-ethyl]-amine

prepared by reaction of 2-(4-difluoromethoxy-3-methoxy-phenyl)-ethylamine with cyclopropanecarbaldehyde. LC-MS (C): t_{R}=0.72 min; [M+H]⁺=272.3.

Cyclopropylmethyl-(2-naphthalen-2-yl-ethyl)-amine

prepared by reaction of 2-naphthalen-2-yl-ethylamine with cyclopropane-carbaldehyde. LC-MS (C): t_{R}=0.78 min; [M+H]⁺=226.4.

Cyclopropylmethyl-[2-(4-trifluoromethyl-phenyl)-ethyl]-amine

prepared by reaction of 2-(4-trifluoromethyl-phenyl)-ethylamine with cyclopropanecarbaldehyde. LC-MS (C): t_{R}=0.77 min; [M+H]⁺=244.3.

Cyclopropylmethyl-[2-(4-methylsulfanyl-phenyl)-ethyl]-amine

prepared by reaction of 2-(4-methylsulfanyl-phenyl)-ethylamine with cyclopropanecarbaldehyde. LC-MS (C): t_{R}=0.70 min; [M+H]⁺=222.3.

Cyclopropylmethyl-[2-(4-trifluoromethoxy-phenyl)-ethyl]-amine

prepared by reaction of 2-(4-trifluoromethoxy-phenyl)-ethylamine with cyclopropanecarbaldehyde. LC-MS (C): t_{R}=0.81 min; [M+H]⁺=260.3.

Cyclopropylmethyl-[2-(2,2-difluoro-benzo[1,3]dioxol-5-yl)-ethyl]amine

prepared by reaction of 2-(2,2-difluoro-benzo[1,3]dioxol-5-yl)-ethylamine with cyclopropanecarbaldehyde. LC-MS (C): t_{R}=0.78 min; [M+H]⁺=256.3.

Cyclopropylmethyl-[2-(4-methoxy-3-methylsulfanyl-phenyl)-ethyl]-amine

prepared by reaction of 2-(4-methoxy-3-methylsulfanyl-phenyl)-ethylamine with cyclopropanecarbaldehyde. LC-MS (C): t_{R}=0.69 min; [M+H]⁺=252.4.

Cyclopropylmethyl-[1-(3,4-dimethoxy-benzyl)-propyl]amine

prepared by reaction of 1-(3,4-dimethoxy-benzyl)-propylamine with cyclopropanecarbaldehyde. LC-MS (C): t_{R}=0.65 min; [M+H]⁺=264.4.

Cyclopropylmethyl-[1-(3,4-dimethoxy-phenyl)-prop-2-yl]amine

prepared by reaction of 1-(3,4-dimethoxy-phenyl)-prop-2-ylamine with cyclopropanecarbaldehyde. LC-MS (B): t_{R}=0.92 min; [M+H]⁺=250.3.

Cyclopropylmethyl-[2-(4-fluoro-phenyl)-ethyl]amine

prepared by reaction of 2-(4-fluoro-phenyl)-ethylamine with cyclopropane-carbaldehyde. LC-MS (C): t_{R}=0.59 min; [M+H]⁺=194.4.

[2-(3-Bromo-phenyl)-ethyl]cyclopropylmethyl-amine

prepared by reaction of 2-(3-bromo-phenyl)-ethylamine with cyclopropane-carbaldehyde. LC-MS (B): t_{R}=0.92 min; [M+H]⁺=254.0.

Cyclopropylmethyl-[2-(3,4-dimethoxy-phenyl)-ethyl]-amine

prepared by reaction of 2-(3,4-dimethoxy-phenyl)-ethylamine with cyclopropane-carbaldehyde. LC-MS (B): t_{R}=0.85 min; [M+H]⁺=236.2.

2-(Cyclopropylmethyl-amino)-1-(3,4-dimethoxy-phenyl)-ethanol

prepared by reaction of 2-amino-1-(3,4-dimethoxy-phenyl)-ethanol (M. Kihara et al. Chem. Pharm. Bull. 1989, 37, 870-876) with cyclopropanecarbaldehyde. LC-MS (B): t_{R}=0.68 min; [M+H]⁺=252.1. ¹H NMR (CDCl₃): δ=0.11 (m, 2H), 0.48 (m, 2H), 0.95 (m, 1H), 2.47 (dd, J=12.3, 7.0 Hz, 1H), 2.57 (dd, J=12.3, 6.8 Hz, 1H), 2.71 (dd, J=11.8, 9.5 Hz, 1H), 2.91 (dd, J=12.3, 3.0 Hz, 1H), 3.86 (s, 3H), 3.89 (s, 3 H), 4.65 (dd, J=8.8, 3.0 Hz, 1H), 6.83 (d, J=8.3 Hz, 1H), 6.88 (d, J=8.3 Hz, 1H), 6.94 (s, 1H).

Cyclopropylmethyl-[2-(1H-indol-3-yl)-ethyl]-amine

prepared by reaction of 2-(1H-indol-3-yl)-ethylamine with cyclopropane-carbaldehyde. LC-MS (C): t_{R}=0.62 min; [M+H]⁺=215.4.

[2-(1H-Benzoimidazol-2-yl)-ethyl]-cyclopropylmethyl-amine

prepared by reaction of 2-(1H-benzoimidazol-2-yl)-ethylamine with cyclopropane-carbaldehyde. LC-MS (C): t_{R}=0.34 min; [M+H]⁺=216.4.

Cyclopropylmethyl-[2-(2-ethyl-4-iodo-imidazol-1-yl)-ethyl]-amine

prepared by reaction of 2-(2-ethyl-4-iodo-imidazol-1-yl)-ethylamine with cyclopropane-carbaldehyde. LC-MS (C): t_{R}=0.27 min; [M+H]⁺=320.2.

Cyclopropylmethyl-[2-(5,6-dimethyl-1H-benzoimidazol-2-yl)-ethyl]-amine

prepared by reaction of 2-(5,6-dimethyl-1H-benzoimidazol-2-yl)-ethylamine with cyclopropane-carbaldehyde. LC-MS (C): t_{R}=0.35 min; [M+H]⁺=244.3.

[2-(6-Chloro-1H-benzoimidazol-2-yl)-ethyl]-cyclopropylmethyl-amine

prepared by reaction of 2-(6-chloro-1H-benzoimidazol-2-yl)-ethylamine with cyclopropane-carbaldehyde. LC-MS (C): t_{R}=0.37 min; [M+H]⁺=250.3.

Cyclopropylmethyl-[2-(6-methoxy-1H-benzoimidazol-2-yl)-ethyl]-amine

prepared by reaction of 2-(6-methoxy-1H-benzoimidazol-2-yl)-ethylamine with cyclopropane-carbaldehyde. LC-MS (C): t_{R}=0.29 min; [M+H]⁺=246.3.

Cyclopropylmethyl-[2-(6-methyl-1H-benzoimidazol-2-yl)-ethyl]-amine

prepared by reaction of 2-(6-methyl-1H-benzoimidazol-2-yl)-ethylamine with cyclopropane-carbaldehyde. LC-MS (C): t_{R}=0.31 min; [M+H]⁺=230.3.

Cyclopropylmethyl-(2-indol-1-yl-ethyl)-amine

prepared by reaction of 2-indol-1-yl-ethylamine with cyclopropane-carbaldehyde. LC-MS (C): t_{R}=0.45 min; [M+H]⁺=215.4.

[2-(5-Bromo-1H-indol-3-yl)-ethyl]-cyclopropylmethyl-amine

prepared by reaction of 2-(5-bromo-1H-indol-3-yl)-ethylamine with cyclopropane-carbaldehyde. LC-MS (C): t_{R}=0.51 min; [M+H]⁺=293.2.

[2-(6-Chloro-1H-indol-3-yl)-ethyl]-cyclopropylmethyl-amine

prepared by reaction of 2-(6-chloro-1H-indol-3-yl)-ethylamine with cyclopropane-carbaldehyde. LC-MS (C): t_{R}=0.50 min; [M+H]⁺=249.3.

Cyclopropylmethyl-[2-(7-methoxy-1H-indol-3-yl)-ethyl]amine

prepared by reaction of 2-(7-methoxy-1H-indol-3-yl)-ethylamine with cyclopropane-carbaldehyde. LC-MS (C): t_{R}=0.45 min; [M+H]⁺=245.3.

Cyclopropylmethyl-[2-(5-methoxy-1H-indol-3-yl)-ethyl]amine

prepared by reaction of 2-(5-methoxy-1H-indol-3-yl)-ethylamine with cyclopropane-carbaldehyde. LC-MS (C): t_{R}=0.42 min; [M+H]⁺=245.3.

Cyclopropylmethyl-[2-(6-methoxy-1H-indol-3-yl)-ethyl]amine

prepared by reaction of 2-(6-methoxy-1H-indol-3-yl)-ethylamine with cyclopropane-carbaldehyde. LC-MS (C): t_{R}=0.42 min; [M+H]⁺=245.3.

Cyclopropylmethyl-[2-(6-methyl-1H-indol-3-yl)-ethyl]amine

prepared by reaction of 2-(6-methyl-1H-indol-3-yl)-ethylamine with cyclopropane-carbaldehyde. LC-MS (C): t_{R}=0.47 min; [M+H]⁺=229.4.

Cyclopropylmethyl-[2-(7-methyl-1H-indol-3-yl)-ethyl]amine

prepared by reaction of 2-(7-methyl-1H-indol-3-yl)-ethylamine with cyclopropane-carbaldehyde. LC-MS (C): t_{R}=0.47 min; [M+H]⁺=229.3.

Cyclopropylmethyl-[2-(4-fluoro-1H-indol-3-yl)-ethyl]-amine

prepared by reaction of 2-(4-fluoro-1H-indol-3-yl)-ethylamine with cyclopropane-carbaldehyde. LC-MS (C): t_{R}=0.46 min; [M+H]⁺=233.3.

Cyclopropylmethyl-[2-(5-fluoro-1H-indol-3-yl)-ethyl]-amine

prepared by reaction of 2-(5-fluoro-1H-indol-3-yl)-ethylamine with cyclopropane-carbaldehyde. LC-MS (C): t_{R}=0.45 min; [M+H]⁺=233.3.

Cyclopropylmethyl-[2-(6-fluoro-1H-indol-3-yl)-ethyl]-amine

prepared by reaction of 2-(6-fluoro-1H-indol-3-yl)-ethylamine with cyclopropane-carbaldehyde. LC-MS (C): t_{R}=0.45 min; [M+H]⁺=233.3.

Cyclopropylmethyl-[2-(7-fluoro-1H-indol-3-yl)-ethyl]-amine

prepared by reaction of 2-(7-fluoro-1H-indol-3-yl)-ethylamine with cyclopropane-carbaldehyde. LC-MS (C): t_{R}=0.45 min; [M+H]⁺=233.3.

Cyclopropylmethyl-[2-(1-methyl-1H-indol-3-yl)-ethyl]amine

prepared by reaction of 2-(1-methyl-1H-indol-3-yl)-ethylamine with cyclopropane-carbaldehyde. LC-MS (C): t_{R}=0.48 min; [M+H]⁺=229.4.

Cyclopropylmethyl-[2-(5-methyl-1H-indol-3-yl)-ethyl]amine

prepared by reaction of 2-(5-methyl-1H-indol-3-yl)-ethylamine with cyclopropane-carbaldehyde. LC-MS (C): t_{R}=0.47 min; [M+H]⁺=229.4.

Cyclopropylmethyl-[2-(6-methoxy-pyridin-3-yl)-ethyl]-amine

prepared by reaction of 2-(6-methoxy-pyridin-3-yl)-ethylamine with cyclopropane-carbaldehyde. LC-MS (C): t_{R}=0.31 min; [M+H]⁺=207.4.

Cyclopropylmethyl-phenethyl-amine

prepared by reaction of phenethylamine with cyclopropane-carbaldehyde. LC-MS (C): t_{R}=0.55 min; [M+H]⁺=176.5.

[2-(2-Chloro-phenyl)-ethyl]cyclopropylmethyl-amine

prepared by reaction of 2-(2-chloro-phenyl)-ethylamine with cyclopropane-carbaldehyde. LC-MS (C): t_{R}=0.66 min; [M+H]⁺=210.3.

Cyclopropylmethyl-[2-(2-methoxy-phenyl)-ethyl]-amine

prepared by reaction of 2-(2-methoxy-phenyl)-ethylamine with cyclopropane-carbaldehyde. LC-MS (C): t_{R}=0.63 min; [M+H]⁺=206.4.

Cyclopropylmethyl-[2-(2-fluoro-phenyl)-ethyl]-amine

prepared by reaction of 2-(2-fluoro-phenyl)-ethylamine with cyclopropane-carbaldehyde. LC-MS (C): t_{R}=0.58 min; [M+H]⁺=194.4.

Cyclopropylmethyl-(2-o-tolyl-ethyl)-amine

prepared by reaction of 2-o-tolyl-ethylamine with cyclopropane-carbaldehyde. LC-MS (C): t_{R}=0.65 min; [M+H]⁺=190.4.

Cyclopropylmethyl-(2-m-tolyl-ethyl)-amine

prepared by reaction of 2-m-tolyl-ethylamine with cyclopropane-carbaldehyde. LC-MS (C): t_{R}=0.67 min; [M+H]⁺=190.4.

Cyclopropylmethyl-[2-(3-methoxy-phenyl)-ethyl]-amine

prepared by reaction of 2-(3-methoxy-phenyl)-ethylamine with cyclopropane-carbaldehyde. LC-MS (C): t_{R}=0.60 min; [M+H]⁺=206.4.

[2-(4-Chloro-phenyl)-ethyl]cyclopropylmethyl-amine

prepared by reaction of 2-(4-chloro-phenyl)-ethylamine with cyclopropane-carbaldehyde. LC-MS (C): t_{R}=0.70 min; [M+H]⁺=210.3.

Cyclopropylmethyl-(2-p-tolyl-ethyl)-amine

prepared by reaction of 2-p-tolyl-ethylamine with cyclopropane-carbaldehyde. LC-MS (C): t_{R}=0.67 min; [M+H]⁺=190.5.

Cyclopropylmethyl-[2-(4-ethyl-phenyl)-ethyl]-amine

prepared by reaction of 2-(4-ethyl-phenyl)-ethylamine with cyclopropane-carbaldehyde. LC-MS (C): t_{R}=0.77 min; [M+H]⁺=204.4.

Cyclopropylmethyl-[2-(4-methoxy-phenyl)-ethyl]-amine

prepared by reaction of 2-(4-methoxy-phenyl)-ethylamine with cyclopropane-carbaldehyde. LC-MS (C): t_{R}=0.59 min; [M+H]⁺=206.4.

4-[2-(Cyclopropylmethyl-amino)-ethyl]-phenol

prepared by reaction of 4-(2-amino-ethyl)-phenol with cyclopropane-carbaldehyde. LC-MS (C): t_{R}=0.41 min; [M+H]⁺=192.4.

Cyclopropylmethyl-[2-(2,4-dimethyl-phenyl)-ethyl]-amine

prepared by reaction of 2-(2,4-dimethyl-phenyl)-ethylamine with cyclopropane-carbaldehyde. LC-MS (C): t_{R}=0.75 min; [M+H]⁺=204.4.

Cyclopropylmethyl-[2-(2,5-dimethoxy-phenyl)-ethyl]-amine

prepared by reaction of 2-(2,5-dimethoxy-phenyl)-ethylamine with cyclopropane-carbaldehyde. LC-MS (C): t_{R}=0.65 min; [M+H]⁺=236.4.

Cyclopropylmethyl-[2-(2,5-dimethyl-phenyl)-ethyl]-amine

prepared by reaction of 2-(2,5-dimethyl-phenyl)-ethylamine with cyclopropane-carbaldehyde. LC-MS (C): t_{R}=0.75 min; [M+H]⁺=204.4.

[2-(5-Bromo-2-methoxy-phenyl)-ethyl]cyclopropylmethyl-amine

prepared by reaction of 2-(5-bromo-2-methoxy-phenyl)-ethylamine with cyclopropane-carbaldehyde. LC-MS (C): t_{R}=0.77 min; [M+H]⁺=284.3.

(2-Benzo[1,3]dioxol-5-yl-ethyl)-cyclopropylmethyl-amine

prepared by reaction of 2-benzo[1,3]dioxol-5-yl-ethylamine with cyclopropane-carbaldehyde. LC-MS (C): t_{R}=0.57 min; [M+H]⁺=220.3.

Cyclopropylmethyl-[2-(2,3-dihydro-benzo[1,4]dioxin-6-yl)-ethyl]-amine

prepared by reaction of 2-(2,3-dihydro-benzo[1,4]dioxin-6-yl)-ethylamine (A. S. Capilla et al. Tetrahedron 2001, 57, 8297-8304) with cyclopropane-carbaldehyde. LC-MS (C): t_{R}=0.58 min; [M+H]⁺=234.4.

Cyclopropylmethyl-[2-(4-ethoxy-3-methoxy-phenyl)-ethyl]-amine

prepared by reaction of 2-(4-ethoxy-3-methoxy-phenyl)-ethylamine with cyclopropane-carbaldehyde. LC-MS (C): t_{R}=0.63 min; [M+H]⁺=250.4.

Cyclopropylmethyl-[2-(3-ethoxy-4-methoxy-phenyl)-ethyl]-amine

prepared by reaction of 2-(3-ethoxy-4-methoxy-phenyl)-ethylamine with cyclopropane-carbaldehyde. LC-MS (C): t_{R}=0.62 min; [M+H]⁺=250.4.

Cyclopropylmethyl-[2-(4-methoxy-3-methyl-phenyl)-ethyl]-amine

prepared by reaction of 2-(4-methoxy-3-methyl-phenyl)-ethylamine with cyclopropane-carbaldehyde. LC-MS (C): t_{R}=0.70 min; [M+H]⁺=220.4.

[2-(3-Bromo-4-methoxy-phenyl)-ethyl]cyclopropylmethyl-amine

prepared by reaction of 2-(3-bromo-4-methoxy-phenyl)-ethylamine with cyclopropane-carbaldehyde. LC-MS (C): t_{R}=0.71 min; [M+H]⁺=284.2.

Cyclopropylmethyl-[2-(3,4-dimethyl-phenyl)-ethyl]-amine

prepared by reaction of 2-(3,4-dimethyl-phenyl)-ethylamine with cyclopropane-carbaldehyde. LC-MS (C): t_{R}=0.75 min; [M+H]⁺=204.4.

4-[2-(Cyclopropylmethyl-amino)-ethyl]-2-methoxy-phenol

prepared by reaction of 4-(2-amino-ethyl)-2-methoxy-phenol with cyclopropane-carbaldehyde. LC-MS (C): t_{R}=0.44 min; [M+H]⁺=222.3.

Cyclopropylmethyl-[2-(3,5-dimethoxy-phenyl)-ethyl]-amine

prepared by reaction of 2-(3,5-dimethoxy-phenyl)-ethylamine with cyclopropane-carbaldehyde. LC-MS (C): t_{R}=0.64 min; [M+H]⁺=236.4.

Cyclopropylmethyl-[2-(2,6-dichloro-phenyl)-ethyl]amine

prepared by reaction of 2-(2,6-dichloro-phenyl)-ethylamine with cyclopropane-carbaldehyde. LC-MS (C): t_{R}=0.71 min; [M+H]⁺=244.3.

Cyclopropylmethyl-[2-(3,4,5-trimethoxy-phenyl)-ethyl]-amine

prepared by reaction of 2-(3,4,5-trimethoxy-phenyl)-ethylamine (S.-I. Murahashi et al. Bull. Chem. Soc. Jpn. 1990, 63, 1252-1254) with cyclopropane-carbaldehyde. LC-MS (C): t_{R}=0.58 min; [M+H]⁺=266.4.

Cyclopropylmethyl-[2-(4-isopropoxy-3,5-dimethoxy-phenyl)-ethyl]-amine

prepared by reaction of 2-(4-isopropoxy-3,5-dimethoxy-phenyl)-ethylamine (D. E. Nichols et al. J. Med. Chem. 1977, 20, 299-301) with cyclopropane-carbaldehyde. LC-MS (C): t_{R}=0.74 min; [M+H]⁺=294.3.

Cyclopropylmethyl-[2-(4-iodo-2,5-dimethoxy-phenyl)-ethyl]-amine

prepared by reaction of 2-(4-iodo-2,5-dimethoxy-phenyl)-ethylamine (T. Sargent III et al. J. Med. Chem. 1977, 20, 1543-1546) with cyclopropane-carbaldehyde. LC-MS (C): t_{R}=0.82 min; [M+H]⁺=362.2.

Cyclopropylmethyl-[2-(6-methoxy-1H-benzoimidazol-2-yl)-ethyl]-amine

prepared by reaction of 2-(6-methoxy-1H-benzoimidazol-2-yl)-ethylamine with cyclopropane-carbaldehyde. LC-MS (C): t_{R}=0.29 min; [M+H]⁺=246.3.

Cyclopropylmethyl-[2-(5,6-dimethyl-1H-benzoimidazol-2-yl)-ethyl]-amine

prepared by reaction of 2-(5,6-dimethyl-1H-benzoimidazol-2-yl)-ethylamine with cyclopropane-carbaldehyde. LC-MS (C): t_{R}=0.35 min; [M+H]⁺=244.3.

Cyclopropylmethyl-[2-(1-methyl-1H-indol-3-yl)-ethyl]-amine

prepared by reaction of 2-(1-methyl-1H-indol-3-yl)-ethylamine with cyclopropane-carbaldehyde. LC-MS (C): t_{R}=0.48 min; [M+H]⁺=229.4.

[2-(6-Chloro-1H-indol-3-yl)-ethyl]-cyclopropylmethyl-amine

prepared by reaction of 2-(6-chloro-1H-indol-3-yl)-ethylamine with cyclopropane-carbaldehyde. LC-MS (C): t_{R}=0.50 min; [M+H]⁺=249.3.

Cyclopropylmethyl-[2-(7-methoxy-1H-indol-3-yl)-ethyl]-amine

prepared by reaction of 2-(7-methoxy-1H-indol-3-yl)-ethylamine with cyclopropane-carbaldehyde. LC-MS (C): t_{R}=0.45 min; [M+H]⁺=245.3.

Cyclopropylmethyl-[2-(5-methoxy-1H-indol-3-yl)-ethyl]-amine

prepared by reaction of 2-(5-methoxy-1H-indol-3-yl)-ethylamine with cyclopropane-carbaldehyde. LC-MS (C): t_{R}=0.42 min; [M+H]⁺=245.3.

Cyclopropylmethyl-[2-(6-methoxy-1H-indol-3-yl)-ethyl]-amine

prepared by reaction of 2-(6-methoxy-1H-indol-3-yl)-ethylamine with cyclopropane-carbaldehyde. LC-MS (C): t_{R}=0.42 min; [M+H]⁺=245.3.

Cyclopropylmethyl-[2-(5-methyl-1H-indol-3-yl)-ethyl]amine

prepared by reaction of 2-(5-methyl-1H-indol-3-yl)-ethylamine with cyclopropane-carbaldehyde. LC-MS (C): t_{R}=0.47 min; [M+H]⁺=229.4.

Cyclopropylmethyl-[2-(6-methyl-1H-indol-3-yl)-ethyl]amine

prepared by reaction of 2-(6-methyl-1H-indol-3-yl)-ethylamine with cyclopropane-carbaldehyde. LC-MS (C): t_{R}=0.47 min; [M+H]⁺=229.4.

Cyclopropylmethyl-[2-(7-methyl-1H-indol-3-yl)-ethyl]amine

prepared by reaction of 2-(7-methyl-1H-indol-3-yl)-ethylamine with cyclopropane-carbaldehyde. LC-MS (C): t_{R}=0.47 min; [M+H]⁺=229.3.

Cyclopropylmethyl-[2-(4-fluoro-1H-indol-3-yl)-ethyl]-amine

prepared by reaction of 2-(4-fluoro-1H-indol-3-yl)-ethylamine with cyclopropane-carbaldehyde. LC-MS (C): t_{R}=0.46 min; [M+H]⁺=233.3.

Cyclopropylmethyl-[2-(6-fluoro-1H-indol-3-yl)-ethyl]-amine

prepared by reaction of 2-(6-fluoro-1H-indol-3-yl)-ethylamine with cyclopropane-carbaldehyde. LC-MS (C): t_{R}=0.45 min; [M+H]⁺=233.3.

Cyclopropylmethyl-[2-(7-fluoro-1H-indol-3-yl)-ethyl]-amine

prepared by reaction of 2-(7-fluoro-1H-indol-3-yl)-ethylamine with cyclopropane-carbaldehyde. LC-MS (C): t_{R}=0.45 min; [M+H]⁺=233.3.

A.6.8 Synthesis of Sec.-Amines by Alkylation with Alkyl Halides (General Procedure)

TEA (0.63 mmol) and the respective alkyl halide (0.63 mmol) are successively added to a solution of the respective aryl-ethylamine (free base, 0.63 mmol) in a mixture of THF (2.0 mL) and DMF (1.0 mL). The mixture is stirred at 50° C. for 17 h, diluted with MeOH (1.0 mL), filtered and purified by prep. HPLC (basic gradient) to give the desired product. The ammonia is removed in vacuo, hydrochloric acid (10%, 1.0 mL) is added and the solvents are removed in vacuo to give the desired product as a hydrochloride salt.

4-[2-(Cyclopropylmethyl-amino)-ethyl]-thiazol-2-ylamine

prepared by reaction of 4-(2-amino-ethyl)-thiazol-2-ylamine (J. C. Eriks et al. J. Med. Chem., 1992, 35, 3239-3246) with bromomethyl-cyclopropane. LC-MS (C): t_{R}=0.14 min; [M+H]⁺=198.4.

A.6.9 Synthesis of Sec.-Amines by Red. Amination of benzyl-[2-(3,4-dimethoxy-phenyl)-ethyl]-amine and subsequent benzyl-deprotection

Benzyl-[2-(3,4-dimethoxy-phenyl)-ethyl]amine

Benzaldehyde (55.2 mmol) is added to a mixture of 2-(3,4-dimethoxy-phenyl)-ethylamine (55.2 mmol) and molecular sieve (3 Å, 12.5 g) in MeOH (125 mL). After 60 min sodium borohydride (66.2 mmol) is added portionwise. The mixture is stirred for 30 min and filtered to remove the molecular sieve. Water (5.0 mL) is added and the organic volatiles are removed in vacuo. TBME and water are added, the layers are separated and the aqueous layer is extracted twice with TBME. The combined organic layers are washed three times with water, dried over MgSO₄ and concentrated in vacuo to give the desired product which is used without further purification. LC-MS (B): t_{R}=0.84 min; [M+H]⁺=272.2.

Alkyl-benzyl-[2-(3,4-dimethoxy-phenyl)-ethyl]-amine derivatives (General Procedure)

Sodium triacetoxyborohydride (5.16 mmol) is added to a mixture of benzyl-[2-(3,4-dimethoxy-phenyl)-ethyl]-amine (3.69 mmol) and the respective carbonyl compound (4.42 mmol) in DCM (10 mL). The mixture is stirred for 2 h, diluted with water (10 mL) and stirred for additional 60 min. An aqueous NaOH solution (1.0 M) is added to a final pH 8-9, the layers are separated and the aqueous layer is extracted twice with DCM (2×20 mL). The combined organic layers are concentrated in vacuo, diluted with CH₃CN (4.0 mL) and purified by prep. HPLC using a basic gradient to give the desired product.

Remark: In case acetone is used as carbonyl compound a second aliquote of acetone

(4.42 mmol) and sodium triacetoxyborohydride (5.16 mmol) is added 2 h after the first addition and the mixture is stirred for additional 16 h prior to work-up.

Benzyl-[2-(3,4-dimethoxy-phenyl)-ethyl]-ethyl-amine

prepared by reaction of benzyl-[2-(3,4-dimethoxy-phenyl)-ethyl]-amine with acetaldehyde. LC-MS (B): t_{R}=1.02 min; [M+H]⁺=300.1.

Benzyl-[2-(3,4-dimethoxy-phenyl)-ethyl]-propyl-amine

prepared by reaction of benzyl-[2-(3,4-dimethoxy-phenyl)-ethyl]-amine with propionaldehyde. LC-MS (B): t_{R}=1.09 min; [M+H]⁺=314.2.

Benzyl-[2-(3,4-dimethoxy-phenyl)-ethyl]isobutyl-amine

prepared by reaction of benzyl-[2-(3,4-dimethoxy-phenyl)-ethyl]-amine with 2-methyl-propionaldehyde. LC-MS (B): t_{R}=1.16 min; [M+H]⁺=328.2.

Benzyl-[2-(3,4-dimethoxy-phenyl)-ethyl]isopropyl-amine

prepared by reaction of benzyl-[2-(3,4-dimethoxy-phenyl)-ethyl]-amine with acetone. LC-MS (B): t_{R}=1.10 min; [M+H]⁺=314.2.

Alkyl-[2-(3,4-dimethoxy-phenyl)-ethyl]amine derivatives (General Procedure)

A mixture of the respective alkyl-benzyl-[2-(3,4-dimethoxy-phenyl)-ethyl]-amine derivative (2.14 mmol) in EtOH (15 mL) is treated with Pd/C (10%, 500 mg) and stirred under a hydrogen atmosphere (1 bar) for 17 h. After filtration through celite the solvents are removed in vacuo and the residue is diluted by addition of ether (30 mL) and isopropanol (0.2 mL). A solution of HCl in ether (2.0 M) is added under vigorous stirring, the organic volatiles are removed in vacuo and the residue is treated with ether (5.0 mL). The suspension is decanted, ether (5.0 mL) is added to the remaining solid and the obtained suspension is decanted again. The solid is dried in vacuo to give the desired product as a hydrochloride salt.

[2-(3,4-Dimethoxy-phenyl)-ethyl]ethyl-amine

prepared by deprotection of benzyl-[2-(3,4-dimethoxy-phenyl)-ethyl]-ethyl-amine. LC-MS (B): t_{R}=0.90 min; [M+H]⁺=210.3.

[2-(3,4-Dimethoxy-phenyl)-ethyl]propyl-amine

prepared by deprotection of benzyl-[2-(3,4-dimethoxy-phenyl)-ethyl]-propyl-amine. LC-MS (B): t_{R}=0.88 min; [M+H]⁺=224.3.

[2-(3,4-Dimethoxy-phenyl)-ethyl]isobutyl-amine

prepared by deprotection of benzyl-[2-(3,4-dimethoxy-phenyl)-ethyl]-isobutyl-amine. LC-MS (B): t_{R}=0.89 min; [M+H]⁺=238.3.

[2-(3,4-Dimethoxy-phenyl)-ethyl]isopropyl-amine

prepared by deprotection of benzyl-[2-(3,4-dimethoxy-phenyl)-ethyl]-isopropyl-amine. LC-MS (B): t_{R}=0.87 min; [M+H]⁺=224.3.

A.6.10 Synthesis of 2-[2-(3,4-Dimethoxy-phenyl)-ethylamino]-acetamide

2-{Benzyl-[2-(3,4-dimethoxy-phenyl)-ethyl]-amino}-acetamide

A mixture of benzyl-[2-(3,4-dimethoxy-phenyl)-ethyl]-amine (3.69 mmol), 2-bromo-acetamide (3.87 mmol) and DIPEA (4.05 mmol) in THF (20 mL) is stirred at 60° C. for 22 h. Additional DIPEA (0.92 mmol) and 2-bromo-acetamide (0.92 mmol) are added and the mixture is stirred for further 6 h at 60° C. The mixture is filtered, the residue is washed with THF, the filtrates are combined and the solvents are removed in vacuo. The residue is dissolved in acetonitrile (5.0 mL) and purified by prep. HPLC using a basic gradient to give the desired product as a white solid. LC-MS (B): t_{R}=0.79 min; [M+H]⁺=329.1; ¹H NMR (CDCl₃): δ=2.77 (s, 4H), 3.08 (s, 2H), 3.69 (s, 2H), 3.82 (s, 3H), 3.86 (s, 3H), 6.64 (d, J=1.8 Hz, 1H), 6.70 (dd, J=8.3, 2.0 Hz, 1H), 6.79 (d, J=8.3 Hz, 1H), 7.20 (m, 2H), 7.29 (m, 3H).

2-[2-(3,4-Dimethoxy-phenyl)-ethylamino]-acetamide

A mixture of 2-{benzyl-[2-(3,4-dimethoxy-phenyl)-ethyl]-amino}-acetamide (2.83 mmol) in EtOH (15 mL) is treated with Pd/C (10%, 500 mg) and stirred under a hydrogen atmosphere (1 bar) for 3 d. After filtration through celite the solvents are removed in vacuo and the residue is diluted by addition of MeOH (3.0 mL) and ether (50 mL). A solution of HCl in ether (2.0 M) is added under vigorous stirring, the organic volatiles are removed in vacuo and the residue is treated with ether (5.0 mL). The suspension is decanted, ether (5.0 mL) is added to the remaining solid and the obtained suspension is decanted again. The solid is dried in vacuo to give the desired product as a hydrochloride salt. LC-MS (B): t_{R}=0.57 min; [M+H]⁺=239.2.

### A.6.11 Synthesis of 2-[2-(3,4-Dimethoxy-phenyl)-ethylamino]-N,N-dimethyl-acetamide

2-{Benzyl-[2-(3,4-dimethoxy-phenyl)-ethyl]-amino}-N,N-dimethyl-acetamide

A mixture of benzyl-[2-(3,4-dimethoxy-phenyl)-ethyl]-amine (3.69 mmol), 2-chloro-N,N-dimethylacetamide (3.87 mmol) and DIPEA (4.05 mmol) in THF (20 mL) is stirred at 60° C. for 22 h. Additional DIPEA (3.69 mmol), 2-chloro-N,N-dimethyl-acetamide (3.69 mmol) and DMF (1.0 mL) are added and the mixture is stirred for further 24 h at 60° C. The mixture is filtered, the residue is washed with THF, the filtrates are combined and the solvents are removed in vacuo. The residue is dissolved in acetonitrile (5.0 mL) and purified by prep. HPLC using a basic gradient to give the desired product as a viscous oil. LC-MS (B): t_{R}=0.86 min; [M+H]⁺=357.2; ¹H NMR (CDCl₃): δ=2.74 (m, 2H), 2.79 (s, 3H), 2.82 (s, 3H), 2.85 (m, 2H), 3.28 (s, 2 H), 3.72 (s, 2H), 3.83 (s, 3H), 3.84 (s, 3H), 6.68 (m, 2H), 6.76 (d, J=8.0 Hz, 1H), 7.24 (m, 1H), 7.29 (d, J=4.3 Hz, 4H).

2-[2-(3,4-Dimethoxy-phenyl)-ethylamino]-N,N-dimethyl-acetamide

A mixture of 2-{benzyl-[2-(3,4-dimethoxy-phenyl)-ethyl]-amino}-N,N-dimethyl-acetamide (2.40 mmol) in EtOH (15 mL) is treated with Pd/C (10%, 500 mg) and stirred under a hydrogen atmosphere (1 bar) for 3 d. After filtration through celite the solvents are removed in vacuo and the residue is diluted by addition of ether (30 mL) and isopropanol (0.2 mL). A solution of HCl in ether (2.0 M) is added under vigorous stirring. The suspension is decanted, ether (5.0 mL) is added to the remaining solid and the obtained suspension is decanted again. The solid is dried in vacuo to give the desired product as a hydrochloride salt. LC-MS (B): t_{R}=0.62 min; [M+H]⁺=267.0.

A.6.12 Synthesis of [2-(3,4-Dimethoxy-phenyl)-ethyl]-(2,2,2-trifluoro-ethyl)-amine

N-[2-(3,4-Dimethoxy-phenyl)-ethyl]-2,2,2-trifluoro-acetamide

Trifluoro-acetic acid ethyl ester (20.7 mmol) is added dropwise to a solution of 2-(3,4-dimethoxy-phenyl)-ethylamine (18.8 mmol) and TEA (22.6 mmol) in MeOH (40 mL). After 30 min the volatiles are removed in vacuo and the residue is dissolved in TBME (100 mL). The mixture is washed three times with hydrochloric acid (0.5 M, 3×50 mL), twice with water (2×50 mL) and once with brine (30 mL), dried over MgSO₄ and concentrated in vacuo to give the desired product as a white solid. LC-MS (B): t_{R}=0.77 min; [M+NH₃+H]⁺=295.0; ¹H NMR (CDCl₃): δ=2.82 (t, J=6.5 Hz, 2H), 3.59 (q, J=6.5 Hz, 2H), 3.86 (s, 6H), 6.26 (bs, 1H), 6.68 (s, 1H), 6.71 (d, J=8.3 Hz, 1H), 6.82 (d, J=8.0 Hz, 1H).

[2-(3,4-Dimethoxy-phenyl)-ethyl]-(2,2,2-trifluoro-ethyl)-amine

At 0° C. a solution of borane tetrahydrofuran complex in THF (1.0 M, 39.9 mmol) is added to a solution of N-[2-(3,4-dimethoxy-phenyl)-ethyl]-2,2,2-trifluoro-acetamide (17.1 mmol) in THF (20.0 mL). After 1 h the mixture is heated to reflux for 22 h, cooled to 0° C. and diluted with water (20 mL). The volatiles are removed in vacuo, TBME (50 mL) and water (30 mL) are added and the layers are separated. The aqueous layer is extracted twice with TBME (2×20 mL) and the combined organic layers are extracted three times with hydrochloric acid (0.5 M, 3×20 mL). The combined aqueous layers are made basic by addition of aqueous NaOH solution (2.0 M) and extracted four times with DCM (4×30 mL). The combined organic layers are dried over MgSO₄ and concentrated in vacuo. The residue is dissolved in ether (100 mL) and isopropanol (0.5 mL) and the mixture is carefully acidified by addition of a solution of HCl in ether (2.0 M). The obtained suspension is filtered and the residue is washed with ether and dried in vacuo to give the desired product as a hydrochloride salt. LC-MS (B): t_{R}=0.81 min; [M+CH₃CN+H]⁺=305.2; ¹H NMR (D₃O): δ=2.96 (t, J=7.8 Hz, 2H), 3.38 (t, J=7.8 Hz, 2H), 3.77 (s, 3H), 3.78 (s, 3H), 3.92 (q, J=8.5 Hz, 2H), 6.85 (d, J=8.3 Hz, 1H), 6.91 (s, 1H), 6.95 (d, J=8.0 Hz, 1H).

A.6.13 Synthesis of 2-(3,4-Dimethoxy-phenyl)-acetamide derivatives (General Procedure)

TBTU (5.61 mmol) is added to a mixture of (3,4-dimethoxy-phenyl)-acetic acid (5.10 mmol), the respective amine (5.61 mmol) and DIPEA (10.2 mmol) in DMF (10 mL). The mixture is stirred for 10 min and purified by prep HPLC using a basic gradient to give the desired amide derivative.

N-Cyclopropyl-2-(3,4-dimethoxy-phenyl)-acetamide

prepared by reaction of (3,4-dimethoxy-phenyl)-acetic acid with cyclopropylamine. LC-MS (B): t_{R}=0.62 min; [M+H]⁺=236.2; ¹H NMR (CDCl₃): δ=0.38 (m, 2H), 0.72 (m, 2H), 2.65 (m, 1H), 3.47 (s, 2H), 3.86 (s, 3H), 3.87 (s, 3H), 5.46 (bs, 1H), 6.74 (m, 2H), 6.82 (m, 1H).

2-(3,4-Dimethoxy-phenyl)-N-(2-hydroxy-ethyl)-acetamide

prepared by reaction of (3,4-dimethoxy-phenyl)-acetic acid with 2-amino-ethanol. LC-MS (B): t_{R}=0.53 min; [M+H]⁺=240.2; ¹H NMR (CDCl₃): δ=3.36 (φq, J=5.3 Hz, 2H), 3.52 (s, 2H), 3.66 (t, J=5.0 Hz, 2H), 3.86 (s, 6H), 5.91 (bs, 1H), 6.78 (m, 2H), 6.83 (d, J=7.8 Hz, 1H).

2-(3,4-Dimethoxy-phenyl)-N-(2-methoxy-ethyl)-acetamide

prepared by reaction of (3,4-dimethoxy-phenyl)-acetic acid with 2-methoxy-ethylamine. LC-MS (B): t_{R}=0.59 min; [M+H]⁺=254.2; ¹H NMR (CDCl₃): δ=3.28 (s, 3H), 3.39 (m, 4H), 3.50 (s, 2H), 3.87 (s, 6H), 5.79 (bs, 1H), 6.78 (m, 2H), 6.83 (d, J=8.5 Hz, 1H).

2-(3,4-Dimethoxy-phenyl)-N-(2-dimethylamino-ethyl)-acetamide

prepared by reaction of (3,4-dimethoxy-phenyl)-acetic acid with N,N-dimethyl-ethane-1,2-diamine. LC-MS (B): t_{R}=0.60 min; [M+H]⁺=267.2; ¹H NMR (CDCl₃): δ=2.15 (s, 6H), 2.33 (t, J=6.0 Hz, 2H), 3.27 (oq, J=5.8 Hz, 2H), 3.48 (s, 2H), 3.86 (s, 3H), 3.87 (s, 3H), 5.99 (bs, 1H), 6.78 (m, 2H), 6.82 (d, J=8.0 Hz, 1H).

A.6.14 Synthesis of 2-(3,4-dimethoxy-phenyl)-ethylamine derivatives (General Procedure)

Under a nitrogen atmosphere a solution of the respective amide derivative (3.37 mmol) in THF (10 mL) is added dropwise (10 min) to a refluxing suspension of LAH (12.0 mmol) in THF (20 mL). The mixture is stirred at reflux for 20 h and cooled to 0° C. Isopropanol (2.46 mL) and an aqueous NaOH solution (2.0 M, 1.72 mL) are added dropwise. The mixture is diluted with additional THF, filtered and concentrated in vacuo to give a crude product which is purified by prep. HPLC (basic gradient). The combined fractions are dried in vacuo, the residue is dissolved in ether (30 mL) and isopropanol (0.3 mL) and the solution is made acidic by addition of a solution of HCl in ether (2.0 M). The obtained suspension is filtered and the residue is dried in vacuo to give the desired product as a hydrochloride salt.

Cyclopropyl-[2-(3,4-dimethoxy-phenyl)-ethyl]-amine

prepared by reduction of N-cyclopropyl-2-(3,4-dimethoxy-phenyl)-acetamide; the mixture is heated to reflux for only 60 min. LC-MS (B): t_{R}=0.76 min; [M+H]⁺=222.3.

2-[2-(3,4-Dimethoxy-phenyl)-ethylamino]-ethanol

prepared by reduction of 2-(3,4-dimethoxy-phenyl)-N-(2-hydroxy-ethyl)-acetamide. LC-MS (B): t_{R}=0.61 min; [M+H]⁺=226.3.

[2-(3,4-Dimethoxy-phenyl)-ethyl]-(2-methoxy-ethyl)-amine

prepared by reduction of 2-(3,4-dimethoxy-phenyl)-N-(2-methoxy-ethyl)-acetamide. LC-MS (B): t_{R}=0.70 min; [M+H]⁺=240.2.

N′-[2-(3,4-Dimethoxy-phenyl)-ethyl]-N,N-dimethyl-ethane-1,2-diamine

prepared by reduction of 2-(3,4-dimethoxy-phenyl)-N-(2-dimethylamino-ethyl)-acetamide.

A.6.15 Synthesis of 2-(1H-indol-3-yl)-2-oxo-acetamide derivatives (General Procedure)

At 0° C. oxalyl chloride (40.0 mmol) is added dropwise to a suspension of the respective indole derivative (22.2 mmol) in ether (45 mL). The mixture is stirred for 10 min at 0° C., allowed to reach RT and stirred for additional 80 to 120 min (warming to RT is not necessary in all cases). The obtained suspension is cooled to 0° C. and filtered. The residue is washed with ice-cold ether. A suspension of the residue in ether (60 mL) is cooled to 0° C. and treated dropwise with the respective amine (40.0 mmol). Work-up: after 30 min the suspension is filtered and the residue is washed with three portions of ether (40 mL each), two portions of water (30 mL each) and additional two portions of ether (40 mL each). The residue is dried in vacuo to give the respective product. Alternative work-up: after 90 min TBME (500 mL) and sat. aqueous NaHCO₃ solution (200 mL) are added, the layers are separated and the aqueous layer is extracted twice with TBME (2×100 mL). The combined organic layers are dried over MgSO₄ and concentrated in vacuo to give the desired product.

N-Benzyl-2-(5-fluoro-1H-indol-3-yl)-2-oxo-acetamide

prepared by reaction of 5-fluoroindole with oxalyl chloride and benzylamine. LC-MS (C): t_{R}=0.73 min; [M+H]⁺=297.2.

N-[2-(tert-Butyl-dimethyl-silanyloxy)-ethyl]-2-(5-fluoro-1H-indol-3-yl)-2-oxo-acetamide

prepared by reaction of 5-fluoroindole with oxalyl chloride and 2-(tert-butyl-dimethyl-silanyloxy)-ethylamine (C. Palomo, Org. Lett. 2007, 9, 101-104). ¹H-NMR (DMSO-d₆): δ=0.04 (s, 6H), 0.86 (s, 9H), 3.33 (m, 2H), 3.70 (t, J=6.3 Hz, 2H), 7.14 (td, J=9.3, 2.8 Hz, 1H), 7.56 (dd, J=8.8, 4.5 Hz, 1H), 7.90 (dd, J=9.8, 2.5 Hz, 1H), 8.64 (t, J=6.0 Hz, 1H), 8.83 (d, J=3.3 Hz, 1H).

N-Cyclopropylmethyl-2-(5-methoxy-4-methyl-1H-indol-3-yl)-2-oxo-acetamide

prepared by reaction of 5-methoxy-4-methyl-1H-indole with oxalyl chloride and aminomethyl-cyclopropane. LC-MS (C): t_{R}=0.65 min; [M+H]⁺=287.3.

N-Cyclopropylmethyl-2-(5H-[1,3]dioxolo[4,5-f]indol-7-yl)-2-oxo-acetamide

prepared by reaction of 5H-[1,3]dioxolo[4,5-f]indole with oxalyl chloride and aminomethyl-cyclopropane. LC-MS (C): t_{R}=0.62 min; [M+H]⁺=287.2.

N-Cyclopropylmethyl-2-(5,6-difluoro-1H-indol-3-yl)-2-oxo-acetamide

prepared by reaction of 5,6-difluoro-1H-indole with oxalyl chloride and aminomethyl-cyclopropane. ¹H-NMR (DMSO-d₆): δ=0.25 (m, 2H), 0.43 (m, 2H), 1.04 (m, 1H), 3.10 (t, J=6.3 Hz, 2H), 7.60 (dd, J=10.8, 7.0 Hz, 1H), 8.07 (dd, J=11.0, 8.0 Hz, 1H), 8.81 (d, J=3.3 Hz, 1H), 8.82 (bt, J=5.8 Hz, 1H), 12.35 (bs, 1H).

A.6.16 Synthesis of 2-(1H-indol-3-yl)-ethylamine derivatives (General Procedure)

A solution of the respective 2-(M-indol-3-yl)-2-oxo-acetamide derivative (1.18 mmol) in THF (10 mL) is added dropwise to a heated (around 65° C.) suspension of LAH in THF (15 mL) under inert atmosphere (alternatively the respective 2H-indol-3-yl)-2-oxo-acetamide derivative is added portionwise as a solid). The mixture is stirred at around 65° C. for additional 2d, cooled to 0° C. and treated with isopropanol and aqueous NaOH solution (2.0 M) respectively. THF is added, the suspension is filtered and the residue is rinsed three times with THF (20 mL each). The combined filtrates are concentrated in vacuo and the residue is used without further purification or purified by prep. HPLC or FC (gradient: DCM to DCM/MeOH 96/4) to give the desired product.

Benzyl-[2-(5-fluoro-1H-indol-3-yl)-ethyl]amine

prepared by reduction of N-benzyl-2-(5-fluoro-1H-indol-3-yl)-2-oxo-acetamide. LC-MS (C): t_{R}=0.51 min; [M+H]⁺=269.3.

2-[2-(5-Fluoro-1H-indol-3-yl)-ethylamino]-ethanol

prepared by reduction of N-[2-(tert-Butyl-dimethyl-silanyloxy)-ethyl]-2-(5-fluoro-1H-indol-3-yl)-2-oxo-acetamide. LC-MS (C): t_{R}=0.37 min; [M+H]⁺=223.3.

Cyclopropylmethyl-[2-(5-methoxy-4-methyl-1H-indol-3-yl)-ethyl]-amine

prepared by reduction of N-cyclopropylmethyl-2-(5-methoxy-4-methyl-1H-indol-3-yl)-2-oxo-acetamide. LC-MS (C): t_{R}=0.46 min; [M+H]⁺=259.3.

Cyclopropylmethyl-[2-(5H-[1,3]dioxolo[4,5-f]indol-7-yl)-ethyl]-amine

prepared by reduction of N-cyclopropylmethyl-2-(5H-[1,3]dioxolo[4,5-f]indol-7-yl)-2-oxo-acetamide. LC-MS (C): t_{R}=0.42 min; [M+H]⁺=259.2.

Cyclopropylmethyl-[2-(5,6-difluoro-1H-indol-3-yl)-ethyl]-amine

prepared by reduction of N-cyclopropylmethyl-2-(5,6-difluoro-1H-indol-3-yl)-2-oxo-acetamide. LC-MS (C): t_{R}=0.48 min; [M+H]⁺=251.2.

A.6.17 Synthesis of benzyl-[2-(5-fluoro-1H-indol-3-yl)-ethyl]-amine derivatives (General Procedure)

A mixture of benzyl-[2-(5-fluoro-1H-indol-3-yl)-ethyl]-amine (0.43 mmol), DIPEA (0.47 mmol or 0.94 mmol) and the respective alkyl halide or alkyl triflate (0.45 mmol) in THF (1.5 mL) is heated to 60° C. and stirred for 20 h. In case LC-MS indicated residual starting material an additional portion of the electrophile (0.43 mmol) is added and the mixture is stirred at 60° C. for further 24 h. The volatiles are removed in vacuo and the residue is diluted with DMF (3.0 mL) and purified by prep. HPLC to give the respective product.

Benzyl-[2-(5-fluoro-1H-indol-3-yl)-ethyl]-methyl-amine

prepared by reaction of benzyl-[2-(5-fluoro-1H-indol-3-yl)-ethyl]-amine with methyl iodide. LC-MS (B): t_{R}=0.96 min; [M+H]⁺=283.0.

Benzyl-ethyl-[2-(5-fluoro-1H-indol-3-yl)-ethyl]-amine

prepared by reaction of benzyl-[2-(5-fluoro-1H-indol-3-yl)-ethyl]-amine with ethyl iodide. LC-MS (B): t_{R}=1.02 min; [M+H]⁺=296.9.

Benzyl-[2-(5-fluoro-1H-indol-3-yl)-ethyl]-propyl-amine

prepared by reaction of benzyl-[2-(5-fluoro-1H-indol-3-yl)-ethyl]-amine with n-propyl iodide. LC-MS (B): t_{R}=1.07 min; [M+H]⁺=311.0.

Benzyl-[2-(5-fluoro-1H-indol-3-yl)-ethyl]-(2,2,2-trifluoro-ethyl)-amine

prepared by reaction of benzyl-[2-(5-fluoro-1H-indol-3-yl)-ethyl]-amine with trifluoro-methanesulfonic acid 2,2,2-trifluoro-ethyl ester. LC-MS (B): t_{R}=1.03 min; [M+H]⁺=351.1.

2-{Benzyl-[2-(5-fluoro-1H-indol-3-yl)-ethyl]-amino}-acetamide

prepared by reaction of benzyl-[2-(5-fluoro-1H-indol-3-yl)-ethyl]-amine with 2-bromo-acetamide. LC-MS (B): t_{R}=0.82 min; [M+H]⁺=326.0.

2-{Benzyl-[2-(5-fluoro-1H-indol-3-yl)-ethyl]-amino}-N,N-dimethyl-acetamide

prepared by reaction of benzyl-[2-(5-fluoro-1H-indol-3-yl)-ethyl]-amine with 2-chloro-N,N-dimethylacetamide. LC-MS (B): t_{R}=0.88 min; [M+H]⁺=353.9.

N-Benzyl-N-[2-(5-fluoro-1H-indol-3-yl)-ethyl]-N,N′-dimethyl-ethane-1,2-diamine

prepared by reaction of benzyl-[2-(5-fluoro-1H-indol-3-yl)-ethyl]-amine with (2-chloro-ethyl)-dimethyl-amine hydrochloride. LC-MS (B): t_{R}=1.07 min; [M+H]⁺=339.9.

{Benzyl-[2-(5-fluoro-1H-indol-3-yl)-ethyl]amino}-acetic acid methyl ester

prepared by reaction of benzyl-[2-(5-fluoro-1H-indol-3-yl)-ethyl]-amine with methyl bromoacetate. LC-MS (B): t_{R}=0.96 min; [M+H]⁺=341.0.

(2-{Benzyl-[2-(5-fluoro-1H-indol-3-yl)-ethyl]-amino}-ethyl)-carbamic acid tert-butyl ester

prepared by reaction of benzyl-[2-(5-fluoro-1H-indol-3-yl)-ethyl]-amine with (2-bromo-ethyl)-carbamic acid tert-butyl ester. LC-MS (B): t_{R}=1.01 min; [M+H]⁺=411.8.

A.6.18 Synthesis of N-alkylated 2-(5-fluoro-1H-indol-3-yl)-ethyl-amine derivatives (General Procedure)

A mixture of the respective benzyl-[2-(5-fluoro-1H-indol-3-yl)-ethyl]-amine derivative (0.27 mmol) in EtOH (2.0 mL) is treated with Pd/C (10%, 20 mg) and stirred vigorously under a hydrogen atmosphere (1 bar) for 18 h. After filtration through PTFE filters (0.45 μm) the solvents are removed in vacuo to give the respective product.

[2-(5-Fluoro-1H-indol-3-yl)-ethyl]-methyl-amine

prepared by hydrogenation of benzyl-[2-(5-fluoro-1H-indol-3-yl)-ethyl]-methyl-amine. LC-MS (B): t_{R}=1.03 min; [M+H]⁺=193.2.

Ethyl-[2-(5-fluoro-1H-indol-3-yl)-ethyl]amine

prepared by hydrogenation of benzyl-ethyl-[2-(5-fluoro-1H-indol-3-yl)-ethyl]-amine. LC-MS (B): t_{R}=0.98 min; [M+H]⁺=207.2.

[2-(5-Fluoro-1H-indol-3-yl)-ethyl]propyl-amine

prepared by hydrogenation of benzyl-[2-(5-fluoro-1H-indol-3-yl)-ethyl]-propyl-amine. LC-MS (B): t_{R}=0.98 min; [M+H]⁺=221.2.

[2-(5-Fluoro-1H-indol-3-yl)-ethyl]-(2,2,2-trifluoro-ethyl)-amine

prepared by hydrogenation of benzyl-[2-(5-fluoro-1H-indol-3-yl)-ethyl]-(2,2,2-trifluoro-ethyl)-amine. LC-MS (B): t_{R}=0.85 min; [M+H]⁺=261.1.

2-[2-(5-Fluoro-1H-indol-3-yl)-ethylamino]-acetamide

prepared by hydrogenation of 2-{benzyl-[2-(5-fluoro-1H-indol-3-yl)-ethyl]-amino}-acetamide. LC-MS (B): t_{R}=0.64 min; [M+H]⁺=236.2.

2-[2-(5-Fluoro-1H-indol-3-yl)-ethylamino]-N,N-dimethyl-acetamide

prepared by hydrogenation of 2-{benzyl-[2-(5-fluoro-1H-indol-3-yl)-ethyl]-amino}-N,N-dimethyl-acetamide. LC-MS (B): t_{R}=0.68 min; [M+H]⁺=264.0.

N′-[2-(5-Fluoro-1H-indol-3-yl)-ethyl]-N,N-dimethyl-ethane-1,2-diamine

prepared by hydrogenation of N-benzyl-N-[2-(5-fluoro-H-1-indol-3-yl)-ethyl]-N′,N′-dimethyl-ethane-1,2-diamine. LC-MS (B): t_{R}=0.97 min; [M+H]⁺=250.0.

[2-(5-Fluoro-1H-indol-3-yl)-ethylamino]-acetic acid methyl ester

prepared by hydrogenation of {benzyl-[2-(5-fluoro-H-1-indol-3-yl)-ethyl]-amino}-acetic acid methyl ester. LC-MS (B): t_{R}=0.74 min; [M+H]⁺=251.0.

{2-[2-(5-Fluoro-1H-indol-3-yl)-ethylamino]-ethyl}-carbamic acid tert-butyl ester

prepared by hydrogenation of (2-{benzyl-[2-(5-fluoro-1H-indol-3-yl)-ethyl]-amino}-ethyl)-carbamic acid tert-butyl ester. LC-MS (B): t_{R}=0.83 min; [M+H]⁺=322.0.

A.6.19 Synthesis of 2-(5-fluoro-1H-indol-3-yl)-ethyl-amine derivatives by alkylation (General Procedure)

A mixture of 5-fluoro-tryptamine hydrochloride (0.39 mmol), DIPEA (0.97 mmol) and the respective alkyl halide (0.43 mmol) in THF (1.0 mL) is stirred at 60° C. for 18 h, diluted with DMF (0.5 mL) and MeOH (0.5 mL) and stirred for further 24 h at 60° C. The volatiles are removed in vacuo, DMF (3.0 mL) is added and the mixture is purified by prep. HPLC (basic gradient) to give the desired product.

[2-(5-Fluoro-1H-indol-3-yl)-ethyl]-isopropyl-amine

prepared by reaction of 5-fluoro-tryptamine hydrochloride with 2-iodopropane. LC-MS (B): t_{R}=1.01 min; [M+H]⁺=221.2.

(2,2-Difluoro-ethyl)-[2-(5-fluoro-1H-indol-3-yl)-ethyl]-amine

prepared by reaction of 5-fluoro-tryptamine hydrochloride with 1,1-difluoro-2-iodoethane. LC-MS (B): t_{R}=0.79 min; [M+H]⁺=242.9.

A.7 Synthesis of Chloro- and Bromo-heterocyclyl-carboxylic amide derivatives (General Procedure)

TBTU (0.81 mmol) is added to a mixture of the respective secondary amine (0.74 mmol), the respective carboxylic acid derivative (0.81 mmol) and DIPEA (1.69 mmol) in DMF (2.0 mL). The mixture is stirred for 10 min and either purified directly by prep. HPLC, or diluted with TBME (30 mL), washed twice with water (2×20 mL), once with aqueous NaOH solution (0.5 M, 20 mL), once with aqueous citric acid solution (5%, 20 mL) and twice with water (2×20 mL), dried over MgSO₄ and concentrated in vacuo to give the desired product.

3-Bromo-N-cyclopropylmethyl-N-[2-(3,4-dimethoxy-phenyl)-ethyl]-isonicotinamide

prepared by reaction of cyclopropylmethyl-[2-(3,4-dimethoxy-phenyl)-ethyl]-amine with 3-bromo-isonicotinic acid. LC-MS (B): t_{R}=0.82 min; [M+H]⁺=419.0.

3-Bromo-pyridine-2-carboxylic acid cyclopropylmethyl-[2-(3,4-dimethoxy-phenyl)-ethyl]-amide

prepared by reaction of cyclopropylmethyl-[2-(3,4-dimethoxy-phenyl)-ethyl]-amine with 3-bromo-pyridine-2-carboxylic acid. LC-MS (B): t_{R}=0.84 min; [M+H]⁺=419.0.

2-Bromo-N-cyclopropylmethyl-N-[2-(3,4-dimethoxy-phenyl)-ethyl]-nicotinamide

prepared by reaction of cyclopropylmethyl-[2-(3,4-dimethoxy-phenyl)-ethyl]-amine with 2-bromo-nicotinic acid. LC-MS (B): t_{R}=0.82 min; [M+H]⁺=419.0.

3-Bromo-pyridine-2-carboxylic acid cyclopropylmethyl-[2-(5-fluoro-1H-indol-3-yl)-ethyl]-amide

prepared by reaction of cyclopropylmethyl-[2-(5-fluoro-1H-indol-3-yl)-ethyl]-amine with 3-bromo-pyridine-2-carboxylic acid. LC-MS (B): t_{R}=0.88 min; [M+H]⁺=415.8.

5-Bromo-2-methyl-thiazole-4-carboxylic acid cyclopropylmethyl-[2-(5-fluoro-1H-indol-3-yl)-ethyl]-amide

prepared by reaction of cyclopropylmethyl-[2-(5-fluoro-1H-indol-3-yl)-ethyl]-amine with 5-bromo-2-methyl-thiazole-4-carboxylic acid. LC-MS (B): t_{R}=0.91 min; [M+H]⁺=436.0.

3-Chloro-pyrazine-2-carboxylic acid cyclopropylmethyl-[2-(7-methyl-1H-indol-3-yl)-ethyl]-amide

prepared by reaction of cyclopropylmethyl-[2-(7-methyl-1H-indol-3-yl)-ethyl]-amine with 3-chloro-pyrazine-2-carboxylic acid. LC-MS (C): t_{R}=0.76 min; [M+H]⁺=369.1.

A.8 Synthesis of (4-{Cyclopropylmethyl-[2-(5-fluoro-1H-indol-3-yl)-ethyl]-carbamoyl}-5-m-tolyl-thiazol-2-ylmethyl)-carbamic acid tert-butyl ester

A solution of cyclopropylmethyl-[2-(5-fluoro-1H-indol-3-yl)-ethyl]-amine (0.035 mmol) in DMF (0.25 mL) is added to a mixture of 2-(tert-butoxycarbonylamino-methyl)-5-m-tolyl-thiazole-4-carboxylic acid (0.035 mmol), TBTU (0.037 mmol) and DIPEA (0.070 mmol) in DMF (0.25 mL). The mixture is stirred for 16 h and purified by prep. HPLC (basic gradient) to give the desired product. LC-MS (B): t_{R}=1.00 min; [M+H]⁺=563.0.

A.9 Synthesis of (2-{[3-(3,4-Dimethyl-phenyl)-pyrazine-2-carbonyl]-[2-(5-fluoro-1H-indol-3-yl)-ethyl]-amino}-ethyl)-carbamic acid tert-butyl ester

A solution of {2-[2-(5-fluoro-1H-indol-3-yl)-ethylamino]-ethyl}-carbamic acid tert-butyl ester (0.023 mmol) in DMF (0.25 mL) is added to a mixture of 3-(3,4-dimethyl-phenyl)-pyrazine-2-carboxylic acid (0.044 mmol), TBTU (0.026 mmol) and DIPEA (0.070 mmol) in DMF (0.25 mL). The mixture is stirred for 16 h and purified by prep. HPLC (basic gradient) to give the desired product. LC-MS (B): t_{R}=0.94 min; [M+H]⁺=532.0.

A.10 Synthesis of 2-Bromo-5-m-tolyl-thiazole-4-carboxylic acid cyclopropyl-methyl-[2-(5-fluoro-1H-indol-3-yl)-ethyl]-amide

TBTU (0.095 mmol) is added to a mixture of cyclopropylmethyl-[2-(5-fluoro-1H-indol-3-yl)-ethyl]-amine (0.086 mmol), 2-bromo-5-m-tolyl-thiazole-4-carboxylic acid (0.086 mmol) and DIPEA (0.194 mmol) in DMF (0.50 mL). The mixture is stirred for 16 h and purified by prep. HPLC (basic gradient) to give the desired product. LC-MS (C): t_{R}=0.96 min; [M+H]⁺=512.1.

A.11 Synthesis of 4-Phenyl-pyrimidine-5-carboxylic acid derivatives
A.11.1 Synthesis of 4-Phenyl-pyrimidine-5-carboxylic acid
A.11.1.1 Synthesis of 2-Benzoyl-3-dimethylamino-acrylic acid ethyl ester

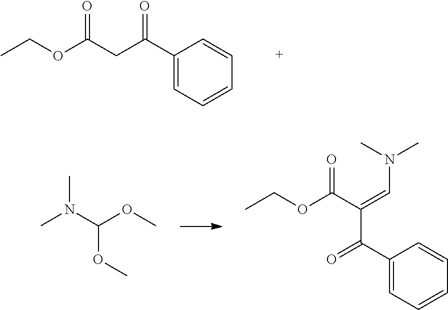

Benzoylacetic acid ethylester (commercially available; 1.0 g; 5.1 mmol) was dissolved in cyclohexane (10 ml) followed by the addition of N,N-dimethylformamid-dimethylacetale (commercially available; 1.0 g; 8.16 mmol) dissolved in cyclohexane (5 ml) via syringe over 30 minutes. The reaction mixture was heated to reflux for 30 minutes, cooled to rt and the solvent was evaporated to give 1.47 g of 2-benzoyl-3-dimethylamino-acrylic acid ethyl ester which was used in the next step without further purification. LC-MS (C): t_{R}=0.86 min; [M+H]⁺=248.45.

A.11.1.2. Synthesis of 4-Phenyl-pyrimidine-5-carboxylic acid ethyl ester

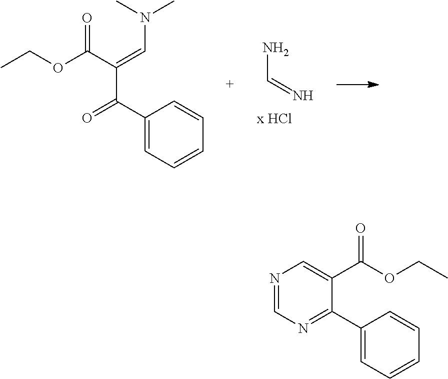

In an inert atmosphere, dry ethanol (50 ml) was placed in a round-bottomed flask and a solution of sodium ethylate (21% in ethanol; 14 ml) was added, followed by the addition of formamidine hydrochloride (3.1 g; 37 mmol). Stirring was continued for 30 minutes, then the precipitated solid was filtered off. The filtercake was washed with ethanol (15 ml). This solution was carefully added to a solution of 2-benzoyl-3-dimethylamino-acrylic acid ethyl ester (7.2 g; 25 mmol) in ethanol (100 ml). The resulting reaction mixture was refluxed overnight, cooled to rt and the solvent was evaporated to give 6.22 g of 4-phenyl-pyrimidine-5-carboxylic acid ethyl ester as a yellow oil which was used in the next step without further purification. LC-MS (C): t_{R}=0.95 min; [M+H]⁺=229.46.

A.11.1.3 Synthesis of 4-Phenyl-pyrimidine-5-carboxylic acid

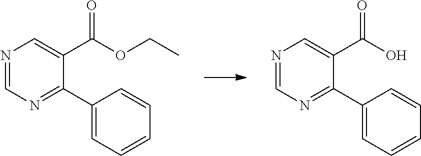

4-Phenyl-pyrimidine-5-carboxylic acid ethyl ester (6.2 g; 25 mmol) was dissolved in methanol (30 ml) followed by the addition of aqueous sodium hydroxide solution (2M; 25 ml). Stirring was continued for 3 h. The reaction mixture was then concentrated, the residue diluted with water followed by the addition of aqueous hydrochloric acid (2M) to pH=1-2. Stirring was continued for 1 h. The precipitate was filtered off and washed with diethylether to give 1.9 g of 4-phenyl-pyrimidine-5-carboxylic acid as a white solid. LC-MS (C): t_{R}=0.72 min; [M+H]⁺=201.49.

A.11.2.1 Synthesis of 2-Methyl-4-phenyl-pyrimidine-5-carboxylic acid ethyl ester

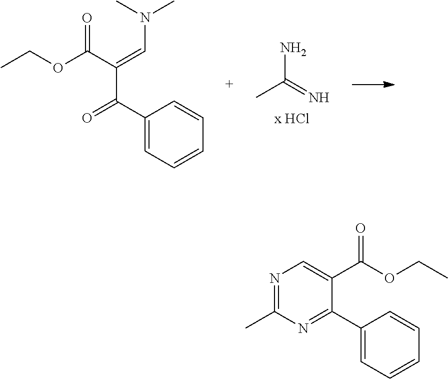

In an inert atmosphere, dry ethanol (50 ml) was placed in a round-bottomed flask and a solution of sodium ethylate (21% in ethanol; 14 ml) was added, followed by the addition of acetamidine hydrochloride (3.7 g; 37 mmol). Stirring was continued for 30 minutes, then the precipitated solid was filtered off. The filtercake was washed with ethanol (15 ml). This solution was carefully added to a solution of 2-benzoyl-3-dimethylamino-acrylic acid ethyl ester (7.2 g; 25 mmol) in ethanol (100 ml). The resulting reaction mixture was refluxed overnight, cooled to rt and the solvent was evaporated to give 4.53 g of 2-methyl-4-phenyl-pyrimidine-5-carboxylic acid ethyl ester as a yellow oil which was used in the next step without further purification. LC-MS (C): t_{R}=0.95 min; [M+H]⁺=243.37.

A.11.2.2 Synthesis of 2-Methyl-4-phenyl-pyrimidine-5-carboxylic acid

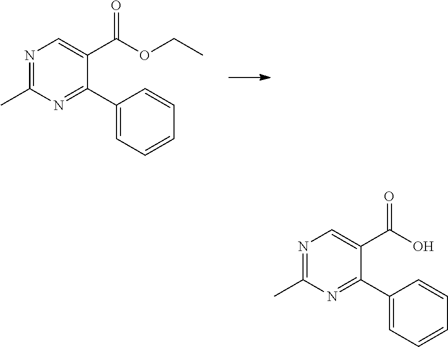

2-Methyl-4-phenyl-pyrimidine-5-carboxylic acid ethyl ester (4.5 g; 18.7 mmol) was dissolved in methanol (30 ml) followed by the addition of aqueous sodium hydroxide solution (2M; 18 ml). Stirring was continued for 4 h. The reaction mixture was then concentrated, the residue diluted with water followed by the addition of aqueous hydrochloric acid (2M) to pH=1-2. Stirring was continued for 1 h. The precipitate was filtered off and washed with diethylether to give 2.42 g of 2-methyl-4-phenyl-pyrimidine-5-carboxylic acid as a white solid. LC-MS (C): t_{R}=0.74 min; [M+H]⁺=215.47.

According to the procedures described above or in the literature, the following 4-phenyl-pyrimidine carboxylic acid derivatives could be prepared:

4-(3-Methoxy-phenyl)-pyrimidine-5-carboxylic acid; LC-MS (C): t_{R}=0.76 min; [M+H]⁺=231.11.

4-(3,5-Dichloro-phenyl)-pyrimidine-5-carboxylic acid; LC-MS (C): t_{R}=0.89 min; [M+H]⁺=269.22.

4-(3,4-Dimethyl-phenyl)-pyrimidine-5-carboxylic acid; LC-MS (C): t_{R}=0.85 min; [M+H]⁺=229.41.

4-(3-Chloro-phenyl)-pyrimidine-5-carboxylic acid; LC-MS (C): t_{R}=0.82 min; [M+H]⁺=275.98.

4-(4-Bromo-3-chloro-phenyl)-pyrimidine-5-carboxylic acid; LC-MS (C): t_{R}=0.90 min; [M+H]⁺=356.08.

4-(3,4-Dichloro-phenyl)-pyrimidine-5-carboxylic acid; LC-MS (C): t_{R}=0.89 min; [M+H]⁺=269.21.

4-m-Tolyl-pyrimidine-5-carboxylic acid; LC-MS (C): t_{R}=0.80 min; [M+H]=215.54.

4-(4-Fluoro-phenyl)-pyrimidine-5-carboxylic acid; LC-MS (C): t_{R}=0.75 min; [M+H]⁺=219.48.

4-p-Tolyl-pyrimidine-5-carboxylic acid; LC-MS (C): t_{R}=0.80 min; [M+H]⁺=215.38.

4-(3-Fluoro-phenyl)-pyrimidine-5-carboxylic acid; LC-MS (C): t_{R}=0.76 min; [M+H]⁺=219.47.

2-Methyl-4-(3-Methoxy-phenyl)-pyrimidine-5-carboxylic acid; LC-MS (C): t_{R}=0.77 min; [M+H]⁺=247.47.

2-Methyl-4-(3,5-Dichloro-phenyl)-pyrimidine-5-carboxylic acid; LC-MS (C): t_{R}=0.91 min; [M+H]⁺=282.85.

2-Methyl-4-(3,4-Dimethyl-phenyl)-pyrimidine-5-carboxylic acid; LC-MS (C): t_{R}=0.84 min; [M+H]⁺=243.45.

2-Methyl-4-(3-Chloro-phenyl)-pyrimidine-5-carboxylic acid; LC-MS (C): t_{R}=0.83 min; [M+H]⁺=249.32.

2-Methyl-4-(4-Bromo-3-chloro-phenyl)-pyrimidine-5-carboxylic acid; LC-MS (C): t_{R}=0.91 min; [M+H]⁺=370.91.

2-Methyl-4-(3,4-Dichloro-phenyl)-pyrimidine-5-carboxylic acid; LC-MS (C): t_{R}=0.90 min; [M+H]⁺=283.07.

2-Methyl-4-m-Tolyl-pyrimidine-5-carboxylic acid; LC-MS (C): t_{R}=0.80 min; [M+H]⁺=229.51.

2-Methyl-4-(4-Fluoro-phenyl)-pyrimidine-5-carboxylic acid; LC-MS (C): t_{R}=0.77 min; [M+H]⁺=233.47.

2-Methyl-4-p-Tolyl-pyrimidine-5-carboxylic acid; LC-MS (C): t_{R}=0.79 min; [M+H]⁺=229.46.

2-Methyl-4-(3-Fluoro-phenyl)-pyrimidine-5-carboxylic acid; LC-MS (C): t_{R}=0.78 min; [M+H]⁺=233.46.

### B. Preparation of Examples

### B.1 Synthesis of Carboxylic Amide Derivatives (General Procedure A)

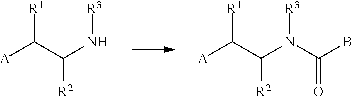

A solution of the respective amine (0.038 mmol) and DIPEA (0.114 mmol) in DMF (0.5 mL) is added to a mixture of the respective carboxylic acid (0.046 mmol) and TBTU (0.046 mmol). The mixture is stirred for 16 h and purified by prep. HPLC using a basic gradient to give the desired amides.

| | | LC-MS | | |
|---|---|---|---|---|
| Example | Name | method | t_{R} [min] | [M + H]⁺ |
| 1 | 2-Amino-5-(3-fluoro-phenyl)-thiazole-4- | (B) | 0.98 | 473.8 |
| | carboxylic acid [2-(3-bromo-phenyl)-ethyl]- | | | |
| | cyclopropylmethyl-amide | | | |
| 2 | 5-(3-Fluoro-phenyl)-2-methyl-thiazole-4- | (B) | 0.96 | 455.0 |
| | carboxylic acid cyclopropylmethyl-[2-(3,4- | | | |
| | dimethoxy-phenyl)-ethyl]-amide | | | |
| 3 | 2-Methyl-5-m-tolyl-thiazole-4-carboxylic acid | (B) | 0.99 | 451.0 |
| | cyclopropylmethyl-[2-(3,4-dimethoxy-phenyl)- | | | |
| | ethyl]-amide | | | |
| 4 | 2-Bromo-5-m-tolyl-thiazole-4-carboxylic acid | (B) | 1.05 | 514.7 |
| | cyclopropylmethyl-[2-(3,4-dimethoxy-phenyl)- | | | |
| | ethyl]-amide | | | |
| 5 | 2-Amino-5-(3-fluoro-phenyl)-thiazole-4- | (B) | 0.86 | 455.9 |
| | carboxylic acid cyclopropylmethyl-[2-(3,4- | | | |
| | dimethoxy-phenyl)-ethyl]-amide | | | |
| 6 | 2-Amino-5-m-tolyl-thiazole-4-carboxylic acid | (B) | 0.89 | 452.1 |
| | cyclopropylmethyl-[2-(3,4-dimethoxy-phenyl)- | | | |
| | ethyl]-amide | | | |
| 7 | 2-Amino-5-(3-chloro-phenyl)-thiazole-4- | (B) | 0.90 | 471.9 |
| | carboxylic acid cyclopropylmethyl-[2-(3,4- | | | |
| | dimethoxy-phenyl)-ethyl]-amide | | | |
| 8 | 5-(4-Cyano-phenyl)-2-methyl-thiazole-4- | (B) | 0.89 | 462.0 |
| | carboxylic acid cyclopropylmethyl-[2-(3,4- | | | |
| | dimethoxy-phenyl)-ethyl]-amide | | | |
| 9 | 5-(3,5-Dimethyl-phenyl)-2-methyl-thiazole-4- | (B) | 1.02 | 465.0 |
| | carboxylic acid cyclopropylmethyl-[2-(3,4- | | | |
| | dimethoxy-phenyl)-ethyl]-amide | | | |
| 10 | 5-(3,5-Difluoro-phenyl)-2-methyl-thiazole-4- | (B) | 0.96 | 473.0 |
| | carboxylic acid cyclopropylmethyl-[2-(3,4- | | | |
| | dimethoxy-phenyl)-ethyl]-amide | | | |
| 11 | 5-(3-Fluoro-5-trifluoromethyl-phenyl)-2- | (B) | 1.01 | 522.7 |
| | methyl-thiazole-4-carboxylic acid | | | |
| | cyclopropylmethyl-[2-(3,4-dimethoxy-phenyl)- | | | |
| | ethyl]-amide | | | |
| 12 | 5-(2,4-Dimethyl-phenyl)-2-methyl-thiazole-4- | (B) | 1.01 | 465.0 |
| | carboxylic acid cyclopropylmethyl-[2-(3,4- | | | |
| | dimethoxy-phenyl)-ethyl]-amide | | | |
| 13 | 5-(3-Fluoro-2-methyl-phenyl)-2-methyl- | (B) | 0.98 | 469.0 |
| | thiazole-4-carboxylic acid cyclopropylmethyl- | | | |
| | [2-(3,4-dimethoxy-phenyl)-ethyl]-amide | | | |
| 14 | 5-(2,3-Dimethyl-phenyl)-2-methyl-thiazole-4- | (B) | 1.00 | 465.1 |
| | carboxylic acid cyclopropylmethyl-[2-(3,4- | | | |
| | dimethoxy-phenyl)-ethyl]-amide | | | |
| 15 | 5-(3,4-Dichloro-phenyl)-2-methyl-thiazole-4- | (B) | 1.04 | 504.9 |
| | carboxylic acid cyclopropylmethyl-[2-(3,4- | | | |
| | dimethoxy-phenyl)-ethyl]-amide | | | |
| 16 | 5-(3-Fluoro-4-methyl-phenyl)-2-methyl- | (B) | 0.99 | 469.0 |
| | thiazole-4-carboxylic acid cyclopropylmethyl- | | | |
| | [2-(3,4-dimethoxy-phenyl)-ethyl]-amide | | | |
| 17 | 5-(3,4-Dimethyl-phenyl)-2-methyl-thiazole-4- | (B) | 1.01 | 465.1 |
| | carboxylic acid cyclopropylmethyl-[2-(3,4- | | | |
| | dimethoxy-phenyl)-ethyl]-amide | | | |
| 18 | 2-Methyl-5-phenyl-thiazole-4-carboxylic acid | (B) | 0.93 | 437.0 |
| | cyclopropylmethyl-[2-(3,4-dimethoxy-phenyl)- | | | |
| | ethyl]-amide | | | |
| 19 | 5-(3-Cyano-phenyl)-2-methyl-thiazole-4- | (B) | 0.89 | 462.0 |
| | carboxylic acid cyclopropylmethyl-[2-(3,4- | | | |
| | dimethoxy-phenyl)-ethyl]-amide | | | |
| 20 | 5-(4-Ethyl-phenyl)-2-methyl-thiazole-4- | (B) | 1.01 | 465.0 |
| | carboxylic acid cyclopropylmethyl-[2-(3,4- | | | |
| | dimethoxy-phenyl)-ethyl]-amide | | | |
| 21 | 5-(3,4-Difluoro-phenyl)-2-methyl-thiazole-4- | (B) | 0.95 | 473.0 |
| | carboxylic acid cyclopropylmethyl-[2-(3,4- | | | |
| | dimethoxy-phenyl)-ethyl]-amide | | | |
| 22 | 2-Cyclopropyl-5-phenyl-thiazole-4-carboxylic | (B) | 0.99 | 463.0 |
| | acid cyclopropylmethyl-[2-(3,4-dimethoxy- | | | |
| | phenyl)-ethyl]-amide | | | |
| 23 | 2-Cyclopropyl-5-p-tolyl-thiazole-4-carboxylic | (B) | 1.02 | 477.0 |
| | acid cyclopropylmethyl-[2-(3,4-dimethoxy- | | | |
| | phenyl)-ethyl]-amide | | | |
| 24 | 2-Cyclopropyl-5-(4-fluoro-phenyl)-thiazole-4- | (B) | 1.01 | 481.0 |
| | carboxylic acid cyclopropylmethyl-[2-(3,4- | | | |
| | dimethoxy-phenyl)-ethyl]-amide | | | |
| 25 | 2-Cyclopropyl-5-(3-fluoro-phenyl)-thiazole-4- | (B) | 1.00 | 481.0 |
| | carboxylic acid cyclopropylmethyl-[2-(3,4- | | | |
| | dimethoxy-phenyl)-ethyl]-amide | | | |
| 26 | 2-Cyclopropyl-5-(3-trifluoromethyl-phenyl)- | (B) | 1.04 | 531.1 |
| | thiazole-4-carboxylic acid cyclopropylmethyl- | | | |
| | [2-(3,4-dimethoxy-phenyl)-ethyl]-amide | | | |
| 27 | 2-Cyclopropyl-5-(3-fluoro-4-methyl-phenyl)- | (B) | 1.04 | 495.0 |
| | thiazole-4-carboxylic acid cyclopropylmethyl- | | | |
| | [2-(3,4-dimethoxy-phenyl)-ethyl]-amide | | | |
| 28 | 2-Cyclopropyl-5-(3-fluoro-5-trifluoromethyl- | (B) | 1.06 | 549.1 |
| | phenyl)-thiazole-4-carboxylic acid cyclopropyl- | | | |
| | methyl-[2-(3,4-dimethoxy-phenyl)-ethyl]-amide | | | |
| 29 | 2-Methoxy-5-m-tolyl-thiazole-4-carboxylic acid | (B) | 1.02 | 467.0 |
| | cyclopropylmethyl-[2-(3,4-dimethoxy-phenyl)- | | | |
| | ethyl]-amide | | | |
| 30 | 2-Dimethylamino-5-m-tolyl-thiazole-4- | (B) | 0.99; | 480.0 |
| | carboxylic acid cyclopropylmethyl-[2-(3,4- | | 1.02 | |
| | dimethoxy-phenyl)-ethyl]-amide | | | |
| 31 | 2-Amino-5-(2-fluoro-phenyl)-thiazole-4- | (B) | 0.85 | 456.0 |
| | carboxylic acid cyclopropylmethyl-[2-(3,4- | | | |
| | dimethoxy-phenyl)-ethyl]-amide | | | |
| 32 | 2-Amino-5-phenyl-thiazole-4-carboxylic acid | (B) | 0.85 | 438.0 |
| | cyclopropylmethyl-[2-(3,4-dimethoxy-phenyl)- | | | |
| | ethyl]-amide | | | |
| 33 | 2-Amino-5-p-tolyl-thiazole-4-carboxylic acid | (B) | 0.88 | 452.0 |
| | cyclopropylmethyl-[2-(3,4-dimethoxy-phenyl)- | | | |
| | ethyl]-amide | | | |
| 34 | 5-m-Tolyl-thiazole-4-carboxylic acid | (B) | 0.94 | 437.1 |
| | cyclopropylmethyl-[2-(3,4-dimethoxy-phenyl)- | | | |
| | ethyl]-amide | | | |
| 35 | 5-(3-Chloro-phenyl)-thiazole-4-carboxylic acid | (B) | 0.95 | 456.8 |
| | cyclopropylmethyl-[2-(3,4-dimethoxy-phenyl)- | | | |
| | ethyl]-amide | | | |
| 36 | 5-(3-Trifluoromethyl-phenyl)-thiazole-4- | (B) | 0.96 | 490.7 |
| | carboxylic acid cyclopropylmethyl-[2-(3,4- | | | |
| | dimethoxy-phenyl)-ethyl]-amide | | | |
| 37 | 5-(2-Fluoro-phenyl)-thiazole-4-carboxylic acid | (B) | 0.91 | 441.0 |
| | cyclopropylmethyl-[2-(3,4-dimethoxy-phenyl)- | | | |
| | ethyl]-amide | | | |
| 38 | 5-(4-Fluoro-phenyl)-thiazole-4-carboxylic acid | (B) | 0.91 | 441.0 |
| | cyclopropylmethyl-[2-(3,4-dimethoxy-phenyl)- | | | |
| | ethyl]-amide | | | |
| 39 | 5-(3-Methoxy-phenyl)-thiazole-4-carboxylic | (B) | 0.90 | 453.0 |
| | acid cyclopropylmethyl-[2-(3,4-dimethoxy- | | | |
| | phenyl)-ethyl]-amide | | | |
| 40 | 5-Phenyl-thiazole-4-carboxylic acid | (B) | 0.90 | 423.0 |
| | cyclopropylmethyl-[2-(3,4-dimethoxy-phenyl)- | | | |
| | ethyl]-amide | | | |
| 41 | 5-(3-Fluoro-phenyl)-thiazole-4-carboxylic acid | (B) | 0.91 | 441.0 |
| | cyclopropylmethyl-[2-(3,4-dimethoxy-phenyl)- | | | |
| | ethyl]-amide | | | |
| 42 | 5-(3-Methoxy-phenyl)-2-methyl-oxazole-4- | (B) | 0.92 | 451.0 |
| | carboxylic acid cyclopropylmethyl-[2-(3,4- | | | |
| | dimethoxy-phenyl)-ethyl]-amide | | | |
| 43 | 2-Methyl-5-(3-trifluoromethyl-phenyl)-oxazole- | (B) | 0.98 | 488.8 |
| | 4-carboxylic acid cyclopropylmethyl-[2-(3,4- | | | |
| | dimethoxy-phenyl)-ethyl]-amide | | | |
| 44 | 4-(3-Chloro-phenyl)-2-methyl-thiazole-5- | (B) | 0.99 | 471.1 |
| | carboxylic acid cyclopropylmethyl-[2-(3,4- | | | |
| | dimethoxy-phenyl)-ethyl]-amide | | | |
| 45 | 2-Methyl-4-(3-trifluoromethyl-phenyl)-thiazole- | (B) | 1.00 | 505.0 |
| | 5-carboxylic acid cyclopropylmethyl-[2-(3,4- | | | |
| | dimethoxy-phenyl)-ethyl]-amide | | | |
| 46 | 4-(3-Methoxy-phenyl)-2-methyl-thiazole-5- | (B) | 0.92 | 467.0 |
| | carboxylic acid cyclopropylmethyl-[2-(3,4- | | | |
| | dimethoxy-phenyl)-ethyl]-amide | | | |
| 47 | 2-Methyl-4-(4-trifluoromethyl-phenyl)-thiazole- | (B) | 1.00 | 505.0 |
| | 5-carboxylic acid cyclopropylmethyl-[2-(3,4- | | | |
| | dimethoxy-phenyl)-ethyl]-amide | | | |
| 48 | 4-(4-Chloro-phenyl)-2-methyl-thiazole-5- | (B) | 0.99 | 471.0 |
| | carboxylic acid cyclopropylmethyl-[2-(3,4- | | | |
| | dimethoxy-phenyl)-ethyl]-amide | | | |
| 49 | 2-Methyl-4-p-tolyl-thiazole-5-carboxylic acid | (B) | 0.96 | 451.1 |
| | cyclopropylmethyl-[2-(3,4-dimethoxy-phenyl)- | | | |
| | ethyl]-amide | | | |
| 50 | 4-(4-Fluoro-phenyl)-2-methyl-thiazole-5- | (B) | 0.94 | 455.1 |
| | carboxylic acid cyclopropylmethyl-[2-(3,4- | | | |
| | dimethoxy-phenyl)-ethyl]-amide | | | |
| 51 | 3-Phenyl-cinnoline-4-carboxylic acid | (B) | 0.89; | 468.0 |
| | cyclopropylmethyl-[2-(3,4-dimethoxy-phenyl)- | | 0.91 | |
| | ethyl]-amide | | | |
| 52 | 6-Chloro-2-phenyl-imidazo[1,2-a]pyridine-3- | (B) | 0.93 | 489.7 |
| | carboxylic acid cyclopropylmethyl-[2-(3,4- | | | |
| | dimethoxy-phenyl)-ethyl]-amide | | | |
| 53 | 4-Phenyl-[1,2,3]thiadiazole-5-carboxylic acid | (B) | 0.94 | 424.0 |
| | cyclopropylmethyl-[2-(3,4-dimethoxy-phenyl)- | | | |
| | ethyl]-amide | | | |
| 54 | 3-Phenyl-pyrazine-2-carboxylic acid | (B) | 0.87 | 418.1 |
| | cyclopropylmethyl-[2-(3,4-dimethoxy-phenyl)- | | | |
| | ethyl]-amide | | | |
| 55 | 2-Methyl-5-m-tolyl-thiazole-4-carboxylic acid | (B) | 0.90 | 467.0 |
| | cyclopropylmethyl-[2-(3,4-dimethoxy-phenyl)- | | | |
| | 2-hydroxy-ethyl]-amide | | | |
| 56 | 2-Bromo-5-m-tolyl-thiazole-4-carboxylic acid | (B) | 0.96 | 530.8 |
| | cyclopropylmethyl-[2-(3,4-dimethoxy-phenyl)- | | | |
| | 2-hydroxy-ethyl]-amide | | | |
| 57 | 2-Amino-5-(3-fluoro-phenyl)-thiazole-4- | (B) | 0.81 | 471.9 |
| | carboxylic acid cyclopropylmethyl-[2-(3,4- | | | |
| | dimethoxy-phenyl)-2-hydroxy-ethyl]-amide | | | |
| 58 | 2-Amino-5-m-tolyl-thiazole-4-carboxylic acid | (B) | 0.83 | 468.0 |
| | cyclopropylmethyl-[2-(3,4-dimethoxy-phenyl)- | | | |
| | 2-hydroxy-ethyl]-amide | | | |
| 59 | 2-Amino-5-(3-chloro-phenyl)-thiazole-4- | (B) | 0.85 | 487.7 |
| | carboxylic acid cyclopropylmethyl-[2-(3,4- | | | |
| | dimethoxy-phenyl)-2-hydroxy-ethyl]-amide | | | |
| 60 | 5-(3,5-Dimethyl-phenyl)-2-methyl-thiazole-4- | (B) | 0.94 | 481.1 |
| | carboxylic acid cyclopropylmethyl-[2-(3,4- | | | |
| | dimethoxy-phenyl)-2-hydroxy-ethyl]-amide | | | |
| 61 | 5-(3,5-Difluoro-phenyl)-2-methyl-thiazole-4- | (B) | 0.89 | 488.9 |
| | carboxylic acid cyclopropylmethyl-[2-(3,4- | | | |
| | dimethoxy-phenyl)-2-hydroxy-ethyl]-amide | | | |
| 62 | 5-(2,4-Dimethyl-phenyl)-2-methyl-thiazole-4- | (B) | 0.94 | 481.0 |
| | carboxylic acid cyclopropylmethyl-[2-(3,4- | | | |
| | dimethoxy-phenyl)-2-hydroxy-ethyl]-amide | | | |
| 63 | 5-(3-Fluoro-2-methyl-phenyl)-2-methyl- | (B) | 0.90 | 485.0 |
| | thiazole-4-carboxylic acid cyclopropylmethyl- | | | |
| | [2-(3,4-dimethoxy-phenyl)-2-hydroxy-ethyl]- | | | |
| | amide | | | |
| 64 | 5-(2,3-Dimethyl-phenyl)-2-methyl-thiazole-4- | (B) | 0.93 | 481.0 |
| | carboxylic acid cyclopropylmethyl-[2-(3,4- | | | |
| | dimethoxy-phenyl)-2-hydroxy-ethyl]-amide | | | |
| 65 | 5-(3,4-Dichloro-phenyl)-2-methyl-thiazole-4- | (B) | 0.96 | 520.8 |
| | carboxylic acid cyclopropylmethyl-[2-(3,4- | | | |
| | dimethoxy-phenyl)-2-hydroxy-ethyl]-amide | | | |
| 66 | 5-(3-Fluoro-4-methyl-phenyl)-2-methyl- | (B) | 0.91 | 485.0 |
| | thiazole-4-carboxylic acid cyclopropylmethyl- | | | |
| | [2-(3,4-dimethoxy-phenyl)-2-hydroxy-ethyl]- | | | |
| | amide | | | |
| 67 | 5-(3,4-Dimethyl-phenyl)-2-methyl-thiazole-4- | (B) | 0.93 | 481.0 |
| | carboxylic acid cyclopropylmethyl-[2-(3,4- | | | |
| | dimethoxy-phenyl)-2-hydroxy-ethyl]-amide | | | |
| 68 | 2-Methyl-5-phenyl-thiazole-4-carboxylic acid | (B) | 0.85 | 453.0 |
| | cyclopropylmethyl-[2-(3,4-dimethoxy-phenyl)- | | | |
| | 2-hydroxy-ethyl]-amide | | | |
| 69 | 5-(4-Ethyl-phenyl)-2-methyl-thiazole-4- | (B) | 0.94 | 481.0 |
| | carboxylic acid cyclopropylmethyl-[2-(3,4- | | | |
| | dimethoxy-phenyl)-2-hydroxy-ethyl]-amide | | | |
| 70 | 5-(3,4-Difluoro-phenyl)-2-methyl-thiazole-4- | (B) | 0.88 | 488.8 |
| | carboxylic acid cyclopropylmethyl-[2-(3,4- | | | |
| | dimethoxy-phenyl)-2-hydroxy-ethyl]-amide | | | |
| 71 | 2-Cyclopropyl-5-phenyl-thiazole-4-carboxylic | (B) | 0.92 | 479.0 |
| | acid cyclopropylmethyl-[2-(3,4-dimethoxy- | | | |
| | phenyl)-2-hydroxy-ethyl]-amide | | | |
| 72 | 2-Cyclopropyl-5-p-tolyl-thiazole-4-carboxylic | (B) | 0.96 | 493.0 |
| | acid cyclopropylmethyl-[2-(3,4-dimethoxy- | | | |
| | phenyl)-2-hydroxy-ethyl]-amide | | | |
| 73 | 2-Cyclopropyl-5-(4-fluoro-phenyl)-thiazole-4- | (B) | 0.93 | 497.0 |
| | carboxylic acid cyclopropylmethyl-[2-(3,4- | | | |
| | dimethoxy-phenyl)-2-hydroxy-ethyl]-amide | | | |
| 74 | 2-Cyclopropyl-5-(3-fluoro-phenyl)-thiazole-4- | (B) | 0.93 | 497.0 |
| | carboxylic acid cyclopropylmethyl-[2-(3,4- | | | |
| | dimethoxy-phenyl)-2-hydroxy-ethyl]-amide | | | |
| 75 | 2-Cyclopropyl-5-(3-trifluoromethyl-phenyl)- | (B) | 0.98 | 546.9 |
| | thiazole-4-carboxylic acid cyclopropylmethyl- | | | |
| | [2-(3,4-dimethoxy-phenyl)-2-hydroxy-ethyl]- | | | |
| | amide | | | |
| 76 | 2-Cyclopropyl-5-(3-fluoro-4-methyl-phenyl)- | (B) | 0.98 | 511.0 |
| | thiazole-4-carboxylic acid cyclopropylmethyl- | | | |
| | [2-(3,4-dimethoxy-phenyl)-2-hydroxy-ethyl]- | | | |
| | amide | | | |
| 77 | 2-Cyclopropyl-5-(3-fluoro-5-trifluoromethyl- | (B) | 1.00 | 564.9 |
| | phenyl)-thiazole-4-carboxylic acid | | | |
| | cyclopropylmethyl-[2-(3,4-dimethoxy-phenyl)- | | | |
| | 2-hydroxy-ethyl]-amide | | | |
| 78 | 2-Methoxy-5-m-tolyl-thiazole-4-carboxylic acid | (B) | 0.94 | 483.0 |
| | cyclopropylmethyl-[2-(3,4-dimethoxy-phenyl)- | | | |
| | 2-hydroxy-ethyl]-amide | | | |
| 79 | 2-Dimethylamino-5-m-tolyl-thiazole-4- | (B) | 0.94 | 496.0 |
| | carboxylic acid cyclopropylmethyl-[2-(3,4- | | | |
| | dimethoxy-phenyl)-2-hydroxy-ethyl]-amide | | | |
| 80 | 2-Amino-5-(2-fluoro-phenyl)-thiazole-4- | (B) | 0.80 | 472.0 |
| | carboxylic acid cyclopropylmethyl-[2-(3,4- | | | |
| | dimethoxy-phenyl)-2-hydroxy-ethyl]-amide | | | |
| 81 | 2-Amino-5-phenyl-thiazole-4-carboxylic acid | (B) | 0.80 | 453.9 |
| | cyclopropylmethyl-[2-(3,4-dimethoxy-phenyl)- | | | |
| | 2-hydroxy-ethyl]-amide | | | |
| 82 | 2-Amino-5-p-tolyl-thiazole-4-carboxylic acid | (B) | 0.83 | 468.0 |
| | cyclopropylmethyl-[2-(3,4-dimethoxy-phenyl)- | | | |
| | 2-hydroxy-ethyl]-amide | | | |
| 83 | 5-m-Tolyl-thiazole-4-carboxylic acid | (B) | 0.87 | 452.9 |
| | cyclopropylmethyl-[2-(3,4-dimethoxy-phenyl)- | | | |
| | 2-hydroxy-ethyl]-amide | | | |
| 84 | 5-(3-Chloro-phenyl)-thiazole-4-carboxylic acid | (B) | 0.88 | 472.9 |
| | cyclopropylmethyl-[2-(3,4-dimethoxy-phenyl)- | | | |
| | 2-hydroxy-ethyl]-amide | | | |
| 85 | 5-(3-Trifluoromethyl-phenyl)-thiazole-4- | (B) | 0.89 | 506.9 |
| | carboxylic acid cyclopropylmethyl-[2-(3,4- | | | |
| | dimethoxy-phenyl)-2-hydroxy-ethyl]-amide | | | |
| 86 | 5-(2-Fluoro-phenyl)-thiazole-4-carboxylic acid | (B) | 0.83 | 456.9 |
| | cyclopropylmethyl-[2-(3,4-dimethoxy-phenyl)- | | | |
| | 2-hydroxy-ethyl]-amide | | | |
| 87 | 5-(4-Fluoro-phenyl)-thiazole-4-carboxylic acid | (B) | 0.83 | 456.9 |
| | cyclopropylmethyl-[2-(3,4-dimethoxy-phenyl)- | | | |
| | 2-hydroxy-ethyl]-amide | | | |
| 88 | 5-(3-Methoxy-phenyl)-thiazole-4-carboxylic | (B) | 0.83 | 468.9 |
| | acid cyclopropylmethyl-[2-(3,4-dimethoxy- | | | |
| | phenyl)-2-hydroxy-ethyl]-amide | | | |
| 89 | 5-Phenyl-thiazole-4-carboxylic acid | (B) | 0.82 | 438.9 |
| | cyclopropylmethyl-[2-(3,4-dimethoxy-phenyl)- | | | |
| | 2-hydroxy-ethyl]-amide | | | |
| 90 | 5-(3-Fluoro-phenyl)-thiazole-4-carboxylic acid | (B) | 0.84 | 456.9 |
| | cyclopropylmethyl-[2-(3,4-dimethoxy-phenyl)- | | | |
| | 2-hydroxy-ethyl]-amide | | | |
| 91 | 4-(3-Chloro-phenyl)-2-methyl-thiazole-5- | (B) | 0.90 | 486.9 |
| | carboxylic acid cyclopropylmethyl-[2-(3,4- | | | |
| | dimethoxy-phenyl)-2-hydroxy-ethyl]-amide | | | |
| 92 | 2-Methyl-4-(3-trifluoromethyl-phenyl)-thiazole- | (B) | 0.92 | 520.8 |
| | 5-carboxylic acid cyclopropylmethyl-[2-(3,4- | | | |
| | dimethoxy-phenyl)-2-hydroxy-ethyl]-amide | | | |
| 93 | 4-(3-Methoxy-phenyl)-2-methyl-thiazole-5- | (B) | 0.83 | 483.0 |
| | carboxylic acid cyclopropylmethyl-[2-(3,4- | | | |
| | dimethoxy-phenyl)-2-hydroxy-ethyl]-amide | | | |
| 94 | 4-(4-Chloro-phenyl)-2-methyl-thiazole-5- | (B) | 0.89 | 486.9 |
| | carboxylic acid cyclopropylmethyl-[2-(3,4- | | | |
| | dimethoxy-phenyl)-2-hydroxy-ethyl]-amide | | | |
| 95 | 2-Methyl-4-p-tolyl-thiazole-5-carboxylic acid | (B) | 0.86 | 467.0 |
| | cyclopropylmethyl-[2-(3,4-dimethoxy-phenyl)- | | | |
| | 2-hydroxy-ethyl]-amide | | | |
| 96 | 4-(4-Fluoro-phenyl)-2-methyl-thiazole-5- | (B) | 0.84 | 471.0 |
| | carboxylic acid cyclopropylmethyl-[2-(3,4- | | | |
| | dimethoxy-phenyl)-2-hydroxy-ethyl]-amide | | | |
| 97 | 6-Chloro-2-phenyl-imidazo[1,2-a]pyridine-3- | (B) | 0.49 | 506.1 |
| | carboxylic acid cyclopropylmethyl-[2-(3,4- | | | |
| | dimethoxy-phenyl)-2-hydroxy-ethyl]-amide | | | |
| 98 | 4-Phenyl-[1,2,3]thiadiazole-5-carboxylic acid | (B) | 0.53 | 440.2 |
| | cyclopropylmethyl-[2-(3,4-dimethoxy-phenyl)- | | | |
| | 2-hydroxy-ethyl]-amide | | | |
| 99 | 3-Phenyl-pyrazine-2-carboxylic acid | (B) | 0.48; | 434.2 |
| | cyclopropylmethyl-[2-(3,4-dimethoxy-phenyl)- | | 0.49 | |
| | 2-hydroxy-ethyl]-amide | | | |

### B.2 Synthesis of Carboxylic Amide Derivatives (General Procedure B)

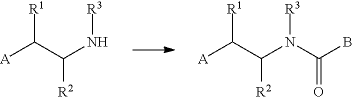

A solution of the respective amine (0.030 mmol) and DIPEA (0 to 3 eq) in DMF (0.25 mL) is added to a mixture of the respective carboxylic acid (0.9 to 1.1 eq), DIPEA (1 to 3 eq) and TBTU (0.9 to 1.1 eq) in DMF (0.25 mL); the total amount of DIPEA is in the range of 2 to 4 equivalents. The mixture is stirred for 16 h and purified by prep. HPLC using a basic gradient to give the desired amides.

| | | LC-MS | | |
|---|---|---|---|---|
| Example | Name | method | t_{R} [min] | [M + H]⁺ |
| 100 | 5-(3-Fluoro-phenyl)-2-methyl-thiazole-4- | (B) | 0.96 | 469.1 |
| | carboxylic acid cyclopropylmethyl-[2-(3,4- | | | |
| | dimethoxy-phenyl)-1-methyl-ethyl]-amide | | | |
| 101 | 2-Amino-5-m-tolyl-thiazole-4-carboxylic acid | (B) | 0.91 | 466.2 |
| | cyclopropylmethyl-[2-(3,4-dimethoxy-phenyl)- | | | |
| | 1-methyl-ethyl]-amide | | | |
| 102 | 5-(3,4-Dimethyl-phenyl)-2-methyl-thiazole-4- | (B) | 1.02 | 479.2 |
| | carboxylic acid cyclopropylmethyl-[2-(3,4- | | | |
| | dimethoxy-phenyl)-1-methyl-ethyl]-amide | | | |
| 103 | 5-(3-Fluoro-phenyl)-2-methyl-thiazole-4- | (B) | 0.85 | 415.1 |
| | carboxylic acid [2-(3,4-dimethoxy-phenyl)- | | | |
| | ethyl]-methyl-amide | | | |
| 104 | 2-Amino-5-m-tolyl-thiazole-4-carboxylic acid | (B) | 0.79 | 412.1 |
| | [2-(3,4-dimethoxy-phenyl)-ethyl]-methyl-amide | | | |
| 105 | 5-(3,4-Dimethyl-phenyl)-2-methyl-thiazole-4- | (B) | 0.90; | 425.0 |
| | carboxylic acid [2-(3,4-dimethoxy-phenyl)- | | 0.93 | |
| | ethyl]-methyl-amide | | | |
| 106 | 5-(3-Fluoro-phenyl)-2-methyl-thiazole-4- | (B) | 0.88; | 429.1 |
| | carboxylic acid [2-(3,4-dimethoxy-phenyl)- | | 0.91 | |
| | ethyl]-ethyl-amide | | | |
| 107 | 2-Amino-5-m-tolyl-thiazole-4-carboxylic acid | (B) | 0.83 | 425.9 |
| | [2-(3,4-dimethoxy-phenyl)-ethyl]-ethyl-amide | | | |
| 108 | 5-(3,4-Dimethyl-phenyl)-2-methyl-thiazole-4- | (B) | 0.94; | 439.1 |
| | carboxylic acid [2-(3,4-dimethoxy-phenyl)- | | 0.98 | |
| | ethyl]-ethyl-amide | | | |
| 109 | 5-(3-Fluoro-phenyl)-2-methyl-thiazole-4- | (B) | 0.92; | 443.1 |
| | carboxylic acid [2-(3,4-dimethoxy-phenyl)- | | 0.95 | |
| | ethyl]-propyl-amide | | | |
| 110 | 2-Amino-5-m-tolyl-thiazole-4-carboxylic acid | (B) | 0.87 | 440.2 |
| | [2-(3,4-dimethoxy-phenyl)-ethyl]-propyl-amide | | | |
| 111 | 5-(3,4-Dimethyl-phenyl)-2-methyl-thiazole-4- | (B) | 0.98; | 453.2 |
| | carboxylic acid [2-(3,4-dimethoxy-phenyl)- | | 1.01 | |
| | ethyl]-propyl-amide | | | |
| 112 | 5-(3-Fluoro-phenyl)-2-methyl-thiazole-4- | (B) | 0.97 | 457.0 |
| | carboxylic acid [2-(3,4-dimethoxy-phenyl)- | | | |
| | ethyl]-isobutyl-amide | | | |
| 113 | 2-Amino-5-m-tolyl-thiazole-4-carboxylic acid | (B) | 0.91 | 454.1 |
| | [2-(3,4-dimethoxy-phenyl)-ethyl]-isobutyl- | | | |
| | amide | | | |
| 114 | 5-(3,4-Dimethyl-phenyl)-2-methyl-thiazole-4- | (B) | 1.02; | 467.2 |
| | carboxylic acid [2-(3,4-dimethoxy-phenyl)- | | 1.04 | |
| | ethyl]-isobutyl-amide | | | |
| 115 | 5-(3-Fluoro-phenyl)-2-methyl-thiazole-4- | (B) | 0.94 | 443.2 |
| | carboxylic acid [2-(3,4-dimethoxy-phenyl)- | | | |
| | ethyl]-isopropyl-amide | | | |
| 116 | 2-Amino-5-m-tolyl-thiazole-4-carboxylic acid | (B) | 0.87 | 440.1 |
| | [2-(3,4-dimethoxy-phenyl)-ethyl]-isopropyl- | | | |
| | amide | | | |
| 117 | 5-(3,4-Dimethyl-phenyl)-2-methyl-thiazole-4- | (B) | 1.01 | 453.2 |
| | carboxylic acid [2-(3,4-dimethoxy-phenyl)- | | | |
| | ethyl]-isopropyl-amide | | | |
| 118 | 5-(3-Fluoro-phenyl)-2-methyl-thiazole-4- | (B) | 0.95 | 483.1 |
| | carboxylic acid [2-(3,4-dimethoxy-phenyl)- | | | |
| | ethyl]-(2,2,2-trifluoro-ethyl)-amide | | | |
| 119 | 2-Amino-5-m-tolyl-thiazole-4-carboxylic acid | (B) | 0.90 | 480.1 |
| | [2-(3,4-dimethoxy-phenyl)-ethyl]-(2,2,2- | | | |
| | trifluoro-ethyl)-amide | | | |
| 120 | 5-(3,4-Dimethyl-phenyl)-2-methyl-thiazole-4- | (B) | 1.01 | 493.1 |
| | carboxylic acid [2-(3,4-dimethoxy-phenyl)- | | | |
| | ethyl]-(2,2,2-trifluoro-ethyl)-amide | | | |
| 121 | 5-(3-Fluoro-phenyl)-2-methyl-thiazole-4- | (B) | 0.92 | 441.1 |
| | carboxylic acid cyclopropyl-[2-(3,4-dimethoxy- | | | |
| | phenyl)-ethyl]-amide | | | |
| 122 | 2-Amino-5-m-tolyl-thiazole-4-carboxylic acid | (B) | 0.85 | 438.1 |
| | cyclopropyl-[2-(3,4-dimethoxy-phenyl)-ethyl]- | | | |
| | amide | | | |
| 123 | 5-(3,4-Dimethyl-phenyl)-2-methyl-thiazole-4- | (B) | 0.98 | 451.2 |
| | carboxylic acid cyclopropyl-[2-(3,4-dimethoxy- | | | |
| | phenyl)-ethyl]-amide | | | |
| 124 | 5-(3-Fluoro-phenyl)-2-methyl-thiazole-4- | (B) | 0.78 | 445.1 |
| | carboxylic acid [2-(3,4-dimethoxy-phenyl)- | | | |
| | ethyl]-(2-hydroxy-ethyl)-amide | | | |
| 125 | 2-Amino-5-m-tolyl-thiazole-4-carboxylic acid | (B) | 0.74 | 442.1 |
| | [2-(3,4-dimethoxy-phenyl)-ethyl]-(2-hydroxy- | | | |
| | ethyl)-amide | | | |
| 126 | 5-(3,4-Dimethyl-phenyl)-2-methyl-thiazole-4- | (B) | 0.84 | 455.1 |
| | carboxylic acid [2-(3,4-dimethoxy-phenyl)- | | | |
| | ethyl]-(2-hydroxy-ethyl)-amide | | | |
| 127 | 5-(3-Fluoro-phenyl)-2-methyl-thiazole-4- | (B) | 0.88 | 459.0 |
| | carboxylic acid [2-(3,4-dimethoxy-phenyl)- | | | |
| | ethyl]-(2-methoxy-ethyl)-amide | | | |
| 128 | 2-Amino-5-m-tolyl-thiazole-4-carboxylic acid | (B) | 0.82 | 456.1 |
| | [2-(3,4-dimethoxy-phenyl)-ethyl]-(2-methoxy- | | | |
| | ethyl)-amide | | | |
| 129 | 5-(3,4-Dimethyl-phenyl)-2-methyl-thiazole-4- | (B) | 0.93 | 469.2 |
| | carboxylic acid [2-(3,4-dimethoxy-phenyl)- | | | |
| | ethyl]-(2-methoxy-ethyl)-amide | | | |
| 130 | 2-Amino-5-m-tolyl-thiazole-4-carboxylic acid | (B) | 0.80 | 469.2 |
| | [2-(3,4-dimethoxy-phenyl)-ethyl]-(2- | | | |
| | dimethylamino-ethyl)-amide | | | |
| 131 | 5-(3-Fluoro-phenyl)-2-methyl-thiazole-4- | (B) | 0.75 | 457.9 |
| | carboxylic acid carbamoylmethyl-[2-(3,4- | | | |
| | dimethoxy-phenyl)-ethyl]-amide | | | |
| 132 | 2-Amino-5-m-tolyl-thiazole-4-carboxylic acid | (B) | 0.72 | 455.1 |
| | carbamoylmethyl-[2-(3,4-dimethoxy-phenyl)- | | | |
| | ethyl]-amide | | | |
| 133 | 5-(3,4-Dimethyl-phenyl)-2-methyl-thiazole-4- | (B) | 0.80 | 468.1 |
| | carboxylic acid carbamoylmethyl-[2-(3,4- | | | |
| | dimethoxy-phenyl)-ethyl]-amide | | | |
| 134 | 5-(3-Fluoro-phenyl)-2-methyl-thiazole-4- | (B) | 0.81 | 486.2 |
| | carboxylic acid [2-(3,4-dimethoxy-phenyl)- | | | |
| | ethyl]-dimethylcarbamoylmethyl-amide | | | |
| 135 | 2-Amino-5-m-tolyl-thiazole-4-carboxylic acid | (B) | 0.77 | 483.2 |
| | [2-(3,4-dimethoxy-phenyl)-ethyl]- | | | |
| | dimethylcarbamoylmethyl-amide | | | |
| 136 | 5-(3,4-Dimethyl-phenyl)-2-methyl-thiazole-4- | (B) | 0.87 | 496.2 |
| | carboxylic acid [2-(3,4-dimethoxy-phenyl)- | | | |
| | ethyl]-dimethylcarbamoylmethyl-amide | | | |
| 137 | 2-Amino-5-m-tolyl-thiazole-4-carboxylic acid | (C) | 0.80 | 392.3 |
| | cyclopropylmethyl-phenethyl-amide | | | |
| 138 | 2-Amino-5-m-tolyl-thiazole-4-carboxylic acid | (C) | 0.83 | 426.2 |
| | [2-(2-chloro-phenyl)-ethyl]-cyclopropylmethyl- | | | |
| | amide | | | |
| 139 | 2-Amino-5-m-tolyl-thiazole-4-carboxylic acid | (C) | 0.78 | 422.2 |
| | cyclopropylmethyl-[2-(2-methoxy-phenyl)- | | | |
| | ethyl]-amide | | | |
| 140 | 2-Amino-5-m-tolyl-thiazole-4-carboxylic acid | (C) | 0.80 | 410.2 |
| | cyclopropylmethyl-[2-(2-fluoro-phenyl)-ethyl]- | | | |
| | amide | | | |
| 141 | 2-Amino-5-m-tolyl-thiazole-4-carboxylic acid | (C) | 0.83 | 406.3 |
| | cyclopropylmethyl-(2-o-tolyl-ethyl)-amide | | | |
| 142 | 2-Amino-5-m-tolyl-thiazole-4-carboxylic acid | (C) | 0.81 | 406.3 |
| | cyclopropylmethyl-(2-m-tolyl-ethyl)-amide | | | |
| 143 | 2-Amino-5-m-tolyl-thiazole-4-carboxylic acid | (C) | 0.79 | 422.3 |
| | cyclopropylmethyl-[2-(3-methoxy-phenyl)- | | | |
| | ethyl]-amide | | | |
| 144 | 2-Amino-5-m-tolyl-thiazole-4-carboxylic acid | (C) | 0.86 | 426.2 |
| | [2-(4-chloro-phenyl)-ethyl]-cyclopropylmethyl- | | | |
| | amide | | | |
| 145 | 2-Amino-5-m-tolyl-thiazole-4-carboxylic acid | (C) | 0.84 | 406.3 |
| | cyclopropylmethyl-(2-p-tolyl-ethyl)-amide | | | |
| 146 | 2-Amino-5-m-tolyl-thiazole-4-carboxylic acid | (C) | 0.89 | 420.3 |
| | cyclopropylmethyl-[2-(4-ethyl-phenyl)-ethyl]- | | | |
| | amide | | | |
| 147 | 2-Amino-5-m-tolyl-thiazole-4-carboxylic acid | (C) | 0.78 | 422.2 |
| | cyclopropylmethyl-[2-(4-methoxy-phenyl)- | | | |
| | ethyl]-amide | | | |
| 148 | 2-Amino-5-m-tolyl-thiazole-4-carboxylic acid | (C) | 0.66 | 408.3 |
| | cyclopropylmethyl-[2-(4-hydroxy-phenyl)- | | | |
| | ethyl]-amide | | | |
| 149 | 2-Amino-5-m-tolyl-thiazole-4-carboxylic acid | (C) | 0.84 | 438.2 |
| | cyclopropylmethyl-[2-(4-methylsulfanyl- | | | |
| | phenyl)-ethyl]-amide | | | |
| 150 | 2-Amino-5-m-tolyl-thiazole-4-carboxylic acid | (C) | 0.88 | 460.2 |
| | cyclopropylmethyl-[2-(4-trifluoromethyl- | | | |
| | phenyl)-ethyl]-amide | | | |
| 151 | 2-Amino-5-m-tolyl-thiazole-4-carboxylic acid | (C) | 0.90 | 476.2 |
| | cyclopropylmethyl-[2-(4-trifluoromethoxy- | | | |
| | phenyl)-ethyl]-amide | | | |
| 152 | 2-Amino-5-m-tolyl-thiazole-4-carboxylic acid | (C) | 0.88 | 420.3 |
| | cyclopropylmethyl-[2-(2,4-dimethyl-phenyl)- | | | |
| | ethyl]-amide | | | |
| 153 | 2-Amino-5-m-tolyl-thiazole-4-carboxylic acid | (C) | 0.78 | 452.2 |
| | cyclopropylmethyl-[2-(2,5-dimethoxy-phenyl)- | | | |
| | ethyl]-amide | | | |
| 154 | 2-Amino-5-m-tolyl-thiazole-4-carboxylic acid | (C) | 0.88 | 420.3 |
| | cyclopropylmethyl-[2-(2,5-dimethyl-phenyl)- | | | |
| | ethyl]-amide | | | |
| 155 | 2-Amino-5-m-tolyl-thiazole-4-carboxylic acid | (C) | 0.87 | 500.2 |
| | [2-(5-bromo-2-methoxy-phenyl)-ethyl]- | | | |
| | cyclopropylmethyl-amide | | | |
| 156 | 2-Amino-5-m-tolyl-thiazole-4-carboxylic acid | (C) | 0.77 | 436.2 |
| | (2-benzo[1,3]dioxol-5-yl-ethyl)- | | | |
| | cyclopropylmethyl-amide | | | |
| 157 | 2-Amino-5-m-tolyl-thiazole-4-carboxylic acid | (C) | 0.88 | 472.2 |
| | cyclopropylmethyl-[2-(2,2-difluoro- | | | |
| | benzo[1,3]dioxol-5-yl)-ethyl]-amide | | | |
| 158 | 2-Amino-5-m-tolyl-thiazole-4-carboxylic acid | (C) | 0.76 | 449.8 |
| | cyclopropylmethyl-[2-(2,3-dihydro- | | | |
| | benzo[1,4]dioxin-6-yl)-ethyl]-amide | | | |
| 159 | 2-Amino-5-m-tolyl-thiazole-4-carboxylic acid | (C) | 0.78 | 465.8 |
| | cyclopropylmethyl-[2-(4-ethoxy-3-methoxy- | | | |
| | phenyl)-ethyl]-amide | | | |
| 160 | 2-Amino-5-m-tolyl-thiazole-4-carboxylic acid | (C) | 0.78 | 465.8 |
| | cyclopropylmethyl-[2-(3-ethoxy-4-methoxy- | | | |
| | phenyl)-ethyl]-amide | | | |
| 161 | 2-Amino-5-m-tolyl-thiazole-4-carboxylic acid | (C) | 0.81 | 468.2 |
| | cyclopropylmethyl-[2-(4-methoxy-3- | | | |
| | methylsulfanyl-phenyl)-ethyl]-amide | | | |
| 162 | 2-Amino-5-m-tolyl-thiazole-4-carboxylic acid | (C) | 0.84 | 436.3 |
| | cyclopropylmethyl-[2-(4-methoxy-3-methyl- | | | |
| | phenyl)-ethyl]-amide | | | |
| 163 | 2-Amino-5-m-tolyl-thiazole-4-carboxylic acid | (C) | 0.83 | 500.1 |
| | [2-(3-bromo-4-methoxy-phenyl)-ethyl]- | | | |
| | cyclopropylmethyl-amide | | | |
| 164 | 2-Amino-5-m-tolyl-thiazole-4-carboxylic acid | (C) | 0.88 | 420.3 |
| | cyclopropylmethyl-[2-(3,4-dimethyl-phenyl)- | | | |
| | ethyl]-amide | | | |
| 165 | 2-Amino-5-m-tolyl-thiazole-4-carboxylic acid | (C) | 0.81 | 488.2 |
| | cyclopropylmethyl-[2-(3-difluoromethoxy-4- | | | |
| | methoxy-phenyl)-ethyl]-amide | | | |
| 166 | 2-Amino-5-m-tolyl-thiazole-4-carboxylic acid | (C) | 0.82 | 488.2 |
| | cyclopropylmethyl-[2-(4-difluoromethoxy-3- | | | |
| | methoxy-phenyl)-ethyl]-amide | | | |
| 167 | 2-Amino-5-m-tolyl-thiazole-4-carboxylic acid | (C) | 0.88 | 442.2 |
| | cyclopropylmethyl-(2-naphthalen-2-yl-ethyl)- | | | |
| | amide | | | |
| 168 | 2-Amino-5-m-tolyl-thiazole-4-carboxylic acid | (C) | 0.67 | 438.2 |
| | cyclopropylmethyl-[2-(4-hydroxy-3-methoxy- | | | |
| | phenyl)-ethyl]-amide | | | |
| 169 | 2-Amino-5-m-tolyl-thiazole-4-carboxylic acid | (C) | 0.78 | 480.3 |
| | cyclopropylmethyl-[1-(3,4-dimethoxy-benzyl)- | | | |
| | propyl]-amide | | | |
| 170 | 2-Amino-5-m-tolyl-thiazole-4-carboxylic acid | (C) | 0.79 | 452.2 |
| | cyclopropylmethyl-[2-(3,5-dimethoxy-phenyl)- | | | |
| | ethyl]-amide | | | |
| 171 | 2-Amino-5-m-tolyl-thiazole-4-carboxylic acid | (C) | 0.88 | 460.2 |
| | cyclopropylmethyl-[2-(2,6-dichloro-phenyl)- | | | |
| | ethyl]-amide | | | |
| 172 | 2-Amino-5-m-tolyl-thiazole-4-carboxylic acid | (C) | 0.74 | 481.8 |
| | cyclopropylmethyl-[2-(3,4,5-trimethoxy- | | | |
| | phenyl)-ethyl]-amide | | | |
| 173 | 2-Amino-5-m-tolyl-thiazole-4-carboxylic acid | (C) | 0.84 | 510.3 |
| | cyclopropylmethyl-[2-(4-isopropoxy-3,5- | | | |
| | dimethoxy-phenyl)-ethyl]-amide | | | |
| 174 | 2-Amino-5-m-tolyl-thiazole-4-carboxylic acid | (C) | 0.89 | 577.7 |
| | cyclopropylmethyl-[2-(4-iodo-2,5-dimethoxy- | | | |
| | phenyl)-ethyl]-amide | | | |
| 175 | 2-Amino-5-m-tolyl-thiazole-4-carboxylic acid | (C) | 0.71 | 431.2 |
| | cyclopropylmethyl-[2-(1H-indol-3-yl)-ethyl]- | | | |
| | amide | | | |
| 176 | 2-Amino-5-m-tolyl-thiazole-4-carboxylic acid | (C) | 0.55 | 432.2 |
| | [2-(1H-benzoimidazol-2-yl)-ethyl]- | | | |
| | cyclopropylmethyl-amide | | | |
| 177 | 2-Amino-5-m-tolyl-thiazole-4-carboxylic acid | (C) | 0.52 | 414.2 |
| | [2-(2-amino-thiazol-4-yl)-ethyl]- | | | |
| | cyclopropylmethyl-amide | | | |
| 178 | 2-Amino-5-m-tolyl-thiazole-4-carboxylic acid | (C) | 0.57 | 536.1 |
| | cyclopropylmethyl-[2-(2-ethyl-4-iodo-imidazol- | | | |
| | 1-yl)-ethyl]-amide | | | |
| 179 | 5-(3,4-Dimethyl-phenyl)-2-methyl-thiazole-4- | (C) | 0.99 | 405.3 |
| | carboxylic acid cyclopropylmethyl-phenethyl- | | | |
| | amide | | | |
| 180 | 5-(3,4-Dimethyl-phenyl)-2-methyl-thiazole-4- | (C) | 1.04 | 439.2 |
| | carboxylic acid [2-(2-chloro-phenyl)-ethyl]- | | | |
| | cyclopropylmethyl-amide | | | |
| 181 | 5-(3,4-Dimethyl-phenyl)-2-methyl-thiazole-4- | (C) | 1.00 | 435.3 |
| | carboxylic acid cyclopropylmethyl-[2-(2- | | | |
| | methoxy-phenyl)-ethyl]-amide | | | |
| 182 | 5-(3,4-Dimethyl-phenyl)-2-methyl-thiazole-4- | (C) | 1.00 | 423.2 |
| | carboxylic acid cyclopropylmethyl-[2-(2-fluoro- | | | |
| | phenyl)-ethyl]-amide | | | |
| 183 | 5-(3,4-Dimethyl-phenyl)-2-methyl-thiazole-4- | (C) | 1.04 | 419.2 |
| | carboxylic acid cyclopropylmethyl-(2-m-tolyl- | | | |
| | ethyl)-amide | | | |
| 184 | 5-(3,4-Dimethyl-phenyl)-2-methyl-thiazole-4- | (C) | 0.98 | 435.2 |
| | carboxylic acid cyclopropylmethyl-[2-(3- | | | |
| | methoxy-phenyl)-ethyl]-amide | | | |
| 185 | 5-(3,4-Dimethyl-phenyl)-2-methyl-thiazole-4- | (C) | 0.99 | 423.3 |
| | carboxylic acid cyclopropylmethyl-[2-(4-fluoro- | | | |
| | phenyl)-ethyl]-amide | | | |
| 186 | 5-(3,4-Dimethyl-phenyl)-2-methyl-thiazole-4- | (C) | 1.04 | 439.2 |
| | carboxylic acid [2-(4-chloro-phenyl)-ethyl]- | | | |
| | cyclopropylmethyl-amide | | | |
| 187 | 5-(3,4-Dimethyl-phenyl)-2-methyl-thiazole-4- | (C) | 1.04 | 419.2 |
| | carboxylic acid cyclopropylmethyl-(2-p-tolyl- | | | |
| | ethyl)-amide | | | |
| 188 | 5-(3,4-Dimethyl-phenyl)-2-methyl-thiazole-4- | (C) | 1.08 | 433.2 |
| | carboxylic acid cyclopropylmethyl-[2-(4-ethyl- | | | |
| | phenyl)-ethyl]-amide | | | |
| 189 | 5-(3,4-Dimethyl-phenyl)-2-methyl-thiazole-4- | (C) | 0.97 | 435.2 |
| | carboxylic acid cyclopropylmethyl-[2-(4- | | | |
| | methoxy-phenyl)-ethyl]-amide | | | |
| 190 | 5-(3,4-Dimethyl-phenyl)-2-methyl-thiazole-4- | (C) | 0.82 | 421.3 |
| | carboxylic acid cyclopropylmethyl-[2-(4- | | | |
| | hydroxy-phenyl)-ethyl]-amide | | | |
| 191 | 5-(3,4-Dimethyl-phenyl)-2-methyl-thiazole-4- | (C) | 1.02 | 451.2 |
| | carboxylic acid cyclopropylmethyl-[2-(4- | | | |
| | methylsulfanyl-phenyl)-ethyl]-amide | | | |
| 192 | 5-(3,4-Dimethyl-phenyl)-2-methyl-thiazole-4- | (C) | 1.04 | 473.2 |
| | carboxylic acid cyclopropylmethyl-[2-(4- | | | |
| | trifluoromethyl-phenyl)-ethyl]-amide | | | |
| 193 | 5-(3,4-Dimethyl-phenyl)-2-methyl-thiazole-4- | (C) | 1.08 | 433.2 |
| | carboxylic acid cyclopropylmethyl-[2-(2,4- | | | |
| | dimethyl-phenyl)-ethyl]-amide | | | |
| 194 | 5-(3,4-Dimethyl-phenyl)-2-methyl-thiazole-4- | (C) | 0.98 | 465.2 |
| | carboxylic acid cyclopropylmethyl-[2-(2,5- | | | |
| | dimethoxy-phenyl)-ethyl]-amide | | | |
| 195 | 5-(3,4-Dimethyl-phenyl)-2-methyl-thiazole-4- | (C) | 1.08 | 433.3 |
| | carboxylic acid cyclopropylmethyl-[2-(2,5- | | | |
| | dimethyl-phenyl)-ethyl]-amide | | | |
| 196 | 5-(3,4-Dimethyl-phenyl)-2-methyl-thiazole-4- | (C) | 1.06 | 513.0 |
| | carboxylic acid [2-(5-bromo-2-methoxy- | | | |
| | phenyl)-ethyl]-cyclopropylmethyl-amide | | | |
| 197 | 5-(3,4-Dimethyl-phenyl)-2-methyl-thiazole-4- | (C) | 0.95 | 449.2 |
| | carboxylic acid (2-benzo[1,3]dioxol-5-yl-ethyl)- | | | |
| | cyclopropylmethyl-amide | | | |
| 198 | 5-(3,4-Dimethyl-phenyl)-2-methyl-thiazole-4- | (C) | 0.94 | 463.2 |
| | carboxylic acid cyclopropylmethyl-[2-(2,3- | | | |
| | dihydro-benzo[1,4]dioxin-6-yl)-ethyl]-amide | | | |
| 199 | 5-(3,4-Dimethyl-phenyl)-2-methyl-thiazole-4- | (C) | 0.96 | 479.3 |
| | carboxylic acid cyclopropylmethyl-[2-(4- | | | |
| | ethoxy-3-methoxy-phenyl)-ethyl]-amide | | | |
| 200 | 5-(3,4-Dimethyl-phenyl)-2-methyl-thiazole-4- | (C) | 0.95 | 479.3 |
| | carboxylic acid cyclopropylmethyl-[2-(3- | | | |
| | ethoxy-4-methoxy-phenyl)-ethyl]-amide | | | |
| 201 | 5-(3,4-Dimethyl-phenyl)-2-methyl-thiazole-4- | (C) | 0.99 | 481.1 |
| | carboxylic acid cyclopropylmethyl-[2-(4- | | | |
| | methoxy-3-methylsulfanyl-phenyl)-ethyl]-amide | | | |
| 202 | 5-(3,4-Dimethyl-phenyl)-2-methyl-thiazole-4- | (C) | 1.03 | 449.2 |
| | carboxylic acid cyclopropylmethyl-[2-(4- | | | |
| | methoxy-3-methyl-phenyl)-ethyl]-amide | | | |
| 203 | 5-(3,4-Dimethyl-phenyl)-2-methyl-thiazole-4- | (C) | 1.01 | 513.1 |
| | carboxylic acid [2-(3-bromo-4-methoxy- | | | |
| | phenyl)-ethyl]-cyclopropylmethyl-amide | | | |
| 204 | 5-(3,4-Dimethyl-phenyl)-2-methyl-thiazole-4- | (C) | 1.07 | 433.4 |
| | carboxylic acid cyclopropylmethyl-[2-(3,4- | | | |
| | dimethyl-phenyl)-ethyl]-amide | | | |
| 205 | 5-(3,4-Dimethyl-phenyl)-2-methyl-thiazole-4- | (C) | 0.97 | 501.2 |
| | carboxylic acid cyclopropylmethyl-[2-(3- | | | |
| | difluoromethoxy-4-methoxy-phenyl)-ethyl]- | | | |
| | amide | | | |
| 206 | 5-(3,4-Dimethyl-phenyl)-2-methyl-thiazole-4- | (C) | 0.97 | 501.2 |
| | carboxylic acid cyclopropylmethyl-[2-(4- | | | |
| | difluoromethoxy-3-methoxy-phenyl)-ethyl]- | | | |
| | amide | | | |
| 207 | 5-(3,4-Dimethyl-phenyl)-2-methyl-thiazole-4- | (C) | 0.84 | 451.2 |
| | carboxylic acid cyclopropylmethyl-[2-(4- | | | |
| | hydroxy-3-methoxy-phenyl)-ethyl]-amide | | | |
| 208 | 5-(3,4-Dimethyl-phenyl)-2-methyl-thiazole-4- | (C) | 0.96; | 493.3 |
| | carboxylic acid cyclopropylmethyl-[1-(3,4- | | 1.01 | |
| | dimethoxy-benzyl)-propyl]-amide | | | |
| 209 | 5-(3,4-Dimethyl-phenyl)-2-methyl-thiazole-4- | (C) | 0.97 | 465.2 |
| | carboxylic acid cyclopropylmethyl-[2-(3,5- | | | |
| | dimethoxy-phenyl)-ethyl]-amide | | | |
| 210 | 5-(3,4-Dimethyl-phenyl)-2-methyl-thiazole-4- | (C) | 1.08 | 473.2 |
| | carboxylic acid cyclopropylmethyl-[2-(2,6- | | | |
| | dichloro-phenyl)-ethyl]-amide | | | |
| 211 | 5-(3,4-Dimethyl-phenyl)-2-methyl-thiazole-4- | (C) | 0.91 | 495.3 |
| | carboxylic acid cyclopropylmethyl-[2-(3,4,5- | | | |
| | trimethoxy-phenyl)-ethyl]-amide | | | |
| 212 | 5-(3,4-Dimethyl-phenyl)-2-methyl-thiazole-4- | (C) | 1.01 | 523.3 |
| | carboxylic acid cyclopropylmethyl-[2-(4- | | | |
| | isopropoxy-3,5-dimethoxy-phenyl)-ethyl]-amide | | | |
| 213 | 5-(3,4-Dimethyl-phenyl)-2-methyl-thiazole-4- | (C) | 1.06 | 591.1 |
| | carboxylic acid cyclopropylmethyl-[2-(4-iodo- | | | |
| | 2,5-dimethoxy-phenyl)-ethyl]-amide | | | |
| 214 | 5-(3,4-Dimethyl-phenyl)-2-methyl-thiazole-4- | (C) | 0.92 | 444.2 |
| | carboxylic acid cyclopropylmethyl-[2-(1H- | | | |
| | indol-3-yl)-ethyl]-amide | | | |
| 215 | 5-(3,4-Dimethyl-phenyl)-2-methyl-thiazole-4- | (C) | 0.64 | 445.2 |
| | carboxylic acid [2-(1H-benzoimidazol-2-yl)- | | | |
| | ethyl]-cyclopropylmethyl-amide | | | |
| 216 | 5-(3,4-Dimethyl-phenyl)-2-methyl-thiazole-4- | (C) | 0.67 | 549.1 |
| | carboxylic acid cyclopropylmethyl-[2-(2-ethyl- | | | |
| | 4-iodo-imidazol-1-yl)-ethyl]-amide | | | |
| 217 | 2-Amino-5-m-tolyl-thiazole-4-carboxylic acid | (C) | 0.56 | 462.2 |
| | cyclopropylmethyl-[2-(6-methoxy-1H- | | | |
| | benzoimidazol-2-yl)-ethyl]-amide | | | |
| 218 | 2-Amino-5-m-tolyl-thiazole-4-carboxylic acid | (C) | 0.60 | 460.2 |
| | cyclopropylmethyl-[2-(5,6-dimethyl-1H- | | | |
| | benzoimidazol-2-yl)-ethyl]-amide | | | |
| 219 | 2-Amino-5-m-tolyl-thiazole-4-carboxylic acid | (C) | 0.57 | 446.2 |
| | cyclopropylmethyl-[2-(6-methyl-1H- | | | |
| | benzoimidazol-2-yl)-ethyl]-amide | | | |
| 220 | 2-Amino-5-m-tolyl-thiazole-4-carboxylic acid | (C) | 0.59 | 465.7 |
| | [2-(6-chloro-1H-benzoimidazol-2-yl)-ethyl]- | | | |
| | cyclopropylmethyl-amide | | | |
| 221 | 2-Amino-5-m-tolyl-thiazole-4-carboxylic acid | (C) | 0.79 | 431.2 |
| | cyclopropylmethyl-(2-indol-1-yl-ethyl)-amide | | | |
| 222 | 2-Amino-5-m-tolyl-thiazole-4-carboxylic acid | (C) | 0.79 | 445.2 |
| | cyclopropylmethyl-[2-(1-methyl-1H-indol-3- | | | |
| | yl)-ethyl]-amide | | | |
| 223 | 2-Amino-5-m-tolyl-thiazole-4-carboxylic acid | (C) | 0.75 | 509.1 |
| | [2-(5-bromo-1H-indol-3-yl)-ethyl]- | | | |
| | cyclopropylmethyl-amide | | | |
| 224 | 2-Amino-5-m-tolyl-thiazole-4-carboxylic acid | (C) | 0.77 | 465.2 |
| | [2-(6-chloro-1H-indol-3-yl)-ethyl]- | | | |
| | cyclopropylmethyl-amide | | | |
| 225 | 2-Amino-5-m-tolyl-thiazole-4-carboxylic acid | (C) | 0.72 | 461.2 |
| | cyclopropylmethyl-[2-(7-methoxy-1H-indol-3- | | | |
| | yl)-ethyl]-amide | | | |
| 226 | 2-Amino-5-m-tolyl-thiazole-4-carboxylic acid | (C) | 0.68 | 461.2 |
| | cyclopropylmethyl-[2-(5-methoxy-1H-indol-3- | | | |
| | yl)-ethyl]-amide | | | |
| 227 | 2-Amino-5-m-tolyl-thiazole-4-carboxylic acid | (C) | 0.69 | 461.2 |
| | cyclopropylmethyl-[2-(6-methoxy-1H-indol-3- | | | |
| | yl)-ethyl]-amide | | | |
| 228 | 2-Amino-5-m-tolyl-thiazole-4-carboxylic acid | (C) | 0.73 | 445.2 |
| | cyclopropylmethyl-[2-(5-methyl-1H-indol-3- | | | |
| | yl)-ethyl]-amide | | | |
| 229 | 2-Amino-5-m-tolyl-thiazole-4-carboxylic acid | (C) | 0.74 | 445.2 |
| | cyclopropylmethyl-[2-(6-methyl-1H-indol-3- | | | |
| | yl)-ethyl]-amide | | | |
| 230 | 2-Amino-5-m-tolyl-thiazole-4-carboxylic acid | (C) | 0.74 | 445.2 |
| | cyclopropylmethyl-[2-(7-methyl-1H-indol-3- | | | |
| | yl)-ethyl]-amide | | | |
| 231 | 2-Amino-5-m-tolyl-thiazole-4-carboxylic acid | (C) | 0.72 | 449.2 |
| | cyclopropylmethyl-[2-(4-fluoro-1H-indol-3-yl)- | | | |
| | ethyl]-amide | | | |
| 232 | 2-Amino-5-m-tolyl-thiazole-4-carboxylic acid | (C) | 0.72 | 449.2 |
| | cyclopropylmethyl-[2-(5-fluoro-1H-indol-3-yl)- | | | |
| | ethyl]-amide | | | |
| 233 | 2-Amino-5-m-tolyl-thiazole-4-carboxylic acid | (C) | 0.73 | 449.2 |
| | cyclopropylmethyl-[2-(6-fluoro-1H-indol-3-yl)- | | | |
| | ethyl]-amide | | | |
| 234 | 2-Amino-5-m-tolyl-thiazole-4-carboxylic acid | (C) | 0.73 | 449.1 |
| | cyclopropylmethyl-[2-(7-fluoro-1H-indol-3-yl)- | | | |
| | ethyl]-amide | | | |
| 235 | 2-Amino-5-m-tolyl-thiazole-4-carboxylic acid | (C) | 0.68 | 423.2 |
| | cyclopropylmethyl-[2-(6-methoxy-pyridin-3- | | | |
| | yl)-ethyl]-amide | | | |
| 236 | 5-(3,4-Dimethyl-phenyl)-2-methyl-thiazole-4- | (C) | 0.65 | 475.2 |
| | carboxylic acid cyclopropylmethyl-[2-(6- | | | |
| | methoxy-1H-benzoimidazol-2-yl)-ethyl]-amide | | | |
| 237 | 5-(3,4-Dimethyl-phenyl)-2-methyl-thiazole-4- | (C) | 0.69 | 473.2 |
| | carboxylic acid cyclopropylmethyl-[2-(5,6- | | | |
| | dimethyl-1H-benzoimidazol-2-yl)-ethyl]-amide | | | |
| 238 | 5-(3,4-Dimethyl-phenyl)-2-methyl-thiazole-4- | (C) | 0.66 | 459.2 |
| | carboxylic acid cyclopropylmethyl-[2-(6- | | | |
| | methyl-1H-benzoimidazol-2-yl)-ethyl]-amide | | | |
| 239 | 5-(3,4-Dimethyl-phenyl)-2-methyl-thiazole-4- | (C) | 0.68 | 479.2 |
| | carboxylic acid [2-(6-chloro-1H-benzoimidazol- | | | |
| | 2-yl)-ethyl]-cyclopropylmethyl-amide | | | |
| 240 | 5-(3,4-Dimethyl-phenyl)-2-methyl-thiazole-4- | (C) | 1.00 | 444.2 |
| | carboxylic acid cyclopropylmethyl-(2-indol-1- | | | |
| | yl-ethyl)-amide | | | |
| 241 | 5-(3,4-Dimethyl-phenyl)-2-methyl-thiazole-4- | (C) | 1.00 | 458.2 |
| | carboxylic acid cyclopropylmethyl-[2-(1- | | | |
| | methyl-1H-indol-3-yl)-ethyl]-amide | | | |
| 242 | 5-(3,4-Dimethyl-phenyl)-2-methyl-thiazole-4- | (C) | 0.97 | 522.1 |
| | carboxylic acid [2-(5-bromo-1H-indol-3-yl)- | | | |
| | ethyl]-cyclopropylmethyl-amide | | | |
| 243 | 5-(3,4-Dimethyl-phenyl)-2-methyl-thiazole-4- | (C) | 0.97 | 478.1 |
| | carboxylic acid [2-(6-chloro-1H-indol-3-yl)- | | | |
| | ethyl]-cyclopropylmethyl-amide | | | |
| 244 | 5-(3,4-Dimethyl-phenyl)-2-methyl-thiazole-4- | (C) | 0.92 | 474.2 |
| | carboxylic acid cyclopropylmethyl-[2-(7- | | | |
| | methoxy-1H-indol-3-yl)-ethyl]-amide | | | |
| 245 | 5-(3,4-Dimethyl-phenyl)-2-methyl-thiazole-4- | (C) | 0.89 | 474.2 |
| | carboxylic acid cyclopropylmethyl-[2-(5- | | | |
| | methoxy-1H-indol-3-yl)-ethyl]-amide | | | |
| 246 | 5-(3,4-Dimethyl-phenyl)-2-methyl-thiazole-4- | (C) | 0.89 | 474.2 |
| | carboxylic acid cyclopropylmethyl-[2-(6- | | | |
| | methoxy-1H-indol-3-yl)-ethyl]-amide | | | |
| 247 | 5-(3,4-Dimethyl-phenyl)-2-methyl-thiazole-4- | (C) | 0.95 | 458.2 |
| | carboxylic acid cyclopropylmethyl-[2-(5- | | | |
| | methyl-1H-indol-3-yl)-ethyl]-amide | | | |
| 248 | 5-(3,4-Dimethyl-phenyl)-2-methyl-thiazole-4- | (C) | 0.95 | 458.2 |
| | carboxylic acid cyclopropylmethyl-[2-(6- | | | |
| | methyl-1H-indol-3-yl)-ethyl]-amide | | | |
| 249 | 5-(3,4-Dimethyl-phenyl)-2-methyl-thiazole-4- | (C) | 0.95 | 458.2 |
| | carboxylic acid cyclopropylmethyl-[2-(7- | | | |
| | methyl-1H-indol-3-yl)-ethyl]-amide | | | |
| 250 | 5-(3,4-Dimethyl-phenyl)-2-methyl-thiazole-4- | (C) | 0.93 | 462.2 |
| | carboxylic acid cyclopropylmethyl-[2-(4-fluoro- | | | |
| | 1H-indol-3-yl)-ethyl]-amide | | | |
| 251 | 5-(3,4-Dimethyl-phenyl)-2-methyl-thiazole-4- | (C) | 0.92 | 462.2 |
| | carboxylic acid cyclopropylmethyl-[2-(5-fluoro- | | | |
| | 1H-indol-3-yl)-ethyl]-amide | | | |
| 252 | 5-(3,4-Dimethyl-phenyl)-2-methyl-thiazole-4- | (C) | 0.92 | 462.2 |
| | carboxylic acid cyclopropylmethyl-[2-(6-fluoro- | | | |
| | 1H-indol-3-yl)-ethyl]-amide | | | |
| 253 | 5-(3,4-Dimethyl-phenyl)-2-methyl-thiazole-4- | (C) | 0.94 | 462.2 |
| | carboxylic acid cyclopropylmethyl-[2-(7-fluoro- | | | |
| | 1H-indol-3-yl)-ethyl]-amide | | | |
| 254 | 5-(3,4-Dimethyl-phenyl)-2-methyl-thiazole-4- | (C) | 0.86 | 436.2 |
| | carboxylic acid cyclopropylmethyl-[2-(6- | | | |
| | methoxy-pyridin-3-yl)-ethyl]-amide | | | |
| 255 | 3-p-Tolyl-pyrazine-2-carboxylic acid | (B) | 0.92; | 429.1 |
| | cyclopropylmethyl-[2-(5-fluoro-1H-indol-3-yl)- | | 0.94 | |
| | ethyl]-amide | | | |
| 256 | 3-(3,4-Dimethyl-phenyl)-pyrazine-2-carboxylic | (B) | 0.95 | 443.1 |
| | acid cyclopropylmethyl-[2-(5-fluoro-1H-indol- | | | |
| | 3-yl)-ethyl]-amide | | | |
| 257 | 3-m-Tolyl-pyrazine-2-carboxylic acid | (B) | 0.92; | 429.1 |
| | cyclopropylmethyl-[2-(5-fluoro-1H-indol-3-yl)- | | 0.94 | |
| | ethyl]-amide | | | |
| 258 | 3-(3-Methoxy-phenyl)-pyrazine-2-carboxylic | (B) | 0.89; | 445.1 |
| | acid cyclopropylmethyl-[2-(5-fluoro-1H-indol- | | 0.91 | |
| | 3-yl)-ethyl]-amide | | | |
| 259 | 5-(3,4-Dimethyl-phenyl)-2-methyl-oxazole-4- | (B) | 0.99 | 446.1 |
| | carboxylic acid cyclopropylmethyl-[2-(5-fluoro- | | | |
| | 1H-indol-3-yl)-ethyl]-amide | | | |
| 260 | 5-(3,4-Dimethyl-phenyl)-oxazole-4-carboxylic | (B) | 0.97 | 432.1 |
| | acid cyclopropylmethyl-[2-(5-fluoro-1H-indol- | | | |
| | 3-yl)-ethyl]-amide | | | |
| 261 | 5-(3-Dimethylamino-phenyl)-oxazole-4- | (B) | 0.95 | 447.1 |
| | carboxylic acid cyclopropylmethyl-[2-(5-fluoro- | | | |
| | 1H-indol-3-yl)-ethyl]-amide | | | |
| 262 | 4-(4-Chloro-phenyl)-2-methyl-thiazole-5- | (B) | 0.99 | 468.0 |
| | carboxylic acid cyclopropylmethyl-[2-(5-fluoro- | | | |
| | 1H-indol-3-yl)-ethyl]-amide | | | |
| 263 | 5-(4-Fluoro-phenyl)-2-methyl-thiazole-4- | (B) | 0.95 | 452.1 |
| | carboxylic acid cyclopropylmethyl-[2-(5-fluoro- | | | |
| | 1H-indol-3-yl)-ethyl]-amide | | | |
| 264 | 5-(4-Ethyl-phenyl)-2-methyl-thiazole-4- | (B) | 1.01 | 462.1 |
| | carboxylic acid cyclopropylmethyl-[2-(5-fluoro- | | | |
| | 1H-indol-3-yl)-ethyl]-amide | | | |
| 265 | 5-(3-Chloro-phenyl)-2-methyl-thiazole-4- | (B) | 0.99 | 468.0 |
| | carboxylic acid cyclopropylmethyl-[2-(5-fluoro- | | | |
| | 1H-indol-3-yl)-ethyl]-amide | | | |
| 266 | 2-Methyl-5-p-tolyl-thiazole-4-carboxylic acid | (B) | 0.98 | 448.0 |
| | cyclopropylmethyl-[2-(5-fluoro-1H-indol-3-yl)- | | | |
| | ethyl]-amide | | | |
| 267 | 5-(3,5-Dimethyl-phenyl)-2-methyl-thiazole-4- | (B) | 1.01 | 462.1 |
| | carboxylic acid cyclopropylmethyl-[2-(5-fluoro- | | | |
| | 1H-indol-3-yl)-ethyl]-amide | | | |
| 268 | 5-(3-Cyano-phenyl)-2-methyl-thiazole-4- | (B) | 0.91 | 458.9 |
| | carboxylic acid cyclopropylmethyl-[2-(5-fluoro- | | | |
| | 1H-indol-3-yl)-ethyl]-amide | | | |
| 269 | 5-(4-Chloro-phenyl)-2-methyl-thiazole-4- | (B) | 0.99 | 468.0 |
| | carboxylic acid cyclopropylmethyl-[2-(5-fluoro- | | | |
| | 1H-indol-3-yl)-ethyl]-amide | | | |
| 270 | 5-(3,4-Difluoro-phenyl)-2-methyl-thiazole-4- | (B) | 0.97 | 470.0 |
| | carboxylic acid cyclopropylmethyl-[2-(5-fluoro- | | | |
| | 1H-indol-3-yl)-ethyl]-amide | | | |
| 271 | 5-(3,4-Dichloro-phenyl)-2-methyl-thiazole-4- | (B) | 1.04 | 501.9 |
| | carboxylic acid cyclopropylmethyl-[2-(5-fluoro- | | | |
| | 1H-indol-3-yl)-ethyl]-amide | | | |
| 272 | 5-(3-Fluoro-4-methyl-phenyl)-2-methyl- | (B) | 0.99 | 466.0 |
| | thiazole-4-carboxylic acid cyclopropylmethyl- | | | |
| | [2-(5-fluoro-1H-indol-3-yl)-ethyl]-amide | | | |
| 273 | 5-(2,3-Difluoro-4-methyl-phenyl)-2-methyl- | (B) | 1 | 484.0 |
| | thiazole-4-carboxylic acid cyclopropylmethyl- | | | |
| | [2-(5-fluoro-1H-indol-3-yl)-ethyl]-amide | | | |
| 274 | 5-(3,4-Dimethyl-phenyl)-thiazole-4-carboxylic | (B) | 0.98 | 448.0 |
| | acid cyclopropylmethyl-[2-(5-fluoro-1H-indol- | | | |
| | 3-yl)-ethyl]-amide | | | |
| 275 | 2-Methoxy-5-m-tolyl-thiazole-4-carboxylic acid | (B) | 1.01 | 464.0 |
| | cyclopropylmethyl-[2-(5-fluoro-1H-indol-3-yl)- | | | |
| | ethyl]-amide | | | |
| 276 | 2-Cyclopropyl-5-(3-fluoro-4-methyl-phenyl)- | (B) | 1.04 | 492.0 |
| | thiazole-4-carboxylic acid cyclopropylmethyl- | | | |
| | [2-(5-fluoro-1H-indol-3-yl)-ethyl]-amide | | | |
| 277 | 2-Dimethylamino-5-m-tolyl-thiazole-4- | (B) | 0.99; | 477.1 |
| | carboxylic acid cyclopropylmethyl-[2-(5-fluoro- | | 1.03 | |
| | 1H-indol-3-yl)-ethyl]-amide | | | |
| 278 | 2-Dimethylamino-5-(3,4-dimethyl-phenyl)- | (B) | 1.02; | 491.0 |
| | thiazole-4-carboxylic acid cyclopropylmethyl- | | 1.05 | |
| | [2-(5-fluoro-1H-indol-3-yl)-ethyl]-amide | | | |
| 279 | 2-Dimethylaminomethyl-5-m-tolyl-thiazole-4- | (B) | 0.99 | 491.0 |
| | carboxylic acid cyclopropylmethyl-[2-(5-fluoro- | | | |
| | 1H-indol-3-yl)-ethyl]-amide | | | |
| 280 | 3-Phenyl-pyrazine-2-carboxylic acid | (B) | 0.89; | 414.9 |
| | cyclopropylmethyl-[2-(5-fluoro-1H-indol-3-yl)- | | 091 | |
| | ethyl]-amide | | | |
| 281 | 3-Phenyl-pyrazine-2-carboxylic acid [2-(5- | (B) | 0.81; | 375.1 |
| | fluoro-1H-indol-3-yl)-ethyl]-methyl-amide | | 0.83 | |
| 282 | 3-Phenyl-pyrazine-2-carboxylic acid ethyl-[2- | (B) | 0.84; | 389.0 |
| | (5-fluoro-1H-indol-3-yl)-ethyl]-amide | | 0.86 | |
| 283 | 3-Phenyl-pyrazine-2-carboxylic acid [2-(5- | (B) | 0.88; | 403.1 |
| | fluoro-1H-indol-3-yl)-ethyl]-propyl-amide | | 0.90 | |
| 284 | 3-Phenyl-pyrazine-2-carboxylic acid [2-(5- | (B) | 0.9 | 442.9 |
| | fluoro-1H-indol-3-yl)-ethyl]-(2,2,2-trifluoro- | | | |
| | ethyl)-amide | | | |
| 285 | 3-Phenyl-pyrazine-2-carboxylic acid | (B) | 0.72 | 417.9 |
| | carbamoylmethyl-[2-(5-fluoro-1H-indol-3-yl)- | | | |
| | ethyl]-amide | | | |
| 286 | [[2-(5-Fluoro-1H-indol-3-yl)-ethyl]-(3-phenyl- | (B) | 0.83 | 433.0 |
| | pyrazine-2-carbonyl)-amino]-acetic acid methyl | | | |
| | ester | | | |
| 287 | 3-Phenyl-pyrazine-2-carboxylic acid [2-(5- | (B) | 0.89 | 403.0 |
| | fluoro-1H-indol-3-yl)-ethyl]-isopropyl-amide | | | |
| 288 | 3-Phenyl-pyrazine-2-carboxylic acid (2,2- | (B) | 0.87 | 424.9 |
| | difluoro-ethyl)-[2-(5-fluoro-1H-indol-3-yl)- | | | |
| | ethyl]-amide | | | |
| 289 | 3-Phenyl-pyrazine-2-carboxylic acid [2-(5- | (B) | 0.74; | 405.0 |
| | fluoro-1H-indol-3-yl)-ethyl]-(2-hydroxy-ethyl)- | | 0.76 | |
| | amide | | | |
| 290 | 3-(3,4-Dimethyl-phenyl)-pyrazine-2-carboxylic | (B) | 0.87; | 403.0 |
| | acid [2-(5-fluoro-1H-indol-3-yl)-ethyl]-methyl- | | 0.89 | |
| | amide | | | |
| 291 | 3-(3,4-Dimethyl-phenyl)-pyrazine-2-carboxylic | (B) | 0.90; | 417.1 |
| | acid ethyl-[2-(5-fluoro-1H-indol-3-yl)-ethyl]- | | 0.92 | |
| | amide | | | |
| 292 | 3-(3,4-Dimethyl-phenyl)-pyrazine-2-carboxylic | (B) | 0.93; | 430.7 |
| | acid [2-(5-fluoro-1H-indol-3-yl)-ethyl]-propyl- | | 0.95 | |
| | amide | | | |
| 293 | 3-(3,4-Dimethyl-phenyl)-pyrazine-2-carboxylic | (B) | 0.95 | 471.0 |
| | acid [2-(5-fluoro-1H-indol-3-yl)-ethyl]-(2,2,2- | | | |
| | trifluoro-ethyl)-amide | | | |
| 294 | 3-(3,4-Dimethyl-phenyl)-pyrazine-2-carboxylic | (B) | 0.78 | 446.0 |
| | acid carbamoylmethyl-[2-(5-fluoro-1H-indol-3- | | | |
| | yl)-ethyl]-amide | | | |
| 295 | {[3-(3,4-Dimethyl-phenyl)-pyrazine-2- | (B) | 0.89 | 461.0 |
| | carbonyl]-[2-(5-fluoro-1H-indol-3-yl)-ethyl]- | | | |
| | amino}-acetic acid methyl ester | | | |
| 296 | 3-(3,4-Dimethyl-phenyl)-pyrazine-2-carboxylic | (B) | 0.95 | 430.8 |
| | acid [2-(5-fluoro-1H-indol-3-yl)-ethyl]- | | | |
| | isopropyl-amide | | | |
| 297 | 3-(3,4-Dimethyl-phenyl)-pyrazine-2-carboxylic | (B) | 0.93 | 452.9 |
| | acid (2,2-difluoro-ethyl)-[2-(5-fluoro-1H-indol- | | | |
| | 3-yl)-ethyl]-amide | | | |
| 298 | 5-(6-Methoxy-pyridin-3-yl)-2-methyl-thiazole- | (B) | 0.81 | 424.9 |
| | 4-carboxylic acid [2-(5-fluoro-1H-indol-3-yl)- | | | |
| | ethyl]-methyl-amide | | | |
| 299 | 5-(6-Methoxy-pyridin-3-yl)-2-methyl-thiazole- | (B) | 0.85 | 439.0 |
| | 4-carboxylic acid ethyl-[2-(5-fluoro-1H-indol-3- | | | |
| | yl)-ethyl]-amide | | | |
| 300 | 5-(6-Methoxy-pyridin-3-yl)-2-methyl-thiazole- | (B) | 0.89 | 452.8 |
| | 4-carboxylic acid [2-(5-fluoro-1H-indol-3-yl)- | | | |
| | ethyl]-propyl-amide | | | |
| 301 | 5-(6-Methoxy-pyridin-3-yl)-2-methyl-thiazole- | (B) | 0.93 | 492.9 |
| | 4-carboxylic acid [2-(5-fluoro-1H-indol-3-yl)- | | | |
| | ethyl]-(2,2,2-trifluoro-ethyl)-amide | | | |
| 302 | 5-(6-Methoxy-pyridin-3-yl)-2-methyl-thiazole- | (B) | 0.74 | 468.0 |
| | 4-carboxylic acid carbamoylmethyl-[2-(5- | | | |
| | fluoro-1H-indol-3-yl)-ethyl]-amide | | | |
| 303 | 5-(6-Methoxy-pyridin-3-yl)-2-methyl-thiazole- | (B) | 0.78 | 495.9 |
| | 4-carboxylic acid dimethylcarbamoylmethyl-[2- | | | |
| | (5-fluoro-1H-indol-3-yl)-ethyl]-amide | | | |
| 304 | 5-(6-Methoxy-pyridin-3-yl)-2-methyl-thiazole- | (B) | 0.83 | 481.9 |
| | 4-carboxylic acid (2-dimethylamino-ethyl)-[2- | | | |
| | (5-fluoro-1H-indol-3-yl)-ethyl]-amide | | | |
| 305 | {[2-(5-Fluoro-1H-indol-3-yl)-ethyl]-[5-(6- | (B) | 0.85 | 483.0 |
| | methoxy-pyridin-3-yl)-2-methyl-thiazole-4- | | | |
| | carbonyl]-amino}-acetic acid methyl ester | | | |
| 306 | 5-(6-Methoxy-pyridin-3-yl)-2-methyl-thiazole- | (B) | 0.9 | 453.0 |
| | 4-carboxylic acid [2-(5-fluoro-1H-indol-3-yl)- | | | |
| | ethyl]-isopropyl-amide | | | |
| 307 | 5-(6-Methoxy-pyridin-3-yl)-2-methyl-thiazole- | (B) | 0.9 | 474.9 |
| | 4-carboxylic acid (2,2-difluoro-ethyl)-[2-(5- | | | |
| | fluoro-1H-indol-3-yl)-ethyl]-amide | | | |
| 308 | 5-(6-Methoxy-pyridin-3-yl)-2-methyl-thiazole- | (B) | 0.76 | 454.9 |
| | 4-carboxylic acid [2-(5-fluoro-1H-indol-3-yl)- | | | |
| | ethyl]-(2-hydroxy-ethyl)-amide | | | |
| 309 | 6′-Methoxy-[3,3′]bipyridinyl-2-carboxylic acid | (B) | 0.78; | 405.1 |
| | [2-(5-fluoro-1H-indol-3-yl)-ethyl]-methyl- | | 0.79 | |
| | amide | | | |
| 310 | 6′-Methoxy-[3,3′]bipyridinyl-2-carboxylic acid | (B) | 0.80; | 419.0 |
| | ethyl-[2-(5-fluoro-1H-indol-3-yl)-ethyl]-amide | | 0.83 | |
| 311 | 6′-Methoxy-[3,3′]bipyridinyl-2-carboxylic acid | (B) | 0.84; | 433.0 |
| | [2-(5-fluoro-1H-indol-3-yl)-ethyl]-propyl-amide | | 0.86 | |
| 312 | 6′-Methoxy-[3,3′]bipyridinyl-2-carboxylic acid | (B) | 0.87 | 472.9 |
| | [2-(5-fluoro-1H-indol-3-yl)-ethyl]-(2,2,2- | | | |
| | trifluoro-ethyl)-amide | | | |
| 313 | 6′-Methoxy-[3,3′]bipyridinyl-2-carboxylic acid | (B) | 0.71 | 447.9 |
| | carbamoylmethyl-[2-(5-fluoro-1H-indol-3-yl)- | | | |
| | ethyl]-amide | | | |
| 314 | 6′-Methoxy-[3,3′]bipyridinyl-2-carboxylic acid | (B) | 0.75 | 476.0 |
| | dimethylcarbamoylmethyl-[2-(5-fluoro-1H- | | | |
| | indol-3-yl)-ethyl]-amide | | | |
| 315 | [[2-(5-Fluoro-1H-indol-3-yl)-ethyl]-(6′- | (B) | 0.81 | 463.0 |
| | methoxy-[3,3′]bipyridinyl-2-carbonyl)-amino]- | | | |
| | acetic acid methyl ester | | | |
| 316 | 6′-Methoxy-[3,3′]bipyridinyl-2-carboxylic acid | (B) | 0.85 | 433.0 |
| | [2-(5-fluoro-1H-indol-3-yl)-ethyl]-isopropyl- | | | |
| | amide | | | |
| 317 | 6′-Methoxy-[3,3′]bipyridinyl-2-carboxylic acid | (B) | 0.85 | 454.9 |
| | (2,2-difluoro-ethyl)-[2-(5-fluoro-1H-indol-3-yl)- | | | |
| | ethyl]-amide | | | |
| 318 | 6′-Methoxy-[3,3′]bipyridinyl-2-carboxylic acid | (B) | 0.73 | 435.0 |
| | [2-(5-fluoro-1H-indol-3-yl)-ethyl]-(2-hydroxy- | | | |
| | ethyl)-amide | | | |
| 319 | 3-Phenyl-pyrazine-2-carboxylic acid | (C) | 0.85; | 411.1 |
| | cyclopropylmethyl-[2-(1-methyl-1H-indol-3- | | 0.87 | |
| | yl)-ethyl]-amide | | | |
| 320 | 3-Phenyl-pyrazine-2-carboxylic acid [2-(6- | (C) | 0.83; | 431.1 |
| | chloro-1H-indol-3-yl)-ethyl]- | | 0.85 | |
| | cyclopropylmethyl-amide | | | |
| 321 | 3-Phenyl-pyrazine-2-carboxylic acid | (C) | 0.78; | 427.1 |
| | cyclopropylmethyl-[2-(7-methoxy-1H-indol-3- | | 0.80 | |
| | yl)-ethyl]-amide | | | |
| 322 | 3-Phenyl-pyrazine-2-carboxylic acid | (C) | 0.74; | 427.1 |
| | cyclopropylmethyl-[2-(5-methoxy-1H-indol-3- | | 0.76 | |
| | yl)-ethyl]-amide | | | |
| 323 | 3-Phenyl-pyrazine-2-carboxylic acid | (C) | 0.75; | 427.1 |
| | cyclopropylmethyl-[2-(6-methoxy-1H-indol-3- | | 0.77 | |
| | yl)-ethyl]-amide | | | |
| 324 | 3-Phenyl-pyrazine-2-carboxylic acid | (C) | 0.81; | 411.1 |
| | cyclopropylmethyl-[2-(5-methyl-1H-indol-3- | | 0.83 | |
| | yl)-ethyl]-amide | | | |
| 325 | 3-Phenyl-pyrazine-2-carboxylic acid | (C) | 0.81; | 411.1 |
| | cyclopropylmethyl-[2-(6-methyl-1H-indol-3- | | 0.83 | |
| | yl)-ethyl]-amide | | | |
| 326 | 3-Phenyl-pyrazine-2-carboxylic acid | (C) | 0.81; | 411.1 |
| | cyclopropylmethyl-[2-(7-methyl-1H-indol-3- | | 0.83 | |
| | yl)-ethyl]-amide | | | |
| 327 | 3-Phenyl-pyrazine-2-carboxylic acid | (C) | 0.78; | 415.1 |
| | cyclopropylmethyl-[2-(4-fluoro-1H-indol-3-yl)- | | 0.81 | |
| | ethyl]-amide | | | |
| 328 | 3-Phenyl-pyrazine-2-carboxylic acid | (C) | 0.78; | 415.1 |
| | cyclopropylmethyl-[2-(6-fluoro-1H-indol-3-yl)- | | 0.80 | |
| | ethyl]-amide | | | |
| 329 | 3-Phenyl-pyrazine-2-carboxylic acid | (C) | 0.79; | 415.2 |
| | cyclopropylmethyl-[2-(7-fluoro-1H-indol-3-yl)- | | 0.81 | |
| | ethyl]-amide | | | |
| 330 | 3-(3,4-Dimethyl-phenyl)-pyrazine-2-carboxylic | (C) | 0.92; | 439.1 |
| | acid cyclopropylmethyl-[2-(1-methyl-1H-indol- | | 0.94 | |
| | 3-yl)-ethyl]-amide | | | |
| 331 | 3-(3,4-Dimethyl-phenyl)-pyrazine-2-carboxylic | (C) | 0.90; | 459.1 |
| | acid [2-(6-chloro-1H-indol-3-yl)-ethyl]- | | 0.91 | |
| | cyclopropylmethyl-amide | | | |
| 332 | 3-(3,4-Dimethyl-phenyl)-pyrazine-2-carboxylic | (C) | 0.85; | 455.1 |
| | acid cyclopropylmethyl-[2-(7-methoxy-1H- | | 0.87 | |
| | indol-3-yl)-ethyl]-amide | | | |
| 333 | 3-(3,4-Dimethyl-phenyl)-pyrazine-2-carboxylic | (C) | 0.81; | 455.2 |
| | acid cyclopropylmethyl-[2-(5-methoxy-1H- | | 0.83 | |
| | indol-3-yl)-ethyl]-amide | | | |
| 334 | 3-(3,4-Dimethyl-phenyl)-pyrazine-2-carboxylic | (C) | 0.82; | 455.2 |
| | acid cyclopropylmethyl-[2-(6-methoxy-1H- | | 0.84 | |
| | indol-3-yl)-ethyl]-amide | | | |
| 335 | 3-(3,4-Dimethyl-phenyl)-pyrazine-2-carboxylic | (C) | 0.88; | 439.2 |
| | acid cyclopropylmethyl-[2-(5-methyl-1H-indol- | | 0.90 | |
| | 3-yl)-ethyl]-amide | | | |
| 336 | 3-(3,4-Dimethyl-phenyl)-pyrazine-2-carboxylic | (C) | 0.88; | 439.2 |
| | acid cyclopropylmethyl-[2-(6-methyl-1H-indol- | | 0.90 | |
| | 3-yl)-ethyl]-amide | | | |
| 337 | 3-(3,4-Dimethyl-phenyl)-pyrazine-2-carboxylic | (C) | 0.88; | 439.2 |
| | acid cyclopropylmethyl-[2-(7-methyl-1H-indol- | | 0.90 | |
| | 3-yl)-ethyl]-amide | | | |
| 338 | 3-(3,4-Dimethyl-phenyl)-pyrazine-2-carboxylic | (C) | 0.86; | 443.1 |
| | acid cyclopropylmethyl-[2-(4-fluoro-1H-indol- | | 0.88 | |
| | 3-yl)-ethyl]-amide | | | |
| 339 | 3-(3,4-Dimethyl-phenyl)-pyrazine-2-carboxylic | (C) | 0.85; | 443.2 |
| | acid cyclopropylmethyl-[2-(6-fluoro-1H-indol- | | 0.87 | |
| | 3-yl)-ethyl]-amide | | | |
| 340 | 3-(3,4-Dimethyl-phenyl)-pyrazine-2-carboxylic | (C) | 0.86; | 443.1 |
| | acid cyclopropylmethyl-[2-(7-fluoro-1H-indol- | | 0.88 | |
| | 3-yl)-ethyl]-amide | | | |
| 341 | 5-(6-Methoxy-pyridin-3-yl)-2-methyl-thiazole- | (C) | 0.88 | 461.1 |
| | 4-carboxylic acid cyclopropylmethyl-[2-(1- | | | |
| | methyl-1H-indol-3-yl)-ethyl]-amide | | | |
| 342 | 5-(6-Methoxy-pyridin-3-yl)-2-methyl-thiazole- | (C) | 0.86 | 481.0 |
| | 4-carboxylic acid [2-(6-chloro-1H-indol-3-yl)- | | | |
| | ethyl]-cyclopropylmethyl-amide | | | |
| 343 | 5-(6-Methoxy-pyridin-3-yl)-2-methyl-thiazole- | (C) | 0.81 | 477.1 |
| | 4-carboxylic acid cyclopropylmethyl-[2-(7- | | | |
| | methoxy-1H-indol-3-yl)-ethyl]-amide | | | |
| 344 | 5-(6-Methoxy-pyridin-3-yl)-2-methyl-thiazole- | (C) | 0.77 | 477.1 |
| | 4-carboxylic acid cyclopropylmethyl-[2-(5- | | | |
| | methoxy-1H-indol-3-yl)-ethyl]-amide | | | |
| 345 | 5-(6-Methoxy-pyridin-3-yl)-2-methyl-thiazole- | (C) | 0.77 | 477.1 |
| | 4-carboxylic acid cyclopropylmethyl-[2-(6- | | | |
| | methoxy-1H-indol-3-yl)-ethyl]-amide | | | |
| 346 | 5-(6-Methoxy-pyridin-3-yl)-2-methyl-thiazole- | (C) | 0.84 | 461.1 |
| | 4-carboxylic acid cyclopropylmethyl-[2-(5- | | | |
| | methyl-1H-indol-3-yl)-ethyl]-amide | | | |
| 347 | 5-(6-Methoxy-pyridin-3-yl)-2-methyl-thiazole- | (C) | 0.84 | 461.1 |
| | 4-carboxylic acid cyclopropylmethyl-[2-(6- | | | |
| | methyl-1H-indol-3-yl)-ethyl]-amide | | | |
| 348 | 5-(6-Methoxy-pyridin-3-yl)-2-methyl-thiazole- | (C) | 0.84 | 461.1 |
| | 4-carboxylic acid cyclopropylmethyl-[2-(7- | | | |
| | methyl-1H-indol-3-yl)-ethyl]-amide | | | |
| 349 | 5-(6-Methoxy-pyridin-3-yl)-2-methyl-thiazole- | (C) | 0.81 | 465.1 |
| | 4-carboxylic acid cyclopropylmethyl-[2-(4- | | | |
| | fluoro-1H-indol-3-yl)-ethyl]-amide | | | |
| 350 | 5-(6-Methoxy-pyridin-3-yl)-2-methyl-thiazole- | (C) | 0.81 | 465.0 |
| | 4-carboxylic acid cyclopropylmethyl-[2-(6- | | | |
| | fluoro-1H-indol-3-yl)-ethyl]-amide | | | |
| 351 | 5-(6-Methoxy-pyridin-3-yl)-2-methyl-thiazole- | (C) | 0.82 | 465.0 |
| | 4-carboxylic acid cyclopropylmethyl-[2-(7- | | | |
| | fluoro-1H-indol-3-yl)-ethyl]-amide | | | |
| 352 | 6′-Methoxy-[3,3′]bipyridinyl-2-carboxylic acid | (C) | 0.80; | 441.1 |
| | cyclopropylmethyl-[2-(1-methyl-1H-indol-3- | | 0.82 | |
| | yl)-ethyl]-amide | | | |
| 353 | 6′-Methoxy-[3,3′]bipyridinyl-2-carboxylic acid | (C) | 0.78; | 461.1 |
| | [2-(6-chloro-1H-indol-3-yl)-ethyl]- | | 0.81 | |
| | cyclopropylmethyl-amide | | | |
| 354 | 6′-Methoxy-[3,3′]bipyridinyl-2-carboxylic acid | (C) | 0.73; | 457.1 |
| | cyclopropylmethyl-[2-(7-methoxy-1H-indol-3- | | 0.76 | |
| | yl)-ethyl]-amide | | | |
| 355 | 6′-Methoxy-[3,3′]bipyridinyl-2-carboxylic acid | (C) | 0.69; | 457.2 |
| | cyclopropylmethyl-[2-(5-methoxy-1H-indol-3- | | 0.72 | |
| | yl)-ethyl]-amide | | | |
| 356 | 6′-Methoxy-[3,3′]bipyridinyl-2-carboxylic acid | (C) | 0.70; | 457.1 |
| | cyclopropylmethyl-[2-(6-methoxy-1H-indol-3- | | 0.73 | |
| | yl)-ethyl]-amide | | | |
| 357 | 6′-Methoxy-[3,3′]bipyridinyl-2-carboxylic acid | (C) | 0.75; | 441.1 |
| | cyclopropylmethyl-[2-(5-methyl-1H-indol-3- | | 0.79 | |
| | yl)-ethyl]-amide | | | |
| 358 | 6′-Methoxy-[3,3′]bipyridinyl-2-carboxylic acid | (C) | 0.76; | 441.1 |
| | cyclopropylmethyl-[2-(6-methyl-1H-indol-3- | | 0.79 | |
| | yl)-ethyl]-amide | | | |
| 359 | 6′-Methoxy-[3,3′]bipyridinyl-2-carboxylic acid | (C) | 0.76; | 441.2 |
| | cyclopropylmethyl-[2-(7-methyl-1H-indol-3- | | 0.79 | |
| | yl)-ethyl]-amide | | | |
| 360 | 6′-Methoxy-[3,3′]bipyridinyl-2-carboxylic acid | (C) | 0.74; | 445.1 |
| | cyclopropylmethyl-[2-(4-fluoro-1H-indol-3-yl)- | | 0.77 | |
| | ethyl]-amide | | | |
| 361 | 6′-Methoxy-[3,3′]bipyridinyl-2-carboxylic acid | (C) | 0.74, | 445.1 |
| | cyclopropylmethyl-[2-(6-fluoro-1H-indol-3-yl)- | | 0.76 | |
| | ethyl]-amide | | | |
| 362 | 6′-Methoxy-[3,3′]bipyridinyl-2-carboxylic acid | (C) | 0.74; | 445.1 |
| | cyclopropylmethyl-[2-(7-fluoro-1H-indol-3-yl)- | | 0.77 | |
| | ethyl]-amide | | | |
| 363 | 3-Phenyl-pyrazine-2-carboxylic acid | (C) | 0.51 | 428.1 |
| | cyclopropylmethyl-[2-(6-methoxy-1H- | | | |
| | benzoimidazol-2-yl)-ethyl]-amide | | | |
| 364 | 3-m-Tolyl-pyrazine-2-carboxylic acid | (C) | 0.56 | 442.2 |
| | cyclopropylmethyl-[2-(6-methoxy-1H- | | | |
| | benzoimidazol-2-yl)-ethyl]-amide | | | |
| 365 | 3-(3,4-Dimethyl-phenyl)-pyrazine-2-carboxylic | (C) | 0.59 | 456.1 |
| | acid cyclopropylmethyl-[2-(6-methoxy-1H- | | | |
| | benzoimidazol-2-yl)-ethyl]-amide | | | |
| 366 | 2-Methyl-5-m-tolyl-thiazole-4-carboxylic acid | (C) | 0.62 | 461.1 |
| | cyclopropylmethyl-[2-(6-methoxy-1H- | | | |
| | benzoimidazol-2-yl)-ethyl]-amide | | | |
| 367 | 2-Dimethylamino-5-(3-methoxy-phenyl)- | (C) | 0.63 | 506.1 |
| | thiazole-4-carboxylic acid cyclopropylmethyl- | | | |
| | [2-(6-methoxy-1H-benzoimidazol-2-yl)-ethyl]- | | | |
| | amide | | | |
| 368 | 2-Dimethylamino-5-(3,4-dimethyl-phenyl)- | (C) | 0.69 | 504.2 |
| | thiazole-4-carboxylic acid cyclopropylmethyl- | | | |
| | [2-(6-methoxy-1H-benzoimidazol-2-yl)-ethyl]- | | | |
| | amide | | | |
| 369 | 2-Dimethylamino-5-m-tolyl-thiazole-4- | (C) | 0.66 | 490.1 |
| | carboxylic acid cyclopropylmethyl-[2-(6- | | | |
| | methoxy-1H-benzoimidazol-2-yl)-ethyl]-amide | | | |
| 370 | 5-(6-Methoxy-pyridin-3-yl)-2-methyl-thiazole- | (C) | 0.55 | 478.1 |
| | 4-carboxylic acid cyclopropylmethyl-[2-(6- | | | |
| | methoxy-1H-benzoimidazol-2-yl)-ethyl]-amide | | | |
| 371 | 6′-Methoxy-[3,3′]bipyridinyl-2-carboxylic acid | (C) | 0.52 | 458.2 |
| | cyclopropylmethyl-[2-(6-methoxy-1H- | | | |
| | benzoimidazol-2-yl)-ethyl]-amide | | | |
| 372 | 3-Phenyl-pyrazine-2-carboxylic acid | (C) | 0.57 | 426.1 |
| | cyclopropylmethyl-[2-(5,6-dimethyl-1H- | | | |
| | benzoimidazol-2-yl)-ethyl]-amide | | | |
| 373 | 3-m-Tolyl-pyrazine-2-carboxylic acid | (C) | 0.6 | 440.2 |
| | cyclopropylmethyl-[2-(5,6-dimethyl-1H- | | | |
| | benzoimidazol-2-yl)-ethyl]-amide | | | |
| 374 | 3-(3,4-Dimethyl-phenyl)-pyrazine-2-carboxylic | (C) | 0.63 | 454.2 |
| | acid cyclopropylmethyl-[2-(5,6-dimethyl-1H- | | | |
| | benzoimidazol-2-yl)-ethyl]-amide | | | |
| 375 | 2-Methyl-5-m-tolyl-thiazole-4-carboxylic acid | (C) | 0.66 | 459.2 |
| | cyclopropylmethyl-[2-(5,6-dimethyl-1H- | | | |
| | benzoimidazol-2-yl)-ethyl]-amide | | | |
| 376 | 2-Dimethylamino-5-(3-methoxy-phenyl)- | (C) | 0.67 | 504.1 |
| | thiazole-4-carboxylic acid cyclopropylmethyl- | | | |
| | [2-(5,6-dimethyl-1H-benzoimidazol-2-yl)- | | | |
| | ethyl]-amide | | | |
| 377 | 2-Dimethylamino-5-(3,4-dimethyl-phenyl)- | (C) | 0.72 | 502.1 |
| | thiazole-4-carboxylic acid cyclopropylmethyl- | | | |
| | [2-(5,6-dimethyl-1H-benzoimidazol-2-yl)- | | | |
| | ethyl]-amide | | | |
| 378 | 2-Dimethylamino-5-m-tolyl-thiazole-4- | (C) | 0.69 | 488.1 |
| | carboxylic acid cyclopropylmethyl-[2-(5,6- | | | |
| | dimethyl-1H-benzoimidazol-2-yl)-ethyl]-amide | | | |
| 379 | 5-(6-Methoxy-pyridin-3-yl)-2-methyl-thiazole- | (C) | 0.6 | 476.1 |
| | 4-carboxylic acid cyclopropylmethyl-[2-(5,6- | | | |
| | dimethyl-1H-benzoimidazol-2-yl)-ethyl]-amide | | | |
| 380 | 6′-Methoxy-[3,3′]bipyridinyl-2-carboxylic acid | (C) | 0.56 | 456.2 |
| | cyclopropylmethyl-[2-(5,6-dimethyl-1H- | | | |
| | benzoimidazol-2-yl)-ethyl]-amide | | | |
| 381 | 5-(6-Methoxy-pyridin-3-yl)-2-methyl-thiazole- | (C) | 0.80 | 491.0 |
| | 4-carboxylic acid cyclopropylmethyl-[2-(5- | | | |
| | methoxy-4-methyl-1H-indol-3-yl)-ethyl]-amide | | | |
| 382 | 5-(6-Methoxy-pyridin-3-yl)-2-methyl-thiazole- | (C) | 0.75 | 491.1 |
| | 4-carboxylic acid cyclopropylmethyl-[2-(5H- | | | |
| | [1,3]dioxolo[4,5-f]indol-7-yl)-ethyl]-amide | | | |
| 383 | 5-(6-Methoxy-pyridin-3-yl)-2-methyl-thiazole- | (C) | 0.82 | 483.0 |
| | 4-carboxylic acid cyclopropylmethyl-[2-(5,6- | | | |
| | difluoro-1H-indol-3-yl)-ethyl]-amide | | | |
| 384 | 5-(6-Methoxy-pyridin-3-yl)-2-methyl-thiazole- | (C) | 0.85 | 498.9 |
| | 4-carboxylic acid [2-(5-chloro-6-fluoro-1H- | | | |
| | indol-3-yl)-ethyl]-cyclopropylmethyl-amide | | | |
| 385 | rac-5-(6-Methoxy-pyridin-3-yl)-2-methyl- | (C) | 0.78 | 491.1 |
| | thiazole-4-carboxylic acid cyclopropylmethyl- | | | |
| | [2-(5-methoxy-1H-indol-3-yl)-1-methyl-ethyl]- | | | |
| | amide | | | |

### B.3 Synthesis of Compounds of Formula (I) by Suzuki Reaction (General Procedure)

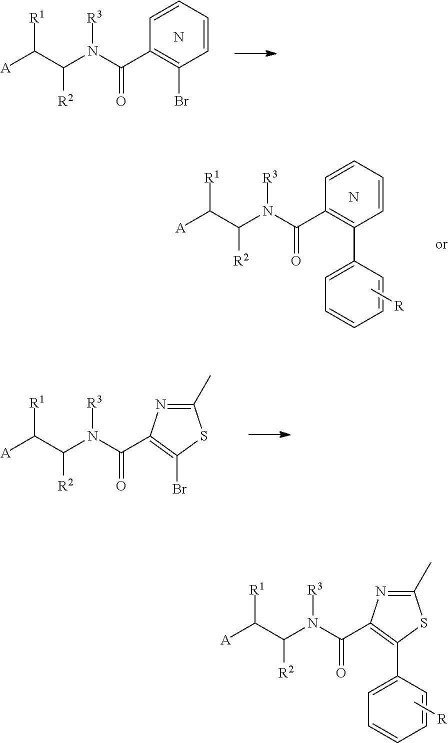

A mixture of the respective bromo-heterocyclyl-carboxylic amide derivative (0.029 mmol) and the respective boronic acid derivative (1.0-1.2 eq) is dissolved (or suspended) in a mixture of toluene (0.20 mL) and EtOH (0.20 mL). A freshly prepared aqueous Na₂CO₃ solution (2.0 M, 0.30 mL) is added and argon is passed through the mixture to remove oxygen. Tetrakis(triphenylphosphine)palladium(0) (1.05 mg) is added under argon and the mixture is vigorously stirred at around 75° C. until LC-MS indicated complete reaction (45 to 300 min). DMF (1.0 mL) is added and the mixture is purified by prep. HPLC (basic conditions) to give the desired product.

prepared by reaction of 3-bromo-N-cyclopropylmethyl-N-[2-(3,4-dimethoxy-phenyl)-ethyl]-isonicotinamide with arylboronic acid derivatives

| | | LC-MS | | |
|---|---|---|---|---|
| | | | t_{R} | |
| Example | Name | method | [min] | [M + H]⁺ |
| 386 | N-Cyclopropylmethyl-N-[2- | (B) | 0.89 | 431.2 |
| | (3,4-dimethoxy-phenyl)-ethyl]- | | | |
| | 3-m-tolyl-isonicotinamide | | | |
| 387 | N-Cyclopropylmethyl-N-[2- | (B) | 0.89 | 431.2 |
| | (3,4-dimethoxy-phenyl)-ethyl]- | | | |
| | 3-p-tolyl-isonicotinamide | | | |
| 388 | N-Cyclopropylmethyl-N-[2- | (B) | 0.91 | 445.2 |
| | (3,4-dimethoxy-phenyl)-ethyl]- | | | |
| | 3-(3,4-dimethyl-phenyl)- | | | |
| | isonicotinamide | | | |
| 389 | N-Cyclopropylmethyl-N-[2-(3,4- | (B) | 0.85 | 447.1 |
| | dimethoxy-phenyl)-ethyl]-3- | | | |
| | (3-methoxy-phenyl)- | | | |
| | isonicotinamide | | | |

prepared by reaction of 3-bromo-pyridine-2-carboxylic acid cyclopropylmethyl-[2-(3,4-dimethoxy-phenyl)-ethyl]-amide with arylboronic acid derivatives

| | | LC-MS | | |
|---|---|---|---|---|
| | | | t_{R} | |
| Example | Name | method | [min] | [M + H]⁺ |
| 390 | 3-m-Tolyl-pyridine-2-carboxylic | (B) | 0.93 | 431.1 |
| | acid cyclopropylmethyl-[2-(3,4- | | | |
| | dimethoxy-phenyl)-ethyl]-amide | | | |
| 391 | 3-p-Tolyl-pyridine-2-carboxylic | (B) | 0.93 | 431.2 |
| | acid cyclopropylmethyl-[2-(3,4- | | | |
| | dimethoxy-phenyl)-dimethoxy- | | | |
| | phenyl)-ethyl]-amide | | | |
| 392 | 3-(3,4-Dimethyl-phenyl)- | (B) | 0.95 | 445.2 |
| | pyridine-2-carboxylic acid | | | |
| | cyclopropyl-methyl-[2- | | | |
| | (3,4-dimethoxy-phenyl)- | | | |
| | ethyl]-amide | | | |
| 393 | 3-(3-Methoxy-phenyl)-pyridine- | (B) | 0.88 | 447.2 |
| | 2-carboxylic acid cyclopropyl- | | | |
| | methyl-[2-(3,4-dimethoxy- | | | |
| | phenyl)-ethyl]-amide | | | |

prepared by reaction of 2-bromo-N-cyclopropylmethyl-N-[2-(3,4-dimethoxy-phenyl)-ethyl]-nicotinamide with arylboronic acid derivatives

| | | LC-MS | | |
|---|---|---|---|---|
| Example | Name | method | t_{R} [min] | [M + H]⁺ |
| 394 | N-Cyclopropylmethyl-N-[2-(3,4-dimethoxy- | (B) | 0.89 | 431.2 |
| | phenyl)-ethyl]-2-m-tolyl-nicotinamide | | | |
| 395 | N-Cyclopropylmethyl-N-[2-(3,4-dimethoxy- | (B) | 0.89 | 431.2 |
| | phenyl)-ethyl]-2-p-tolyl-nicotinamide | | | |
| 396 | N-Cyclopropylmethyl-N-[2-(3,4-dimethoxy- | (B) | 0.92 | 445.2 |
| | phenyl)-ethyl]-2-(3,4-dimethyl-phenyl)- | | | |
| | nicotinamide | | | |
| 397 | N-Cyclopropylmethyl-N-[2-(3,4-dimethoxy- | (B) | 0.85 | 447.2 |
| | phenyl)-ethyl]-2-(3-methoxy-phenyl)- | | | |
| | nicotinamide | | | |

prepared by reaction of 3-bromo-pyridine-2-carboxylic acid cyclopropylmethyl-[2-(5-fluoro-1H-indol-3-yl)-ethyl]-amide with boronic acid derivatives

| | | LC-MS | | |
|---|---|---|---|---|
| Example | Name | method | t_{R} [min] | [M + H]⁺ |
| 398 | 3-m-Tolyl-pyridine-2-carboxylic acid | (C) | 0.82; | 428.3 |
| | cyclopropylmethyl-[2-(5-fluoro-1H-indol-3-yl)- | | 0.85 | |
| | ethyl]-amide | | | |
| 399 | 3-(3,4-Dimethyl-phenyl)-pyridine-2-carboxylic | (C) | 0.85; | 442.2 |
| | acid cyclopropylmethyl-[2-(5-fluoro-1H-indol- | | 0.88 | |
| | 3-yl)-ethyl]-amide | | | |
| 400 | 6′-Methoxy-[3,3′]bipyridinyl-2-carboxylic acid | (C) | 0.73; | 445.2 |
| | cyclopropylmethyl-[2-(5-fluoro-1H-indol-3-yl)- | | 0.76 | |
| | ethyl]-amide | | | |
| 401 | 6′-Fluoro-[3,3′]bipyridinyl-2-carboxylic acid | (C) | 0.72; | 433.1 |
| | cyclopropylmethyl-[2-(5-fluoro-1H-indol-3-yl)- | | 0.74 | |
| | ethyl]-amide | | | |
| 402 | 5′-Methyl-[3,3′]bipyridinyl-2-carboxylic acid | (C) | 0.57; | 429.2 |
| | cyclopropylmethyl-[2-(5-fluoro-1H-indol-3-yl)- | | 0.59 | |
| | ethyl]-amide | | | |
| 403 | 5′-Chloro-2′-fluoro-[3,3′]bipyridinyl-2- | (C) | 0.80; | 467.1 |
| | carboxylic acid cyclopropylmethyl-[2-(5-fluoro- | | 0.82 | |
| | 1H-indol-3-yl)-ethyl]-amide | | | |
| 404 | 3-Quinolin-3-yl-pyridine-2-carboxylic acid | (C) | 0.68; | 465.2 |
| | cyclopropylmethyl-[2-(5-fluoro-1H-indol-3-yl)- | | 0.71 | |
| | ethyl]-amide | | | |
| 405 | 6′-Methyl-[3,3′]bipyridinyl-2-carboxylic acid | (C) | 0.55; | 429.2 |
| | cyclopropylmethyl-[2-(5-fluoro-1H-indol-3-yl)- | | 0.57 | |
| | ethyl]-amide | | | |
| 406 | 5′-Methoxy-[3,3′]bipyridinyl-2-carboxylic acid | (C) | 0.62; | 445.2 |
| | cyclopropylmethyl-[2-(5-fluoro-1H-indol-3-yl)- | | 0.65 | |
| | ethyl]-amide | | | |

prepared by reaction of 5-bromo-2-methyl-thiazole-4-carboxylic acid cyclopropyl-methyl-[2-(5-fluoro-1H-indol-3-yl)-ethyl]-amide with boronic acid derivatives

| | | LC-MS | | |
|---|---|---|---|---|
| Example | Name | method | t_{R} [min] | [M + H]⁺ |
| 407 | 5-(3-Chloro-4-methyl-phenyl)-2-methyl- | (C) | 0.95 | 481.7 |
| | thiazole-4-carboxylic acid cyclopropylmethyl- | | | |
| | [2-(5-fluoro-1H-indol-3-yl)-ethyl]-amide | | | |
| 408 | 5-(3-Chloro-4-methoxy-phenyl)-2-methyl- | (C) | 0.88 | 497.7 |
| | thiazole-4-carboxylic acid cyclopropylmethyl- | | | |
| | [2-(5-fluoro-1H-indol-3-yl)-ethyl]-amide | | | |
| 409 | 2-Methyl-5-(6-methyl-pyridin-3-yl)-thiazole-4- | (C) | 0.59 | 449.3 |
| | carboxylic acid cyclopropylmethyl-[2-(5-fluoro- | | | |
| | 1H-indol-3-yl)-ethyl]-amide | | | |
| 410 | 5-(4-Methoxy-3-methyl-phenyl)-2-methyl- | (C) | 0.89 | 478.2 |
| | thiazole-4-carboxylic acid cyclopropylmethyl- | | | |
| | [2-(5-fluoro-1H-indol-3-yl)-ethyl]-amide | | | |
| 411 | 5-(3-Chloro-4-fluoro-phenyl)-2-methyl- | (C) | 0.91 | 486.1 |
| | thiazole-4-carboxylic acid cyclopropylmethyl- | | | |
| | [2-(5-fluoro-1H-indol-3-yl)-ethyl]-amide | | | |
| 412 | 5-(4-Fluoro-3-methyl-phenyl)-2-methyl- | (C) | 0.9 | 465.7 |
| | thiazole-4-carboxylic acid cyclopropylmethyl- | | | |
| | [2-(5-fluoro-1H-indol-3-yl)-ethyl]-amide | | | |
| 413 | 5-(3-Fluoro-4-methoxy-phenyl)-2-methyl- | (C) | 0.84 | 481.7 |
| | thiazole-4-carboxylic acid cyclopropylmethyl- | | | |
| | [2-(5-fluoro-1H-indol-3-yl)-ethyl]-amide | | | |
| 414 | 5-(4-Chloro-3-fluoro-phenyl)-2-methyl- | (C) | 0.92 | 486.1 |
| | thiazole-4-carboxylic acid cyclopropylmethyl- | | | |
| | [2-(5-fluoro-1H-indol-3-yl)-ethyl]-amide | | | |
| 415 | 5-(3-Cyano-4-fluoro-phenyl)-2-methyl-thiazole- | (C) | 0.83 | 477.1 |
| | 4-carboxylic acid cyclopropylmethyl-[2-(5- | | | |
| | fluoro-1H-indol-3-yl)-ethyl]-amide | | | |
| 416 | 5-(4-Fluoro-3-methoxy-phenyl)-2-methyl- | (C) | 0.86 | 481.7 |
| | thiazole-4-carboxylic acid cyclopropylmethyl- | | | |
| | [2-(5-fluoro-1H-indol-3-yl)-ethyl]-amide | | | |
| 417 | 5-(4-Chloro-3-cyano-phenyl)-2-methyl-thiazole- | (C) | 0.86 | 493.2 |
| | 4-carboxylic acid cyclopropylmethyl-[2-(5- | | | |
| | fluoro-1H-indol-3-yl)-ethyl]-amide | | | |
| 418 | 5-(4-Fluoro-3-hydroxymethyl-phenyl)-2- | (C) | 0.75 | 481.7 |
| | methyl-thiazole-4-carboxylic acid | | | |
| | cyclopropylmethyl-[2-(5-fluoro-1H-indol-3-yl)- | | | |
| | ethyl]-amide | | | |
| 419 | 5-(4-Cyano-3-fluoro-phenyl)-2-methyl-thiazole- | (C) | 0.84 | 477.2 |
| | 4-carboxylic acid cyclopropylmethyl-[2-(5- | | | |
| | fluoro-1H-indol-3-yl)-ethyl]-amide | | | |
| 420 | 5-(3-Chloro-2-methoxy-pyridin-4-yl)-2-methyl- | (C) | 0.87 | 499.1 |
| | thiazole-4-carboxylic acid cyclopropylmethyl- | | | |
| | [2-(5-fluoro-1H-indol-3-yl)-ethyl]-amide | | | |
| 421 | 5-(6-Methoxy-pyridin-3-yl)-2-methyl-thiazole- | (C) | 0.81 | 465.2 |
| | 4-carboxylic acid cyclopropylmethyl-[2-(5- | | | |
| | fluoro-1H-indol-3-yl)-ethyl]-amide | | | |
| 422 | 5-(6-Fluoro-pyridin-3-yl)-2-methyl-thiazole-4- | (C) | 0.78 | 453.2 |
| | carboxylic acid cyclopropylmethyl-[2-(5-fluoro- | | | |
| | 1H-indol-3-yl)-ethyl]-amide | | | |
| 423 | 5-(6-Hydroxymethyl-pyridin-3-yl)-2-methyl- | (C) | 0.58 | 465.1 |
| | thiazole-4-carboxylic acid cyclopropylmethyl- | | | |
| | [2-(5-fluoro-1H-indol-3-yl)-ethyl]-amide | | | |
| 424 | 2-Methyl-5-(5-methylsulfanyl-pyridin-3-yl)- | (C) | 0.77 | 481.2 |
| | thiazole-4-carboxylic acid cyclopropylmethyl- | | | |
| | [2-(5-fluoro-1H-indol-3-yl)-ethyl]-amide | | | |
| 425 | 5-(5-Fluoro-pyridin-3-yl)-2-methyl-thiazole-4- | (C) | 0.76 | 453.1 |
| | carboxylic acid cyclopropylmethyl-[2-(5-fluoro- | | | |
| | 1H-indol-3-yl)-ethyl]-amide | | | |
| 426 | 2-Methyl-5-(5-methyl-pyridin-3-yl)-thiazole-4- | (C) | 0.61 | 449.2 |
| | carboxylic acid cyclopropylmethyl-[2-(5-fluoro- | | | |
| | 1H-indol-3-yl)-ethyl]-amide | | | |
| 427 | 5-(5-Chloro-2-fluoro-pyridin-3-yl)-2-methyl- | (C) | 0.86 | 487.1 |
| | thiazole-4-carboxylic acid cyclopropylmethyl- | | | |
| | [2-(5-fluoro-1H-indol-3-yl)-ethyl]-amide | | | |
| 428 | 2-Methyl-5-quinolin-3-yl-thiazole-4-carboxylic | (C) | 0.77 | 485.2 |
| | acid cyclopropylmethyl-[2-(5-fluoro-1H-indol- | | | |
| | 3-yl)-ethyl]-amide | | | |
| 429 | 5-(1H-Indol-5-yl)-2-methyl-thiazole-4- | (C) | 0.77 | 473.2 |
| | carboxylic acid cyclopropylmethyl-[2-(5-fluoro- | | | |
| | 1H-indol-3-yl)-ethyl]-amide | | | |
| 430 | 5-(1H-Indol-6-yl)-2-methyl-thiazole-4- | (C) | 0.8 | 473.2 |
| | carboxylic acid cyclopropylmethyl-[2-(5-fluoro- | | | |
| | 1H-indol-3-yl)-ethyl]-amide | | | |
| 431 | 2-Methyl-5-(1-methyl-1H-indol-2-yl)-thiazole- | (C) | 0.9 | 487.2 |
| | 4-carboxylic acid cyclopropylmethyl-[2-(5- | | | |
| | fluoro-1H-indol-3-yl)-ethyl]-amide | | | |

B.4 Synthesis of 2-Methyl-5-m-tolyl-thiazole-4-carboxylic acid [2-cyclopropyl-amino-2-(3,4-dimethoxy-phenyl)-ethyl]-cyclopropylmethyl-amide (example 432)

At 0° C. a solution of methanesulfonyl chloride (0.038 mmol) in ether (0.1 mL) is added to a mixture of 2-methyl-5-m-tolyl-thiazole-4-carboxylic acid cyclopropyl-methyl-[2-(3,4-dimethoxy-phenyl)-2-hydroxy-ethyl]-amide (0.038 mmol) and TEA (0.114 mmol) in ether (0.25 mL). After 10 min additional TEA (0.076 mmol) and a solution of cyclopropylamine (0.38 mmol) in EtOH (0.1 mL) are added and the mixture is allowed to reach RT under stirring. After 14 h most of the ether is removed by a stream of nitrogen gas, DMF (0.5 mL) is added and the mixture is purified twice by prep HPLC using basic and acidic conditions respectively. Hydrochloric acid (1.0 M, 0.15 mL) is added and the solvents are removed in vacuo to give the desired product as a HCl salt. LC-MS (B): t_{R}=1.10 min; [M+H]⁺=506.2; (C): t_{R}=0.68 min; [M+H]⁺=506.2.

B.5 Synthesis of 2-Aminomethyl-5-m-tolyl-thiazole-4-carboxylic acid cyclopropylmethyl-[2-(5-fluoro-1H-indol-3-yl)-ethyl]-amide (example 433)

A solution of HCl in dioxane (4.0 M, 0.10 mL) is added to a solution of (4-{Cyclopropylmethyl-[2-(5-fluoro-1H-indol-3-yl)-ethyl]-carbamoyl}-5-m-tolyl-thiazol-2-ylmethyl)-carbamic acid tert-butyl ester (0.015 mmol) in dioxane (0.1 mL). The mixture is stirred for 16 h and concentrated in vacuo to give the desired product as a hydrochloride salt. LC-MS (B): t_{R}=0.87 min; [M+H]⁺=463.0.

B.6 Synthesis of 3-(3,4-Dimethyl-phenyl)-pyrazine-2-carboxylic acid (2-amino-ethyl)-[2-(5-fluoro-1H-indol-3-yl)-ethyl]amide (example 434)

A solution of HCl in dioxane (4.0 M, 0.50 mL) is added to a solution of (2-{[3-(3,4-dimethyl-phenyl)-pyrazine-2-carbonyl]-[2-(5-fluoro-1H-indol-3-yl)-ethyl]-amino}-ethyl)-carbamic acid tert-butyl ester (0.018 mmol) in dioxane (0.5 mL). The mixture is stirred for 2 h and concentrated in vacuo to give the desired product as a hydrochloride salt. LC-MS (C): t_{R}=0.56 min; [M+H]⁺=432.2.

B.7 Synthesis of 2-Methylamino-5-m-tolyl-thiazole-4-carboxylic acid cyclopropylmethyl-[2-(5-fluoro-1H-indol-3-yl)-ethyl]amide (example 435)

A solution of methylamine in THF (2.0 M, 0.20 mL) is added to 2-bromo-5-m-tolyl-thiazole-4-carboxylic acid cyclopropylmethyl-[2-(5-fluoro-1H-indol-3-yl)-ethyl]-amide (0.055 mmol). The solution is heated to 50° C., stirred for 5 h and treated with a solution of methylamine in THF (2.0 M, 0.40 mL). The mixture is heated to 70° C. in a closed vial, stirred for 17 h and concentrated in vacuo. The residue is diluted in DMF (1.0 mL) and purified by prep. HPLC (basic gradient) to give the desired product. LC-MS (C): t_{R}=0.75 min; [M+H]⁺=463.1.

### B.8 Synthesis of Compounds of Formula (I) by Suzuki Reaction (General Procedure II)

A mixture of 3-chloro-pyrazine-2-carboxylic acid cyclopropylmethyl-[2-(7-methyl-1H-indol-3-yl)-ethyl]-amide (0.024 mmol) and the respective boronic acid derivative (0.024 mmol) is dissolved in DME (0.14 mL). A aqueous K₂CO₃ solution (2.0 M, 0.08 mL) is added and nitrogen gas is passed through the mixture to remove oxygen. Triphenylphosphine (1.0 mg) and palladium(II) acetate (0.27 mg) are added under nitrogen and the mixture is vigorously stirred at around 90° C. for 1 h. DMF (1.0 mL) is added and the mixture is purified by prep. HPLC (basic conditions) to give the desired product.

| | | LC-MS | | |
|---|---|---|---|---|
| Example | Name | method | t_{R} [min] | [M + H]⁺ |
| 436 | 3-(4-Methoxy-phenyl)-pyrazine-2-carboxylic | (C) | 0.80 | 441.1 |
| | acid cyclopropylmethyl-[2-(7-methyl-1H-indol- | | | |
| | 3-yl)-ethyl]-amide | | | |
| 437 | 3-(6-Methoxy-pyridin-3-yl)-pyrazine-2- | (C) | 0.77 | 442.1 |
| | carboxylic acid cyclopropylmethyl-[2-(7- | | | |
| | methyl-1H-indol-3-yl)-ethyl]-amide | | | |

### B.9 Synthesis of Compounds of Formula (I) by Suzuki Reaction (General Procedure III)

A mixture of 3-chloro-pyrazine-2-carboxylic acid cyclopropylmethyl-[2-(7-methyl-1H-indol-3-yl)-ethyl]-amide (0.024 mmol) and the respective pyrimidine-5-boronic acid derivative (0.024 mmol) is dissolved in DME (0.14 mL). A aqueous K₂CO₃ solution (2.0 M, 0.08 mL) is added and nitrogen gas is passed through the mixture to remove oxygen. Triphenylphosphine (1.0 mg) and palladium(II) acetate (0.27 mg) are added under nitrogen and the mixture is vigorously stirred at around 90° C. for 3 h. Additional pyrimidine-5-boronic acid derivative (0.036 mmol), triphenylphosphine (1.0 mg) and palladium(II) acetate (0.27 mg) are added under nitrogen and the mixture is vigorously stirred at around 80° C. for 20 min. DMF (1.0 mL) is added and the mixture is purified by prep. HPLC (basic conditions) to give the desired product.

| | | LC-MS | | |
|---|---|---|---|---|
| Example | Name | method | t_{R} [min] | [M + H]⁺ |
| 438 | 3-Pyrimidin-5-yl-pyrazine-2-carboxylic acid | (C) | 0.66 | 413.1 |
| | cyclopropylmethyl-[2-(7-methyl-1H-indol-3- | | | |
| | yl)-ethyl]-amide | | | |
| 439 | 3-(2-Methoxy-pyrimidin-5-yl)-pyrazine-2- | (C) | 0.72 | 443.1 |
| | carboxylic acid cyclopropylmethyl-[2-(7- | | | |
| | methyl-1H-indol-3-yl)-ethyl]-amide | | | |

The following examples 440 to 607 were synthesized by applying procedures described above:

| | | LC-MS | | |
|---|---|---|---|---|
| Example | Name | method | t_{R} [min] | [M + H]⁺ |
| 440 | 3-(4-Fluoro-phenyl)-pyrazine-2-carboxylic acid | (C) | 1.04 | 445.39 |
| | cyclopropylmethyl-[2-(7-methoxy-1H-indol-3- | | | |
| | yl)-ethyl]-amide | | | |
| 441 | 3-(4-Fluoro-3-methyl-phenyl)-pyrazine-2- | (C) | 1.06 | 459.53 |
| | carboxylic acid cyclopropylmethyl-[2-(7- | | | |
| | methoxy-1H-indol-3-yl)-ethyl]-amide | | | |
| 442 | 3-(2-Fluoro-5-methoxy-phenyl)-pyrazine-2- | (C) | 1.04 | 475.53 |
| | carboxylic acid cyclopropylmethyl-[2-(7- | | | |
| | methoxy-1H-indol-3-yl)-ethyl]-amide | | | |
| 443 | 3-(3-Fluoro-5-methyl-phenyl)-pyrazine-2- | (C) | 1.07 | 459.4 |
| | carboxylic acid cyclopropylmethyl-[2-(7- | | | |
| | methoxy-1H-indol-3-yl)-ethyl]-amide | | | |
| 444 | 3-(3-Trifluoromethyl-phenyl)-pyrazine-2- | (C) | 1.09 | 495.56 |
| | carboxylic acid cyclopropylmethyl-[2-(7- | | | |
| | methoxy-1H-indol-3-yl)-ethyl]-amide | | | |
| 445 | 3-(2,3-Dimethyl-phenyl)-pyrazine-2-carboxylic | (C) | 1.07 | 455.64 |
| | acid cyclopropylmethyl-[2-(7-methoxy-1H- | | | |
| | indol-3-yl)-ethyl]-amide | | | |
| 446 | 3-(3-Methoxy-phenyl)-pyrazine-2-carboxylic | (C) | 1.04 | 457.46 |
| | acid cyclopropylmethyl-[2-(7-methoxy-1H- | | | |
| | indol-3-yl)-ethyl]-amide | | | |
| 447 | 3-m-Tolyl-pyrazine-2-carboxylic acid | (C) | 1.06 | 441.62 |
| | cyclopropylmethyl-[2-(7-methoxy-1H-indol-3- | | | |
| | yl)-ethyl]-amide | | | |
| 448 | 3-(4-Fluoro-phenyl)-pyrazine-2-carboxylic acid | (C) | 1.04 | 433.07 |
| | cyclopropylmethyl-[2-(5-fluoro-1H-indol-3-yl)- | | | |
| | ethyl]-amide | | | |
| 449 | 3-(4-Fluoro-3-methyl-phenyl)-pyrazine-2- | (C) | 1.07 | 447.11 |
| | carboxylic acid cyclopropylmethyl-[2-(5-fluoro- | | | |
| | 1H-indol-3-yl)-ethyl]-amide | | | |
| 450 | 3-(2-Fluoro-5-methoxy-phenyl)-pyrazine-2- | (C) | 1.04 | 463.32 |
| | carboxylic acid cyclopropylmethyl-[2-(5-fluoro- | | | |
| | 1H-indol-3-yl)-ethyl]-amide | | | |
| 451 | 3-(3-Fluoro-5-methyl-phenyl)-pyrazine-2- | (C) | 1.07 | 447.14 |
| | carboxylic acid cyclopropylmethyl-[2-(5-fluoro- | | | |
| | 1H-indol-3-yl)-ethyl]-amide | | | |
| 452 | 3-(3-Trifluoromethyl-phenyl)-pyrazine-2- | (C) | 1.08 | 483.47 |
| | carboxylic acid cyclopropylmethyl-[2-(5-fluoro- | | | |
| | 1H-indol-3-yl)-ethyl]-amide | | | |
| 453 | 3-(2,3-Dimethyl-phenyl)-pyrazine-2-carboxylic | (C) | 1.07 | 443.82 |
| | acid cyclopropylmethyl-[2-(5-fluoro-1H-indol- | | | |
| | 3-yl)-ethyl]-amide | | | |
| 454 | 2-Methyl-4-phenyl-pyrimidine-5-carboxylic | (C) | 1.01 | 429.13 |
| | acid cyclopropylmethyl-[2-(5-fluoro-1H-indol- | | | |
| | 3-yl)-ethyl]-amide | | | |
| 455 | 4-Phenyl-pyrimidine-5-carboxylic acid | (C) | 1.02 | 415.28 |
| | cyclopropylmethyl-[2-(5-fluoro-1H-indol-3-yl)- | | | |
| | ethyl]-amide | | | |
| 456 | 3-(4-Fluoro-phenyl)-pyrazine-2-carboxylic acid | (C) | 1.06 | 451.21 |
| | cyclopropylmethyl-[2-(5,6-difluoro-1H-indol-3- | | | |
| | yl)-ethyl]-amide | | | |
| 457 | 3-(4-Fluoro-3-methyl-phenyl)-pyrazine-2- | (C) | 1.07 | 465.52 |
| | carboxylic acid cyclopropylmethyl-[2-(5,6- | | | |
| | difluoro-1H-indol-3-yl)-ethyl]-amide | | | |
| 458 | 3-(2-Fluoro-5-methoxy-phenyl)-pyrazine-2- | (C) | 1.06 | 481.35 |
| | carboxylic acid cyclopropylmethyl-[2-(5,6- | | | |
| | difluoro-1H-indol-3-yl)-ethyl]-amide | | | |
| 459 | 3-(3-Fluoro-5-methyl-phenyl)-pyrazine-2- | (C) | 1.09 | 465.05 |
| | carboxylic acid cyclopropylmethyl-[2-(5,6- | | | |
| | difluoro-1H-indol-3-yl)-ethyl]-amide | | | |
| 460 | 3-(3-Trifluoromethyl-phenyl)-pyrazine-2- | (C) | 1.11 | 501.39 |
| | carboxylic acid cyclopropylmethyl-[2-(5,6- | | | |
| | difluoro-1H-indol-3-yl)-ethyl]-amide | | | |
| 461 | 3-(2,3-Dimethyl-phenyl)-pyrazine-2-carboxylic | (C) | 1.09 | 461.32 |
| | acid cyclopropylmethyl-[2-(5,6-difluoro-1H- | | | |
| | indol-3-yl)-ethyl]-amide | | | |
| 462 | 2-Methyl-4-phenyl-pyrimidine-5-carboxylic | (C) | 1.04 | 447.15 |
| | acid cyclopropylmethyl-[2-(5,6-difluoro-1H- | | | |
| | indol-3-yl)-ethyl]-amide | | | |
| 463 | 4-Phenyl-pyrimidine-5-carboxylic acid | (C) | 1.04 | 433.42 |
| | cyclopropylmethyl-[2-(5,6-difluoro-1H-indol-3- | | | |
| | yl)-ethyl]-amide | | | |
| 464 | 3-(4-Fluoro-phenyl)-pyrazine-2-carboxylic acid | (C) | 1.08 | 467.34 |
| | [2-(5-chloro-6-fluoro-1H-indol-3-yl)-ethyl]- | | | |
| | cyclopropylmethyl-amide | | | |
| 465 | 3-(4-Fluoro-3-methyl-phenyl)-pyrazine-2- | (C) | 1.10 | 480.52 |
| | carboxylic acid [2-(5-chloro-6-fluoro-1H-indol- | | | |
| | 3-yl)-ethyl]-cyclopropylmethyl-amide | | | |
| 466 | 3-(2-Fluoro-5-methoxy-phenyl)-pyrazine-2- | (C) | 1.07 | 497.36 |
| | carboxylic acid [2-(5-chloro-6-fluoro-1H-indol- | | | |
| | 3-yl)-ethyl]-cyclopropylmethyl-amide | | | |
| 467 | 3-(3-Fluoro-5-methyl-phenyl)-pyrazine-2- | (C) | 1.10 | 481.33 |
| | carboxylic acid [2-(5-chloro-6-fluoro-1H-indol- | | | |
| | 3-yl)-ethyl]-cyclopropylmethyl-amide | | | |
| 468 | 3-(3-Trifluoromethyl-phenyl)-pyrazine-2- | (C) | 1.12 | 517.36 |
| | carboxylic acid [2-(5-chloro-6-fluoro-1H-indol- | | | |
| | 3-yl)-ethyl]-cyclopropylmethyl-amide | | | |
| 469 | 3-(2,3-Dimethyl-phenyl)-pyrazine-2-carboxylic | (C) | 1.10 | 477.36 |
| | acid [2-(5-chloro-6-fluoro-1H-indol-3-yl)- | | | |
| | ethyl]-cyclopropylmethyl-amide | | | |
| 470 | 2-Methyl-4-phenyl-pyrimidine-5-carboxylic | (C) | 1.05 | 463.3 |
| | acid [2-(5-chloro-6-fluoro-1H-indol-3-yl)- | | | |
| | ethyl]-cyclopropylmethyl-amide | | | |
| 471 | 4-Phenyl-pyrimidine-5-carboxylic acid [2-(5- | (C) | 1.06 | 448.25 |
| | chloro-6-fluoro-1H-indol-3-yl)-ethyl]- | | | |
| | cyclopropylmethyl-amide | | | |
| 472 | 2-Dimethylamino-5-phenyl-thiazole-4- | (C) | 1.08 | 497.42 |
| | carboxylic acid [2-(5-chloro-6-fluoro-1H-indol- | | | |
| | 3-yl)-ethyl]-cyclopropylmethyl-amide | | | |
| 473 | 2-Dimethylamino-5-m-tolyl-thiazole-4- | (C) | 1.09 | 511.22 |
| | carboxylic acid [2-(5-chloro-6-fluoro-1H-indol- | | | |
| | 3-yl)-ethyl]-cyclopropylmethyl-amide | | | |
| 474 | 2-Dimethylamino-5-(3-fluoro-4-methyl- | (C) | 1.12 | 529.42 |
| | phenyl)-thiazole-4-carboxylic acid [2-(5-chloro- | | | |
| | 6-fluoro-1H-indol-3-yl)-ethyl]- | | | |
| | cyclopropylmethyl-amide | | | |
| 475 | 2-Dimethylamino-5-(4-fluoro-phenyl)-thiazole- | (C) | 1.09 | 515.36 |
| | 4-carboxylic acid [2-(5-chloro-6-fluoro-1H- | | | |
| | indol-3-yl)-ethyl]-cyclopropylmethyl-amide | | | |
| 476 | 3-(4-Fluoro-phenyl)-pyrazine-2-carboxylic acid | (C) | 1.04 | 459.67 |
| | cyclopropylmethyl-[2-(5-methoxy-4-methyl- | | | |
| | 1H-indol-3-yl)-ethyl]-amide | | | |
| 477 | 3-(4-Fluoro-3-methyl-phenyl)-pyrazine-2- | (C) | 1.06 | 473.46 |
| | carboxylic acid cyclopropylmethyl-[2-(5- | | | |
| | methoxy-4-methyl-1H-indol-3-yl)-ethyl]-amide | | | |
| 478 | 3-(3-Fluoro-5-methyl-phenyl)-pyrazine-2- | (C) | 1.07 | 473.56 |
| | carboxylic acid cyclopropylmethyl-[2-(5- | | | |
| | methoxy-4-methyl-1H-indol-3-yl)-ethyl]-amide | | | |
| 479 | 2-Methyl-4-phenyl-pyrimidine-5-carboxylic | (C) | 1.01 | 455.46 |
| | acid cyclopropylmethyl-[2-(5-methoxy-4- | | | |
| | methyl-1H-indol-3-yl)-ethyl]-amide | | | |
| 480 | 4-Phenyl-pyrimidine-5-carboxylic acid | (C) | 1.01 | 440.98 |
| | cyclopropylmethyl-[2-(5-methoxy-4-methyl- | | | |
| | 1H-indol-3-yl)-ethyl]-amide | | | |
| 481 | 2-Dimethylamino-5-phenyl-thiazole-4- | (C) | 1.01 | 489.46 |
| | carboxylic acid cyclopropylmethyl-[2-(5- | | | |
| | methoxy-4-methyl-1H-indol-3-yl)-ethyl]-amide | | | |
| 482 | 2-Dimethylamino-5-m-tolyl-thiazole-4- | (C) | 1.02 | 503.59 |
| | carboxylic acid cyclopropylmethyl-[2-(5- | | | |
| | methoxy-4-methyl-1H-indol-3-yl)-ethyl]-amide | | | |
| 483 | 2-Dimethylamino-5-(3-fluoro-4-methyl- | (C) | 1.07 | 521.62 |
| | phenyl)-thiazole-4-carboxylic acid | | | |
| | cyclopropylmethyl-[2-(5-methoxy-4-methyl- | | | |
| | 1H-indol-3-yl)-ethyl]-amide | | | |
| 484 | 2-Dimethylamino-5-(4-fluoro-phenyl)-thiazole- | (C) | 1.03 | 507.43 |
| | 4-carboxylic acid cyclopropylmethyl-[2-(5- | | | |
| | methoxy-4-methyl-1H-indol-3-yl)-ethyl]-amide | | | |
| 485 | 2-Dimethylamino-5-(3-fluoro-phenyl)-thiazole- | (C) | 1.08 | 481.12 |
| | 4-carboxylic acid cyclopropylmethyl-[2-(7- | | | |
| | fluoro-1H-indol-3-yl)-ethyl]-amide | | | |
| 486 | 2-Dimethylamino-5-(3-fluoro-phenyl)-thiazole- | (C) | 1.05 | 481.23 |
| | 4-carboxylic acid cyclopropylmethyl-[2-(4- | | | |
| | fluoro-1H-indol-3-yl)-ethyl]-amide | | | |
| 487 | 2-Dimethylamino-5-(3-fluoro-phenyl)-thiazole- | (C) | 1.08 | 481.27 |
| | 4-carboxylic acid cyclopropylmethyl-[2-(6- | | | |
| | fluoro-1H-indol-3-yl)-ethyl]-amide | | | |
| 488 | 3-(4-Fluoro-phenyl)-pyrazine-2-carboxylic acid | (C) | 1.03 | 415.15 |
| | cyclopropylmethyl-[2-(1H-indol-3-yl)-ethyl]- | | | |
| | amide | | | |
| 489 | 3-(4-Fluoro-3-methyl-phenyl)-pyrazine-2- | (C) | 1.07 | 429.30 |
| | carboxylic acid cyclopropylmethyl-[2-(1H- | | | |
| | indol-3-yl)-ethyl]-amide | | | |
| 490 | 3-(2-Fluoro-5-methoxy-phenyl)-pyrazine-2- | (C) | 1.03 | 445.11 |
| | carboxylic acid cyclopropylmethyl-[2-(1H- | | | |
| | indol-3-yl)-ethyl]-amide | | | |
| 491 | 3-(3-Fluoro-5-methyl-phenyl)-pyrazine-2- | (C) | 1.06 | 429.12 |
| | carboxylic acid cyclopropylmethyl-[2-(1H- | | | |
| | indol-3-yl)-ethyl]-amide | | | |
| 492 | 3-(3-Trifluoromethyl-phenyl)-pyrazine-2- | (C) | 1.09 | 465.33 |
| | carboxylic acid cyclopropylmethyl-[2-(1H- | | | |
| | indol-3-yl)-ethyl]-amide | | | |
| 493 | 3-(2,3-Dimethyl-phenyl)-pyrazine-2-carboxylic | (C) | 1.06 | 425.33 |
| | acid cyclopropylmethyl-[2-(1H-indol-3-yl)- | | | |
| | ethyl]-amide | | | |
| 494 | 3-(3-Methoxy-phenyl)-pyrazine-2-carboxylic | (C) | 1.02 | 427.43 |
| | acid cyclopropylmethyl-[2-(1H-indol-3-yl)- | | | |
| | ethyl]-amide | | | |
| 495 | 3-m-Tolyl-pyrazine-2-carboxylic acid | (C) | 1.04 | 411.32 |
| | cyclopropylmethyl-[2-(1H-indol-3-yl)-ethyl]- | | | |
| | amide | | | |
| 496 | 2-Methyl-4-phenyl-pyrimidine-5-carboxylic | (C) | 1.0 | 411.41 |
| | acid cyclopropylmethyl-[2-(1H-indol-3-yl)- | | | |
| | ethyl]-amide | | | |
| 497 | 4-Phenyl-pyrimidine-5-carboxylic acid | (C) | 1.00 | 397.47 |
| | cyclopropylmethyl-[2-(1H-indol-3-yl)-ethyl]- | | | |
| | amide | | | |
| 498 | 3-(3,4-Dimethyl-phenyl)-pyrazine-2-carboxylic | (C) | 1.06 | 425.13 |
| | acid cyclopropylmethyl-[2-(1H-indol-3-yl)- | | 1.07 | 425.59 |
| | ethyl]-amide | | | |
| 499 | 3-Phenyl-pyrazine-2-carboxylic acid | (C) | 1.01 | 397.59 |
| | cyclopropylmethyl-[2-(1H-indol-3-yl)-ethyl]- | | 1.02 | 397.52 |
| | amide | | | |
| 500 | 2-(Ethyl-methyl-amino)-5-(2-fluoro-phenyl)- | (C) | 1.08 | 477.44 |
| | thiazole-4-carboxylic acid cyclopropylmethyl- | | | |
| | [2-(1H-indol-3-yl)-ethyl]-amide | | | |
| 501 | 2-Methyl-5-(4-propionylamino-phenyl)- | (C) | 0.99 | 487.24 |
| | thiazole-4-carboxylic acid cyclopropylmethyl- | | | |
| | [2-(1H-indol-3-yl)-ethyl]-amide | | | |
| 502 | 4-(3-Chloro-phenyl)-pyrimidine-5-carboxylic | (C) | 1.05 | 430.88 |
| | acid cyclopropylmethyl-[2-(1H-indol-3-yl)- | | | |
| | ethyl]-amide | | | |
| 503 | 4-(3-Chloro-phenyl)-2-methyl-pyrimidine-5- | (C) | 1.04 | 445.76 |
| | carboxylic acid cyclopropylmethyl-[2-(1H- | | | |
| | indol-3-yl)-ethyl]-amide | | | |
| 504 | 4-(3,4-Dimethyl-phenyl)-pyrimidine-5- | (C) | 1.05 | 425.13 |
| | carboxylic acid cyclopropylmethyl-[2-(1H- | | | |
| | indol-3-yl)-ethyl]-amide | | | |
| 505 | 4-(3,4-Dimethyl-phenyl)-2-methyl-pyrimidine- | (C) | 1.05 | 438.99 |
| | 5-carboxylic acid cyclopropylmethyl-[2-(1H- | | | |
| | indol-3-yl)-ethyl]-amide | | | |
| 506 | 4-(3-Methoxy-phenyl)-pyrimidine-5-carboxylic | (C) | 1.00 | 426.42 |
| | acid cyclopropylmethyl-[2-(1H-indol-3-yl)- | | | |
| | ethyl]-amide | | | |
| 507 | 4-(3-Methoxy-phenyl)-2-methyl-pyrimidine-5- | (C) | 1.00 | 441.84 |
| | carboxylic acid cyclopropylmethyl-[2-(1H- | | | |
| | indol-3-yl)-ethyl]-amide | | | |
| 508 | 4-(3,4-Dichloro-phenyl)-pyrimidine-5- | (C) | 1.08 | 464.93 |
| | carboxylic acid cyclopropylmethyl-[2-(1H- | | | |
| | indol-3-yl)-ethyl]-amide | | | |
| 509 | 4-(3,4-Dichloro-phenyl)-2-methyl-pyrimidine-5- | (C) | 1.09 | 479.08 |
| | carboxylic acid cyclopropylmethyl-[2-(1H- | | | |
| | indol-3-yl)-ethyl]-amide | | | |
| 510 | 4-(3-Fluoro-phenyl)-pyrimidine-5-carboxylic | (C) | 1.01 | 415.03 |
| | acid cyclopropylmethyl-[2-(1H-indol-3-yl)- | | | |
| | ethyl]-amide | | | |
| 511 | 4-(3-Fluoro-phenyl)-2-methyl-pyrimidine-5- | (C) | 1.02 | 428.95 |
| | carboxylic acid cyclopropylmethyl-[2-(1H- | | | |
| | indol-3-yl)-ethyl]-amide | | | |
| 512 | 4-(4-Bromo-3-chloro-phenyl)-2-methyl- | (C) | 1.09 | 525.45 |
| | pyrimidine-5-carboxylic acid | | | |
| | cyclopropylmethyl-[2-(1H-indol-3-yl)-ethyl]- | | | |
| | amide | | | |
| 513 | 4-(4-Bromo-3-chloro-phenyl)-pyrimidine-5- | (C) | 1.09 | 511.56 |
| | carboxylic acid cyclopropylmethyl-[2-(1H- | | | |
| | indol-3-yl)-ethyl]-amide | | | |
| 514 | 4-m-Tolyl-pyrimidine-5-carboxylic acid | (C) | 1.03 | 411.31 |
| | cyclopropylmethyl-[2-(1H-indol-3-yl)-ethyl]- | | | |
| | amide | | | |
| 515 | 2-Methyl-4-m-tolyl-pyrimidine-5-carboxylic | (C) | 1.01 | 425.09 |
| | acid cyclopropylmethyl-[2-(1H-indol-3-yl)- | | | |
| | ethyl]-amide | | | |
| 516 | 2-Methyl-4-p-tolyl-pyrimidine-5-carboxylic | (C) | 1.03 | 425.01 |
| | acid cyclopropylmethyl-[2-(1H-indol-3-yl)- | | | |
| | ethyl]-amide | | | |
| 517 | 4-p-Tolyl-pyrimidine-5-carboxylic acid | (C) | 1.03 | 411.16 |
| | cyclopropylmethyl-[2-(1H-indol-3-yl)-ethyl]- | | | |
| | amide | | | |
| 518 | 4-(4-Fluoro-phenyl)-pyrimidine-5-carboxylic | (C) | 1.01 | 415.04 |
| | acid cyclopropylmethyl-[2-(1H-indol-3-yl)- | | | |
| | ethyl]-amide | | | |
| 519 | 4-(4-Fluoro-phenyl)-2-methyl-pyrimidine-5- | (C) | 1.01 | 429.27 |
| | carboxylic acid cyclopropylmethyl-[2-(1H- | | | |
| | indol-3-yl)-ethyl]-amide | | | |
| 520 | 3-Phenyl-pyrazine-2-carboxylic acid ethyl-[2- | (C) | 0.98 | 371.29 |
| | (1H-indol-3-yl)-ethyl]-amide | | | |
| 521 | 4-Phenyl-pyrimidine-5-carboxylic acid ethyl-[2- | (C) | 0.95 | 371.47 |
| | (1H-indol-3-yl)-ethyl]-amide | | | |
| 522 | 2-Methyl-4-phenyl-pyrimidine-5-carboxylic | (C) | 0.96 | 385.58 |
| | acid ethyl-[2-(1H-indol-3-yl)-ethyl]-amide | | | |
| 523 | 3-m-Tolyl-pyrazine-2-carboxylic acid ethyl-[2- | (C) | 1.00 | 385.39 |
| | (1H-indol-3-yl)-ethyl]-amide | | | |
| 524 | 4-m-Tolyl-pyrimidine-5-carboxylic acid ethyl- | (C) | 0.99 | 385.35 |
| | [2-(1H-indol-3-yl)-ethyl]-amide | | | |
| 525 | 2-Methyl-4-m-tolyl-pyrimidine-5-carboxylic | (C) | 0.99 | 399.39 |
| | acid ethyl-[2-(1H-indol-3-yl)-ethyl]-amide | | | |
| 526 | 3-(4-Fluoro-phenyl)-pyrazine-2-carboxylic acid | (C) | 0.99 | 389.28 |
| | ethyl-[2-(1H-indol-3-yl)-ethyl]-amide | | | |
| 527 | 4-(4-Fluoro-phenyl)-pyrimidine-5-carboxylic | (C) | 0.97 | 389.09 |
| | acid ethyl-[2-(1H-indol-3-yl)-ethyl]-amide | | | |
| 528 | 4-(4-Fluoro-phenyl)-2-methyl-pyrimidine-5- | (C) | 0.97 | 403.36 |
| | carboxylic acid ethyl-[2-(1H-indol-3-yl)-ethyl]- | | | |
| | amide | | | |
| 529 | 3-(4-Fluoro-3-methyl-phenyl)-pyrazine-2- | (C) | 1.02 | 403.18 |
| | carboxylic acid ethyl-[2-(1H-indol-3-yl)-ethyl]- | | | |
| | amide | | | |
| 530 | 4-(3-Fluoro-phenyl)-pyrimidine-5-carboxylic | (C) | 0.98 | 389.20 |
| | acid ethyl-[2-(1H-indol-3-yl)-ethyl]-amide | | | |
| 531 | 4-(3-Fluoro-phenyl)-2-methyl-pyrimidine-5- | (C) | 0.98 | 403.21 |
| | carboxylic acid ethyl-[2-(1H-indol-3-yl)-ethyl]- | | | |
| | amide | | | |
| 532 | 3-(3-Fluoro-5-methyl-phenyl)-pyrazine-2- | (C) | 1.03 | 403.60 |
| | carboxylic acid ethyl-[2-(1H-indol-3-yl)-ethyl]- | | | |
| | amide | | | |
| 533 | 3-(3-Methoxy-phenyl)-pyrazine-2-carboxylic | (C) | 0.98 | 401.04 |
| | acid ethyl-[2-(1H-indol-3-yl)-ethyl]-amide | | | |
| 534 | 3-(3,4-Dimethyl-phenyl)-pyrazine-2-carboxylic | (C) | 1.03 | 399.37 |
| | acid ethyl-[2-(1H-indol-3-yl)-ethyl]-amide | | | |
| 535 | 4-(4-Bromo-3-chloro-phenyl)-2-methyl- | (C) | 1.06 | 497.16 |
| | pyrimidine-5-carboxylic acid ethyl-[2-(1H- | | | |
| | indol-3-yl)-ethyl]-amide | | | |
| 536 | 4-(4-Bromo-3-chloro-phenyl)-pyrimidine-5- | (C) | 1.06 | 485.03 |
| | carboxylic acid ethyl-[2-(1H-indol-3-yl)-ethyl]- | | | |
| | amide | | | |
| 537 | 2-Methyl-4-p-tolyl-pyrimidine-5-carboxylic | (C) | 0.99 | 399.41 |
| | acid ethyl-[2-(1H-indol-3-yl)-ethyl]-amide | | | |
| 538 | 4-p-Tolyl-pyrimidine-5-carboxylic acid ethyl- | (C) | 0.99 | 385.57 |
| | [2-(1H-indol-3-yl)-ethyl]-amide | | | |
| 539 | 4-(3,5-Dichloro-phenyl)-2-methyl-pyrimidine-5- | (C) | 1.07 | 452.90 |
| | carboxylic acid ethyl-[2-(1H-indol-3-yl)-ethyl]- | | | |
| | amide | | | |
| 540 | 4-(3,5-Dichloro-phenyl)-pyrimidine-5- | (C) | 1.06 | 439.59 |
| | carboxylic acid ethyl-[2-(1H-indol-3-yl)-ethyl]- | | | |
| | amide | | | |
| 541 | 4-(3-Methoxy-phenyl)-pyrimidine-5-carboxylic | (C) | 0.97 | 401.36 |
| | acid ethyl-[2-(1H-indol-3-yl)-ethyl]-amide | | | |
| 542 | 4-(3,4-Dimethyl-phenyl)-2-methyl-pyrimidine- | (C) | 1.01 | 413.18 |
| | 5-carboxylic acid ethyl-[2-(1H-indol-3-yl)- | | | |
| | ethyl]-amide | | | |
| 543 | 4-(3,4-Dimethyl-phenyl)-pyrimidine-5- | (C) | 1.01 | 399.46 |
| | carboxylic acid ethyl-[2-(1H-indol-3-yl)-ethyl]- | | | |
| | amide | | | |
| 544 | 4-(3,4-Dichloro-phenyl)-pyrimidine-5- | (C) | 1.05 | 438.73 |
| | carboxylic acid ethyl-[2-(1H-indol-3-yl)-ethyl]- | | | |
| | amide | | | |
| 545 | 3-Phenyl-pyrazine-2-carboxylic acid [2-(1H- | (C) | 1.02 | 424.99 |
| | indol-3-yl)-ethyl]-(2,2,2-trifluoro-ethyl)-amide | | | |
| 546 | 4-Phenyl-pyrimidine-5-carboxylic acid [2-(1H- | (C) | 1.00 | 425.01 |
| | indol-3-yl)-ethyl]-(2,2,2-trifluoro-ethyl)-amide | | | |
| 547 | 2-Methyl-4-phenyl-pyrimidine-5-carboxylic | (C) | 1.00 | 439.13 |
| | acid [2-(1H-indol-3-yl)-ethyl]-(2,2,2-trifluoro- | | | |
| | ethyl)-amide | | | |
| 548 | 3-m-Tolyl-pyrazine-2-carboxylic acid [2-(1H- | (C) | 1.05 | 438.90 |
| | indol-3-yl)-ethyl]-(2,2,2-trifluoro-ethyl)-amide | | | |
| 549 | 4-m-Tolyl-pyrimidine-5-carboxylic acid [2-(1H- | (C) | 1.02 | 439.02 |
| | indol-3-yl)-ethyl]-(2,2,2-trifluoro-ethyl)-amide | | | |
| 550 | 2-Methyl-4-m-tolyl-pyrimidine-5-carboxylic | (C) | 1.03 | 453.79 |
| | acid [2-(1H-indol-3-yl)-ethyl]-(2,2,2-trifluoro- | | | |
| | ethyl)-amide | | | |
| 551 | 3-(4-Fluoro-phenyl)-pyrazine-2-carboxylic acid | (C) | 1.03 | 442.92 |
| | [2-(1H-indol-3-yl)-ethyl]-(2,2,2-trifluoro-ethyl)- | | | |
| | amide | | | |
| 552 | 4-(4-Fluoro-phenyl)-pyrimidine-5-carboxylic | (C) | 1.01 | 442.87 |
| | acid [2-(1H-indol-3-yl)-ethyl]-(2,2,2-trifluoro- | | | |
| | ethyl)-amide | | | |
| 553 | 4-(4-Fluoro-phenyl)-2-methyl-pyrimidine-5- | (C) | 1.01 | 456.90 |
| | carboxylic acid [2-(1H-indol-3-yl)-ethyl]-(2,2,2- | | 1.02 | 456.78 |
| | trifluoro-ethyl)-amide | | | |
| 554 | 3-(4-Fluoro-3-methyl-phenyl)-pyrazine-2- | (C) | 1.06 | 456.91 |
| | carboxylic acid [2-(1H-indol-3-yl)-ethyl]-(2,2,2- | | | |
| | trifluoro-ethyl)-amide | | | |
| 555 | 4-(3-Fluoro-phenyl)-2-methyl-pyrimidine-5- | (C) | 1.01 | 457.08 |
| | carboxylic acid [2-(1H-indol-3-yl)-ethyl]-(2,2,2- | | | |
| | trifluoro-ethyl)-amide | | | |
| 556 | 3-(3-Fluoro-5-methyl-phenyl)-pyrazine-2- | (C) | 1.06 | 456.99 |
| | carboxylic acid [2-(1H-indol-3-yl)-ethyl]-(2,2,2- | | | |
| | trifluoro-ethyl)-amide | | | |
| 557 | 3-(3-Methoxy-phenyl)-pyrazine-2-carboxylic | (C) | 1.06 | 454.94 |
| | acid [2-(1H-indol-3-yl)-ethyl]-(2,2,2-trifluoro- | | | |
| | ethyl)-amide | | | |
| 558 | 3-(3,4-Dimethyl-phenyl)-pyrazine-2-carboxylic | (C) | 1.07 | 452.95 |
| | acid [2-(1H-indol-3-yl)-ethyl]-(2,2,2-trifluoro- | | | |
| | ethyl)-amide | | | |
| 559 | 4-(4-Bromo-3-chloro-phenyl)-2-methyl- | (C) | 1.08 | 551.27 |
| | pyrimidine-5-carboxylic acid [2-(1H-indol-3- | | | |
| | yl)-ethyl]-(2,2,2-trifluoro-ethyl)-amide | | | |
| 560 | 4-p-Tolyl-pyrimidine-5-carboxylic acid [2-(1H- | (C) | 1.03 | 439.93 |
| | indol-3-yl)-ethyl]-(2,2,2-trifluoro-ethyl)-amide | | | |
| 561 | 4-(3,4-Dimethyl-phenyl)-2-methyl-pyrimidine- | (C) | 1.05 | 467.19 |
| | 5-carboxylic acid [2-(1H-indol-3-yl)-ethyl]- | | | |
| | (2,2,2-trifluoro-ethyl)-amide | | | |
| 562 | 4-(3,4-Dimethyl-phenyl)-pyrimidine-5- | (C) | 1.05 | 452.93 |
| | carboxylic acid [2-(1H-indol-3-yl)-ethyl]-(2,2,2- | | | |
| | trifluoro-ethyl)-amide | | | |
| 563 | {[2-Dimethylamino-5-(3-fluoro-4-methyl- | (C) | 1.04 | 495.43 |
| | phenyl)-thiazole-4-carbonyl]-[2-(1H-indol-3- | | | |
| | yl)-ethyl]-amino}-acetic acid methyl ester | | | |
| 564 | {[5-(3-Bromo-4-fluoro-phenyl)-2- | (C) | 1.07 | 559.17 |
| | dimethylamino-thiazole-4-carbonyl]-[2-(1H- | | | |
| | indol-3-yl)-ethyl]-amino}-acetic acid methyl | | | |
| | ester | | | |
| 565 | {(2-Dimethylamino-5-p-tolyl-thiazole-4- | (C) | 0.99 | 478.04 |
| | carbonyl)-[2-(1H-indol-3-yl)-ethyl]-amino}- | | | |
| | acetic acid methyl ester | | | |
| 566 | {[2-Dimethylamino-5-(2-fluoro-phenyl)- | (C) | 1.01 | 481.15 |
| | thiazole-4-carbonyl]-[2-(1H-indol-3-yl)-ethyl]- | | | |
| | amino}-acetic acid methyl ester | | | |
| 567 | {[2-Dimethylamino-5-(4-fluoro-phenyl)- | (C) | 1.0 | 481.07 |
| | thiazole-4-carbonyl]-[2-(1H-indol-3-yl)-ethyl]- | | | |
| | amino}-acetic acid methyl ester | | | |
| 568 | {[2-(Ethyl-methyl-amino)-5-(4-fluoro-phenyl)- | (C) | 1.03 | 495.01 |
| | thiazole-4-carbonyl]-[2-(1H-indol-3-yl)-ethyl]- | | | |
| | amino}-acetic acid methyl ester | | | |
| 569 | {[2-(Ethyl-methyl-amino)-5-(3-methoxy- | (C) | 1.02 | 507.21 |
| | phenyl)-thiazole-4-carbonyl]-[2-(1H-indol-3- | | | |
| | yl)-ethyl]-amino}-acetic acid methyl ester | | | |
| 570 | {(2-Dimethylamino-5-m-tolyl-thiazole-4- | (C) | 1.00 | 477.01 |
| | carbonyl)-[2-(1H-indol-3-yl)-ethyl]-amino}- | | | |
| | acetic acid methyl ester | | | |
| 571 | {[5-(3-Fluoro-5-trifluoromethyl-phenyl)-2- | (C) | 1.08 | 520.2 |
| | methyl-thiazole-4-carbonyl]-[2-(1H-indol-3-yl)- | | | |
| | ethyl]-amino}-acetic acid methyl ester | | | |
| 572 | {[2-Cyclopropyl-5-(3-fluoro-5-trifluoromethyl- | (C) | 1.12 | 546.29 |
| | phenyl)-thiazole-4-carbonyl]-[2-(1H-indol-3- | | | |
| | yl)-ethyl]-amino}-acetic acid methyl ester | | | |
| 573 | {[2-Cyclopropyl-5-(3-fluoro-phenyl)-thiazole-4- | (C) | 1.07 | 478.88 |
| | carbonyl]-[2-(1H-indol-3-yl)-ethyl]-amino}- | | | |
| | acetic acid methyl ester | | | |
| 574 | [[2-(1H-Indol-3-yl)-ethyl]-(2-methyl-5-p-tolyl- | (C) | 1.08 | 447.98 |
| | thiazole-4-carbonyl)-amino]-acetic acid methyl | | | |
| | ester | | | |
| 575 | {[2-Cyclopropyl-5-(3-trifluoromethyl-phenyl)- | (C) | 1.11 | 528.33 |
| | thiazole-4-carbonyl]-[2-(1H-indol-3-yl)-ethyl]- | | | |
| | amino}-acetic acid methyl ester | | | |
| 576 | {[5-(4-Bromo-phenyl)-2-methyl-thiazole-4- | (C) | 1.05 | 514.58 |
| | carbonyl]-[2-(1H-indol-3-yl)-ethyl]-amino}- | | | |
| | acetic acid methyl ester | | | |
| 577 | {[2-(1H-Indol-3-yl)-ethyl]-[2-methyl-5-(3- | (C) | 1.06 | 502.29 |
| | trifluoromethyl-phenyl)-thiazole-4-carbonyl]- | | | |
| | amino}-acetic acid methyl ester | | | |
| 578 | {[5-(3,5-Dimethyl-phenyl)-2-methyl-thiazole-4- | (C) | 1.06 | 462.00 |
| | carbonyl]-[2-(1H-indol-3-yl)-ethyl]-amino}- | | | |
| | acetic acid methyl ester | | | |
| 579 | {[5-(2,4-Dimethyl-phenyl)-2-methyl-thiazole-4- | (C) | 1.07 | 461.91 |
| | carbonyl]-[2-(1H-indol-3-yl)-ethyl]-amino}- | | | |
| | acetic acid methyl ester | | | |
| 580 | {[5-(3-Cyano-phenyl)-2-methyl-thiazole-4- | (C) | 0.99 | 458.18 |
| | carbonyl]-[2-(1H-indol-3-yl)-ethyl]-amino}- | | | |
| | acetic acid methyl ester | | | |
| 581 | {[5-(3,4-Difluoro-phenyl)-2-methyl-thiazole-4- | (C) | 1.03 | 470.00 |
| | carbonyl]-[2-(1H-indol-3-yl)-ethyl]-amino}- | | | |
| | acetic acid methyl ester | | | |
| 582 | {[5-(2,3-Dichloro-phenyl)-2-methyl-thiazole-4- | (C) | 1.08 | 501.79 |
| | carbonyl]-[2-(1H-indol-3-yl)-ethyl]-amino}- | | | |
| | acetic acid methyl ester | | | |
| 583 | {[5-(2-Chloro-6-fluoro-phenyl)-2-methyl- | (C) | 1.05 | 486.03 |
| | thiazole-4-carbonyl]-[2-(1H-indol-3-yl)-ethyl]- | | | |
| | amino}-acetic acid methyl ester | | | |
| 584 | {[2-Cyclopropyl-5-(4-fluoro-phenyl)-thiazole-4- | (C) | 1.06 | 477.98 |
| | carbonyl]-[2-(1H-indol-3-yl)-ethyl]-amino}- | | | |
| | acetic acid methyl ester | | | |
| 585 | {[5-(3,4-Dichloro-phenyl)-2-methyl-thiazole-4- | (C) | 1.08 | 502.03 |
| | carbonyl]-[2-(1H-indol-3-yl)-ethyl]-amino}- | | | |
| | acetic acid methyl ester | | | |
| 586 | {[5-(3,5-Difluoro-phenyl)-2-methyl-thiazole-4- | (C) | 1.03 | 469.85 |
| | carbonyl]-[2-(1H-indol-3-yl)-ethyl]-amino}- | | | |
| | acetic acid methyl ester | | | |
| 587 | ([2-(1H-Indol-3-yl)-ethyl]-{5-[3-(2-methoxy- | (C) | 1.00 | 508.13 |
| | ethoxy)-phenyl]-2-methyl-thiazole-4-carbonyl}- | | | |
| | amino)-acetic acid methyl ester | | | |
| 588 | {[5-(3-Fluoro-4-methyl-phenyl)-2-methyl- | (C) | 1.04 | 465.86 |
| | thiazole-4-carbonyl]-[2-(1H-indol-3-yl)-ethyl]- | | | |
| | amino}-acetic acid methyl ester | | | |
| 589 | {[5-(3-Bromo-phenyl)-2-cyclopropyl-thiazole- | (C) | 1.09 | 538.04 |
| | 4-carbonyl]-[2-(1H-indol-3-yl)-ethyl]-amino}- | | | |
| | acetic acid methyl ester | | | |
| 590 | {[5-(3-Bromo-phenyl)-2-methyl-thiazole-4- | (C) | 1.05 | 513.91 |
| | carbonyl]-[2-(1H-indol-3-yl)-ethyl]-amino}- | | | |
| | acetic acid methyl ester | | | |
| 591 | {[2-Dimethylamino-5-(3,4-dimethyl-phenyl)- | (C) | 1.02 | 490.54 |
| | thiazole-4-carbonyl]-[2-(1H-indol-3-yl)-ethyl]- | | | |
| | amino}-acetic acid methyl ester | | | |
| 592 | {[2-Dimethylamino-5-(3-fluoro-phenyl)- | (C) | 1.02 | 481.53 |
| | thiazole-4-carbonyl]-[2-(1H-indol-3-yl)-ethyl]- | | | |
| | amino}-acetic acid methyl ester | | | |
| 593 | {[2-Dimethylamino-5-(3-trifluoromethyl- | (C) | 1.07 | 531.48 |
| | phenyl)-thiazole-4-carbonyl]-[2-(1H-indol-3- | | | |
| | yl)-ethyl]-amino}-acetic acid methyl ester | | | |
| 594 | {[5-(3-Chloro-phenyl)-2-dimethylamino- | (C) | 1.05 | 497.10 |
| | thiazole-4-carbonyl]-[2-(1H-indol-3-yl)-ethyl]- | | | |
| | amino}-acetic acid methyl ester | | | |
| 595 | {[5-(3,4-Dimethyl-phenyl)-2-methyl-thiazole-4- | (C) | 1.06 | 479.89 |
| | carbonyl]-[2-(5-fluoro-1H-indol-3-yl)-ethyl]- | | | |
| | amino}-acetic acid methyl ester | | | |
| 596 | {[2-(5-Fluoro-1H-indol-3-yl)-ethyl]-[3-(4- | (C) | 1.02 | 465.55 |
| | fluoro-3-methyl-phenyl)-pyrazine-2-carbonyl]- | | | |
| | amino}-acetic acid methyl ester | | | |
| 597 | {[4-(3,4-Dichloro-phenyl)-pyrimidine-5- | (C) | 1.04 | 500.88 |
| | carbonyl]-[2-(5-fluoro-1H-indol-3-yl)-ethyl]- | | | |
| | amino}-acetic acid methyl ester | | | |
| 598 | {[2-Dimethylamino-5-(4-fluoro-phenyl)- | (C) | 1.02 | 498.86 |
| | thiazole-4-carbonyl]-[2-(5-fluoro-1H-indol-3- | | | |
| | yl)-ethyl]-amino}-acetic acid methyl ester | | | |
| 599 | {[3-(4-Ethoxy-phenyl)-pyrazine-2-carbonyl]-[2- | (C) | 1.01 | 477.90 |
| | (5-fluoro-1H-indol-3-yl)-ethyl]-amino}-acetic | | | |
| | acid methyl ester | | | |
| 600 | {[2-Dimethylamino-5-(3,4-dimethyl-phenyl)- | (C) | 1.03 | 509.46 |
| | thiazole-4-carbonyl]-[2-(5-fluoro-1H-indol-3- | | | |
| | yl)-ethyl]-amino}-acetic acid methyl ester | | | |
| 601 | [[2-(5-Fluoro-1H-indol-3-yl)-ethyl]-(3-p-tolyl- | (C) | 1.00 | 447.77 |
| | pyrazine-2-carbonyl)-amino]-acetic acid methyl | | | |
| | ester | | | |
| 602 | {[2-(5-Fluoro-1H-indol-3-yl)-ethyl]-[3-(6- | (C) | 0.95 | 464.64 |
| | methoxy-pyridin-3-yl)-pyrazine-2-carbonyl]- | | | |
| | amino}-acetic acid methyl ester | | | |
| 603 | [[2-(5-Fluoro-1H-indol-3-yl)-ethyl]-(2-methyl- | (C) | 1.01 | 452.57 |
| | 5-phenyl-thiazole-4-carbonyl)-amino]-acetic | | | |
| | acid methyl ester | | | |
| 604 | {[4-(3,4-Dichloro-phenyl)-2-methyl-pyrimidine- | (C) | 1.04 | 514.83 |
| | 5-carbonyl]-[2-(5-fluoro-1H-indol-3-yl)-ethyl]- | | | |
| | amino}-acetic acid methyl ester | | | |
| 605 | {[2-(5-Fluoro-1H-indol-3-yl)-ethyl]-[3-(4- | (C) | 0.99 | 451.81 |
| | fluoro-phenyl)-pyrazine-2-carbonyl]-amino}- | | | |
| | acetic acid methyl ester | | | |
| 606 | [[2-(5-Fluoro-1H-indol-3-yl)-ethyl]-(4-p-tolyl- | (C) | 0.98 | 447.75 |
| | pyrimidine-5-carbonyl)-amino]-acetic acid | | | |
| | methyl ester | | | |
| 607 | 2-Cyclopropyl-5-m-tolyl-oxazole-4-carboxylic | (C) | 1.06 | 440.13 |
| | acid cyclopropylmethyl-[2-(1H-indol-3-yl)- | | | |
| | ethyl]-amide | | | |

II. Biological assays

### In Vitro Assay

The orexin receptor antagonistic activity of the compounds of formula (I) is determined in accordance with one of the following experimental methods.

### Experimental Method:

### Intracellular Calcium Measurements:

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 U/ml penicillin, 100 μg/ml streptomycin and 10% heat inactivated fetal calf serum (FCS). The cells are seeded at 20,000 cells/well into 384-well black clear bottom sterile plates (Greiner). The seeded plates are incubated overnight at 37° C. in 5% CO₂.

Human orexin-A as an agonist is prepared as 1 mM stock solution in MeOH:water (1:1), diluted in HBSS containing 0.1% bovine serum albumin (BSA), NaHCO₃: 0.375 g/l and 20 mM HEPES for use in the assay at a final concentration of 3 nM. Antagonists are prepared as 10 mM stock solution in DMSO, then diluted in 384-well plates, first in DMSO, then in HBSS containing 0.1% bovine serum albumin (BSA), NaHCO₃: 0.375 g/l and 20 mM HEPES.

On the day of the assay, 50 μl of staining buffer (HBSS containing 1% FCS, 20 mM HEPES, NaHCO₃: 0.375 g/l, 5 mM probenecid (Sigma) and 3 μM of the fluorescent calcium indicator fluo-4 AM (1 mM stock solution in DMSO, containing 10% pluronic) is added to each well.

The 384-well cell-plates are incubated for 50 min at 37° C. in 5% CO₂ followed by equilibration at RT for 30-120 min before measurement.

Within the Fluorescent Imaging Plate Reader (FLIPR Tetra, Molecular Devices), antagonists are added to the plate in a volume of 10 μl/well, incubated for 10 min and finally 10 μl/well of agonist is added. Fluorescence is measured for each well at 1 second intervals, and the height of each fluorescence peak is compared to the height of the fluorescence peak induced by 3 nM orexin-A with vehicle in place of antagonist. For each antagonist, the IC₅₀ value (the concentration of compound needed to inhibit 50% of the agonistic response) is determined and may be normalized using the obtained IC₅₀ value of a on-plate reference compound (normalized values in Table 1 are indicated by an asterisk *). With the FLIPR Tetra, two different conditions (conditions A and conditions B) were used, differing in adjustment of pipetting speed and cell splitting regime. The calculated IC₅₀ values of the compounds may fluctuate depending on the daily cellular assay performance. Fluctuations of this kind are known to those skilled in the art.

Antagonistic activities (IC₅₀ values) of 533 exemplified compounds are in the range of 4-4247 nM with respect to the OX1 receptor; 74 compounds have been measured with an IC₅₀ value >4250 nM in this assay. IC₅₀ values of all exemplified compounds are in the range of 2-1350 nM with an average of 138 nM with respect to the OX2 receptor. Antagonistic activities of selected compounds are displayed in Table 1.

TABLE 1

| Compound of Example | OX₁ IC₅₀ (nM) | OX₂ IC₅₀ (nM) |
|---|---|---|
| 18¹⁾ | 925 | 4 |
| 19¹⁾ | 2201 | 40 |
| 20¹⁾ | 2129 | 15 |
| 22¹⁾ | 570 | 35 |
| 30¹⁾ | 206 | 30 |
| 37¹⁾ | 2693 | 5 |
| 42¹⁾ | 1008 | 20 |
| 43¹⁾ | 3122 | 18 |
| 48¹⁾ | 4920 | 17 |
| 49¹⁾ | 4234 | 22 |
| 50¹⁾ | >10000 | 10 |
| 51¹⁾ | 1443 | 34 |
| 53¹⁾ | 8668 | 81 |
| 56¹⁾ | 697 | 299 |
| 78¹⁾ | 1121 | 141 |
| 79¹⁾ | 276 | 158 |
| 85¹⁾ | >10000 | 551 |
| 97¹⁾ | 4925 | 263 |
| 99¹⁾ | >10000 | 41 |
| 104²⁾ | 405 | 5 |
| 107²⁾ | 41 | 2 |
| 108²⁾ | 36 | 5 |
| 109²⁾ | 479 | 6 |
| 110²⁾ | 21 | 2 |
| 111²⁾ | 18 | 2 |
| 113²⁾ | 328 | 8 |
| 119²⁾ | 149 | 4 |
| 120²⁾ | 119 | 6 |
| 122²⁾ | 15 | 2 |
| 123²⁾ | 26 | 4 |
| 124²⁾ | 1473 | 82 |
| 126²⁾ | 170 | 4 |
| 127²⁾ | 1124 | 16 |
| 128²⁾ | 71 | 4 |
| 129²⁾ | 40 | 5 |
| 130²⁾ | >10000 | 279 |
| 131²⁾ | >10000 | 49 |
| 135²⁾ | >10000 | 145 |
| 157²⁾ | 8513 | 745 |
| 161²⁾ | 10 | 6 |
| 163²⁾ | 81 | 18 |
| 165²⁾ | 35* | 18* |
| 167²⁾ | 171 | 45 |
| 169²⁾ | 25 | 27 |
| 173²⁾ | 147 | 12 |
| 177²⁾ | >10000 | 954 |
| 178²⁾ | 739 | 943 |
| 180²⁾ | 4358 | 240 |
| 192²⁾ | >10000 | 719 |
| 198²⁾ | 1071 | 65 |
| 199²⁾ | 404 | 9 |
| 201²⁾ | 21 | 15 |
| 202²⁾ | 447 | 22 |
| 207²⁾ | 538 | 12 |
| 213²⁾ | 157 | 153 |
| 220²⁾ | 139 | 31 |
| 221²⁾ | 243 | 201 |
| 223²⁾ | 5 | 135 |
| 224²⁾ | 8 | 26 |
| 227²⁾ | 7 | 17 |
| 230²⁾ | 9 | 18 |
| 231²⁾ | 9 | 27 |
| 232²⁾ | 7 | 16 |
| 233²⁾ | 37* | 22* |
| 234²⁾ | 5 | 16 |
| 254²⁾ | 1774 | 232 |
| 261²⁾ | 576* | 185* |
| 262²⁾ | 9212* | 470* |
| 273²⁾ | 407* | 64* |
| 274²⁾ | 155* | 66* |
| 275²⁾ | 201* | 70* |
| 276²⁾ | 2257* | 601* |
| 279²⁾ | 5619* | 283* |
| 283²⁾ | 192* | 16* |
| 286²⁾ | 2730* | 322* |
| 287²⁾ | 2443* | 585* |
| 289²⁾ | >4265* | 627* |
| 292²⁾ | 163* | 10* |
| 294²⁾ | 3820* | 706* |
| 297²⁾ | 702* | 136* |
| 303²⁾ | 2346* | 551* |
| 307²⁾ | 345* | 20* |
| 308²⁾ | 357* | 25* |
| 311²⁾ | 147* | 16* |
| 312²⁾ | 346* | 76* |
| 313²⁾ | 5260* | 526* |
| 315²⁾ | 847* | 116* |
| 317²⁾ | 760* | 162* |
| 318²⁾ | >5420* | 848* |
| 322²⁾ | 190* | 24* |
| 324²⁾ | 77* | 8* |
| 326²⁾ | 76* | 8* |
| 330²⁾ | 18* | 8* |
| 331²⁾ | 62* | 22* |
| 337²⁾ | 16* | 5* |
| 340²⁾ | 99* | 53* |
| 341²⁾ | 18* | 36* |
| 350²⁾ | 4* | 17* |
| 352²⁾ | 20* | 37* |
| 353²⁾ | 87* | 23* |
| 357²⁾ | 14* | 30* |
| 361²⁾ | 35* | 60* |
| 371²⁾ | 1856* | 89* |
| 374²⁾ | 102* | 35* |
| 377²⁾ | 114* | 261* |
| 382²⁾ | 427* | 12* |
| 385²⁾ | 23* | 25* |
| 386²⁾ | 234 | 5 |
| 389²⁾ | 282 | 6 |
| 390²⁾ | 182 | 4 |
| 391²⁾ | 388 | 4 |
| 393²⁾ | 108 | 3 |
| 394²⁾ | 156 | 4 |
| 395²⁾ | 286 | 10 |
| 397²⁾ | 266 | 7 |
| 400²⁾ | 62* | 11* |
| 402²⁾ | 700* | 72* |
| 403²⁾ | 3831* | 230* |
| 404²⁾ | 58* | 12* |
| 410²⁾ | 3649* | 145* |
| 418²⁾ | 148* | 13* |
| 419²⁾ | 2515* | 100* |
| 420²⁾ | 373* | 83* |
| 421²⁾ | 34* | 4* |
| 423²⁾ | 1614* | 53* |
| 424²⁾ | 180* | 47* |
| 426²⁾ | 292* | 50* |
| 428²⁾ | 83* | 18* |
| 430²⁾ | 104* | 21* |
| 432²⁾ | 234 | 75 |
| 433²⁾ | 280* | 239* |
| 434²⁾ | >5420* | 816* |
| 435²⁾ | 54* | 32* |
| 436²⁾ | 24* | 2* |
| 437²⁾ | 43* | 3* |
| 438²⁾ | >11090* | 50* |
| 439²⁾ | 461* | 28* |
| 444²⁾ | 5998* | 214* |
| 471²⁾ | 97* | 25* |
| 472²⁾ | 28* | 211* |
| 479²⁾ | 1384* | 48* |
| 497²⁾ | 227* | 10* |
| 500²⁾ | 257* | 190* |
| 501²⁾ | 246* | 330* |
| 504²⁾ | 31* | 17* |
| 506²⁾ | 259* | 15* |
| 510²⁾ | 352* | 25* |
| 513²⁾ | 36* | 17* |
| 533²⁾ | 2370* | 258* |
| 536²⁾ | 262* | 9* |
| 541²⁾ | 2935* | 151* |
| 544²⁾ | 177* | 15* |
| 549²⁾ | 1160* | 130* |
| 556²⁾ | 425* | 212* |
| 587²⁾ | 404* | 38* |
| 590²⁾ | 468* | 10* |
| 604²⁾ | 421* | 196* |
| 607²⁾ | 383* | 35* |

Values in table 1 are measured using the following conditions:
¹⁾FLIPR Tetra, conditions A; or
²⁾FLIPR Tetra, conditions B.

1. A compound of formula (I) 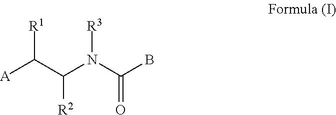
wherein
R¹ represents hydrogen, hydroxy or (C₃₋₆)cycloalkyl-amino;
R² represents hydrogen or (C₁₋₄)alkyl;
R³ represents (C₃₋₆)cycloalkyl or (C₃₋₆)cycloalkyl-(C₁₋₄)alkyl; or a (C₁₋₄)alkyl-group, which group is unsubstituted or monosubstituted with (C₁₋₄)alkoxy, hydroxy, NR⁴R⁵, C(O)NR⁴R⁵ or COOR⁶;
or a (C₁₋₄)fluoroalkyl-group;
R⁴ represents hydrogen or (C₁₋₄)alkyl;
R⁵ represents hydrogen or (C₁₋₄)alkyl;
R⁶ represents (C₁₋₄)alkyl;
A represents aryl or heterocyclyl, wherein the aryl or heterocyclyl is independently unsubstituted or mono-, di-, or tri-substituted, wherein the substituents are indepen-dently selected from the group consisting of (C₁₋₄)alkyl, (C₁₋₄)alkoxy, (C₁₋₄)alkylthio, hydroxy, amino, halogen, (C₁₋₄)fluoroalkyl, and (C₁₋₄)fluoroalkoxy; or A represents a benzo[1,3]dioxolyl- or a 2,3-dihydro-benzo[1,4]dioxinyl-group wherein said groups are unsubstituted, mono- or di-substituted with halogen; or A represents a 5H-[1,3]dioxolo[4,5-f]indole group;
B represents a group selected from 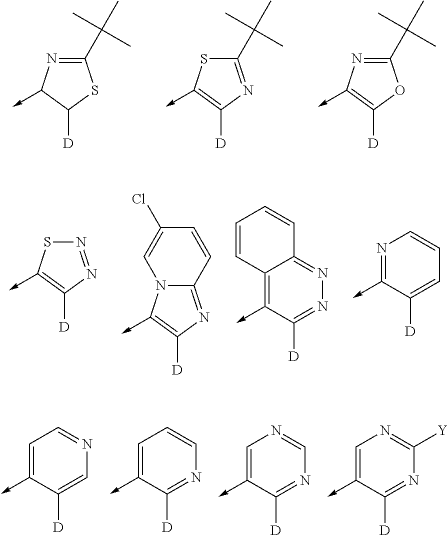
wherein
X represents hydrogen, (C₁₋₄)alkyl, (C₃₋₆)cycloalkyl, (C₁₋₄)alkoxy, R⁴R⁵N—CH₂—, NR⁴R⁵, or halogen;
Y represents hydrogen or (C₁₋₄)alkyl;
D represents aryl, wherein the aryl is unsubstituted or mono-, di-, or tri-substituted, wherein the substituents are independently selected from the group consisting of (C₁₋₄)alkyl, (C₁₋₄)alkoxy, hydroxy-(C₁₋₄)alkyl, (C₁₋₂)alkoxy-(C₁₋₄)alkoxy, halogen, (C₁₋₄)fluoroalkyl, NMe₂, (C₁₋₄)alkyl-C(O)NH— and cyano; or D represents heterocyclyl, wherein the heterocyclyl is unsubstituted or mono- or di-substituted, wherein the substituents are independently selected from the group consisting of (C₁₋₄)alkyl, (C₁₋₄)alkoxy, hydroxy-(C₁₋₄)alkyl, halogen, and (C₁₋₄)alkyl-thio;
with the proviso that A represents an optionally mono- or disubstituted indol-3-yl group, wherein the substituents are independently selected from the group consisting of (C₁₋₄)alkyl, (C₁₋₄)alkoxy and halogen, if B represents a group of formula 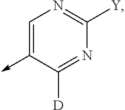
in a free or a pharmaceutically acceptable salt form.

2. A compound according to claim 1, wherein
R¹ represents hydrogen;
R² represents hydrogen or (C₁₋₄)alkyl;
R³ represents (C₃₋₆)cycloalkyl-(C₁₋₄)alkyl; or a (C₁₋₄)alkyl-group, which group is unsubstituted or monosubstituted with hydroxy, NR⁴R⁵, C(O)NR⁴R⁵ or COOR⁶; or a (C₁₋₄)fluoroalkyl group;
R⁴ represents hydrogen or (C₁₋₄)alkyl;
R⁵ represents hydrogen or (C₁₋₄)alkyl;
R⁶ represents (C₁₋₄)alkyl;
A represents heterocyclyl, wherein the heterocyclyl is unsubstituted or mono-, or di-substituted, wherein the substituents are independently selected from the group consisting of (C₁₋₄)alkyl, (C₁₋₄)alkoxy, amino, and halogen; or A represents a 5H-[1,3]dioxolo[4,5-f]indole group;
B represents a group selected from 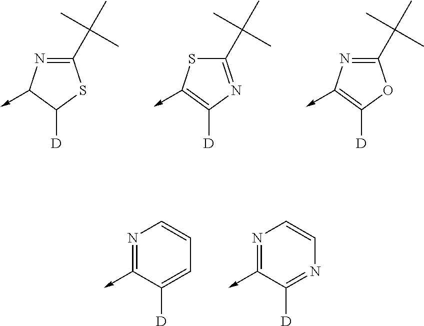
wherein
X represents hydrogen, (C₁₋₄)alkyl, (C₃₋₆)cycloalkyl, (C₁₋₄)alkoxy, R⁴R⁵N—CH₂—, or NR⁴R⁵;
D represents aryl, wherein the aryl is unsubstituted or mono-, di-, or tri-substituted, wherein the substituents are independently selected from the group consisting of (C₁₋₄alkyl, (C₁₋₄)alkoxy, hydroxy-(C₁₋₄)alkyl, halogen, NMe₂, and cyano; or D represents heterocyclyl, wherein the heterocyclyl is unsubstituted or mono- or di-substituted, wherein the substituents are independently selected from the group consisting of (C₁₋₄)alkyl, (C₁₋₄)alkoxy, hydroxy-(C₁₋₄)alkyl, halogen, and (C₁₋₄alkyl-thio,
in a free or a pharmaceutically acceptable salt form.

3. The compound according to claim 1, wherein
R¹ represents hydrogen,
in a free or a pharmaceutically acceptable salt form.

4. The compound according to claim 1, wherein
R² represents hydrogen,
in a free or a pharmaceutically acceptable salt form.

5. The compound according to claim 1, wherein R³ represents (C₃₋₆)cycloalkyl or (C₃₋₆)cycloalkyl-(C₁₋₄)alkyl; or a (C₁₋₄)alkyl-group, which group is monosubstituted with (C₁₋₄)alkoxy, hydroxy, NR⁴R⁵, C(O)NR⁴R⁵ or COOR⁶; or a (C₁₋₄)fluoroalkyl group,
in a free or a pharmaceutically acceptable salt form.

6. The compound according to claim 1, wherein A represents phenyl, wherein the phenyl is di- or tri-substituted, wherein the substituents are independently selected from the group consisting of (C₁₋₄alkyl, (C₁₋₄)alkoxy, (C₁₋₄)alkylthio, halogen, and (C₁₋₄)fluoroalkoxy,
in a free or a pharmaceutically acceptable salt form.

7. The compound according to claim 1, wherein A represents an indolyl radical or a benzimidazolyl radical which radicals are unsubstituted or mono-, or di-substituted, wherein the substituents are independently selected from the group consisting of (C₁₋₄)alkyl, (C₁₋₄)alkoxy, and halogen,
in a free or a pharmaceutically acceptable salt form.

8. The compound according to claim 1, wherein B represents a group selected from 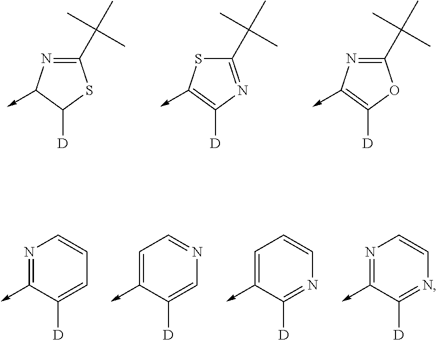
in a free or a pharmaceutically acceptable salt form thereof.

9. The compound according to claim 1, wherein B represents a group selected from 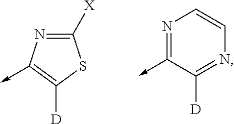
in a free or a pharmaceutically acceptable salt form.

10. The compound according to claim 1, wherein
X represents hydrogen, (C₁₋₄)alkyl, or NR⁴R⁵,
in a free or a pharmaceutically acceptable salt form.

11. A compound according to claim 1, wherein D represents phenyl, wherein the phenyl is unsubstituted or mono-, di-, or tri-substituted, wherein the substituents are independently selected from the group consisting of (C₁₋₄)alkyl, (C₁₋₄)alkoxy, and halogen,
in a free or a pharmaceutically acceptable salt form.

12. A compound according to claim 1, wherein D represents heterocyclyl, wherein the heterocyclyl is unsubstituted or mono- or di-substituted, wherein the substituents are independently selected from the group consisting of (C₁₋₄)alkyl, (C₁₋₄)alkoxy, hydroxy-(C₁₋₄)alkyl, halogen, and (C₁₋₄)alkyl-thio,
in a free or a pharmaceutically acceptable salt form.

13. A compound according to claim 1 selected from the group consisting of:
2-Amino-5-(3-fluoro-phenyl)-thiazole-4-carboxylic acid [2-(3-bromo-phenyl)-ethyl]-cyclopropylmethyl-amide;
5-(3-Fluoro-phenyl)-2-methyl-thiazole-4-carboxylic acid cyclopropylmethyl-[2-(3,4-dimethoxy-phenyl)-ethyl]-amide;
2-Methyl-5-m-tolyl-thiazole-4-carboxylic acid cyclopropylmethyl-[2-(3,4-dimethoxy-phenyl)-ethyl]-amide;
2-Bromo-5-m-tolyl-thiazole-4-carboxylic acid cyclopropylmethyl-[2-(3,4-dimethoxy-phenyl)-ethyl]-amide;
2-Amino-5-(3-fluoro-phenyl)-thiazole-4-carboxylic acid cyclopropylmethyl-[2-(3,4-dimethoxy-phenyl)-ethyl]-amide;
2-Amino-5-m-tolyl-thiazole-4-carboxylic acid cyclopropylmethyl-[2-(3,4-dimethoxy-phenyl)-ethyl]-amide;
2-Amino-5-(3-chloro-phenyl)-thiazole-4-carboxylic acid cyclopropylmethyl-[2-(3,4-dimethoxy-phenyl)-ethyl]-amide;
5-(4-Cyano-phenyl)-2-methyl-thiazole-4-carboxylic acid cyclopropylmethyl-[2-(3,4-dimethoxy-phenyl)-ethyl]-amide;
5-(3,5-Dimethyl-phenyl)-2-methyl-thiazole-4-carboxylic acid cyclopropylmethyl-[2-(3,4-dimethoxy-phenyl)-ethyl]-amide;
5-(3,5-Difluoro-phenyl)-2-methyl-thiazole-4-carboxylic acid cyclopropylmethyl-[2-(3,4-dimethoxy-phenyl)-ethyl]-amide;
5-(3-Fluoro-5-trifluoromethyl-phenyl)-2-methyl-thiazole-4-carboxylic acid cyclopropylmethyl-[2-(3,4-dimethoxy-phenyl)-ethyl]-amide;
5-(2,4-Dimethyl-phenyl)-2-methyl-thiazole-4-carboxylic acid cyclopropylmethyl-[2-(3,4-dimethoxy-phenyl)-ethyl]-amide;
5-(3-Fluoro-2-methyl-phenyl)-2-methyl-thiazole-4-carboxylic acid cyclopropylmethyl-[2-(3,4-dimethoxy-phenyl)-ethyl]-amide;
5-(2,3-Dimethyl-phenyl)-2-methyl-thiazole-4-carboxylic acid cyclopropylmethyl-[2-(3,4-dimethoxy-phenyl)-ethyl]-amide;
5-(3,4-Dichloro-phenyl)-2-methyl-thiazole-4-carboxylic acid cyclopropylmethyl-[2-(3,4-dimethoxy-phenyl)-ethyl]-amide;
5-(3-Fluoro-4-methyl-phenyl)-2-methyl-thiazole-4-carboxylic acid cyclopropylmethyl-[2-(3,4-dimethoxy-phenyl)-ethyl]-amide;
5-(3,4-Dimethyl-phenyl)-2-methyl-thiazole-4-carboxylic acid cyclopropylmethyl-[2-(3,4-dimethoxy-phenyl)-ethyl]-amide;
2-Methyl-5-phenyl-thiazole-4-carboxylic acid cyclopropylmethyl-[2-(3,4-dimethoxy-phenyl)-ethyl]-amide;
5-(3-Cyano-phenyl)-2-methyl-thiazole-4-carboxylic acid cyclopropylmethyl-[2-(3,4-dimethoxy-phenyl)-ethyl]-amide;
5-(4-Ethyl-phenyl)-2-methyl-thiazole-4-carboxylic acid cyclopropylmethyl-[2-(3,4-dimethoxy-phenyl)-ethyl]-amide;
5-(3,4-Difluoro-phenyl)-2-methyl-thiazole-4-carboxylic acid cyclopropylmethyl-[2-(3,4-dimethoxy-phenyl)-ethyl]-amide;
2-Cyclopropyl-5-phenyl-thiazole-4-carboxylic acid cyclopropylmethyl-[2-(3,4-dimethoxy-phenyl)-ethyl]-amide;
2-Cyclopropyl-5-p-tolyl-thiazole-4-carboxylic acid cyclopropylmethyl-[2-(3,4-dimethoxy-phenyl)-ethyl]-amide;
2-Cyclopropyl-5-(4-fluoro-phenyl)-thiazole-4-carboxylic acid cyclopropylmethyl-[2-(3,4-dimethoxy-phenyl)-ethyl]-amide;
2-Cyclopropyl-5-(3-fluoro-phenyl)-thiazole-4-carboxylic acid cyclopropylmethyl-[2-(3,4-dimethoxy-phenyl)-ethyl]-amide;
2-Cyclopropyl-5-(3-trifluoromethyl-phenyl)-thiazole-4-carboxylic acid cyclopropylmethyl-[2-(3,4-dimethoxy-phenyl)-ethyl]-amide;
2-Cyclopropyl-5-(3-fluoro-4-methyl-phenyl)-thiazole-4-carboxylic acid cyclopropylmethyl-[2-(3,4-dimethoxy-phenyl)-ethyl]-amide;
2-Cyclopropyl-5-(3-fluoro-5-trifluoromethyl-phenyl)-thiazole-4-carboxylic acid cyclopropyl-methyl-[2-(3,4-dimethoxy-phenyl)-ethyl]-amide;
2-Methoxy-5-m-tolyl-thiazole-4-carboxylic acid cyclopropylmethyl-[2-(3,4-dimethoxy-phenyl)-ethyl]-amide;
2-Dimethylamino-5-m-tolyl-thiazole-4-carboxylic acid cyclopropylmethyl-[2-(3,4-dimethoxy-phenyl)-ethyl]-amide;
2-Amino-5-(2-fluoro-phenyl)-thiazole-4-carboxylic acid cyclopropylmethyl-[2-(3,4-dimethoxy-phenyl)-ethyl]-amide;
2-Amino-5-phenyl-thiazole-4-carboxylic acid cyclopropylmethyl-[2-(3,4-dimethoxy-phenyl)-ethyl]-amide;
2-Amino-5-p-tolyl-thiazole-4-carboxylic acid cyclopropylmethyl-[2-(3,4-dimethoxy-phenyl)-ethyl]-amide;
5-m-Tolyl-thiazole-4-carboxylic acid cyclopropylmethyl-[2-(3,4-dimethoxy-phenyl)-ethyl]-amide;
5-(3-Chloro-phenyl)-thiazole-4-carboxylic acid cyclopropylmethyl-[2-(3,4-dimethoxy-phenyl)-ethyl]-amide;
5-(3-Trifluoromethyl-phenyl)-thiazole-4-carboxylic acid cyclopropylmethyl-[2-(3,4-dimethoxy-phenyl)-ethyl]amide;
5-(2-Fluoro-phenyl)-thiazole-4-carboxylic acid cyclopropylmethyl-[2-(3,4-dimethoxy-phenyl)-ethyl]-amide;
5-(4-Fluoro-phenyl)-thiazole-4-carboxylic acid cyclopropylmethyl-[2-(3,4-dimethoxy-phenyl)-ethyl]-amide;
5-(3-Methoxy-phenyl)-thiazole-4-carboxylic acid cyclopropylmethyl-[2-(3,4-dimethoxy-phenyl)-ethyl]-amide;
5-Phenyl-thiazole-4-carboxylic acid cyclopropylmethyl-[2-(3,4-dimethoxy-phenyl)-ethyl]-amide;
5-(3-Fluoro-phenyl)-thiazole-4-carboxylic acid cyclopropylmethyl-[2-(3,4-dimethoxy-phenyl)-ethyl]-amide;
5-(3-Methoxy-phenyl)-2-methyl-oxazole-4-carboxylic acid cyclopropylmethyl-[2-(3,4-dimethoxy-phenyl)-ethyl]-amide;
2-Methyl-5-(3-trifluoromethyl-phenyl)-oxazole-4-carboxylic acid cyclopropylmethyl-[2-(3,4-dimethoxy-phenyl)-ethyl]-amide;
4-(3-Chloro-phenyl)-2-methyl-thiazole-5-carboxylic acid cyclopropylmethyl-[2-(3,4-dimethoxy-phenyl)-ethyl]-amide;
2-Methyl-4-(3-trifluoromethyl-phenyl)-thiazole-5-carboxylic acid cyclopropylmethyl-[2-(3,4-dimethoxy-phenyl)-ethyl]-amide;
4-(3-Methoxy-phenyl)-2-methyl-thiazole-5-carboxylic acid cyclopropylmethyl-[2-(3,4-dimethoxy-phenyl)-ethyl]-amide;
2-Methyl-4-(4-trifluoromethyl-phenyl)-thiazole-5-carboxylic acid cyclopropylmethyl-[2-(3,4-dimethoxy-phenyl)-ethyl]-amide;
4-(4-Chloro-phenyl)-2-methyl-thiazole-5-carboxylic acid cyclopropylmethyl-[2-(3,4-dimethoxy-phenyl)-ethyl]-amide;
2-Methyl-4-p-tolyl-thiazole-5-carboxylic acid cyclopropylmethyl-[2-(3,4-dimethoxy-phenyl)-ethyl]-amide;
4-(4-Fluoro-phenyl)-2-methyl-thiazole-5-carboxylic acid cyclopropylmethyl-[2-(3,4-dimethoxy-phenyl)-ethyl]-amide;
3-Phenyl-cinnoline-4-carboxylic acid cyclopropylmethyl-[2-(3,4-dimethoxy-phenyl)-ethyl]-amide;
6-Chloro-2-phenyl-imidazo[1,2-a]pyridine-3-carboxylic acid cyclopropylmethyl-[2-(3,4-dimethoxy-phenyl)-ethyl]-amide;
4-Phenyl-[1,2,3]thiadiazole-5-carboxylic acid cyclopropylmethyl-[2-(3,4-dimethoxy-phenyl)-ethyl]-amide;
3-Phenyl-pyrazine-2-carboxylic acid cyclopropylmethyl-[2-(3,4-dimethoxy-phenyl)-ethyl]-amide;
2-Methyl-5-m-tolyl-thiazole-4-carboxylic acid cyclopropylmethyl-[2-(3,4-dimethoxy-phenyl)-2-hydroxy-ethyl]-amide;
2-Bromo-5-m-tolyl-thiazole-4-carboxylic acid cyclopropylmethyl-[2-(3,4-dimethoxy-phenyl)-2-hydroxy-ethyl]-amide;
2-Amino-5-(3-fluoro-phenyl)-thiazole-4-carboxylic acid cyclopropylmethyl-[2-(3,4-dimethoxy-phenyl)-2-hydroxy-ethyl]-amide;
2-Amino-5-m-tolyl-thiazole-4-carboxylic acid cyclopropylmethyl-[2-(3,4-dimethoxy-phenyl)-2-hydroxy-ethyl]-amide;
2-Amino-5-(3-chloro-phenyl)-thiazole-4-carboxylic acid cyclopropylmethyl-[2-(3,4-dimethoxy-phenyl)-2-hydroxy-ethyl]-amide;
5-(3,5-Dimethyl-phenyl)-2-methyl-thiazole-4-carboxylic acid cyclopropylmethyl-[2-(3,4-dimethoxy-phenyl)-2-hydroxy-ethyl]-amide;
5-(3,5-Difluoro-phenyl)-2-methyl-thiazole-4-carboxylic acid cyclopropylmethyl-[2-(3,4-dimethoxy-phenyl)-2-hydroxy-ethyl]-amide;
5-(2,4-Dimethyl-phenyl)-2-methyl-thiazole-4-carboxylic acid cyclopropylmethyl-[2-(3,4-dimethoxy-phenyl)-2-hydroxy-ethyl]-amide;
5-(3-Fluoro-2-methyl-phenyl)-2-methyl-thiazole-4-carboxylic acid cyclopropylmethyl-[2-(3,4-dimethoxy-phenyl)-2-hydroxy-ethyl]-amide;
5-(2,3-Dimethyl-phenyl)-2-methyl-thiazole-4-carboxylic acid cyclopropylmethyl-[2-(3,4-dimethoxy-phenyl)-2-hydroxy-ethyl]-amide;
5-(3,4-Dichloro-phenyl)-2-methyl-thiazole-4-carboxylic acid cyclopropylmethyl-[2-(3,4-dimethoxy-phenyl)-2-hydroxy-ethyl]-amide;
5-(3-Fluoro-4-methyl-phenyl)-2-methyl-thiazole-4-carboxylic acid cyclopropylmethyl-[2-(3,4-dimethoxy-phenyl)-2-hydroxy-ethyl]-amide;
5-(3,4-Dimethyl-phenyl)-2-methyl-thiazole-4-carboxylic acid cyclopropylmethyl-[2-(3,4-dimethoxy-phenyl)-2-hydroxy-ethyl]-amide;
2-Methyl-5-phenyl-thiazole-4-carboxylic acid cyclopropylmethyl-[2-(3,4-dimethoxy-phenyl)-2-hydroxy-ethyl]-amide;
5-(4-Ethyl-phenyl)-2-methyl-thiazole-4-carboxylic acid cyclopropylmethyl-[2-(3,4-dimethoxy-phenyl)-2-hydroxy-ethyl]-amide;
5-(3,4-Difluoro-phenyl)-2-methyl-thiazole-4-carboxylic acid cyclopropylmethyl-[2-(3,4-dimethoxy-phenyl)-2-hydroxy-ethyl]-amide;
2-Cyclopropyl-5-phenyl-thiazole-4-carboxylic acid cyclopropylmethyl-[2-(3,4-dimethoxy-phenyl)-2-hydroxy-ethyl]-amide;
2-Cyclopropyl-5-p-tolyl-thiazole-4-carboxylic acid cyclopropylmethyl-[2-(3,4-dimethoxy-phenyl)-2-hydroxy-ethyl]-amide;
2-Cyclopropyl-5-(4-fluoro-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-phenyl)-thiazole-4-carboxylic acid cyclopropylmethyl-[2-(3,4-dimethoxy-phenyl)-2-hydroxy-ethyl]-amide;
2-Cyclopropyl-5-(3-trifluoromethyl-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-4-methyl-phenyl)-thiazole-4-carboxylic acid cyclopropylmethyl-[2-(3,4-dimethoxy-phenyl)-2-hydroxy-ethyl]-amide;
2-Cyclopropyl-5-(3-fluoro-5-trifluoromethyl-phenyl)-thiazole-4-carboxylic acid cyclopropylmethyl-[2-(3,4-dimethoxy-phenyl)-2-hydroxy-ethyl]-amide;
2-Methoxy-5-m-tolyl-thiazole-4-carboxylic acid cyclopropylmethyl-[2-(3,4-dimethoxy-phenyl)-2-hydroxy-ethyl]-amide;
2-Dimethylamino-5-m-tolyl-thiazole-4-carboxylic acid cyclopropylmethyl-[2-(3,4-dimethoxy-phenyl)-2-hydroxy-ethyl]-amide;
2-Amino-5-(2-fluoro-phenyl)-thiazole-4-carboxylic acid cyclopropylmethyl-[2-(3,4-dimethoxy-phenyl)-2-hydroxy-ethyl]-amide;
2-Amino-5-phenyl-thiazole-4-carboxylic acid cyclopropylmethyl-[2-(3,4-dimethoxy-phenyl)-2-hydroxy-ethyl]-amide;
2-Amino-5-p-tolyl-thiazole-4-carboxylic acid cyclopropylmethyl-[2-(3,4-dimethoxy-phenyl)-2-hydroxy-ethyl]-amide;
5-m-Tolyl-thiazole-4-carboxylic acid cyclopropylmethyl-[2-(3,4-dimethoxy-phenyl)-2-hydroxy-ethyl]-amide;
5-(3-Chloro-phenyl)-thiazole-4-carboxylic acid cyclopropylmethyl-[2-(3,4-dimethoxy-phenyl)-2-hydroxy-ethyl]-amide;
5-(3-Trifluoromethyl-phenyl)-thiazole-4-carboxylic acid cyclopropylmethyl-[2-(3,4-dimethoxy-phenyl)-2-hydroxy-ethyl]-amide;
5-(2-Fluoro-phenyl)-thiazole-4-carboxylic acid cyclopropylmethyl-[2-(3,4-dimethoxy-phenyl)-2-hydroxy-ethyl]-amide;
5-(4-Fluoro-phenyl)-thiazole-4-carboxylic acid cyclopropylmethyl-[2-(3,4-dimethoxy-phenyl)-2-hydroxy-ethyl]-amide;
5-(3-Methoxy-phenyl)-thiazole-4-carboxylic acid cyclopropylmethyl-[2-(3,4-dimethoxy-phenyl)-2-hydroxy-ethyl]-amide;
5-Phenyl-thiazole-4-carboxylic acid cyclopropylmethyl-[2-(3,4-dimethoxy-phenyl)-2-hydroxy-ethyl]-amide;
5-(3-Fluoro-phenyl)-thiazole-4-carboxylic acid cyclopropylmethyl-[2-(3,4-dimethoxy-phenyl)-2-hydroxy-ethyl]-amide;
4-(3-Chloro-phenyl)-2-methyl-thiazole-5-carboxylic acid cyclopropylmethyl-[2-(3,4-dimethoxy-phenyl)-2-hydroxy-ethyl]-amide;
2-Methyl-4-(3-trifluoromethyl-phenyl)-thiazole-5-carboxylic acid cyclopropylmethyl-[2-(3,4-dimethoxy-phenyl)-2-hydroxy-ethyl]-amide;
4-(3-Methoxy-phenyl)-2-methyl-thiazole-5-carboxylic acid cyclopropylmethyl-[2-(3,4-dimethoxy-phenyl)-2-hydroxy-ethyl]-amide;
4-(4-Chloro-phenyl)-2-methyl-thiazole-5-carboxylic acid cyclopropylmethyl-[2-(3,4-dimethoxy-phenyl)-2-hydroxy-ethyl]-amide;
2-Methyl-4-p-tolyl-thiazole-5-carboxylic acid cyclopropylmethyl-[2-(3,4-dimethoxy-phenyl)-2-hydroxy-ethyl]-amide;
4-(4-Fluoro-phenyl)-2-methyl-thiazole-5-carboxylic acid cyclopropylmethyl-[2-(3,4-dimethoxy-phenyl)-2-hydroxy-ethyl]-amide;
6-Chloro-2-phenyl-imidazo[1,2-a]pyridine-3-carboxylic acid cyclopropylmethyl-[2-(3,4-dimethoxy-phenyl)-2-hydroxy-ethyl]-amide;
4-Phenyl-[1,2,3]thiadiazole-5-carboxylic acid cyclopropylmethyl-[2-(3,4-dimethoxy-phenyl)-2-hydroxy-ethyl]-amide;
3-Phenyl-pyrazine-2-carboxylic acid cyclopropylmethyl-[2-(3,4-dimethoxy-phenyl)-2-hydroxy-ethyl]-amide;
5-(3-Fluoro-phenyl)-2-methyl-thiazole-4-carboxylic acid cyclopropylmethyl-[2-(3,4-dimethoxy-phenyl)-1-methyl-ethyl]-amide;
2-Amino-5-m-tolyl-thiazole-4-carboxylic acid cyclopropylmethyl-[2-(3,4-dimethoxy-phenyl)-1-methyl-ethyl]-amide;
5-(3,4-Dimethyl-phenyl)-2-methyl-thiazole-4-carboxylic acid cyclopropylmethyl-[2-(3,4-dimethoxy-phenyl)-1-methyl-ethyl]-amide;
5-(3-Fluoro-phenyl)-2-methyl-thiazole-4-carboxylic acid [2-(3,4-dimethoxy-phenyl)-ethyl]-methyl-amide;
2-Amino-5-m-tolyl-thiazole-4-carboxylic acid [2-(3,4-dimethoxy-phenyl)-ethyl]methyl-amide;
5-(3,4-Dimethyl-phenyl)-2-methyl-thiazole-4-carboxylic acid [2-(3,4-dimethoxy-phenyl)-ethyl]-methyl-amide;
5-(3-Fluoro-phenyl)-2-methyl-thiazole-4-carboxylic acid [2-(3,4-dimethoxy-phenyl)-ethyl]-ethyl-amide;
2-Amino-5-m-tolyl-thiazole-4-carboxylic acid [2-(3,4-dimethoxy-phenyl)-ethyl]-ethyl-amide;
5-(3,4-Dimethyl-phenyl)-2-methyl-thiazole-4-carboxylic acid [2-(3,4-dimethoxy-phenyl)-ethyl]-ethyl-amide;
5-(3-Fluoro-phenyl)-2-methyl-thiazole-4-carboxylic acid [2-(3,4-dimethoxy-phenyl)-ethyl]-propyl-amide;
2-Amino-5-m-tolyl-thiazole-4-carboxylic acid [2-(3,4-dimethoxy-phenyl)-ethyl]-propyl-amide;
5-(3,4-Dimethyl-phenyl)-2-methyl-thiazole-4-carboxylic acid [2-(3,4-dimethoxy-phenyl)-ethyl]-propyl-amide;
5-(3-Fluoro-phenyl)-2-methyl-thiazole-4-carboxylic acid [2-(3,4-dimethoxy-phenyl)-ethyl]-isobutyl-amide;
2-Amino-5-m-tolyl-thiazole-4-carboxylic acid [2-(3,4-dimethoxy-phenyl)-ethyl]-isobutyl-amide;
5-(3,4-Dimethyl-phenyl)-2-methyl-thiazole-4-carboxylic acid [2-(3,4-dimethoxy-phenyl)-ethyl]-isobutyl-amide;
5-(3-Fluoro-phenyl)-2-methyl-thiazole-4-carboxylic acid [2-(3,4-dimethoxy-phenyl)-ethyl]-isopropyl-amide;
2-Amino-5-m-tolyl-thiazole-4-carboxylic acid [2-(3,4-dimethoxy-phenyl)-ethyl]-isopropyl-amide;
5-(3,4-Dimethyl-phenyl)-2-methyl-thiazole-4-carboxylic acid [2-(3,4-dimethoxy-phenyl)-ethyl]-isopropyl-amide;
5-(3-Fluoro-phenyl)-2-methyl-thiazole-4-carboxylic acid [2-(3,4-dimethoxy-phenyl)-ethyl]-(2,2,2-trifluoro-ethyl)-amide;
2-Amino-5-m-tolyl-thiazole-4-carboxylic acid [2-(3,4-dimethoxy-phenyl)-ethyl]-(2,2,2-trifluoro-ethyl)-amide;
5-(3,4-Dimethyl-phenyl)-2-methyl-thiazole-4-carboxylic acid [2-(3,4-dimethoxy-phenyl)-ethyl]-(2,2,2-trifluoro-ethyl)-amide;
5-(3-Fluoro-phenyl)-2-methyl-thiazole-4-carboxylic acid cyclopropyl-[2-(3,4-dimethoxy-phenyl)-ethyl]-amide;
2-Amino-5-m-tolyl-thiazole-4-carboxylic acid cyclopropyl-[2-(3,4-dimethoxy-phenyl)-ethyl]-amide;
5-(3,4-Dimethyl-phenyl)-2-methyl-thiazole-4-carboxylic acid cyclopropyl-[2-(3,4-dimethoxy-phenyl)-ethyl]-amide;
5-(3-Fluoro-phenyl)-2-methyl-thiazole-4-carboxylic acid [2-(3,4-dimethoxy-phenyl)-ethyl]-(2-hydroxy-ethyl)-amide;
2-Amino-5-m-tolyl-thiazole-4-carboxylic acid [2-(3,4-dimethoxy-phenyl)-ethyl]-(2-hydroxy-ethyl)-amide;
5-(3,4-Dimethyl-phenyl)-2-methyl-thiazole-4-carboxylic acid [2-(3,4-dimethoxy-phenyl)-ethyl]-(2-hydroxy-ethyl)-amide;
5-(3-Fluoro-phenyl)-2-methyl-thiazole-4-carboxylic acid [2-(3,4-dimethoxy-phenyl)-ethyl]-(2-methoxy-ethyl)-amide;
2-Amino-5-m-tolyl-thiazole-4-carboxylic acid [2-(3,4-dimethoxy-phenyl)-ethyl]-(2-methoxy-ethyl)-amide;
5-(3,4-Dimethyl-phenyl)-2-methyl-thiazole-4-carboxylic acid [2-(3,4-dimethoxy-phenyl)-ethyl]-(2-methoxy-ethyl)-amide;
2-Amino-5-m-tolyl-thiazole-4-carboxylic acid [2-(3,4-dimethoxy-phenyl)-ethyl]-(2-dimethylamino-ethyl)-amide;
5-(3-Fluoro-phenyl)-2-methyl-thiazole-4-carboxylic acid carbamoylmethyl-[2-(3,4-dimethoxy-phenyl)-ethyl]-amide;
2-Amino-5-m-tolyl-thiazole-4-carboxylic acid carbamoylmethyl-[2-(3,4-dimethoxy-phenyl)-ethyl]-amide;
5-(3,4-Dimethyl-phenyl)-2-methyl-thiazole-4-carboxylic acid carbamoylmethyl-[2-(3,4-dimethoxy-phenyl)-ethyl]-amide;
5-(3-Fluoro-phenyl)-2-methyl-thiazole-4-carboxylic acid [2-(3,4-dimethoxy-phenyl)-ethyl]-dimethylcarbamoylmethyl-amide;
2-Amino-5-m-tolyl-thiazole-4-carboxylic acid [2-(3,4-dimethoxy-phenyl)-ethyl]-dimethylcarbamoylmethyl-amide;
5-(3,4-Dimethyl-phenyl)-2-methyl-thiazole-4-carboxylic acid [2-(3,4-dimethoxy-phenyl)-ethyl]-dimethylcarbamoylmethyl-amide;
2-Amino-5-m-tolyl-thiazole-4-carboxylic acid cyclopropylmethyl-phenethyl-amide;
2-Amino-5-m-tolyl-thiazole-4-carboxylic acid [2-(2-chloro-phenyl)-ethyl]-cyclopropylmethyl-amide;
2-Amino-5-m-tolyl-thiazole-4-carboxylic acid cyclopropylmethyl-[2-(2-methoxy-phenyl)-ethyl]-amide;
2-Amino-5-m-tolyl-thiazole-4-carboxylic acid cyclopropylmethyl-[2-(2-fluoro-phenyl)-ethyl]-amide;
2-Amino-5-m-tolyl-thiazole-4-carboxylic acid cyclopropylmethyl-(2-o-tolyl-ethyl)-amide;
2-Amino-5-m-tolyl-thiazole-4-carboxylic acid cyclopropylmethyl-(2-m-tolyl-ethyl)-amide;
2-Amino-5-m-tolyl-thiazole-4-carboxylic acid cyclopropylmethyl-[2-(3-methoxy-phenyl)-ethyl]-amide;
2-Amino-5-m-tolyl-thiazole-4-carboxylic acid [2-(4-chloro-phenyl)-ethyl]-cyclopropylmethyl-amide;
2-Amino-5-m-tolyl-thiazole-4-carboxylic acid cyclopropylmethyl-(2-p-tolyl-ethyl)-amide;
2-Amino-5-m-tolyl-thiazole-4-carboxylic acid cyclopropylmethyl-[2-(4-ethyl-phenyl)-ethyl]-amide;
2-Amino-5-m-tolyl-thiazole-4-carboxylic acid cyclopropylmethyl-[2-(4-methoxy-phenyl)-ethyl]-amide;
2-Amino-5-m-tolyl-thiazole-4-carboxylic acid cyclopropylmethyl-[2-(4-hydroxy-phenyl)-ethyl]-amide;
2-Amino-5-m-tolyl-thiazole-4-carboxylic acid cyclopropylmethyl-[2-(4-methylsulfanyl-phenyl)-ethyl]-amide;
2-Amino-5-m-tolyl-thiazole-4-carboxylic acid cyclopropylmethyl-[2-(4-trifluoromethyl-phenyl)-ethyl]-amide;
2-Amino-5-m-tolyl-thiazole-4-carboxylic acid cyclopropylmethyl-[2-(4-trifluoromethoxy-phenyl)-ethyl]-amide;
2-Amino-5-m-tolyl-thiazole-4-carboxylic acid cyclopropylmethyl-[2-(2,4-dimethyl-phenyl)-ethyl]-amide;
2-Amino-5-m-tolyl-thiazole-4-carboxylic acid cyclopropylmethyl-[2-(2,5-dimethoxy-phenyl)-ethyl]-amide;
2-Amino-5-m-tolyl-thiazole-4-carboxylic acid cyclopropylmethyl-[2-(2,5-dimethyl-phenyl)-ethyl]-amide;
2-Amino-5-m-tolyl-thiazole-4-carboxylic acid [2-(5-bromo-2-methoxy-phenyl)-ethyl]-cyclopropylmethyl-amide;
2-Amino-5-m-tolyl-thiazole-4-carboxylic acid (2-benzo[1,3]dioxol-5-yl-ethyl)-cyclopropylmethyl-amide;
2-Amino-5-m-tolyl-thiazole-4-carboxylic acid cyclopropylmethyl-[2-(2,2-difluoro-benzo[1,3]dioxol-5-yl)-ethyl]-amide;
2-Amino-5-m-tolyl-thiazole-4-carboxylic acid cyclopropylmethyl-[2-(2,3-dihydro-benzo[1,4]dioxin-6-yl)-ethyl]-amide;
2-Amino-5-m-tolyl-thiazole-4-carboxylic acid cyclopropylmethyl-[2-(4-ethoxy-3-methoxy-phenyl)-ethyl]-amide;
2-Amino-5-m-tolyl-thiazole-4-carboxylic acid cyclopropylmethyl-[2-(3-ethoxy-4-methoxy-phenyl)-ethyl]-amide;
2-Amino-5-m-tolyl-thiazole-4-carboxylic acid cyclopropylmethyl-[2-(4-methoxy-3-methylsulfanyl-phenyl)-ethyl]-amide;
2-Amino-5-m-tolyl-thiazole-4-carboxylic acid cyclopropylmethyl-[2-(4-methoxy-3-methyl-phenyl)-ethyl]-amide;
2-Amino-5-m-tolyl-thiazole-4-carboxylic acid [2-(3-bromo-4-methoxy-phenyl)-ethyl]-cyclopropylmethyl-amide;
2-Amino-5-m-tolyl-thiazole-4-carboxylic acid cyclopropylmethyl-[2-(3,4-dimethyl-phenyl)-ethyl]-amide;
2-Amino-5-m-tolyl-thiazole-4-carboxylic acid cyclopropylmethyl-[2-(3-difluoromethoxy-4-methoxy-phenyl)-ethyl]-amide;
2-Amino-5-m-tolyl-thiazole-4-carboxylic acid cyclopropylmethyl-[2-(4-difluoromethoxy-3-methoxy-phenyl)-ethyl]-amide;
2-Amino-5-m-tolyl-thiazole-4-carboxylic acid cyclopropylmethyl-(2-naphthalen-2-yl-ethyl)-amide;
2-Amino-5-m-tolyl-thiazole-4-carboxylic acid cyclopropylmethyl-[2-(4-hydroxy-3-methoxy-phenyl)-ethyl]-amide;
2-Amino-5-m-tolyl-thiazole-4-carboxylic acid cyclopropylmethyl-[1-(3,4-dimethoxy-benzyl)-propyl]-amide;
2-Amino-5-m-tolyl-thiazole-4-carboxylic acid cyclopropylmethyl-[2-(3,5-dimethoxy-phenyl)-ethyl]-amide;
2-Amino-5-m-tolyl-thiazole-4-carboxylic acid cyclopropylmethyl-[2-(2,6-dichloro-phenyl)-ethyl]-amide;
2-Amino-5-m-tolyl-thiazole-4-carboxylic acid cyclopropylmethyl-[2-(3,4,5-trimethoxy-phenyl)-ethyl]-amide;
2-Amino-5-m-tolyl-thiazole-4-carboxylic acid cyclopropylmethyl-[2-(4-isopropoxy-3,5-dimethoxy-phenyl)-ethyl]-amide;
2-Amino-5-m-tolyl-thiazole-4-carboxylic acid cyclopropylmethyl-[2-(4-iodo-2,5-dimethoxy-phenyl)-ethyl]-amide;
5-(3,4-Dimethyl-phenyl)-2-methyl-thiazole-4-carboxylic acid cyclopropylmethyl-phenethyl-amide;
5-(3,4-Dimethyl-phenyl)-2-methyl-thiazole-4-carboxylic acid [2-(2-chloro-phenyl)-ethyl]-cyclopropylmethyl-amide;
5-(3,4-Dimethyl-phenyl)-2-methyl-thiazole-4-carboxylic acid cyclopropylmethyl-[2-(2-methoxy-phenyl)-ethyl]-amide;
5-(3,4-Dimethyl-phenyl)-2-methyl-thiazole-4-carboxylic acid cyclopropylmethyl-[2-(2-fluoro-phenyl)-ethyl]-amide;
5-(3,4-Dimethyl-phenyl)-2-methyl-thiazole-4-carboxylic acid cyclopropylmethyl-(2-m-tolyl-ethyl)-amide;
5-(3,4-Dimethyl-phenyl)-2-methyl-thiazole-4-carboxylic acid cyclopropylmethyl-[2-(3-methoxy-phenyl)-ethyl]-amide;
5-(3,4-Dimethyl-phenyl)-2-methyl-thiazole-4-carboxylic acid cyclopropylmethyl-[2-(4-fluoro-phenyl)-ethyl]-amide;
5-(3,4-Dimethyl-phenyl)-2-methyl-thiazole-4-carboxylic acid [2-(4-chloro-phenyl)-ethyl]-cyclopropylmethyl-amide;
5-(3,4-Dimethyl-phenyl)-2-methyl-thiazole-4-carboxylic acid cyclopropylmethyl-(2-p-tolyl-ethyl)-amide;
5-(3,4-Dimethyl-phenyl)-2-methyl-thiazole-4-carboxylic acid cyclopropylmethyl-[2-(4-ethyl-phenyl)-ethyl]-amide;
5-(3,4-Dimethyl-phenyl)-2-methyl-thiazole-4-carboxylic acid cyclopropylmethyl-[2-(4-methoxy-phenyl)-ethyl]-amide;
5-(3,4-Dimethyl-phenyl)-2-methyl-thiazole-4-carboxylic acid cyclopropylmethyl-[2-(4-hydroxy-phenyl)-ethyl]-amide;
5-(3,4-Dimethyl-phenyl)-2-methyl-thiazole-4-carboxylic acid cyclopropylmethyl-[2-(4-methylsulfanyl-phenyl)-ethyl]-amide;
5-(3,4-Dimethyl-phenyl)-2-methyl-thiazole-4-carboxylic acid cyclopropylmethyl-[2-(4-trifluoromethyl-phenyl)-ethyl]-amide;
5-(3,4-Dimethyl-phenyl)-2-methyl-thiazole-4-carboxylic acid cyclopropylmethyl-[2-(2,4-dimethyl-phenyl)-ethyl]-amide;
5-(3,4-Dimethyl-phenyl)-2-methyl-thiazole-4-carboxylic acid cyclopropylmethyl-[2-(2,5-dimethoxy-phenyl)-ethyl]-amide;
5-(3,4-Dimethyl-phenyl)-2-methyl-thiazole-4-carboxylic acid cyclopropylmethyl-[2-(2,5-dimethyl-phenyl)-ethyl]-amide;
5-(3,4-Dimethyl-phenyl)-2-methyl-thiazole-4-carboxylic acid [2-(5-bromo-2-methoxy-phenyl)-ethyl]-cyclopropylmethyl-amide;
5-(3,4-Dimethyl-phenyl)-2-methyl-thiazole-4-carboxylic acid (2-benzo[1,3]dioxol-5-yl-ethyl)-cyclopropylmethyl-amide;
5-(3,4-Dimethyl-phenyl)-2-methyl-thiazole-4-carboxylic acid cyclopropylmethyl-[2-(2,3-dihydro-benzo[1,4]dioxin-6-yl)-ethyl]-amide;
5-(3,4-Dimethyl-phenyl)-2-methyl-thiazole-4-carboxylic acid cyclopropylmethyl-[2-(4-ethoxy-3-methoxy-phenyl)-ethyl]-amide;
5-(3,4-Dimethyl-phenyl)-2-methyl-thiazole-4-carboxylic acid cyclopropylmethyl-[2-(3-ethoxy-4-methoxy-phenyl)-ethyl]-amide;
5-(3,4-Dimethyl-phenyl)-2-methyl-thiazole-4-carboxylic acid cyclopropylmethyl-[2-(4-methoxy-3-methylsulfanyl-phenyl)-ethyl]-amide;
5-(3,4-Dimethyl-phenyl)-2-methyl-thiazole-4-carboxylic acid cyclopropylmethyl-[2-(4-methoxy-3-methyl-phenyl)-ethyl]-amide;
5-(3,4-Dimethyl-phenyl)-2-methyl-thiazole-4-carboxylic acid [2-(3-bromo-4-methoxy-phenyl)-ethyl]-cyclopropylmethyl-amide;
5-(3,4-Dimethyl-phenyl)-2-methyl-thiazole-4-carboxylic acid cyclopropylmethyl-[2-(3,4-dimethyl-phenyl)-ethyl]-amide;
5-(3,4-Dimethyl-phenyl)-2-methyl-thiazole-4-carboxylic acid cyclopropylmethyl-[2-(3-difluoromethoxy-4-methoxy-phenyl)-ethyl]-amide;
5-(3,4-Dimethyl-phenyl)-2-methyl-thiazole-4-carboxylic acid cyclopropylmethyl-[2-(4-difluoromethoxy-3-methoxy-phenyl)-ethyl]-amide;
5-(3,4-Dimethyl-phenyl)-2-methyl-thiazole-4-carboxylic acid cyclopropylmethyl-[2-(4-hydroxy-3-methoxy-phenyl)-ethyl]-amide;
5-(3,4-Dimethyl-phenyl)-2-methyl-thiazole-4-carboxylic acid cyclopropylmethyl-[dimethoxy-benzyl)-propyl]-amide;
5-(3,4-Dimethyl-phenyl)-2-methyl-thiazole-4-carboxylic acid cyclopropylmethyl-[2-(3,5-dimethoxy-phenyl)-ethyl]-amide;
5-(3,4-Dimethyl-phenyl)-2-methyl-thiazole-4-carboxylic acid cyclopropylmethyl-[2-(2,6-dichloro-phenyl)-ethyl]-amide;
5-(3,4-Dimethyl-phenyl)-2-methyl-thiazole-4-carboxylic acid cyclopropylmethyl-[2-(3,4,5-trimethoxy-phenyl)-ethyl]-amide;
5-(3,4-Dimethyl-phenyl)-2-methyl-thiazole-4-carboxylic acid cyclopropylmethyl-[2-(4-isopropoxy-3,5-dimethoxy-phenyl)-ethyl]-amide;
5-(3,4-Dimethyl-phenyl)-2-methyl-thiazole-4-carboxylic acid cyclopropylmethyl-[2-(4-iodo-2,5-dimethoxy-phenyl)-ethyl]-amide;
N-Cyclopropylmethyl-N-[2-(3,4-dimethoxy-phenyl)-ethyl]-3-m-tolyl-isonicotinamide;
N-Cyclopropylmethyl-N-[2-(3,4-dimethoxy-phenyl)-ethyl]-3-p-tolyl-isonicotinamide;
N-Cyclopropylmethyl-N-[2-(3,4-dimethoxy-phenyl)-ethyl]-3-(3,4-dimethyl-phenyl)-isonicotinamide;
N-Cyclopropylmethyl-N-[2-(3,4-dimethoxy-phenyl)-ethyl]-3-(3-methoxy-phenyl)-isonicotinamide;
3-m-Tolyl-pyridine-2-carboxylic acid cyclopropylmethyl-[2-(3,4-dimethoxy-phenyl)-ethyl]-amide;
3-p-Tolyl-pyridine-2-carboxylic acid cyclopropylmethyl-[2-(3,4-dimethoxy-phenyl)-ethyl]-amide;
3-(3,4-Dimethyl-phenyl)-pyridine-2-carboxylic acid cyclopropylmethyl-[2-(3,4-dimethoxy-phenyl)-ethyl]-amide;
3-(3-Methoxy-phenyl)-pyridine-2-carboxylic acid cyclopropylmethyl-[2-(3,4-dimethoxy-phenyl)-ethyl]-amide;
N-Cyclopropylmethyl-N-[2-(3,4-dimethoxy-phenyl)-ethyl]-2-m-tolyl-nicotinamide;
N-Cyclopropylmethyl-N-[2-(3,4-dimethoxy-phenyl)-ethyl]-2-p-tolyl-nicotinamide;
N-Cyclopropylmethyl-N-[2-(3,4-dimethoxy-phenyl)-ethyl]-2-(3,4-dimethyl-phenyl)-nicotinamide;
N-Cyclopropylmethyl-N-[2-(3,4-dimethoxy-phenyl)-ethyl]-2-(3-methoxy-phenyl)-nicotinamide;
2-Methyl-5-m-tolyl-thiazole-4-carboxylic acid [2-cyclopropyl-amino-2-(3,4-dimethoxy-phenyl)-ethyl]-cyclopropylmethyl-amide;
2-Amino-5-m-tolyl-thiazole-4-carboxylic acid cyclopropylmethyl-[2-(1H-indol-3-yl)-ethyl]-amide;
2-Amino-5-m-tolyl-thiazole-4-carboxylic acid [2-(1H-benzoimidazol-2-yl)-ethyl]-cyclopropylmethyl-amide;
2-Amino-5-m-tolyl-thiazole-4-carboxylic acid [2-(2-amino-thiazol-4-yl)-ethyl]-cyclopropylmethyl-amide;
2-Amino-5-m-tolyl-thiazole-4-carboxylic acid cyclopropylmethyl-[2-(2-ethyl-4-iodo-imidazol-1-yl)-ethyl]-amide;
5-(3,4-Dimethyl-phenyl)-2-methyl-thiazole-4-carboxylic acid cyclopropylmethyl-[2-(1H-indol-3-yl)-ethyl]-amide;
5-(3,4-Dimethyl-phenyl)-2-methyl-thiazole-4-carboxylic acid [2-(1H-benzoimidazol-2-yl)-ethyl]-cyclopropylmethyl-amide;
5-(3,4-Dimethyl-phenyl)-2-methyl-thiazole-4-carboxylic acid cyclopropylmethyl-[2-(2-ethyl-4-iodo-imidazol-1-yl)-ethyl]-amide;
2-Amino-5-m-tolyl-thiazole-4-carboxylic acid cyclopropylmethyl-[2-(6-methoxy-1H-benzoimidazol-2-yl)-ethyl]-amide;
2-Amino-5-m-tolyl-thiazole-4-carboxylic acid cyclopropylmethyl-[2-(5,6-dimethyl-1H-benzoimidazol-2-yl)-ethyl]-amide;
2-Amino-5-m-tolyl-thiazole-4-carboxylic acid cyclopropylmethyl-[2-(6-methyl-1H-benzoimidazol-2-yl)-ethyl]-amide;
2-Amino-5-m-tolyl-thiazole-4-carboxylic acid [2-(6-chloro-1H-benzoimidazol-2-yl)-ethyl]-cyclopropylmethyl-amide;
2-Amino-5-m-tolyl-thiazole-4-carboxylic acid cyclopropylmethyl-(2-indol-1-yl-ethyl)-amide;
2-Amino-5-m-tolyl-thiazole-4-carboxylic acid cyclopropylmethyl-[2-(1-methyl-1H-indol-3-yl)-ethyl]-amide;
2-Amino-5-m-tolyl-thiazole-4-carboxylic acid [2-(5-bromo-1H-indol-3-yl)-ethyl]-cyclopropylmethyl-amide;
2-Amino-5-m-tolyl-thiazole-4-carboxylic acid [2-(6-chloro-1H-indol-3-yl)-ethyl]-cyclopropylmethyl-amide;
2-Amino-5-m-tolyl-thiazole-4-carboxylic acid cyclopropylmethyl-[2-(7-methoxy-1H-indol-3-yl)-ethyl]-amide;
2-Amino-5-m-tolyl-thiazole-4-carboxylic acid cyclopropylmethyl-[2-(5-methoxy-1H-indol-3-yl)-ethyl]-amide;
2-Amino-5-m-tolyl-thiazole-4-carboxylic acid cyclopropylmethyl-[2-(6-methoxy-1H-indol-3-yl)-ethyl]-amide;
2-Amino-5-m-tolyl-thiazole-4-carboxylic acid cyclopropylmethyl-[2-(5-methyl-1H-indol-3-yl)-ethyl]-amide;
2-Amino-5-m-tolyl-thiazole-4-carboxylic acid cyclopropylmethyl-[2-(6-methyl-1H-indol-3-yl)-ethyl]-amide;
2-Amino-5-m-tolyl-thiazole-4-carboxylic acid cyclopropylmethyl-[2-(7-methyl-1H-indol-3-yl)-ethyl]-amide;
2-Amino-5-m-tolyl-thiazole-4-carboxylic acid cyclopropylmethyl-[2-(4-fluoro-1H-indol-3-yl)-ethyl]-amide;
2-Amino-5-m-tolyl-thiazole-4-carboxylic acid cyclopropylmethyl-[2-(5-fluoro-1H-indol-3-yl)-ethyl]-amide;
2-Amino-5-m-tolyl-thiazole-4-carboxylic acid cyclopropylmethyl-[2-(6-fluoro-1H-indol-3-yl)-ethyl]-amide;
2-Amino-5-m-tolyl-thiazole-4-carboxylic acid cyclopropylmethyl-[2-(7-fluoro-1H-indol-3-yl)-ethyl]-amide;
2-Amino-5-m-tolyl-thiazole-4-carboxylic acid cyclopropylmethyl-[2-(6-methoxy-pyridin-3-yl)-ethyl]-amide;
5-(3,4-Dimethyl-phenyl)-2-methyl-thiazole-4-carboxylic acid cyclopropylmethyl-[2-(6-methoxy-1H-benzoimidazol-2-yl)-ethyl]-amide;
5-(3,4-Dimethyl-phenyl)-2-methyl-thiazole-4-carboxylic acid cyclopropylmethyl-[2-(5,6-dimethyl-1H-benzoimidazol-2-yl)-ethyl]-amide;
5-(3,4-Dimethyl-phenyl)-2-methyl-thiazole-4-carboxylic acid cyclopropylmethyl-[2-(6-methyl-1H-benzoimidazol-2-yl)-ethyl]-amide;
5-(3,4-Dimethyl-phenyl)-2-methyl-thiazole-4-carboxylic acid [2-(6-chloro-1H-benzoimidazol-2-yl)-ethyl]-cyclopropylmethyl-amide;
5-(3,4-Dimethyl-phenyl)-2-methyl-thiazole-4-carboxylic acid cyclopropylmethyl-(2-indol-1-yl-ethyl)-amide;
5-(3,4-Dimethyl-phenyl)-2-methyl-thiazole-4-carboxylic acid cyclopropylmethyl-[2-(1-methyl-1H-indol-3-yl)-ethyl]-amide;
5-(3,4-Dimethyl-phenyl)-2-methyl-thiazole-4-carboxylic acid [2-(5-bromo-1H-indol-3-yl)-ethyl]-cyclopropylmethyl-amide;
5-(3,4-Dimethyl-phenyl)-2-methyl-thiazole-4-carboxylic acid [2-(6-chloro-1H-indol-3-yl)-ethyl]-cyclopropylmethyl-amide;
5-(3,4-Dimethyl-phenyl)-2-methyl-thiazole-4-carboxylic acid cyclopropylmethyl-[2-(7-methoxy-1H-indol-3-yl)-ethyl]-amide;
5-(3,4-Dimethyl-phenyl)-2-methyl-thiazole-4-carboxylic acid cyclopropylmethyl-[2-(5-methoxy-1H-indol-3-yl)-ethyl]-amide;
5-(3,4-Dimethyl-phenyl)-2-methyl-thiazole-4-carboxylic acid cyclopropylmethyl-[2-(6-methoxy-1H-indol-3-yl)-ethyl]-amide;
5-(3,4-Dimethyl-phenyl)-2-methyl-thiazole-4-carboxylic acid cyclopropylmethyl-[2-(5-methyl-1H-indol-3-yl)-ethyl]-amide;
5-(3,4-Dimethyl-phenyl)-2-methyl-thiazole-4-carboxylic acid cyclopropylmethyl-[2-(6-methyl-1H-indol-3-yl)-ethyl]-amide;
5-(3,4-Dimethyl-phenyl)-2-methyl-thiazole-4-carboxylic acid cyclopropylmethyl-[2-(7-methyl-1H-indol-3-yl)-ethyl]-amide;
5-(3,4-Dimethyl-phenyl)-2-methyl-thiazole-4-carboxylic acid cyclopropylmethyl-[2-(4-fluoro-1H-indol-3-yl)-ethyl]-amide;
5-(3,4-Dimethyl-phenyl)-2-methyl-thiazole-4-carboxylic acid cyclopropylmethyl-[2-(5-fluoro-1H-indol-3-yl)-ethyl]-amide;
5-(3,4-Dimethyl-phenyl)-2-methyl-thiazole-4-carboxylic acid cyclopropylmethyl-[2-(6-fluoro-1H-indol-3-yl)-ethyl]-amide;
5-(3,4-Dimethyl-phenyl)-2-methyl-thiazole-4-carboxylic acid cyclopropylmethyl-[2-(7-fluoro-1H-indol-3-yl)-ethyl]-amide;
5-(3,4-Dimethyl-phenyl)-2-methyl-thiazole-4-carboxylic acid cyclopropylmethyl-[2-(6-methoxy-pyridin-3-yl)-ethyl]-amide;
3-p-Tolyl-pyrazine-2-carboxylic acid cyclopropylmethyl-[2-(5-fluoro-1H-indol-3-yl)-ethyl]-amide;
3-(3,4-Dimethyl-phenyl)-pyrazine-2-carboxylic acid cyclopropylmethyl-[2-(5-fluoro-1H-indol-3-yl)-ethyl]-amide;
3-m-Tolyl-pyrazine-2-carboxylic acid cyclopropylmethyl-[2-(5-fluoro-1H-indol-3-yl)-ethyl]-amide;
3-(3-Methoxy-phenyl)-pyrazine-2-carboxylic acid cyclopropylmethyl-[2-(5-fluoro-1H-indol-3-yl)-ethyl]-amide;
5-(3,4-Dimethyl-phenyl)-2-methyl-oxazole-4-carboxylic acid cyclopropylmethyl-[2-(5-fluoro-1H-indol-3-yl)-ethyl]-amide;
5-(3,4-Dimethyl-phenyl)-oxazole-4-carboxylic acid cyclopropylmethyl-[2-(5-fluoro-1H-indol-3-yl)-ethyl]-amide;
5-(3-Dimethylamino-phenyl)-oxazole-4-carboxylic acid cyclopropylmethyl-[2-(5-fluoro-1H-indol-3-yl)-ethyl]-amide;
4-(4-Chloro-phenyl)-2-methyl-thiazole-5-carboxylic acid cyclopropylmethyl-[2-(5-fluoro-1H-indol-3-yl)-ethyl]-amide;
5-(4-Fluoro-phenyl)-2-methyl-thiazole-4-carboxylic acid cyclopropylmethyl-[2-(5-fluoro-1H-indol-3-yl)-ethyl]-amide;
5-(4-Ethyl-phenyl)-2-methyl-thiazole-4-carboxylic acid cyclopropylmethyl-[2-(5-fluoro-1H-indol-3-yl)-ethyl]-amide;
5-(3-Chloro-phenyl)-2-methyl-thiazole-4-carboxylic acid cyclopropylmethyl-[2-(5-fluoro-1H-indol-3-yl)-ethyl]-amide;
2-Methyl-5-p-tolyl-thiazole-4-carboxylic acid cyclopropylmethyl-[2-(5-fluoro-1H-indol-3-yl)-ethyl]-amide;
5-(3,5-Dimethyl-phenyl)-2-methyl-thiazole-4-carboxylic acid cyclopropylmethyl-[2-(5-fluoro-1H-indol-3-yl)-ethyl]-amide;
5-(3-Cyano-phenyl)-2-methyl-thiazole-4-carboxylic acid cyclopropylmethyl-[2-(5-fluoro-1H-indol-3-yl)-ethyl]-amide;
5-(4-Chloro-phenyl)-2-methyl-thiazole-4-carboxylic acid cyclopropylmethyl-[2-(5-fluoro-1H-indol-3-yl)-ethyl]-amide;
5-(3,4-Difluoro-phenyl)-2-methyl-thiazole-4-carboxylic acid cyclopropylmethyl-[2-(5-fluoro-1H-indol-3-yl)-ethyl]-amide;
5-(3,4-Dichloro-phenyl)-2-methyl-thiazole-4-carboxylic acid cyclopropylmethyl-[2-(5-fluoro-1H-indol-3-yl)-ethyl]-amide;
5-(3-Fluoro-4-methyl-phenyl)-2-methyl-thiazole-4-carboxylic acid cyclopropylmethyl-[2-(5-fluoro-1H-indol-3-yl)-ethyl]-amide;
5-(2,3-Difluoro-4-methyl-phenyl)-2-methyl-thiazole-4-carboxylic acid cyclopropylmethyl-[2-(5-fluoro-1H-indol-3-yl)-ethyl]-amide;
5-(3,4-Dimethyl-phenyl)-thiazole-4-carboxylic acid cyclopropylmethyl-[2-(5-fluoro-1H-indol-3-yl)-ethyl]-amide;
2-Methoxy-5-m-tolyl-thiazole-4-carboxylic acid cyclopropylmethyl-[2-(5-fluoro-1H-indol-3-yl)-ethyl]-amide;
2-Cyclopropyl-5-(3-fluoro-4-methyl-phenyl)-thiazole-4-carboxylic acid cyclopropylmethyl-[2-(5-fluoro-1H-indol-3-yl)-ethyl]-amide;
2-Dimethylamino-5-m-tolyl-thiazole-4-carboxylic acid cyclopropylmethyl-[2-(5-fluoro-1H-indol-3-yl)-ethyl]-amide;
2-Dimethylamino-5-(3,4-dimethyl-phenyl)-thiazole-4-carboxylic acid cyclopropylmethyl-[2-(5-fluoro-1H-indol-3-yl)-ethyl]-amide;
2-Dimethylaminomethyl-5-m-tolyl-thiazole-4-carboxylic acid cyclopropylmethyl-[2-(5-fluoro-1H-indol-3-yl)-ethyl]-amide;
3-Phenyl-pyrazine-2-carboxylic acid cyclopropylmethyl-[2-(5-fluoro-1H-indol-3-yl)-ethyl]-amide;
3-Phenyl-pyrazine-2-carboxylic acid [2-(5-fluoro-1H-indol-3-yl)-ethyl]-methyl-amide;
3-Phenyl-pyrazine-2-carboxylic acid ethyl-[2-(5-fluoro-1H-indol-3-yl)-ethyl]amide;
3-Phenyl-pyrazine-2-carboxylic acid [2-(5-fluoro-1H-indol-3-yl)-ethyl]-propyl-amide;
3-Phenyl-pyrazine-2-carboxylic acid [2-(5-fluoro-1H-indol-3-yl)-ethyl]-(2,2,2-trifluoro-ethyl)-amide;
3-Phenyl-pyrazine-2-carboxylic acid carbamoylmethyl-[2-(5-fluoro-1H-indol-3-yl)-ethyl]amide;
[[2-(5-Fluoro-1H-indol-3-yl)-ethyl]-(3-phenyl-pyrazine-2-carbonyl)-amino]-acetic acid methyl ester;
3-Phenyl-pyrazine-2-carboxylic acid [2-(5-fluoro-1H-indol-3-yl)-ethyl]-isopropyl-amide;
3-Phenyl-pyrazine-2-carboxylic acid (2,2-difluoro-ethyl)-[2-(5-fluoro-1H-indol-3-yl)-ethyl]-amide;
3-Phenyl-pyrazine-2-carboxylic acid [2-(5-fluoro-1H-indol-3-yl)-ethyl]-(2-hydroxy-ethyl)-amide;
3-(3,4-Dimethyl-phenyl)-pyrazine-2-carboxylic acid [2-(5-fluoro-1H-indol-3-yl)-ethyl]-methyl-amide;
3-(3,4-Dimethyl-phenyl)-pyrazine-2-carboxylic acid ethyl-[2-(5-fluoro-1H-indol-3-yl)-ethyl]-amide;
3-(3,4-Dimethyl-phenyl)-pyrazine-2-carboxylic acid [2-(5-fluoro-1H-indol-3-yl)-ethyl]-propyl-amide;
3-(3,4-Dimethyl-phenyl)-pyrazine-2-carboxylic acid [2-(5-fluoro-1H-indol-3-yl)-ethyl]-(2,2,2-trifluoro-ethyl)-amide;
3-(3,4-Dimethyl-phenyl)-pyrazine-2-carboxylic acid carbamoylmethyl-[2-(5-fluoro-1H-indol-3-yl)-ethyl]-amide;
{[3-(3,4-Dimethyl-phenyl)-pyrazine-2-carbonyl]-[2-(5-fluoro-1H-indol-3-yl)-ethyl]-amino}-acetic acid methyl ester;
3-(3,4-Dimethyl-phenyl)-pyrazine-2-carboxylic acid [2-(5-fluoro-1H-indol-3-yl)-ethyl]-isopropyl-amide;
3-(3,4-Dimethyl-phenyl)-pyrazine-2-carboxylic acid (2,2-difluoro-ethyl)-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-methyl-amide;
5-(6-Methoxy-pyridin-3-yl)-2-methyl-thiazole-4-carboxylic acid ethyl-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-propyl-amide;
5-(6-Methoxy-pyridin-3-yl)-2-methyl-thiazole-4-carboxylic acid [2-(5-fluoro-1H-indol-3-yl)-ethyl]-(2,2,2-trifluoro-ethyl)-amide;
5-(6-Methoxy-pyridin-3-yl)-2-methyl-thiazole-4-carboxylic acid carbamoylmethyl-[2-(5-fluoro-1H-indol-3-yl)-ethyl]-amide;
5-(6-Methoxy-pyridin-3-yl)-2-methyl-thiazole-4-carboxylic acid dimethylcarbamoylmethyl-[2-(5-fluoro-1H-indol-3-yl)-ethyl]-amide;
5-(6-Methoxy-pyridin-3-yl)-2-methyl-thiazole-4-carboxylic acid (2-dimethylamino-ethyl)-[2-(5-fluoro-1H-indol-3-yl)-ethyl]-amide;
{[2-(5-Fluoro-1H-indol-3-yl)-ethyl]-[5-(6-methoxy-pyridin-3-yl)-2-methyl-thiazole-4-carbonyl]-amino}-acetic acid methyl ester;
5-(6-Methoxy-pyridin-3-yl)-2-methyl-thiazole-4-carboxylic acid [2-(5-fluoro-1H-indol-3-yl)-ethyl]-isopropyl-amide;
5-(6-Methoxy-pyridin-3-yl)-2-methyl-thiazole-4-carboxylic acid (2,2-difluoro-ethyl)-[2-(5-fluoro-1H-indol-3-yl)-ethyl]-amide;
5-(6-Methoxy-pyridin-3-yl)-2-methyl-thiazole-4-carboxylic acid [2-(5-fluoro-1H-indol-3-yl)-ethyl]-(2-hydroxy-ethyl)-amide;
6′-Methoxy-[3,3]bipyridinyl-2-carboxylic acid [2-(5-fluoro-1H-indol-3-yl)-ethyl]methyl-amide;
6′-Methoxy-[3,3′]bipyridinyl-2-carboxylic acid ethyl-[2-(5-fluoro-1H-indol-3-yl)-ethyl]-amide;
6-Methoxy-[3,3′]bipyridinyl-2-carboxylic acid [2-(5-fluoro-1H-indol-3-yl)-ethyl]-propyl-amide;
6-Methoxy-[3,3′]bipyridinyl-2-carboxylic acid [2-(5-fluoro-1H-indol-3-yl)-ethyl]-(2,2,2-trifluoro-ethyl)-amide;
6′-Methoxy-[3,3′]bipyridinyl-2-carboxylic acid carbamoylmethyl-[2-(5-fluoro-1H-indol-3-yl)-ethyl]-amide;
6′-Methoxy-[3,3]bipyridinyl-2-carboxylic acid dimethylcarbamoylmethyl-[2-(5-fluoro-1H-indol-3-yl)-ethyl]-amide;
[[2-(5-Fluoro-1H-indol-3-yl)-ethyl]-(6-methoxy-[3,3]bipyridinyl-2-carbonyl)-amino]-acetic acid methyl ester;
6-Methoxy-[3,3′]bipyridinyl-2-carboxylic acid [2-(5-fluoro-1H-indol-3-yl)-ethyl]-isopropyl-amide;
6′-Methoxy-[3,3′]bipyridinyl-2-carboxylic acid (2,2-difluoro-ethyl)-[2-(5-fluoro-1H-indol-3-yl)-ethyl]-amide;
6′-Methoxy-[3,3′]bipyridinyl-2-carboxylic acid [2-(5-fluoro-1H-indol-3-yl)-ethyl]-(2-hydroxy-ethyl)-amide;
3-Phenyl-pyrazine-2-carboxylic acid cyclopropylmethyl-[2-(1-methyl-1H-indol-3-yl)-ethyl]-amide;
3-Phenyl-pyrazine-2-carboxylic acid [2-(6-chloro-1H-indol-3-yl)-ethyl]-cyclopropylmethyl-amide;
3-Phenyl-pyrazine-2-carboxylic acid cyclopropylmethyl-[2-(7-methoxy-1H-indol-3-yl)-ethyl]-amide;
3-Phenyl-pyrazine-2-carboxylic acid cyclopropylmethyl-[2-(5-methoxy-1H-indol-3-yl)-ethyl]-amide;
3-Phenyl-pyrazine-2-carboxylic acid cyclopropylmethyl-[2-(6-methoxy-1H-indol-3-yl)-ethyl]-amide;
3-Phenyl-pyrazine-2-carboxylic acid cyclopropylmethyl-[2-(5-methyl-1H-indol-3-yl)-ethyl]-amide;
3-Phenyl-pyrazine-2-carboxylic acid cyclopropylmethyl-[2-(6-methyl-1H-indol-3-yl)-ethyl]-amide;
3-Phenyl-pyrazine-2-carboxylic acid cyclopropylmethyl-[2-(7-methyl-1H-indol-3-yl)-ethyl]-amide;
3-Phenyl-pyrazine-2-carboxylic acid cyclopropylmethyl-[2-(4-fluoro-1H-indol-3-yl)-ethyl]-amide;
3-Phenyl-pyrazine-2-carboxylic acid cyclopropylmethyl-[2-(6-fluoro-1H-indol-3-yl)-ethyl]-amide;
3-Phenyl-pyrazine-2-carboxylic acid cyclopropylmethyl-[2-(7-fluoro-1H-indol-3-yl)-ethyl]-amide;
3-(3,4-Dimethyl-phenyl)-pyrazine-2-carboxylic acid cyclopropylmethyl-[2-(1-methyl-1H-indol-3-yl)-ethyl]-amide;
3-(3,4-Dimethyl-phenyl)-pyrazine-2-carboxylic acid [2-(6-chloro-1H-indol-3-yl)-ethyl]-cyclopropylmethyl-amide;
3-(3,4-Dimethyl-phenyl)-pyrazine-2-carboxylic acid cyclopropylmethyl-[2-(7-methoxy-1H-indol-3-yl)-ethyl]-amide;
3-(3,4-Dimethyl-phenyl)-pyrazine-2-carboxylic acid cyclopropylmethyl-[2-(5-methoxy-1H-indol-3-yl)-ethyl]-amide;
3-(3,4-Dimethyl-phenyl)-pyrazine-2-carboxylic acid cyclopropylmethyl-[2-(6-methoxy-1H-indol-3-yl)-ethyl]-amide;
3-(3,4-Dimethyl-phenyl)-pyrazine-2-carboxylic acid cyclopropylmethyl-[2-(5-methyl-1H-indol-3-yl)-ethyl]-amide;
3-(3,4-Dimethyl-phenyl)-pyrazine-2-carboxylic acid cyclopropylmethyl-[2-(6-methyl-1H-indol-3-yl)-ethyl]-amide;
3-(3,4-Dimethyl-phenyl)-pyrazine-2-carboxylic acid cyclopropylmethyl-[2-(7-methyl-1H-indol-3-yl)-ethyl]-amide;
3-(3,4-Dimethyl-phenyl)-pyrazine-2-carboxylic acid cyclopropylmethyl-[2-(4-fluoro-1H-indol-3-yl)-ethyl]-amide;
3-(3,4-Dimethyl-phenyl)-pyrazine-2-carboxylic acid cyclopropylmethyl-[2-(6-fluoro-1H-indol-3-yl)-ethyl]-amide;
3-(3,4-Dimethyl-phenyl)-pyrazine-2-carboxylic acid cyclopropylmethyl-[2-(7-fluoro-1H-indol-3-yl)-ethyl]-amide;
5-(6-Methoxy-pyridin-3-yl)-2-methyl-thiazole-4-carboxylic acid cyclopropylmethyl-[2-(1-methyl-1H-indol-3-yl)-ethyl]-amide;
5-(6-Methoxy-pyridin-3-yl)-2-methyl-thiazole-4-carboxylic acid [2-(6-chloro-1H-indol-3-yl)-ethyl]-cyclopropylmethyl-amide;
5-(6-Methoxy-pyridin-3-yl)-2-methyl-thiazole-4-carboxylic acid cyclopropylmethyl-[2-(7-methoxy-1H-indol-3-yl)-ethyl]-amide;
5-(6-Methoxy-pyridin-3-yl)-2-methyl-thiazole-4-carboxylic acid cyclopropylmethyl-[2-(5-methoxy-1H-indol-3-yl)-ethyl]-amide;
5-(6-Methoxy-pyridin-3-yl)-2-methyl-thiazole-4-carboxylic acid cyclopropylmethyl-[2-(6-methoxy-1H-indol-3-yl)-ethyl]-amide;
5-(6-Methoxy-pyridin-3-yl)-2-methyl-thiazole-4-carboxylic acid cyclopropylmethyl-[2-(5-methyl-1H-indol-3-yl)-ethyl]-amide;
5-(6-Methoxy-pyridin-3-yl)-2-methyl-thiazole-4-carboxylic acid cyclopropylmethyl-[2-(6-methyl-1H-indol-3-yl)-ethyl]-amide;
5-(6-Methoxy-pyridin-3-yl)-2-methyl-thiazole-4-carboxylic acid cyclopropylmethyl-[2-(7-methyl-1H-indol-3-yl)-ethyl]-amide;
5-(6-Methoxy-pyridin-3-yl)-2-methyl-thiazole-4-carboxylic acid cyclopropylmethyl-[2-(4-fluoro-1H-indol-3-yl)-ethyl]-amide;
5-(6-Methoxy-pyridin-3-yl)-2-methyl-thiazole-4-carboxylic acid cyclopropylmethyl-[2-(6-fluoro-1H-indol-3-yl)-ethyl]-amide;
5-(6-Methoxy-pyridin-3-yl)-2-methyl-thiazole-4-carboxylic acid cyclopropylmethyl-[2-(7-fluoro-1H-indol-3-yl)-ethyl]-amide;
6′-Methoxy-[3,3′]bipyridinyl-2-carboxylic acid cyclopropylmethyl-[2-(1-methyl-1H-indol-3-yl)-ethyl]-amide;
6′-Methoxy-[3,3]bipyridinyl-2-carboxylic acid [2-(6-chloro-1H-indol-3-yl)-ethyl]-cyclopropylmethyl-amide;
6-Methoxy-[3,3′]bipyridinyl-2-carboxylic acid cyclopropylmethyl-[2-(7-methoxy-1H-indol-3-yl)-ethyl]-amide;
6′-Methoxy-[3,3]bipyridinyl-2-carboxylic acid cyclopropylmethyl-[2-(5-methoxy-1H-indol-3-yl)-ethyl]-amide;
6′-Methoxy-[3,3′]bipyridinyl-2-carboxylic acid cyclopropylmethyl-[2-(6-methoxy-1H-indol-3-yl)-ethyl]-amide;
6-Methoxy-[3,3]bipyridinyl-2-carboxylic acid cyclopropylmethyl-[2-(5-methyl-1H-indol-3-yl)-ethyl]-amide;
6′-Methoxy-[3,3]bipyridinyl-2-carboxylic acid cyclopropylmethyl-[2-(6-methyl-1H-indol-3-yl)-ethyl]-amide;
6′-Methoxy-[3,3′]bipyridinyl-2-carboxylic acid cyclopropylmethyl-[2-(7-methyl-1H-indol-3-yl)-ethyl]-amide;
6′-Methoxy-[3,3]bipyridinyl-2-carboxylic acid cyclopropylmethyl-[2-(4-fluoro-1H-indol-3-yl)-ethyl]-amide;
6′-Methoxy-[3,3]bipyridinyl-2-carboxylic acid cyclopropylmethyl-[2-(6-fluoro-1H-indol-3-yl)-ethyl]-amide;
6′-Methoxy-[3,3′]bipyridinyl-2-carboxylic acid cyclopropylmethyl-[2-(7-fluoro-1H-indol-3-yl)-ethyl]-amide;
3-Phenyl-pyrazine-2-carboxylic acid cyclopropylmethyl-[2-(6-methoxy-1H-benzoimidazol-2-yl)-ethyl]-amide;
3-m-Tolyl-pyrazine-2-carboxylic acid cyclopropylmethyl-[2-(6-methoxy-1H-benzoimidazol-2-yl)-ethyl]-amide;
3-(3,4-Dimethyl-phenyl)-pyrazine-2-carboxylic acid cyclopropylmethyl-[2-(6-methoxy-1H-benzoimidazol-2-yl)-ethyl]-amide;
2-Methyl-5-m-tolyl-thiazole-4-carboxylic acid cyclopropylmethyl-[2-(6-methoxy-1H-benzoimidazol-2-yl)-ethyl]-amide;
2-Dimethylamino-5-(3-methoxy-phenyl)-thiazole-4-carboxylic acid cyclopropylmethyl-[2-(6-methoxy-1H-benzoimidazol-2-yl)-ethyl]-amide;
2-Dimethylamino-5-(3,4-dimethyl-phenyl)-thiazole-4-carboxylic acid cyclopropylmethyl-[2-(6-methoxy-1H-benzoimidazol-2-yl)-ethyl]-amide;
2-Dimethylamino-5-m-tolyl-thiazole-4-carboxylic acid cyclopropylmethyl-[2-(6-methoxy-1H-benzoimidazol-2-yl)-ethyl]-amide;
5-(6-Methoxy-pyridin-3-yl)-2-methyl-thiazole-4-carboxylic acid cyclopropylmethyl-[2-(6-methoxy-1H-benzoimidazol-2-yl)-ethyl]-amide;
6′-Methoxy-[3,3′]bipyridinyl-2-carboxylic acid cyclopropylmethyl-[2-(6-methoxy-1H-benzoimidazol-2-yl)-ethyl]-amide;
3-Phenyl-pyrazine-2-carboxylic acid cyclopropylmethyl-[2-(5,6-dimethyl-1H-benzoimidazol-2-yl)-ethyl]-amide;
3-m-Tolyl-pyrazine-2-carboxylic acid cyclopropylmethyl-[2-(5,6-dimethyl-1H-benzoimidazol-2-yl)-ethyl]-amide;
3-(3,4-Dimethyl-phenyl)-pyrazine-2-carboxylic acid cyclopropylmethyl-[2-(5,6-dimethyl-1H-benzoimidazol-2-yl)-ethyl]-amide;
2-Methyl-5-m-tolyl-thiazole-4-carboxylic acid cyclopropylmethyl-[2-(5,6-dimethyl-1H-benzoimidazol-2-yl)-ethyl]-amide;
2-Dimethylamino-5-(3-methoxy-phenyl)-thiazole-4-carboxylic acid cyclopropylmethyl-[2-(5,6-dimethyl-1H-benzoimidazol-2-yl)-ethyl]-amide;
2-Dimethylamino-5-(3,4-dimethyl-phenyl)-thiazole-4-carboxylic acid cyclopropylmethyl-[2-(5,6-dimethyl-1H-benzoimidazol-2-yl)-ethyl]-amide;
2-Dimethylamino-5-m-tolyl-thiazole-4-carboxylic acid cyclopropylmethyl-[2-(5,6-dimethyl-1H-benzoimidazol-2-yl)-ethyl]-amide;
5-(6-Methoxy-pyridin-3-yl)-2-methyl-thiazole-4-carboxylic acid cyclopropylmethyl-[2-(5,6-dimethyl-1H-benzoimidazol-2-yl)-ethyl]-amide;
6′-Methoxy-[3,3]bipyridinyl-2-carboxylic acid cyclopropylmethyl-[2-(5,6-dimethyl-1H-benzoimidazol-2-yl)-ethyl]-amide;
5-(6-Methoxy-pyridin-3-yl)-2-methyl-thiazole-4-carboxylic acid cyclopropylmethyl-[2-(5-methoxy-4-methyl-1H-indol-3-yl)-ethyl]-amide;
5-(6-Methoxy-pyridin-3-yl)-2-methyl-thiazole-4-carboxylic acid cyclopropylmethyl-[2-(5H-[1,3]dioxolo[4,5-f]indol-7-yl)-ethyl]-amide;
5-(6-Methoxy-pyridin-3-yl)-2-methyl-thiazole-4-carboxylic acid cyclopropylmethyl-[2-(5,6-difluoro-1H-indol-3-yl)-ethyl]-amide;
5-(6-Methoxy-pyridin-3-yl)-2-methyl-thiazole-4-carboxylic acid [2-(5-chloro-6-fluoro-1H-indol-3-yl)-ethyl]-cyclopropylmethyl-amide;
5-(6-Methoxy-pyridin-3-yl)-2-methyl-thiazole-4-carboxylic acid cyclopropylmethyl-[2-(5-methoxy-1H-indol-3-yl)-1-methyl-ethyl]-amide;
3-m-Tolyl-pyridine-2-carboxylic acid cyclopropylmethyl-[2-(5-fluoro-1H-indol-3-yl)-ethyl]-amide;
3-(3,4-Dimethyl-phenyl)-pyridine-2-carboxylic acid cyclopropylmethyl-[2-(5-fluoro-1H-indol-3-yl)-ethyl]-amide;
6-Methoxy-[3,3′]bipyridinyl-2-carboxylic acid cyclopropylmethyl-[2-(5-fluoro-1H-indol-3-yl)-ethyl]-amide;
6′-Fluoro-[3,3′]bipyridinyl-2-carboxylic acid cyclopropylmethyl-[2-(5-fluoro-1H-indol-3-yl)-ethyl]-amide;
5′-Methyl-[3,3′]bipyridinyl-2-carboxylic acid cyclopropylmethyl-[2-(5-fluoro-1H-indol-3-yl)-ethyl]-amide;
5′-Chloro-2′-fluoro-[3,3]bipyridinyl-2-carboxylic acid cyclopropylmethyl-[2-(5-fluoro-1H-indol-3-yl)-ethyl]-amide;
3-Quinolin-3-yl-pyridine-2-carboxylic acid cyclopropylmethyl-[2-(5-fluoro-1H-indol-3-yl)-ethyl]-amide;
6′-Methyl-[3,3′]bipyridinyl-2-carboxylic acid cyclopropylmethyl-[2-(5-fluoro-1H-indol-3-yl)-ethyl]-amide;
5′-Methoxy-[3,3]bipyridinyl-2-carboxylic acid cyclopropylmethyl-[2-(5-fluoro-1H-indol-3-yl)-ethyl]-amide;
5-(3-Chloro-4-methyl-phenyl)-2-methyl-thiazole-4-carboxylic acid cyclopropylmethyl-[2-(5-fluoro-1H-indol-3-yl)-ethyl]-amide;
5-(3-Chloro-4-methoxy-phenyl)-2-methyl-thiazole-4-carboxylic acid cyclopropylmethyl-[2-(5-fluoro-1H-indol-3-yl)-ethyl]-amide;
2-Methyl-5-(6-methyl-pyridin-3-yl)-thiazole-4-carboxylic acid cyclopropylmethyl-[2-(5-fluoro-1H-indol-3-yl)-ethyl]-amide;
5-(4-Methoxy-3-methyl-phenyl)-2-methyl-thiazole-4-carboxylic acid cyclopropylmethyl-[2-(5-fluoro-1H-indol-3-yl)-ethyl]-amide;
5-(3-Chloro-4-fluoro-phenyl)-2-methyl-thiazole-4-carboxylic acid cyclopropylmethyl-[2-(5-fluoro-1H-indol-3-yl)-ethyl]-amide;
5-(4-Fluoro-3-methyl-phenyl)-2-methyl-thiazole-4-carboxylic acid cyclopropylmethyl-[2-(5-fluoro-1H-indol-3-yl)-ethyl]-amide;
5-(3-Fluoro-4-methoxy-phenyl)-2-methyl-thiazole-4-carboxylic acid cyclopropylmethyl-[2-(5-fluoro-1H-indol-3-yl)-ethyl]-amide;
5-(4-Chloro-3-fluoro-phenyl)-2-methyl-thiazole-4-carboxylic acid cyclopropylmethyl-[2-(5-fluoro-1H-indol-3-yl)-ethyl]-amide;
5-(3-Cyano-4-fluoro-phenyl)-2-methyl-thiazole-4-carboxylic acid cyclopropylmethyl-[2-(5-fluoro-1H-indol-3-yl)-ethyl]-amide;
5-(4-Fluoro-3-methoxy-phenyl)-2-methyl-thiazole-4-carboxylic acid cyclopropylmethyl-[2-(5-fluoro-1H-indol-3-yl)-ethyl]-amide;
5-(4-Chloro-3-cyano-phenyl)-2-methyl-thiazole-4-carboxylic acid cyclopropylmethyl-[2-(5-fluoro-1H-indol-3-yl)-ethyl]-amide;
5-(4-Fluoro-3-hydroxymethyl-phenyl)-2-methyl-thiazole-4-carboxylic acid cyclopropylmethyl-[2-(5-fluoro-1H-indol-3-yl)-ethyl]-amide;
5-(4-Cyano-3-fluoro-phenyl)-2-methyl-thiazole-4-carboxylic acid cyclopropylmethyl-[2-(5-fluoro-1H-indol-3-yl)-ethyl]-amide;
5-(3-Chloro-2-methoxy-pyridin-4-yl)-2-methyl-thiazole-4-carboxylic acid cyclopropylmethyl-[2-(5-fluoro-1H-indol-3-yl)-ethyl]-amide;
5-(6-Methoxy-pyridin-3-yl)-2-methyl-thiazole-4-carboxylic acid cyclopropylmethyl-[2-(5-fluoro-1H-indol-3-yl)-ethyl]-amide;
5-(6-Fluoro-pyridin-3-yl)-2-methyl-thiazole-4-carboxylic acid cyclopropylmethyl-[2-(5-fluoro-1H-indol-3-yl)-ethyl]-amide;
5-(6-Hydroxymethyl-pyridin-3-yl)-2-methyl-thiazole-4-carboxylic acid cyclopropylmethyl-[2-(5-fluoro-1H-indol-3-yl)-ethyl]-amide;
2-Methyl-5-(5-methylsulfanyl-pyridin-3-yl)-thiazole-4-carboxylic acid cyclopropylmethyl-[2-(5-fluoro-1H-indol-3-yl)-ethyl]-amide;
5-(5-Fluoro-pyridin-3-yl)-2-methyl-thiazole-4-carboxylic acid cyclopropylmethyl-[2-(5-fluoro-1H-indol-3-yl)-ethyl]-amide;
2-Methyl-5-(5-methyl-pyridin-3-yl)-thiazole-4-carboxylic acid cyclopropylmethyl-[2-(5-fluoro-1H-indol-3-yl)-ethyl]-amide;
5-(5-Chloro-2-fluoro-pyridin-3-yl)-2-methyl-thiazole-4-carboxylic acid cyclopropylmethyl-[2-(5-fluoro-1H-indol-3-yl)-ethyl]-amide;
2-Methyl-5-quinolin-3-yl-thiazole-4-carboxylic acid cyclopropylmethyl-[2-(5-fluoro-1H-indol-3-yl)-ethyl]-amide;
5-(1H-Indol-5-yl)-2-methyl-thiazole-4-carboxylic acid cyclopropylmethyl-[2-(5-fluoro-1H-indol-3-yl)-ethyl]-amide;
5-(1H-Indol-6-yl)-2-methyl-thiazole-4-carboxylic acid cyclopropylmethyl-[2-(5-fluoro-1H-indol-3-yl)-ethyl]-amide;
2-Methyl-5-(1-methyl-1H-indol-2-yl)-thiazole-4-carboxylic acid cyclopropylmethyl-[2-(5-fluoro-1H-indol-3-yl)-ethyl]-amide;
2-Aminomethyl-5-m-tolyl-thiazole-4-carboxylic acid cyclopropylmethyl-[2-(5-fluoro-1H-indol-3-yl)-ethyl]-amide;
3-(3,4-Dimethyl-phenyl)-pyrazine-2-carboxylic acid (2-amino-ethyl)-[2-(5-fluoro-1H-indol-3-yl)-ethyl]-amide;
2-Methylamino-5-m-tolyl-thiazole-4-carboxylic acid cyclopropylmethyl-[2-(5-fluoro-1H-indol-3-yl)-ethyl]-amide;
3-(4-Methoxy-phenyl)-pyrazine-2-carboxylic acid cyclopropylmethyl-[2-(7-methyl-1H-indol-3-yl)-ethyl]-amide;
3-(6-Methoxy-pyridin-3-yl)-pyrazine-2-carboxylic acid cyclopropylmethyl-[2-(7-methyl-1H-indol-3-yl)-ethyl]-amide;
3-Pyrimidin-5-yl-pyrazine-2-carboxylic acid cyclopropylmethyl-[2-(7-methyl-1H-indol-3-yl)-ethyl]-amide; and
3-(2-Methoxy-pyrimidin-5-yl)-pyrazine-2-carboxylic acid cyclopropylmethyl-[2-(7-methyl-1H-indol-3-yl)-ethyl]-amide,
in a free or a pharmaceutically acceptable salt form.

14. The compound according to claim 1 selected from the group consisting of:
3-(4-Fluoro-phenyl)-pyrazine-2-carboxylic acid cyclopropylmethyl-[2-(7-methoxy-1H-indol-3-yl)-ethyl]-amide;
3-(4-Fluoro-3-methyl-phenyl)-pyrazine-2-carboxylic acid cyclopropylmethyl-[2-(7-methoxy-1H-indol-3-yl)-ethyl]-amide;
3-(2-Fluoro-5-methoxy-phenyl)-pyrazine-2-carboxylic acid cyclopropylmethyl-[2-(7-methoxy-1H-indol-3-yl)-ethyl]-amide;
3-(3-Fluoro-5-methyl-phenyl)-pyrazine-2-carboxylic acid cyclopropylmethyl-[2-(7-methoxy-1H-indol-3-yl)-ethyl]-amide;
3-(3-Trifluoromethyl-phenyl)-pyrazine-2-carboxylic acid cyclopropylmethyl-[2-(7-methoxy-1H-indol-3-yl)-ethyl]-amide;
3-(2,3-Dimethyl-phenyl)-pyrazine-2-carboxylic acid cyclopropylmethyl-[2-(7-methoxy-1H-indol-3-yl)-ethyl]-amide;
3-(3-Methoxy-phenyl)-pyrazine-2-carboxylic acid cyclopropylmethyl-[2-(7-methoxy-1H-indol-3-yl)-ethyl]-amide;
3-m-Tolyl-pyrazine-2-carboxylic acid cyclopropylmethyl-[2-(7-methoxy-1H-indol-3-yl)-ethyl]-amide;
3-(4-Fluoro-phenyl)-pyrazine-2-carboxylic acid cyclopropylmethyl-[2-(5-fluoro-1H-indol-3-yl)-ethyl]-amide;
3-(4-Fluoro-3-methyl-phenyl)-pyrazine-2-carboxylic acid cyclopropylmethyl-[2-(5-fluoro-1H-indol-3-yl)-ethyl]-amide;
3-(2-Fluoro-5-methoxy-phenyl)-pyrazine-2-carboxylic acid cyclopropylmethyl-[2-(5-fluoro-1H-indol-3-yl)-ethyl]-amide;
3-(3-Fluoro-5-methyl-phenyl)-pyrazine-2-carboxylic acid cyclopropylmethyl-[2-(5-fluoro-1H-indol-3-yl)-ethyl]-amide;
3-(3-Trifluoromethyl-phenyl)-pyrazine-2-carboxylic acid cyclopropylmethyl-[2-(5-fluoro-1H-indol-3-yl)-ethyl]-amide;
3-(2,3-Dimethyl-phenyl)-pyrazine-2-carboxylic acid cyclopropylmethyl-[2-(5-fluoro-1H-indol-3-yl)-ethyl]-amide;
2-Methyl-4-phenyl-pyrimidine-5-carboxylic acid cyclopropylmethyl-[2-(5-fluoro-1H-indol-3-yl)-ethyl]-amide;
4-Phenyl-pyrimidine-5-carboxylic acid cyclopropylmethyl-[2-(5-fluoro-1H-indol-3-yl)-ethyl]-amide;
3-(4-Fluoro-phenyl)-pyrazine-2-carboxylic acid cyclopropylmethyl-[2-(5,6-difluoro-1H-indol-3-yl)-ethyl]-amide;
3-(4-Fluoro-3-methyl-phenyl)-pyrazine-2-carboxylic acid cyclopropylmethyl-[2-(5,6-difluoro-1H-indol-3-yl)-ethyl]-amide;
3-(2-Fluoro-5-methoxy-phenyl)-pyrazine-2-carboxylic acid cyclopropylmethyl-[2-(5,6-difluoro-1H-indol-3-yl)-ethyl]-amide;
3-(3-Fluoro-5-methyl-phenyl)-pyrazine-2-carboxylic acid cyclopropylmethyl-[2-(5,6-difluoro-1H-indol-3-yl)-ethyl]-amide;
3-(3-Trifluoromethyl-phenyl)-pyrazine-2-carboxylic acid cyclopropylmethyl-[2-(5,6-difluoro-1H-indol-3-yl)-ethyl]-amide;
3-(2,3-Dimethyl-phenyl)-pyrazine-2-carboxylic acid cyclopropylmethyl-[2-(5,6-difluoro-1H-indol-3-yl)-ethyl]-amide;
2-Methyl-4-phenyl-pyrimidine-5-carboxylic acid cyclopropylmethyl-[2-(5,6-difluoro-1H-indol-3-yl)-ethyl]-amide;
4-Phenyl-pyrimidine-5-carboxylic acid cyclopropylmethyl-[2-(5,6-difluoro-1H-indol-3-yl)-ethyl]-amide;
3-(4-Fluoro-phenyl)-pyrazine-2-carboxylic acid [2-(5-chloro-6-fluoro-1H-indol-3-yl)-ethyl]-cyclopropylmethyl-amide;
3-(4-Fluoro-3-methyl-phenyl)-pyrazine-2-carboxylic acid [2-(5-chloro-6-fluoro-1H-indol-3-yl)-ethyl]-cyclopropylmethyl-amide;
3-(2-Fluoro-5-methoxy-phenyl)-pyrazine-2-carboxylic acid [2-(5-chloro-6-fluoro-1H-indol-3-yl)-ethyl]-cyclopropylmethyl-amide;
3-(3-Fluoro-5-methyl-phenyl)-pyrazine-2-carboxylic acid [2-(5-chloro-6-fluoro-1H-indol-3-yl)-ethyl]-cyclopropylmethyl-amide;
3-(3-Trifluoromethyl-phenyl)-pyrazine-2-carboxylic acid [2-(5-chloro-6-fluoro-1H-indol-3-yl)-ethyl]-cyclopropylmethyl-amide;
3-(2,3-Dimethyl-phenyl)-pyrazine-2-carboxylic acid [2-(5-chloro-6-fluoro-1H-indol-3-yl)-ethyl]-cyclopropylmethyl-amide;
2-Methyl-4-phenyl-pyrimidine-5-carboxylic acid [2-(5-chloro-6-fluoro-1H-indol-3-yl)-ethyl]-cyclopropylmethyl-amide;
4-Phenyl-pyrimidine-5-carboxylic acid [2-(5-chloro-6-fluoro-1H-indol-3-yl)-ethyl]-cyclopropylmethyl-amide;
2-Dimethylamino-5-phenyl-thiazole-4-carboxylic acid [2-(5-chloro-6-fluoro-1H-indol-3-yl)-ethyl]-cyclopropylmethyl-amide;
2-Dimethylamino-5-m-tolyl-thiazole-4-carboxylic acid [2-(5-chloro-6-fluoro-1H-indol-3-yl)-ethyl]-cyclopropylmethyl-amide;
2-Dimethylamino-5-(3-fluoro-4-methyl-phenyl)-thiazole-4-carboxylic acid [2-(5-chloro-6-fluoro-1H-indol-3-yl)-ethyl]-cyclopropylmethyl-amide;
2-Dimethylamino-5-(4-fluoro-phenyl)-thiazole-4-carboxylic acid [2-(5-chloro-6-fluoro-1H-indol-3-yl)-ethyl]-cyclopropylmethyl-amide;
3-(4-Fluoro-phenyl)-pyrazine-2-carboxylic acid cyclopropylmethyl-[2-(5-methoxy-4-methyl-1H-indol-3-yl)-ethyl]-amide;
3-(4-Fluoro-3-methyl-phenyl)-pyrazine-2-carboxylic acid cyclopropylmethyl-[2-(5-methoxy-4-methyl-1H-indol-3-yl)-ethyl]-amide;
3-(3-Fluoro-5-methyl-phenyl)-pyrazine-2-carboxylic acid cyclopropylmethyl-[2-(5-methoxy-4-methyl-1H-indol-3-yl)-ethyl]-amide;
2-Methyl-4-phenyl-pyrimidine-5-carboxylic acid cyclopropylmethyl-[2-(5-methoxy-4-methyl-1H-indol-3-yl)-ethyl]-amide;
4-Phenyl-pyrimidine-5-carboxylic acid cyclopropylmethyl-[2-(5-methoxy-4-methyl-1H-indol-3-yl)-ethyl]-amide;
2-Dimethylamino-5-phenyl-thiazole-4-carboxylic acid cyclopropylmethyl-[2-(5-methoxy-4-methyl-1H-indol-3-yl)-ethyl]-amide;
2-Dimethylamino-5-m-tolyl-thiazole-4-carboxylic acid cyclopropylmethyl-[2-(5-methoxy-4-methyl-1H-indol-3-yl)-ethyl]-amide;
2-Dimethylamino-5-(3-fluoro-4-methyl-phenyl)-thiazole-4-carboxylic acid cyclopropylmethyl-[2-(5-methoxy-4-methyl-1H-indol-3-yl)-ethyl]-amide;
2-Dimethylamino-5-(4-fluoro-phenyl)-thiazole-4-carboxylic acid cyclopropylmethyl-[2-(5-methoxy-4-methyl-1H-indol-3-yl)-ethyl]-amide;
2-Dimethylamino-5-(3-fluoro-phenyl)-thiazole-4-carboxylic acid cyclopropylmethyl-[2-(7-fluoro-1H-indol-3-yl)-ethyl]-amide;
2-Dimethylamino-5-(3-fluoro-phenyl)-thiazole-4-carboxylic acid cyclopropylmethyl-[2-(4-fluoro-1H-indol-3-yl)-ethyl]-amide;
2-Dimethylamino-5-(3-fluoro-phenyl)-thiazole-4-carboxylic acid cyclopropylmethyl-[2-(6-fluoro-1H-indol-3-yl)-ethyl]-amide;
3-(4-Fluoro-phenyl)-pyrazine-2-carboxylic acid cyclopropylmethyl-[2-(1H-indol-3-yl)-ethyl]-amide;
3-(4-Fluoro-3-methyl-phenyl)-pyrazine-2-carboxylic acid cyclopropylmethyl-[2-(1H-indol-3-yl)-ethyl]-amide;
3-(2-Fluoro-5-methoxy-phenyl)-pyrazine-2-carboxylic acid cyclopropylmethyl-[2-(1H-indol-3-yl)-ethyl]-amide;
3-(3-Fluoro-5-methyl-phenyl)-pyrazine-2-carboxylic acid cyclopropylmethyl-[2-(1H-indol-3-yl)-ethyl]-amide;
3-(3-Trifluoromethyl-phenyl)-pyrazine-2-carboxylic acid cyclopropylmethyl-[2-(1H-indol-3-yl)-ethyl]-amide;
3-(2,3-Dimethyl-phenyl)-pyrazine-2-carboxylic acid cyclopropylmethyl-[2-(1H-indol-3-yl)-ethyl]-amide;
3-(3-Methoxy-phenyl)-pyrazine-2-carboxylic acid cyclopropylmethyl-[2-(1H-indol-3-yl)-ethyl]-amide;
3-m-Tolyl-pyrazine-2-carboxylic acid cyclopropylmethyl-[2-(1H-indol-3-yl)-ethyl]-amide;
2-Methyl-4-phenyl-pyrimidine-5-carboxylic acid cyclopropylmethyl-[2-(1H-indol-3-yl)-ethyl]-amide;
4-Phenyl-pyrimidine-5-carboxylic acid cyclopropylmethyl-[2-(1H-indol-3-yl)-ethyl]amide;
3-(3,4-Dimethyl-phenyl)-pyrazine-2-carboxylic acid cyclopropylmethyl-[2-(1H-indol-3-yl)-ethyl]-amide;
3-Phenyl-pyrazine-2-carboxylic acid cyclopropylmethyl-[2-(1H-indol-3-yl)-ethyl]-amide;
2-(Ethyl-methyl-amino)-5-(2-fluoro-phenyl)-thiazole-4-carboxylic acid cyclopropylmethyl-[2-(1H-indol-3-yl)-ethyl]-amide;
2-Methyl-5-(4-propionylamino-phenyl)-thiazole-4-carboxylic acid cyclopropylmethyl-[2-(1H-indol-3-yl)-ethyl]-amide;
4-(3-Chloro-phenyl)-pyrimidine-5-carboxylic acid cyclopropylmethyl-[2-(1H-indol-3-yl)-ethyl]-amide;
4-(3-Chloro-phenyl)-2-methyl-pyrimidine-5-carboxylic acid cyclopropylmethyl-[2-(1H-indol-3-yl)-ethyl]-amide;
4-(3,4-Dimethyl-phenyl)-pyrimidine-5-carboxylic acid cyclopropylmethyl-[2-(1H-indol-3-yl)-ethyl]-amide;
4-(3,4-Dimethyl-phenyl)-2-methyl-pyrimidine-5-carboxylic acid cyclopropylmethyl-[2-(1H-indol-3-yl)-ethyl]-amide;
4-(3-Methoxy-phenyl)-pyrimidine-5-carboxylic acid cyclopropylmethyl-[2-(1H-indol-3-yl)-ethyl]-amide;
4-(3-Methoxy-phenyl)-2-methyl-pyrimidine-5-carboxylic acid cyclopropylmethyl-[2-(1H-indol-3-yl)-ethyl]-amide;
4-(3,4-Dichloro-phenyl)-pyrimidine-5-carboxylic acid cyclopropylmethyl-[2-(1H-indol-3-yl)-ethyl]-amide;
4-(3,4-Dichloro-phenyl)-2-methyl-pyrimidine-5-carboxylic acid cyclopropylmethyl-[2-(1H-indol-3-yl)-ethyl]-amide;
4-(3-Fluoro-phenyl)-pyrimidine-5-carboxylic acid cyclopropylmethyl-[2-(1H-indol-3-yl)-ethyl]-amide;
4-(3-Fluoro-phenyl)-2-methyl-pyrimidine-5-carboxylic acid cyclopropylmethyl-[2-(1H-indol-3-yl)-ethyl]-amide;
4-(4-Bromo-3-chloro-phenyl)-2-methyl-pyrimidine-5-carboxylic acid cyclopropylmethyl-[2-(1H-indol-3-yl)-ethyl]-amide;
4-(4-Bromo-3-chloro-phenyl)-pyrimidine-5-carboxylic acid cyclopropylmethyl-[2-(1H-indol-3-yl)-ethyl]-amide;
4-m-Tolyl-pyrimidine-5-carboxylic acid cyclopropylmethyl-[2-(1H-indol-3-yl)-ethyl]-amide;
2-Methyl-4-m-tolyl-pyrimidine-5-carboxylic acid cyclopropylmethyl-[2-(1H-indol-3-yl)-ethyl]-amide;
2-Methyl-4-p-tolyl-pyrimidine-5-carboxylic acid cyclopropylmethyl-[2-(1H-indol-3-yl)-ethyl]-amide;
4-p-Tolyl-pyrimidine-5-carboxylic acid cyclopropylmethyl-[2-(1H-indol-3-yl)-ethyl]-amide;
4-(4-Fluoro-phenyl)-pyrimidine-5-carboxylic acid cyclopropylmethyl-[2-(1H-indol-3-yl)-ethyl]-amide;
4-(4-Fluoro-phenyl)-2-methyl-pyrimidine-5-carboxylic acid cyclopropylmethyl-[2-(1H-indol-3-yl)-ethyl]-amide;
3-Phenyl-pyrazine-2-carboxylic acid ethyl-[2-(1H-indol-3-yl)-ethyl]-amide;
4-Phenyl-pyrimidine-5-carboxylic acid ethyl-[2-(1H-indol-3-yl)-ethyl]-amide;
2-Methyl-4-phenyl-pyrimidine-5-carboxylic acid ethyl-[2-(1H-indol-3-yl)-ethyl]-amide;
3-m-Tolyl-pyrazine-2-carboxylic acid ethyl-[2-(1H-indol-3-yl)-ethyl]amide;
4-m-Tolyl-pyrimidine-5-carboxylic acid ethyl-[2-(1H-indol-3-yl)-ethyl]-amide;
2-Methyl-4-m-tolyl-pyrimidine-5-carboxylic acid ethyl-[2-(1H-indol-3-yl)-ethyl]-amide;
3-(4-Fluoro-phenyl)-pyrazine-2-carboxylic acid ethyl-[2-(1H-indol-3-yl)-ethyl]-amide;
4-(4-Fluoro-phenyl)-pyrimidine-5-carboxylic acid ethyl-[2-(1H-indol-3-yl)-ethyl]amide;
4-(4-Fluoro-phenyl)-2-methyl-pyrimidine-5-carboxylic acid ethyl-[2-(1H-indol-3-yl)-ethyl]-amide;
3-(4-Fluoro-3-methyl-phenyl)-pyrazine-2-carboxylic acid ethyl-[2-(1H-indol-3-yl)-ethyl]-amide;
4-(3-Fluoro-phenyl)-pyrimidine-5-carboxylic acid ethyl-[2-(1H-indol-3-yl)-ethyl]-amide;
4-(3-Fluoro-phenyl)-2-methyl-pyrimidine-5-carboxylic acid ethyl-[2-(1H-indol-3-yl)-ethyl]-amide;
3-(3-Fluoro-5-methyl-phenyl)-pyrazine-2-carboxylic acid ethyl-[2-(1H-indol-3-yl)-ethyl]-amide;
3-(3-Methoxy-phenyl)-pyrazine-2-carboxylic acid ethyl-[2-(1H-indol-3-yl)-ethyl]-amide;
3-(3,4-Dimethyl-phenyl)-pyrazine-2-carboxylic acid ethyl-[2-(1H-indol-3-yl)-ethyl]-amide;
4-(4-Bromo-3-chloro-phenyl)-2-methyl-pyrimidine-5-carboxylic acid ethyl-[2-(1H-indol-3-yl)-ethyl]-amide;
4-(4-Bromo-3-chloro-phenyl)-pyrimidine-5-carboxylic acid ethyl-[2-(1H-indol-3-yl)-ethyl]amide;
2-Methyl-4-p-tolyl-pyrimidine-5-carboxylic acid ethyl-[2-(1H-indol-3-yl)-ethyl]amide;
4-p-Tolyl-pyrimidine-5-carboxylic acid ethyl-[2-(1H-indol-3-yl)-ethyl]-amide;
4-(3,5-Dichloro-phenyl)-2-methyl-pyrimidine-5-carboxylic acid ethyl-[2-(1H-indol-3-yl)-ethyl]-amide;
4-(3,5-Dichloro-phenyl)-pyrimidine-5-carboxylic acid ethyl-[2-(1H-indol-3-yl)-ethyl]-amide;
4-(3-Methoxy-phenyl)-pyrimidine-5-carboxylic acid ethyl-[2-(1H-indol-3-yl)-ethyl]-amide;
4-(3,4-Dimethyl-phenyl)-2-methyl-pyrimidine-5-carboxylic acid ethyl-[2-(1H-indol-3-yl)-ethyl]-amide;
4-(3,4-Dimethyl-phenyl)-pyrimidine-5-carboxylic acid ethyl-[2-(1H-indol-3-yl)-ethyl]-amide;
4-(3,4-Dichloro-phenyl)-pyrimidine-5-carboxylic acid ethyl-[2-(1H-indol-3-yl)-ethyl]amide;
3-Phenyl-pyrazine-2-carboxylic acid [2-(1H-indol-3-yl)-ethyl]-(2,2,2-trifluoro-ethyl)-amide;
4-Phenyl-pyrimidine-5-carboxylic acid [2-(1H-indol-3-yl)-ethyl]-(2,2,2-trifluoro-ethyl)-amide;
2-Methyl-4-phenyl-pyrimidine-5-carboxylic acid [2-(1H-indol-3-yl)-ethyl]-(2,2,2-trifluoro-ethyl)-amide;
3-m-Tolyl-pyrazine-2-carboxylic acid [2-(1H-indol-3-yl)-ethyl]-(2,2,2-trifluoro-ethyl)-amide;
4-m-Tolyl-pyrimidine-5-carboxylic acid [2-(1H-indol-3-yl)-ethyl]-(2,2,2-trifluoro-ethyl)-amide;
2-Methyl-4-m-tolyl-pyrimidine-5-carboxylic acid [2-(1H-indol-3-yl)-ethyl]-(2,2,2-trifluoro-ethyl)-amide;
3-(4-Fluoro-phenyl)-pyrazine-2-carboxylic acid [2-(1H-indol-3-yl)-ethyl]-(2,2,2-trifluoro-ethyl)-amide;
4-(4-Fluoro-phenyl)-pyrimidine-5-carboxylic acid [2-(1H-indol-3-yl)-ethyl]-(2,2,2-trifluoro-ethyl)-amide;
4-(4-Fluoro-phenyl)-2-methyl-pyrimidine-5-carboxylic acid [2-(1H-indol-3-yl)-ethyl]-(2,2,2-trifluoro-ethyl)-amide;
3-(4-Fluoro-3-methyl-phenyl)-pyrazine-2-carboxylic acid [2-(1H-indol-3-yl)-ethyl]-(2,2,2-trifluoro-ethyl)-amide;
4-(3-Fluoro-phenyl)-2-methyl-pyrimidine-5-carboxylic acid [2-(1H-indol-3-yl)-ethyl]-(2,2,2-trifluoro-ethyl)-amide;
3-(3-Fluoro-5-methyl-phenyl)-pyrazine-2-carboxylic acid [2-(1H-indol-3-yl)-ethyl]-(2,2,2-trifluoro-ethyl)-amide;
3-(3-Methoxy-phenyl)-pyrazine-2-carboxylic acid [2-(1H-indol-3-yl)-ethyl]-(2,2,2-trifluoro-ethyl)-amide;
3-(3,4-Dimethyl-phenyl)-pyrazine-2-carboxylic acid [2-(1H-indol-3-yl)-ethyl]-(2,2,2-trifluoro-ethyl)-amide;
4-(4-Bromo-3-chloro-phenyl)-2-methyl-pyrimidine-5-carboxylic acid [2-(1H-indol-3-yl)-ethyl]-(2,2,2-trifluoro-ethyl)-amide;
4-p-Tolyl-pyrimidine-5-carboxylic acid [2-(1H-indol-3-yl)-ethyl]-(2,2,2-trifluoro-ethyl)-amide;
4-(3,4-Dimethyl-phenyl)-2-methyl-pyrimidine-5-carboxylic acid [2-(1H-indol-3-yl)-ethyl]-(2,2,2-trifluoro-ethyl)-amide;
4-(3,4-Dimethyl-phenyl)-pyrimidine-5-carboxylic acid [2-(1H-indol-3-yl)-ethyl]-(2,2,2-trifluoro-ethyl)-amide;
{[2-Dimethylamino-5-(3-fluoro-4-methyl-phenyl)-thiazole-4-carbonyl]-[2-(1H-indol-3-yl)-ethyl]-amino}-acetic acid methyl ester;
{[5-(3-Bromo-4-fluoro-phenyl)-2-dimethylamino-thiazole-4-carbonyl]-[2-(1H-indol-3-yl)-ethyl]-amino}-acetic acid methyl ester;
{(2-Dimethylamino-5-p-tolyl-thiazole-4-carbonyl)-[2-(1H-indol-3-yl)-ethyl]-amino}-acetic acid methyl ester;
{[2-Dimethylamino-5-(2-fluoro-phenyl)-thiazole-4-carbonyl]-[2-(1H-indol-3-yl)-ethyl]-amino}-acetic acid methyl ester;
{[2-Dimethylamino-5-(4-fluoro-phenyl)-thiazole-4-carbonyl]-[2-(1H-indol-3-yl)-ethyl]-amino}-acetic acid methyl ester;
{[2-(Ethyl-methyl-amino)-5-(4-fluoro-phenyl)-thiazole-4-carbonyl]-[2-(1H-indol-3-yl)-ethyl]-amino}-acetic acid methyl ester;
{[2-(Ethyl-methyl-amino)-5-(3-methoxy-phenyl)-thiazole-4-carbonyl]-[2-(1H-indol-3-yl)-ethyl]-amino}-acetic acid methyl ester;
{(2-Dimethylamino-5-m-tolyl-thiazole-4-carbonyl)-[2-(1H-indol-3-yl)-ethyl]-amino}-acetic acid methyl ester;
{[5-(3-Fluoro-5-trifluoromethyl-phenyl)-2-methyl-thiazole-4-carbonyl]-[2-(1H-indol-3-yl)-ethyl]-amino}-acetic acid methyl ester;
{[2-Cyclopropyl-5-(3-fluoro-5-trifluoromethyl-phenyl)-thiazole-4-carbonyl]-[2-(1H-indol-3-yl)-ethyl]-amino}-acetic acid methyl ester;
{[2-Cyclopropyl-5-(3-fluoro-phenyl)-thiazole-4-carbonyl]-[2-(1H-indol-3-yl)-ethyl]-amino}-acetic acid methyl ester;
[[2-(1H-Indol-3-yl)-ethyl]-(2-methyl-5-p-tolyl-thiazole-4-carbonyl)-amino]-acetic acid methyl ester;
{[2-Cyclopropyl-5-(3-trifluoromethyl-phenyl)-thiazole-4-carbonyl]-[2-(1H-indol-3-yl)-ethyl]-amino}-acetic acid methyl ester;
{[5-(4-Bromo-phenyl)-2-methyl-thiazole-4-carbonyl]-[2-(1H-indol-3-yl)-ethyl]-amino}-acetic acid methyl ester;
{[2-(1H-Indol-3-yl)-ethyl]-[2-methyl-5-(3-trifluoromethyl-phenyl)-thiazole-4-carbonyl]-amino}-acetic acid methyl ester;
{[5-(3,5-Dimethyl-phenyl)-2-methyl-thiazole-4-carbonyl]-[2-(1H-indol-3-yl)-ethyl]-amino}-acetic acid methyl ester;
{[5-(2,4-Dimethyl-phenyl)-2-methyl-thiazole-4-carbonyl]-[2-(1H-indol-3-yl)-ethyl]-amino}-acetic acid methyl ester;
{[5-(3-Cyano-phenyl)-2-methyl-thiazole-4-carbonyl]-[2-(1H-indol-3-yl)-ethyl]-amino}-acetic acid methyl ester;
{[5-(3,4-Difluoro-phenyl)-2-methyl-thiazole-4-carbonyl]-[2-(1H-indol-3-yl)-ethyl]-amino}-acetic acid methyl ester;
{[5-(2,3-Dichloro-phenyl)-2-methyl-thiazole-4-carbonyl]-[2-(1H-indol-3-yl)-ethyl]-amino}-acetic acid methyl ester;
{[5-(2-Chloro-6-fluoro-phenyl)-2-methyl-thiazole-4-carbonyl]-[2-(1H-indol-3-yl)-ethyl]-amino}-acetic acid methyl ester;
{[2-Cyclopropyl-5-(4-fluoro-phenyl)-thiazole-4-carbonyl]-[2-(1H-indol-3-yl)-ethyl]-amino}-acetic acid methyl ester;
{[5-(3,4-Dichloro-phenyl)-2-methyl-thiazole-4-carbonyl]-[2-(1H-indol-3-yl)-ethyl]-amino}-acetic acid methyl ester;
{[5-(3,5-Difluoro-phenyl)-2-methyl-thiazole-4-carbonyl]-[2-(1H-indol-3-yl)-ethyl]-amino}-acetic acid methyl ester;
([2-(1H-Indol-3-yl)-ethyl]-{5-[3-(2-methoxy-ethoxy)-phenyl]-2-methyl-thiazole-4-carbonyl}-amino)-acetic acid methyl ester;
{[5-(3-Fluoro-4-methyl-phenyl)-2-methyl-thiazole-4-carbonyl]-[2-(1H-indol-3-yl)-ethyl]-amino}-acetic acid methyl ester;
{[5-(3-Bromo-phenyl)-2-cyclopropyl-thiazole-4-carbonyl]-[2-(1H-indol-3-yl)-ethyl]-amino}-acetic acid methyl ester;
{[5-(3-Bromo-phenyl)-2-methyl-thiazole-4-carbonyl]-[2-(1H-indol-3-yl)-ethyl]-amino}-acetic acid methyl ester;
{[2-Dimethylamino-5-(3,4-dimethyl-phenyl)-thiazole-4-carbonyl]-[2-(1H-indol-3-yl)-ethyl]-amino}-acetic acid methyl ester;
{[2-Dimethylamino-5-(3-fluoro-phenyl)-thiazole-4-carbonyl]-[2-(1H-indol-3-yl)-ethyl]-amino}-acetic acid methyl ester;
{[2-Dimethylamino-5-(3-trifluoromethyl-phenyl)-thiazole-4-carbonyl]-[2-(1H-indol-3-yl)-ethyl]-amino}-acetic acid methyl ester;
{[5-(3-Chloro-phenyl)-2-dimethylamino-thiazole-4-carbonyl]-[2-(1H-indol-3-yl)-ethyl]-amino}-acetic acid methyl ester;
{[5-(3,4-Dimethyl-phenyl)-2-methyl-thiazole-4-carbonyl]-[2-(5-fluoro-1H-indol-3-yl)-ethyl]-amino}-acetic acid methyl ester;
{[2-(5-Fluoro-1H-indol-3-yl)-ethyl]-[3-(4-fluoro-3-methyl-phenyl)-pyrazine-2-carbonyl]-amino}-acetic acid methyl ester;
{[4-(3,4-Dichloro-phenyl)-pyrimidine-5-carbonyl]-[2-(5-fluoro-1H-indol-3-yl)-ethyl]-amino}-acetic acid methyl ester;
{[2-Dimethylamino-5-(4-fluoro-phenyl)-thiazole-4-carbonyl]-[2-(5-fluoro-1H-indol-3-yl)-ethyl]-amino}-acetic acid methyl ester;
{[3-(4-Ethoxy-phenyl)-pyrazine-2-carbonyl]-[2-(5-fluoro-1H-indol-3-yl)-ethyl]-amino}-acetic acid methyl ester;
{[2-Dimethylamino-5-(3,4-dimethyl-phenyl)-thiazole-4-carbonyl]-[2-(5-fluoro-1H-indol-3-yl)-ethyl]-amino}-acetic acid methyl ester;
[[2-(5-Fluoro-1H-indol-3-yl)-ethyl]-(3-p-tolyl-pyrazine-2-carbonyl)-amino]-acetic acid methyl ester;
{[2-(5-Fluoro-1H-indol-3-yl)-ethyl]-[3-(6-methoxy-pyridin-3-yl)-pyrazine-2-carbonyl]-amino}-acetic acid methyl ester;
[[2-(5-Fluoro-1H-indol-3-yl)-ethyl]-(2-methyl-5-phenyl-thiazole-4-carbonyl)-amino]-acetic acid methyl ester;
{[4-(3,4-Dichloro-phenyl)-2-methyl-pyrimidine-5-carbonyl]-[2-(5-fluoro-1H-indol-3-yl)-ethyl]-amino}-acetic acid methyl ester;
{[2-(5-Fluoro-1H-indol-3-yl)-ethyl]-[3-(4-fluoro-phenyl)-pyrazine-2-carbonyl]-amino}-acetic acid methyl ester;
[[2-(5-Fluoro-1H-indol-3-yl)-ethyl]-(4-p-tolyl-pyrimidine-5-carbonyl)-amino]-acetic acid methyl ester; and
2-Cyclopropyl-5-m-tolyl-oxazole-4-carboxylic acid cyclopropylmethyl-[2-(1H-indol-3-yl)-ethyl]-amide,
in a free or a pharmaceutically acceptable salt form.

15. A pharmaceutical composition containing, as active principle, a compound of formula (I) according to claim 1, in a free or a pharmaceutically acceptable salt form, and at least one therapeutically inert excipient.

16. (canceled)

17. A method the treatment or prophylaxis of a disease or disorder selected from the group consisting of: sleep disorders; stress related syndromes; psychoactive substance use, abuse, seeking, and reinstatement; cognitive dysfunctions in the healthy population; psychiatric and neurologic disorders; eating disorders; and drinking disorders.

18. (canceled)
